(19) United States
(12) Patent Application Publication
(10) Pub. No.: US 2026/0092917 A1
(43) Pub. Date: Apr. 2, 2026

(54) DIAGNOSIS, PROGNOSIS AND THERAPY OF NEUROINFLAMMATORY AUTOIMMUNE DISEASES USING CELLULAR AND SOLUBLE BLOOD PARAMETERS

(71) Applicant: UNIVERSITÄT MÜNSTER, Münster (DE)

(72) Inventors: Heinz WIENDL, Freiburg (DE); Catharina Christiane GROSS, Muenster (DE); Luisa KLOTZ, Muenster (DE); Andreas SCHULTE-MECKLENBECK, Muenster (DE); Gerd MEYER ZU HOERSTE, Muenster (DE)

(73) Assignee: UNIVERSITÄT MÜNSTER, Münster (DE)

(21) Appl. No.: 19/109,050

(22) PCT Filed: Oct. 5, 2023
(86) PCT No.: PCT/EP2023/077590

§ 371 Date: Mar. 5, 2025

(30) Foreign Application Priority Data
Oct. 5, 2022 (EP) 22199707.5

(51) Int. Cl. G01N 33/564; G01N 33/569; G16B 25/10; G16H 50/20

(52) U.S. Cl. CPC G01N 33/564; G01N 33/56972; G16B 25/10; G16H 50/20; G01N 2570/00

(57) ABSTRACT

The present invention relates to a method for determining distinguishing parameters of a neuro-inflammatory disease, said method comprising (a) obtaining parameters from a group of samples, wherein said group of samples comprises at least (i) a first subgroup of samples, and (ii) a second subgroup of samples, and (b) determining distinguishing parameters of the obtained parameters in step (a) at least between said first subgroup of samples and said second subgroup of samples by conducting analytical determination. Further, the present invention relates to a set of distinguishing parameters as well as distinct uses of such sets. Additionally, the present invention relates to a method for determining a subtype of a neuro-inflammatory autoimmune disease in a subject.

### TECHNICAL FIELD OF THE INVENTION

The present invention relates to a method for determining distinguishing parameters of a neuro-inflammatory disease, said method comprising (a) obtaining parameters from a group of samples, wherein said group of samples comprises at least (i) a first subgroup of samples, and (ii) a second subgroup of samples, and (b) determining distinguishing parameters of the obtained parameters in step (a) at least between said first subgroup of samples and said second subgroup of samples by conducting analytical determination. Further, the present invention relates also to a set of distinguishing parameters of a neuro-inflammatory autoimmune disease, preferably multiple sclerosis (MS), obtainable or obtained by the method of any one of the preceding claims; the set of distinguishing parameters according to the invention for use in determining whether or not a subject suffers from a neuro-inflammatory autoimmune disease; the set of distinguishing parameters according to the invention for use in determining a subtype of a neuro-inflammatory autoimmune disease; the set of distinguishing parameters according to the invention for use in selecting a subject for a distinct treatment; the set of distinguishing parameters according to the present invention for use in predicting progression and/or treatment response to a therapy of a subject; and a method for determining a subtype of a neuro-inflammatory autoimmune disease in a subject.

### BACKGROUND OF THE INVENTION

Neuroinflammatory diseases display a wide array of symptoms that differentially affects each individual patient. Diagnosis of neuro-inflammatory diseases, including neuro-inflammatory autoimmune diseases, is complex and requires a wide array of functional, clinical, radiological, and laboratory tests including withdrawal of cerebrospinal fluid by lumbar puncture. Those diagnostic procedures are time consuming, cost-intensive and require extensive medical training and infrastructure, while lacking sensitivity and specificity. Moreover, not only do patients display individual arrays of symptoms, patients also respond differentially to the increasing number of therapeutic principles and their distinct modes of action. Currently, there is no scientific evidence providing a rationale for stratification and personalization of therapeutic principles based on the patients' individual immune-profile.

Thus, treatment decisions are based on a combination of clinical parameters of diseases activity and severity, safety aspects, individual preferences, and the general treatment concept (Wiendl H et al, Neurology 2018, Wiendl H et al, Ther Adv Neurol Dis 2021, Ontaneda D et al, Lancet Neurology 2019). Since this approach neglects the individual pattern of underlying immune dysregulation, it harbours a considerable risk of a limited individual therapeutic response caused by suboptimal fitting accuracy between the chosen therapeutic principle and the patient-individual immune-pathophysiology. As a consequence, many patients have to switch several times between therapies before a treatment regimen has been established that is suitable to halt disease progression.

Multiple sclerosis (MS) is a prototypical neuro-inflammatory disease, comprising features of CNS autoimmunity including (i) a clinically heterogeneous disease course, (ii) continuous disease evolution over time, and (iii) putative pathophysiological heterogeneity within one disease (e.g. inflammatory versus neurodegenerative components). Moreover, Multiple sclerosis is a demyelinating inflammatory disease of the central nervous system (CNS) that affects more than 2.8 million people worldwide (Koch-Henriksen and Sørensen, 2010; Reich et al., 2018; Thompson et al., 2018). Based on a large body of experimental and clinical evidence, multiple sclerosis is initiated by autoreactive T cells and other immune-cell subsets including B cells, natural killer (NK) cells, and myeloid cells, such as CNS-associated macrophages and microglia (Dendrou et al., 2015; Gross et al., 2021). The early disease phase of relapsing-remitting multiple sclerosis is likely initiated by a peripheral immune response targeting the CNS. In later phases, a more compartmentalized local inflammation within the CNS and neurodegeneration seem to be predominant drivers of pathophysiology (Hemmer et al., 2015). This concept is supported by the clinical observation that current treatment approaches targeting mainly the peripheral immune system are particularly effective in early stages of multiple sclerosis, while their efficacy is limited later on.

Previous research has highlighted multiple sclerosis-related changes in various immune-cell subsets, sometimes with conflicting results. Most of these studies followed a hypothesis-driven approach and focused on distinct immune-cell populations (De Jager et al., 2008; Gross et al., 2017; Haas et al., 2005; Jensen et al., 2004; Lünemann et al., 2011; Mikulkova et al., 2011; Plantone et al., 2013; Prieto et al., 2006). Effective control of inflammatory disease activity by a wide range of immunotherapeutic agents targeting different immune-cell subsets further fueled the concept of a broad involvement of distinct immune-cell subsets beyond T cells in disease pathophysiology. Although clinical success of different immunotherapeutic principles impressively illustrates the relevance of distinct immune-mediated mechanisms in multiple sclerosis pathophysiology (Lünemann et al., 2020), there is currently no scientific evidence providing a rationale for stratification and personalization of each therapeutic principle based on a patient's immune signature. Instead, treatment selection is based on a combination of clinical and subclinical parameters of disease activity and severity, safety aspects, and patient preferences (Liebmann et al., 2021; Ontaneda et al., 2019; Wiendl et al., 2018). However, this approach ignores the putative heterogeneity of the underlying immune dysregulation. The concept of personalized treatment selection—as it has already been implemented in oncology—still needs to be successfully translated into the field of autoimmunity. Treatments tailored to patients' subgroups instead of the one-drug-fits-all will pave the way to precision medicine in multiple sclerosis.

Thus, there is a need to have at hand methods to efficiently identify patient groups based on comparatively easy and/or easier accessible parameters and linking thus identified patient groups to different treatment principles.

The present invention addresses the need for an approach to determine distinguishing parameters of a neuro-inflammatory autoimmune disease like MS to efficiently stratify or determine patients with regard to into disease endophenotypes featuring distinct clinical characteristics and response to different therapeutic principles. This has the advantage that using distinguishing parameters according to the present invention as biomarkers enables fast and accurate diagnostics of neuro-inflammatory diseases and disease subtypes, preferably based on blood samples that are easily obtainable and storable.

### SUMMARY

The present invention relates to a method for determining distinguishing parameters of a neuro-inflammatory disease, said method comprising (a) obtaining parameters from a group of samples, wherein said group of samples comprises at least (i) a first subgroup of samples, and (ii) a second subgroup of samples, and (b) determining distinguishing parameters of the obtained parameters in step (a) at least between said first subgroup of samples and said second subgroup of samples by conducting analytical determination; preferably wherein the neuro-inflammatory disease is a neuro-inflammatory autoimmune disease, preferably multiple sclerosis (MS).

In a further aspect, the present invention relates also to a set of distinguishing parameters of a neuro-inflammatory autoimmune disease, preferably MS, obtainable or obtained by the method of the invention as described herein.

Further, the invention is the set of distinguishing parameters according to the invention for use in determining whether or not a subject suffers from said neuro-inflammatory autoimmune disease.

Further, the invention relates to the set of distinguishing parameters according to the invention for use in determining a subtype of a neuro-inflammatory autoimmune disease in a subject, wherein said subtype is preferably an endophenotype of said neuro-inflammatory disease.

Further, the invention relates to the set of distinguishing parameters according to the invention for use in selecting a subject for a distinct treatment depending on the subtype, preferably an endophenotype, of said neuro-inflammatory disease, and wherein the subject is preferably suffering from a neuro-inflammatory autoimmune disease, preferably from MS.

Further, the invention relates to the set of distinguishing parameters according to the invention for use in predicting disease progression and/or treatment response to a therapeutic or preventive treatment in a subject, wherein the therapeutic or preventive treatment is preferably selected from the group consisting of immune-modulation, immune-trafficking and/or immune cell-depletion, and wherein subject is preferably suffering from a neuro-inflammatory autoimmune disease, preferably from MS.

A further aspect of the invention relates to a method for determining a subtype of a neuro-inflammatory autoimmune disease in a subject, the method comprising (a) comparing values of distinguishing parameters for said neuro-inflammatory autoimmune disease obtained from a sample of said subject with values of the sets of distinguishing parameters according to the invention and/or with values of distinguishing parameters as determined according to a method according to the invention, and (b) determining the subtype of said neuro-inflammatory autoimmune disease based on said comparison, preferably by conducting analytical determination, wherein said subtype is preferably an endophenotype of said neuro-inflammatory disease, preferably an endophenotype of MS selected from the group consisting of i) E1, E2, E3, and E4 or ii) E1, E2, and E3.

The present invention further relates to a use of a set of distinguishing parameters for i) determining whether or not a subject suffers from multiple sclerosis (MS) and/or a MS subtype, and/or ii) electing a subject suffering from MS for a treatment depending on MS subtype, and/or iii) predicting disease progression and/or treatment response to a therapeutic and/or preventive treatment in a subject suffering from MS depending on MS subtype, wherein said MS subtype is preferably a MS endophenotype. Preferably, it is foreseen that said set of distinguishing parameters comprises the set(s) as recited in the respective claim(s).

A further aspect of the invention relates to a set of distinguishing parameters for use in diagnosing multiple sclerosis (MS), wherein the diagnosing comprises one or more of the following: (i) determining whether or not a subject suffers from multiple sclerosis (MS) and/or determining an MS subtype; (ii) electing a subject suffering from MS for a treatment depending on MS subtype; (iii) predicting disease progression and/or treatment response to a therapeutic and/or preventive treatment in a subject suffering from MS depending on MS subtype; wherein said MS subtype is preferably a MS endophenotype.

A further aspect of the invention relates to a method for determining a subtype of MS, wherein the subtype is preferably a MS endophenotype, the method comprising (a) comparing values of distinguishing parameters obtained from a sample with reference values of said distinguishing parameters, and (b) determining the subtype based on said comparison, preferably by analysing said parameters thereby determining, preferably classifying, the subtype of said neuro-inflammatory autoimmune disease in said subject; preferably, wherein the distinguishing parameters have a prediction accuracy of at least 60%.

A further aspect of the invention relates to a computer-implemented method for determining distinguishing parameters of MS, said method comprising (a) obtaining parameters from a group of samples, wherein said group of samples comprises at least (i) a first subgroup of samples obtained from subjects suffering from a first subtype of MS, and (ii) a second subgroup of samples obtained from subjects suffering from a second subtype of MS, wherein preferably said first and said second subtypes are a first MS endophenotype and a second MS endophenotype, and wherein preferably information on the subgroup the samples belong to is obtained by conducting unsupervised machine learning, preferably unsupervised cluster analysis on the parameters of the samples, and (b) determining distinguishing parameters of the obtained parameters in step (a) at least between said first subgroup of samples and said second subgroup of samples by analysing said parameters obtained from the group of samples to determine parameters that differentiate the at least first and second subgroup of samples, thereby identifying distinguishing parameters that determine, preferably classify, the samples as being a sample of the at least first or second subgroup of samples.

### BRIEF DESCRIPTION OF THE FIGURES

FIG. 1. Study design. A discovery cohort of 378 and a validation cohort of 78 therapy-naïve relapsing-remitting multiple sclerosis (MS) patients without relapse and a disease duration ≤2 years, derived from 7 different German centers were included in the study. Seventy-one and 30 age- and sex-matched healthy individuals (healthy donor, HD) served as controls for the discovery and validation cohort, respectively. Patients were longitudinally followed up for 4 years using a standardized study protocol, including expanded disability status scale (EDSS) (Kurtzke, 1983), multiple sclerosis functional composite (MSFC) test (Fischer et al., 1999), multiple sclerosis inventor cognition (MUSIC) test (Calabrese et al., 2004), annual relapse rate (ARR), disease modifying treatment (DMT) as well as classical morphometric cMRI parameters (brain volume, numbers of T1 and T2 lesions, gadolinium enhancement). Rigorous quality assessment resulted in the exclusion of 69 multiple sclerosis patients from the discovery cohort (see methods). Peripheral blood mononuclear cells (PBMC) were analyzed by multi-parameter functional flow cytometry using a combination of 141 different antibodies defining discrete lymphocyte- and monocyte subsets, their activation- and differentiation state, cytokine production and cytolytic activity. A combination of manual and unbiased automated gating resulted in 348 unique cellular phenotypic and functional parameters characterizing the individual peripheral cellular immune-response signature of each donor, which were further analyzed by computational biology. 1 450 proteins were acquired in serum by Olink explore proximity extension assay (PEA) in a sub-cohort of 158 out of 378 multiple sclerosis patients and 40 out of 71 healthy individuals revealing the individual serum protein signatures.

FIG. 2. Cellular immune-response and serum protein signatures of multiple sclerosis patients. Analysis of flow cytometry data derived from 71 healthy individuals (healthy donor, HD) and 309 therapy-naïve multiple sclerosis (MS) patients and proteomics data of 158 MS patients and 45 HD. A. Uniform manifold approximation and projection (UMAP) (Becht et al., 2019) displaying centered and scaled flow cytometry data (HD, ▴; triangles; MS, •) dots) B. Heatmap with hierarchic clustering of normalized flow-cytometry parameters (y-axis), HD (x-axis, left), and MS patients (x-axis, right). C. Changes (increased, decreased) of discrete immune-cell subsets, cell surface markers, cytokines, chemokines, cytolytic granule content, and cytolytic activity—i.e. cellular immune-response signatures—of MS patients compared to HD. (For interactive plot displaying data for each donor please click https://osmzhlab.uni-muenster.de/shiny/ms_signatures/; user: reviewer, password: review258). Data were grouped by distinct cellular compartments (circled). The intensity—grey shades—displays alterations measured as log 2 fold-change of the median frequency from each cell subset, whereas the border style represents the respective p-value analyzed using the Mann-Whitney test corrected for multiple comparisons by the Holm-Sidak approach. Labelling of the cellular compartments indicates the number and proportion of significantly altered cell subsets. Data on the differential expression of cellular phenotypes is available in FIGS. 17 to 20 with Supplementary FIG. 1-4. D. Left: Plot displaying median log 2 fold-change (increase, decrease) of the top 10% most significantly altered cellular parameters sorted by significance. Label indicates the respective compartment (CD4 T cells, CD8 T cells, ILC including CD56^{dim} NK cells, CD56^{bright} NK cells, B cells, monocytes/dendritic cells), whereas the intensity of the bars displays the level of significance after correction for multiple testing. Middle: Plot displaying median log 2 fold-change (increase, decrease) of the top 25 most significantly altered serum protein parameters sorted by significance. Right: PLS-DA analysis displaying the relative difference between cellular immune-response signatures and serum protein signatures based on the top 10% (HM7+HM2 k=70; HM7u+HM2u K=190) and 25 parameters identified, respectively. Prediction accuracy (PA) was quantified by a machine learning pipeline of four distinct classifiers in a nested, 10-fold cross-validated model on SMOTE-balanced data sets (Leenings et al., 2021a).

FIG. 3. Specific cellular immune-response signatures define 4 endophenotypes in multiple sclerosis. A. Automatic grouping of multiple sclerosis patients (n=309) by PhenoGraph algorithm (Levine et al., 2015) into four distinct endophenotypes (E1-4; E1 n=65, E2 n=73, E3 n=64, E4 n=107) based on differential phenotypic data mapped to the UMAP graph from FIG. 2A. Right: Heatmap of the relative frequency of investigated parameters sorted into five parameter groups by PhenoGraph and split by multiple sclerosis endophenotypes. B. Hierarchically clustered heatmap of the relative median expression of cellular parameters consistently differentiating multiple sclerosis endophenotypes (ME8 k=70; E1 n=65, E2 n=73, E3 n=64, E4 n=107) as determined by multi-categorical LASSO with q-values according to Kruskal-Wallis test with correction for multiple testing. C. Changes (increased, decreased) of distinct immune-cells subsets, cell surface markers, cytokines, chemokines, cytolytic granule content, and cytolytic activity of multiple sclerosis patients with distinct endophenotypes in comparison to healthy individuals as median log 2 fold-change. (For interactive plot displaying data for each donor please click https://osmzhlab.uni-muenster.de/shiny/ms_signatures/; user: reviewer, password: review258). Data were grouped by distinct cellular compartments (circled). Colour intensity in grey shades shows alterations measured as log 2 fold-change of the median frequency of each cell subset, whereas the border style represents the respective p-value analysed by Mann-Whitney test corrected for multiple comparisons by Holm-Sidak approach. Further phenotypic parameters are available in FIGS. 21 to 24 with Supplementary FIGS. 5-8

FIG. 4. Differentiation of MS endophenotypes using a combination of cellular and molecular parameters. A. Visualization and quantification of the relative difference between multiple sclerosis patients with the respective endophenotypes (E1 n=65, E2 n=73, E3 n=64, E4 n=107) and healthy individuals (n=71) based on PLS-DA analysis and prediction accuracy (PA, top) of the top 10% most significantly altered flow cytometry parameters (bottom, HE3 k=70). Bar graphs show the median log 2 fold-change to healthy individuals. Label intensity in grey indicates the respective compartment (CD4 T cells, CD8 T cells, ILC including CD56^{dim} NK cells, CD56^{bright} NK cells, B cells, monocytes/dendritic cells), whereas the intensity of grey to black of the bars informs about the level of significance after correction for multiple testing. B. Serum protein parameters of multiple sclerosis patients (n=158) were visualized by UMAP and visualized in grey shades according to cellular immune-response signature derived endophenotype (E1-4; E1 n=30, E2 n=37, E3 n=27, E4 n=64) determined in FIG. 3A. C. Flow cytometric parameters describing the cellular immune-response signatures of 78 multiple sclerosis patients from the validation cohort were visualized by UMAP and automatically grouped using Phenograph resulting in 4 groups V1-4 (E1 n=16, E2 n=15, E3 n=24, E4 n=23). D. Visualization of PLS-DA analyses of cellular parameters (left), soluble parameters (center), and cellular plus soluble parameters (right, ME9 k=70) from 158 multiple sclerosis patients using a set of parameters identified by LASSO (FIG. 28 with Supplementary FIG. 12) capable of differentiating all four endophenotypes (E1, E2, E3, E4) with the indicated prediction accuracy (PA) as quantified according to FIG. 2D.

FIG. 5. HLA background of immunological endophenotypes and healthy individuals. The four-digit HLA-Genotype for HLA-A, -B, -C, and HLA-DR of multiple sclerosis patients (E1 n=21, E2 n=36, E3 n=34, E4 n=49) was assessed and compared to the Germany pop 6 cohort (http://www.allelefrequencies.net/hla6006a.asp?hla_population=2752) (Schmidt et al., 2009) including information of 8862 controls. Frequencies were centered, scaled, and visualized by hierarchic clustering.

FIG. 6. Association of multiple sclerosis endophenotypes with clinical and paraclinical features. Heatmap displaying the relative difference of the median (continuous parameters) or mean (categorical parameters) values in clinical and paraclinical features between multiple sclerosis endophenotypes. Parameters primarily associated with neurodegeneration are: MSFC, Degree of fatigue (MUSIC), T1 lesion vol. BL, Neurofilament light chain, Disability (EDSS>1.5) BL, Cognition deficit (MUSIC cognition <20) BL, Whole brain volume BL, White matter vol. BL; or neuroinflammation are: T1 Gd+ lesions BL, CIS/RRMS conversion, DMT switch due to LoE in 2y, EDSS increase within 2y, High efficacy DMT, T2 lesion number at BL, ARR, ≥2 relapses within 1y from BL. Abbreviations: BL=baseline, LoE=loss of efficacy.

FIG. 7. Differential efficacy of established DMTs on parameters associated with multiple sclerosis endophenotype pathophysiology. A. Investigation of treatment efficacy in the same multiple sclerosis patients at baseline and under treatment (20 IFN-β, 20 glatiramer acetate GA, 8 dimethyl fumarate DMF, 17 fingolimod FTY, 17 alemtuzumab ALEM) for at least 1 year by flow cytometry as well as Olink-PEA for soluble parameters. B. Top: Calculation of the relative treatment efficacy: The extent of endophenotype-specific alteration (=HD-MS, left) in parameters associated with the pathophysiology of distinct endophenotypes was determined to quantify the pathophysiological component. The therapeutic efficacy was quantified by comparing treated and baseline parameters (=DMT-BL, center). Those alterations were set into relation to the median disease-specific difference between multiple sclerosis patients and healthy individuals. As a result, patient individual efficacy scores for each parameter were obtained (right), indicating deterioration (<0) or amelioration (>0). The frequency of improved (rel. efficacy >0) parameters was subtracted by 0.5 (line) to indicate whether more parameters improve than deteriorate as a consequence of DMT treatment.

FIG. 8. Differential impact of distinct DMTs on clinical and paraclinical features of endophenotype 4. Kaplan-Meier plots displaying disability (a) and MRI (b) progression of multiple sclerosis patients of endophenotype 1-3 (dashed lines) and endophenotype 4 (solid lines) following initiation of treatment with GA or DMF or IFN-ß over the course of 4 years. Kaplan-Meyer plots indicate the percentage of patients without EDSS progression by at least 1 point compared to baseline (a) and without MRI progression as further defined in the material and methods section (b) over time. Endophenotype-specific treatment outcomes were analysed by log-rank test and are indicated by brackets.

FIG. 9. Sets of distinguishing parameters. Table showing sets of distinguishing parameters HM1, HM2, HM3, HM4, HM5, HM6, HM7, ME1, ME2, ME3, ME4, ME5, ME6, ME7, ME8, ME9, HE1, HE2, and HE3. Said sets of distinguishing parameters are also disclosed in the schematic overview in FIG. 33. A. Distinguishing parameters differentiating between healthy condition and MS. B. Distinguishing parameters differentiating between MS endophenotypes. C. Distinguishing parameters differentiating between healthy condition and MS endophenotypes.

FIG. 10 shows Table S1: patient demographics.

FIG. 11 shows Table S2: Gating strategy for conventional gating of defined immune-cell subsets.

FIG. 12 shows Table S3: Information on proteomics parameters.

FIG. 13 shows Table S4. Allele frequencies in HD of the Germany 6 cohort and within multiple sclerosis endophenotypes.

FIG. 14 shows Table S5. Flow cytometry panels investigating immune-cell subsets and their phenotype. Abbreviations: PF—Perm/Fix for intra-cellular/nuclear staining

FIG. 15 shows Table S6. Flow cytometric panels for the functional characterization of lymphocyte subsets. Abbreviations: LAC—leukocyte activation cocktail (PMA, lonomycin, Brefeldin), PF—Perm/Fix for intra-cellular/nuclear staining

FIG. 16 shows Table S7. List of antibodies used in the study.

FIG. 17 shows Fig. S1. CD4 T cells (P2), CD4 memory T cells (P13-15) and regulatory T cells (P3) were gated from total PBMC and equal numbers per patient were clustered using the phenotypic markers indicated in the heatmaps by bhSNE followed by automated cluster identification with PhenoGraph. Median differential frequencies of clusters characterizing distinct phenotypic CD4 T-cell signatures between multiple sclerosis patients (n=309) and healthy individuals (n=71) are visualized on panel basis in the central radial plot with the border indicating the level of significance after correction for multiple testing. An interactive version of this plot displaying data for each individual donor is available online under https://osmzhlab.uni-muenster.de/shiny/ms_signatures/.

FIG. 18 shows Fig. S2. CD8 T cells (P2) and CD8 memory T cells (P13-15) were gated from total PBMC and equal numbers per patient were clustered using the phenotypic markers indicated in the heatmaps by bhSNE followed by automated cluster identification with PhenoGraph. Median differential frequencies of clusters characterizing distinct phenotypic CD8 T-cell signatures between multiple sclerosis patients (n=309) and healthy individuals (n=71) are visualized on panel basis in the central radial plot with the border indicating the level of significance after correction for multiple testing. An interactive version of this plot displaying data for each individual donor is available online under https://osmzhlab.uni-muenster.de/shiny/ms_signatures/.

FIG. 19 shows Fig. S3. CD56^{dim}CD16⁺ NK cells were gated from total PBMC and equal numbers per patient were clustered using the phenotypic markers indicated in the heatmaps by bhSNE followed by automated cluster identification with PhenoGraph. Median differential frequencies of clusters characterizing distinct phenotypic CD56^{dim} NK-cell signatures between multiple sclerosis patients (n=309) and healthy individuals (n=71) are visualized on panel basis in the central radial plot with the border indicating the level of significance after correction for multiple testing. An interactive version of this plot displaying data for each individual donor is available online under https://osmzhlab.uni-muenster.de/shiny/ms_signatures/.

FIG. 20 shows Fig. S4. CD56^{bright}CD16^{dim/-} NK cells (P10, 11, 13, 14) were gated from total PBMC and equal numbers per patient were clustered using the phenotypic markers indicated in the heatmaps by bhSNE followed by automated cluster identification with PhenoGraph. Median differential frequencies of clusters characterizing distinct phenotypic CD56^{bright} NK-cell signatures between multiple sclerosis patients (n=309) and healthy individuals (n=71) are visualized on panel basis in the central radial plot with the border indicating the level of significance after correction for multiple testing. An interactive version of this plot displaying data for each individual donor is available online under https://osmzhlab.uni-muenster.de/shiny/ms_signatures/.

FIG. 21 shows Fig. S5. Changes (increased, decreased) of distinct CD4 T-cell phenotypes of multiple sclerosis patients with distinct endophenotypes in comparison to healthy individuals. Color intensity in grey shades shows alterations measured as log 2 fold-change of the median frequency of each cell subset, whereas the border style represents the respective p-value analyzed by Mann-Whitney test corrected for multiple comparisons by Holm-Sidak approach. An interactive version of this plot displaying data for each individual donor is available online under https://osmzhlab.uni-muenster.de/shiny/ms_signatures/.

FIG. 22 shows Fig. S6. Changes (increased, decreased) of distinct CD8 T-cell phenotypes of multiple sclerosis patients with distinct endophenotypes in comparison to healthy individuals. Color intensity in grey shades shows alterations measured as log 2 fold-change of the median frequency of each cell subset, whereas the border style represents the respective p-value analyzed by Mann-Whitney test corrected for multiple comparisons by Holm-Sidak approach. An interactive version of this plot displaying data for each individual donor is available online under https://osmzhlab.uni-muenster.de/shiny/ms_signatures/.

FIG. 23 shows Fig. S7. Changes (increased, decreased) of distinct CD56^{dim} NK-cell phenotypes of multiple sclerosis patients with distinct endophenotypes in comparison to healthy individuals. Color intensity in grey shades shows alterations measured as log 2 fold-change of the median frequency of each cell subset, whereas the border style represents the respective p-value analyzed by Mann-Whitney test corrected for multiple comparisons by Holm-Sidak approach. An interactive version of this plot displaying data for each individual donor is available online under https://osmzhlab.uni-muenster.de/shiny/ms_signatures/.

FIG. 24 shows Fig. S8. Changes (increased, decreased) of distinct CD56^{bright} NK-cell phenotypes of multiple sclerosis patients with distinct endophenotypes in comparison to healthy individuals. Color intensity in grey shades shows alterations measured as log 2 fold-change of the median frequency of each cell subset, whereas the border style represents the respective p-value analyzed by Mann-Whitney test corrected for multiple comparisons by Holm-Sidak approach. An interactive version of this plot displaying data for each individual donor is available online under https://osmzhlab.uni-muenster.de/shiny/ms_signatures/.

FIG. 25 shows Fig. S9. Soluble parameters from healthy individual (HD, n=40) and multiple sclerosis patients of endophenotypes 1˜4 (E1 n=30, E2 n=37, E3 n=27, E4 n=64) were investigated by Kruskal-Wallis test including correction for multiple testing and differentially abundant parameters were mapped by plotting the relative difference between the groups including hierarchic clustering (top). Center: Top 25 serum protein parameters most significantly altered between HD and multiple sclerosis endophenotypes 1˜4 as well as their median log 2 fold-change. The intensity of the bars in grey shades informs about the level of significance after correction for multiple testing. Top 3 parameters consistently altered between all multiple sclerosis patients and healthy individuals are: LTA4H, NEFL, MYOC. Bottom: Visualization of the relative difference by PLS-DA analysis including prediction accuracy (PA, HE3 k=70) as determined by machine learning. Information on relevant proteins is provided in FIG. 12 with Table S3. Detailed information on the expression of each protein is provided in FIG. 26 with Fig. S10.

FIG. 26 shows Fig. S10. Abundance of proteomics parameters identified throughout the manuscript in healthy individuals (HD, median, blue line) and multiple sclerosis endophenotypes.

FIG. 27 shows Fig. S11. Validation of multiple sclerosis endophenotypes. Flow cytometric parameters describing the immune signature of 78 multiple sclerosis patients and 30 healthy individuals were derived from the validation cohort. The relative difference in the top 10% most significantly altered parameters in E1-4 (FIG. 4C) compared to healthy individuals was investigated between V1-4 and healthy individuals of the validation cohort and Phenograph clusters were assigned based on similarity of the alterations. Bar graphs show the median log 2 fold-change to healthy individuals. Grey shades indicate the respective compartment (CD4 T cells, CD8 T cells, ILC including CD56^{dim} NK cells, CD56^{bright} NK cells, B cells, monocytes/dendritic cells).

FIG. 28 shows Fig. S12. Cellular (A), soluble (B) as well as cellular and soluble parameters (C, ME9 k=70) were investigated by LASSO for parameters differentiating multiple sclerosis endophenotypes. Results from 10-fold cross-validation were checked for parameters selected in at least 9/10 runs. The relative median frequency of each parameter was illustrated in heatmaps supplemented by the level of significance according to Kruskal-Wallis test after correction for multiple testing. Parameter labelling in grey shades provides information on the respective compartment (CD4 T cells, CD8 T cells, CD56^{dim} NK cells, CD56^{bright} NK cells, B cells, monocytes/dendritic cells), soluble parameters are underlined. Detailed information on the expression of each protein is provided in FIG. 26 with Fig. S10.

FIG. 29 shows Fig. S13. Association of multiple sclerosis endophenotypes with clinical and paraclinical parameters. Heatmap of the relative difference of the median (continuous parameters) or mean (categorical parameters) values in clinical features between patients from the exploration cohort with distinct multiple sclerosis endophenotypes sorted by hierarchical clustering; for reference, artificial prototypic groups (hatched) with highest degenerative (DEG) or inflammatory (INFL) values and median demographic (black) features were included. All six groups were also hierarchically clustered on the y-axis, providing information on relative similarity of multiple sclerosis endophenotypes to prototypic degenerative or inflammatory clinical presentation.

FIG. 30 shows Fig. S14. Mode of action of investigated disease modifying treatments (DMTs).

FIG. 31 shows Fig. S15. Effect of distinct disease modifying treatment (DMT) on the levels of cellular and soluble parameters linked to multiple sclerosis endophenotype pathophysiology (FIG. 4A and FIG. 27 with Fig. S11). The relative difference between median levels under and before therapy was calculated (DMT/BL), resulting in values <1 for a reduction and >1 for an increase as a consequence of treatment. Finally, log 2 values of those results were rescaled between-1 and 1 and plotted as heatmaps including hierarchic clustering. Parameters labels in grey shades indicate the respective compartment (CD4 T cells, CD8 T cells, CD56^{dim} NK cells, CD56^{bright} NK cells, B cells, monocytes/dendritic cells), soluble parameters are underlined.

FIG. 32 shows Fig. S16. Illustration of the generation of the final data set used in the study. PBMC were isolated from peripheral blood of 71 healthy individuals and 378 treatment-naïve relapsing-remitting multiple sclerosis patients and cryo-preserved in the vapor phase of a liquid nitrogen tank following standardized procedures at each study center. Samples were shipped to a central facility for flow-cytometric investigation using 19 panels of up to 13 colors spanning all lymphocyte and monocyte subsets, transcription factors, regulatory molecules, markers of cellular activation and differentiation, effector molecules and assessing cytokine production as well as cytolytic activity. Panels were investigated by manual gating for defined cell subsets and effector functions, whereas complex expression patterns of phenotypic markers like regulatory molecules was assessed by dimensionality reduction and automated gating. Samples with low viability were excluded. The resulting data set was 99% complete with sporadic missing values due to technical issues. Missing values were imputed using the Amelia algorithm based on similarity with comparable samples. The complete data set was corrected for technical confounding factors based on center-specific and flow-cytometric fluctuations by ComBat algorithm, resulting in a final data set of 384 parameters reflecting the peripheral immune-signature of 71 healthy individuals and 309 multiple sclerosis patients.

FIG. 33 shows a schematic overview of the determining of endophenotypes using distinct sets of distinguishing parameters HM1, HM2, HM3, HM4, HM5, HM6, HM7, ME1, ME2, ME3, ME4, ME5, ME6, ME7, ME8, ME9, HE1, HE2, and HE3. Said sets of distinguishing parameters are in detail disclosed in FIG. 9.

FIG. 34 shows an analysis of cluster stability using adjusted Rand index (ARI). A. Pairwise comparisons of the cluster assignments of MS patients by PhenoGraph based on differential phenotypic data at different k values resulting in a range of distinct cluster assignments. Color intensity in grey shades indicates the ARI value as a measure of similarity as indicated in the respective scale on the right. B. Mean ARI values calculated for each Phenograph iteration at distinct k values in A. The maximal mean ARI corresponds to the Phenograph iteration with a k value of 190.

FIG. 35 shows that specific cellular immune-response signatures define three endophenotypes in multiple sclerosis. A. Automatic grouping of multiple sclerosis patients (n=309) by PhenoGraph algorithm (Levine et al., 2015) into three distinct endophenotypes (E1-3; E1 n=81, E2 n=106, E3 n=122) based on differential phenotypic data mapped to the UMAP graph from FIG. 2A. B. Heatmap of the relative frequency of investigated parameters sorted into five parameter groups by PhenoGraph and split by multiple sclerosis endophenotypes. C. Hierarchically clustered heatmap of the relative median expression of cellular parameters consistently differentiating multiple sclerosis endophenotypes as determined by multi-categorical LASSO with q-values according to Kruskal-Wallis test with correction for multiple testing (ME1u k=190). D. Changes (increased, decreased) of distinct immune-cells subsets, cell surface markers, cytokines, chemokines, cytolytic granule content, and cytolytic activity of multiple sclerosis patients with distinct endophenotypes in comparison to healthy individuals as median log 2 fold-change. (For interactive plot displaying data for each donor please click https://osmzhlab.uni-muenster.de/shiny/ms_signatures/; user: reviewer, password: review258). Data were grouped by distinct cellular compartments (circled). Color intensity in grey shades shows alterations measured as log 2 fold-change of the median frequency of each cell subset, whereas the border style represents the respective p-value analyzed by Mann-Whitney test corrected for multiple comparisons by Holm-Sidak approach. Further phenotypic parameters are shown in FIGS. 42-45.

FIG. 36 shows the differentiation of MS endophenotypes using a combination of cellular and molecular parameters. A. Visualization and quantification of the relative difference between multiple sclerosis patients with the respective endophenotypes and healthy individuals (n=71) based on PLS-DA analysis and prediction accuracy (PA, bottom; HE8u k=190) of the top 10% most significantly altered flow cytometry parameters (top). Middle: relative difference in the identified parameters to healthy individuals in the context of all 3 endophenotypes. Bar graphs show the median log 2 fold-change to healthy individuals. Label intensity in grey indicates the respective compartment (CD4 T cells, CD8 T cells, ILC including CD56dim NK cells, CD56bright NK cells, B cells, monocytes/dendritic cells), whereas the intensity of grey to black of the bars informs about the level of significance after correction for multiple testing. B. Visualization of PLS-DA analyses of cellular parameters (top, HE10u k=190) and cellular plus soluble parameters (ME11u k=190) from 158 multiple sclerosis patients using a set of parameters identified by LASSO capable of differentiating all three endophenotypes (E1, E2, E3) with the indicated prediction accuracy (PA). Heatmaps on the right indicate the respective parameters as well as their relative median expression between endophenotypes.

FIG. 37 shows the HLA background of immunological endophenotypes and healthy individuals. The four-digit HLA-Genotype for HLA-A, -B, -C, and HLA-DR of multiple sclerosis patients (E1, E2, E3) was assessed and compared to the Germany pop 6 cohort (http://www.allelefrequencies.net/hla6006a.asp?hla_population=2752) (Schmidt et al., 2009) including information of 8862 controls. Frequencies were centered, scaled, and visualized by hierarchic clustering.

FIG. 38 shows the association of multiple sclerosis endophenotypes with clinical and paraclinical features. Heatmap displaying the relative difference of the median (continuous parameters) or mean (categorical parameters) values in clinical and paraclinical features between multiple sclerosis endophenotypes. Parameters primarily associated with neurodegeneration are: MSFC, Degree of fatigue (MUSIC), T1 lesion vol. BL, Neurofilament light chain, Disability (EDSS>1.5) BL, Cognition deficit (MUSIC cognition <20) BL, Whole brain volume BL, White matter vol. BL; or neuroinflammation are: T1 Gd+ lesions BL, CIS/RRMS conversion, DMT switch due to LoE in 2y, EDSS increase within 2y, High efficacy DMT, T2 lesion number at BL, ARR, ≥2 relapses within 1y from BL. Abbreviations: BL=baseline, LoE=loss of efficacy.

FIG. 39 shows differential efficacy of established DMTs on parameters associated with multiple sclerosis endophenotype pathophysiology. Top: Calculation of the relative treatment efficacy: The extent of endophenotype-specific alteration (=HD-MS, left) in parameters associated with the pathophysiology of distinct endophenotypes was determined to quantify the pathophysiological component. The therapeutic efficacy was quantified by comparing treated and baseline parameters (=DMT-BL, center). Those alterations were set into relation to the median disease-specific difference between multiple sclerosis patients and healthy individuals. As a result, patient individual efficacy scores for each parameter were obtained (right), indicating deterioration (<0) or amelioration (>0). The frequency of improved (rel. efficacy >0) parameters was subtracted by 0.5 (line) to indicate whether more parameters improve than deteriorate as a consequence of DMT treatment.

FIG. 40 shows differential impact of distinct DMTs on clinical and paraclinical features of endophenotype 3. Kaplan-Meier plots displaying disability (A) and MRI (B) progression of multiple sclerosis patients of endophenotype 1-2 (dashed lines) and endophenotype 3 (solid lines) following initiation of treatment with GA or DMF or IFN-ß over the course of 4 years. Kaplan-Meyer plots indicate the percentage of patients without EDSS progression by at least 1 point compared to baseline (A) and without MRI progression as further defined in the material and methods section (B) over time. Endophenotype-specific treatment outcomes were analyzed by log-rank test and are indicated by brackets.

FIG. 41 shows a table showing sets of distinguishing parameters HM1u, HM2u, HM3u, HM4u, HM5u, HM6u, HM7u, ME1u, ME2u, ME3u, ME4u, ME5u, ME6u, ME7u, ME8u, ME9u, ME10u, ME11u, HE1u, HE2u, HE3u, HE4u, HE5u, HE6u, HE7u, and HE8u. Said sets of distinguishing parameters are also given in the schematic overview in FIG. 47.

FIG. 42 shows changes (increased, decreased) of distinct CD4 T-cell phenotypes of multiple sclerosis patients with distinct endophenotypes in comparison to healthy individuals. Color intensity in grey shades shows alterations measured as log 2 fold-change of the median frequency of each cell subset, whereas the border style represents the respective p-value analyzed by Mann-Whitney test corrected for multiple comparisons by Holm-Sidak approach. An interactive version of this plot displaying data for each individual donor is available online under https://osmzhlab.uni-muenster.de/shiny/ms_signatures/.

FIG. 43 shows changes (increased, decreased) of distinct CD8 T-cell phenotypes of multiple sclerosis patients with distinct endophenotypes in comparison to healthy individuals. Color intensity in grey shades shows alterations measured as log 2 fold-change of the median frequency of each cell subset, whereas the border style represents the respective p-value analyzed by Mann-Whitney test corrected for multiple comparisons by Holm-Sidak approach. An interactive version of this plot displaying data for each individual donor is available online under https://osmzhlab.uni-muenster.de/shiny/ms_signatures/.

FIG. 44 shows changes (increased, decreased) of distinct CD56^{dim} NK-cell phenotypes of multiple sclerosis patients with distinct endophenotypes in comparison to healthy individuals. Color intensity in grey shades shows alterations measured as log 2 fold-change of the median frequency of each cell subset, whereas the border style represents the respective p-value analyzed by Mann-Whitney test corrected for multiple comparisons by Holm-Sidak approach. An interactive version of this plot displaying data for each individual donor is available online under https://osmzhlab.uni-muenster.de/shiny/ms_signatures/.

FIG. 45 shows changes (increased, decreased) of distinct CD56^{bright} NK-cell phenotypes of multiple sclerosis patients with distinct endophenotypes in comparison to healthy individuals. Color intensity in grey shades shows alterations measured as log 2 fold-change of the median frequency of each cell subset, whereas the border style represents the respective p-value analyzed by Mann-Whitney test corrected for multiple comparisons by Holm-Sidak approach. An interactive version of this plot displaying data for each individual donor is available online under https://osmzhlab.uni-muenster.de/shiny/ms_signatures/.

FIG. 46 shows soluble parameters from healthy individual (HD, n=40) and multiple sclerosis patients of endophenotypes 1-3 were investigated by Kruskal-Wallis test including correction for multiple testing. Top: Top 25 serum protein parameters most significantly altered between HD and multiple sclerosis endophenotypes 1-3 as well as their median log 2 fold-change. The intensity of the bars in grey shades informs about the level of significance after correction for multiple testing. Top 3 parameters consistently altered between all multiple sclerosis patients and healthy individuals are: LTA4H, NEFL, MYOC. Center: differentially abundant parameters were mapped by plotting the relative difference to HD including hierarchic clustering. Bottom: Visualization of the relative difference by PLS-DA analysis including prediction accuracy (PA, HE6u k=190) as determined by machine learning.

FIG. 47 shows a schematic overview of the determining of endophenotypes using distinct sets of distinguishing parameters HM1u, HM2u, HM3u, HM4u, HM5u, HM6u, HM7u, ME1u, ME2u, ME3u, ME4u, ME5u, ME6u, ME7u, ME8u, ME9u, ME10u, ME11u, HE1u, HE2u, HE3u, HE4u, HE5u, HE6u, HE7u, and HE8u. Said sets of distinguishing parameters are illustratively given in FIG. 41. A. Distinguishing parameters differentiating between healthy condition and MS. B. Distinguishing parameters differentiating between MS endophenotypes. C. Distinguishing parameters differentiating between healthy condition and MS endophenotypes.

FIG. 48 shows a comparison of results obtained for three and four endophenotypes, respectively. Given are in the table from left to right cellular parameters identified for both examples and thus, in case of three and four endophenotypes; soluble parameters identified for both examples and thus, in case of three and four endophenotypes; cellular parameters identified only in case of four endophenotypes (k=70); soluble parameters identified only in case of four endophenotypes (k=70); cellular parameters identified only in case of three endophenotypes (k=190); soluble parameters identified only in case of three endophenotypes (k=190).

FIG. 49 shows prediction accuracy (PA) for cellular and soluble distinguishing parameters identified in case of both k=70 and k=190 as given in the table shown in FIG. 48. HD. Healthy condition, MS: multiple sclerosis condition, E: MS endophenotype, k70 refers to k=70 (4 MS endophenotypes) and k190 to k=190 (3 MS endophenotypes).

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments; however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one or more such as one, two, three, four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

In particular, the present invention relates to a method for determining distinguishing parameters of a neuro-inflammatory disease, said method comprising (a) obtaining parameters from a group of samples, wherein said group of samples comprises at least (i) a first subgroup of samples, and (ii) a second subgroup of samples, and (b) determining distinguishing parameters of the obtained parameters in step (a) at least between said first subgroup of samples and said second subgroup of samples by conducting analytical determination.

The term “neuro-inflammatory autoimmune disease” as used herein comprises, besides others, multiple sclerosis (short: MS), neuromyelitis optica spectrum disorders (short: NMOSD), Susac's syndrome (short: SuS), and autoimmune encephalitis (short: AIE). Thus, said term is not limited to MS, NMOSD, SuS and AIE. Preferably, MS as one class of the neuro-inflammatory autoimmune diseases comprises relapsing forms of multiple sclerosis (short: RMS) or progressive forms of multiple sclerosis (PMS). The term “inflammatory autoimmune CNS disease” can also be used interchangeably with the term “neuro-inflammatory autoimmune disease”.

More specifically, it has surprisingly been found that using the methods according to the present invention patients suffering from neuro-inflammatory diseases, in particular neuro-inflammatory autoimmune diseases, such as MS can be successfully stratified based on peripheral immune-response signatures obtained from distinguishing parameters. Thus, the methods and the set of distinguishing parameters according to the present invention have the potential to guide personalized treatment decisions in neuro-inflammatory diseases, in particular neuro-inflammatory autoimmune diseases, such as MS.

In particular, by applying the methods according to the present invention for the first time discrete endophenotypes of a neuro-inflammatory (autoimmune) disease like MS could be identified. More specifically, three or four discrete endophenotypes were identified in a prospective, multi-centric cohort of 378 early untreated MS patients. The endophenotypes were characterized by specific alterations of soluble and cellular blood parameters as revealed by high-dimensional flow cytometric analysis of peripheral blood mononuclear cells and proximity extension assay coupled with next generation sequencing (PEA-NGS) followed by unsupervised clustering. Analyses revealed distinct sets of parameters distinguishing healthy subjects, MS patients and so far unknown MS endophenotypes with high prediction accuracy. Association of endophenotypes with longitudinal clinical- and para-clinical disease trajectories pinpointed two endophenotypes differentiating between patients with inflammatory disease characteristics and patients with signs of early neurodegeneration. Investigating the capacity of treatments to normalize MS-associated endophenotype signatures revealed differential fitting accuracy towards distinct immune therapeutic principles; exemplified by reduced treatment response towards interferon beta in patients with the inflammatory endophenotype. The endophenotypes not only predicted the natural disease course but also response to treatment with the inherent potential to tailor treatments to specific patients' needs. Thus, ascertaining a patient's blood signature may provide an innovative clinical tool in predicting clinical disease trajectories and personalizing treatment decisions based on pathobiological principles.

Moreover, the methods according to the present invention are based on blood-derived soluble or cellular parameters. This has several advantages compared to state-of-the-art diagnostic processes: blood-derived soluble or cellular parameters are cheaper, easier, faster and less painful obtainable and more stable than parameters obtained from cerebrospinal fluid (CSF), which needs to be processed within two hours following lumbar puncture. Thus, in contrast to state-of-the-art diagnostic processes, samples can be shipped and laboratory testing can take place in centralized facilities. This can make high-accuracy diagnostics better accessible for peripheral health care providers as well as in countries with less developed health care infrastructure.

The term “detect” or “detecting” can be used interchangeably with the term “determine” or “determining” as used herein. The term “detect” or “detecting” as well as the term “determine” or “determining” when used herein in combination with the words “level”, “amount” or “value”, the words “detect”, “detecting”, “determine” or “determining” are understood to generally refer to a quantitative or a qualitative level. In this regard the words “value,” “amount” or “level” are used interchangeably herein. The level of specific cells may be expressed by the amount of specific cells being detected. It can also be expressed by the strength of a signal measured in the method of detecting the level of specific cells when immunofluorescence may be used, which may be combined with flow cytometry. The above-mentioned can be applied to each cell level being determined by the method of the present invention.

The term “stratification/stratifying” as used herein and throughout the entire description refers to making a (risk) stratification that the subject will suffer/is suspected to suffer from neurological or psychiatric disease manifestation, preferably from neuro-inflammatory autoimmune disease as defined elsewhere herein in the near future, if said distinguishing parameters or a set of distinguishing parameters are being determined in said samples being obtained from said subject. Thus, it comprises that there is a certain risk for the subject to suffer from said manifestation in the near future. Said term “stratify”/“stratifying” may thus refer that a subject is being suspected to suffer from neurological or psychiatric disease manifestation, preferably from neuro-inflammatory autoimmune diseases as defined elsewhere herein. In other words and also being comprised by said term, it refers to assuming that said subject being examined by using the method of the present invention might suffer from neurological or psychiatric disease manifestation, preferably from neuro-inflammatory autoimmune disease based on the general stratification test(s) available in the prior art (e.g., MRI), which has/have already been applied to said subject before the method of the present invention has been applied as a confirmation procedure. The term “determine”/“determining” may be understood synonymous to the term “stratify”/“stratifying” or to the term “classify”/“classifying”.

The term “to have/having neurological or psychiatric disease manifestation, preferably a neuro-inflammatory autoimmune disease” can be used interchangeably with the term “to suffer from/suffering from neurological or psychiatric disease manifestation, preferably a neuro-inflammatory autoimmune disease”. In general, when a subject suffers from a disease, said subject shows specific symptoms of the disease, whereas when a subject has a disease, said subject does not always have to show certain symptoms of the disease, but still is diagnosed with said disease. However, this general concept does not apply to neuro-inflammatory autoimmune diseases according to the present invention.

The term “subject” as used herein and throughout the entire description, also addressed as an individual, refers to a human or non-human animal, generally a mammal. A subject may be a mammalian species such as a rabbit, a mouse, a rat, a Guinea pig, a hamster, a dog, a cat, a pig, a cow, a goat, a sheep, a horse, a monkey, an ape or a human. Preferably, the subject being used in the present invention is a human. More preferably, said subject is an adult. Preferably, said adult is older than 18 years. More preferably, said adult is about 20 to 50 years, about 25 to 45 years, about 30 to 40 years, about 25 years, about 28 years, about 30 years, about 32 years old.

The term “increased” or “decreased” as used herein and throughout the entire description with regard to each stratification as defined herein, relates to an increased level of cells or a decreased level of cells concerning the quantity of the cells in view of their cell number. Thus, any elevation or reduction of the cell number can be considered as a relevant increase or decrease. According to the present invention, any recognizable alteration in form a higher or lower cell count in relation to corresponding levels of cells in said control samples may be considered as an increase or decrease. The increase or decrease of a cell number may be determined using flow cytometry as defined elsewhere herein. As an illustrative example, an increase or decrease may be in the range of at least about 0.4-fold, about 0.5-fold, about 0.6-fold, about 0.7-fold, 0.8-fold, 0.9-fold, 1-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold, or even about 3-fold.

The term “measuring a surface marker/molecule” refers to measuring the signal of a detectable marker, which is attached to a binding partner that recognizes the specific surface marker/molecule expressed by the specific cell when using flow cytometry. Said signal may then be converted by the process called gating, which is known to a person skilled in the art, which then demonstrates the level (or relative amount) of cells being detected in said sample. The gating comprises among other factors measuring of distinct cell populations using forward scatter (FSC) and side scatter (SSC). Measuring FSC and SSC in combination allows to some extend the differentiation of a cell populations within heterogenous cell populations. The determination of FSC allows the discrimination of cells by size. The determination of SCS (more specifically: SSC) allows the determination of internal complexity of the cells (such as granularity). For example, intracellular granules and the nucleus increase SSC.

In general, the term “multiple sclerosis (MS)” as used herein refers to a chronic, inflammatory central nervous system (CNS) disease, characterized pathologically by demyelination. MS has also been classified as a neuro-inflammatory autoimmune disease. It refers to a demyelinating disease in which the insulating covers of nerve cells in the brain and spinal cord are damaged. MS disease activity can be monitored by cranial scans, including magnetic resonance imaging (MRI) of the brain, accumulation of disability, as well as rate and severity of relapses. There are six distinct disease stages and/or types of MS, namely, (1) radiologically isolated syndrome (RIS); (2) clinically isolated syndrome (CIS); (3) relapsing-remitting multiple sclerosis (RRMS); (4) secondary progressive multiple sclerosis (SPMS); (5) progressive relapsing multiple sclerosis (PRMS); and (6) primary progressive multiple sclerosis (PPMS). However, RIS and CIS are pre-stages/pre-types of MS.

### Method for Determining Distinguishing Parameters

In one aspect, the present invention relates to a (computer-implemented) method for determining distinguishing parameters of a neuro-inflammatory disease, said method comprising (a) obtaining parameters from a group of samples, wherein said group of samples comprises at least (i) a first subgroup of samples, and (ii) a second subgroup of samples, and (b) determining distinguishing parameters of the obtained parameters in step (a) at least between said first subgroup of samples and said second subgroup of samples by conducting analytical determination. More specifically, in step (a) the first subgroup of samples is preferably obtained from subjects suffering from a first subtype of MS and the second subgroup of samples from subjects suffering from a second subtype of MS, and in step (b) distinguishing parameters of the obtained parameters in step (a) at least between said first subgroup of samples and said second subgroup of samples are determined by analysing said parameters obtained from the group of samples to determine parameters that differentiate the at least first and second subgroup of samples (cf. conducting “analytical determination” as defined herein below), thereby identifying distinguishing parameters that determine, preferably classify, the samples as being a sample of the at least first or second subgroup of samples.

Herein, the term “neuro-inflammatory disease” refers to a neuro-inflammatory disease, preferably to an autoimmune neuro-inflammatory disease, more preferably to a degenerative autoimmune neuro-inflammatory disease, even more preferably to a degenerative autoimmune neuro-inflammatory disease selected from the group consisting of MS, autoimmune encephalitis, Susac syndrome, and neuromyelitis optica spectrum disorders, even more preferably the neuro-inflammatory disease is MS, preferably early stage MS, e.g. opticus neuritis, clinically isolated syndrome, or relapsing-remitting MS up to 36 months from first symptoms. It is particularly preferred that the neuro-inflammatory disease is a neuro-inflammatory autoimmune disease, preferably multiple sclerosis (MS).

Preferably, information on the subgroup the samples belong to is obtained by conducting unsupervised machine learning, preferably unsupervised cluster analysis on the parameters of the samples. Accordingly, in preferred embodiments, information on the subgroup the samples belong to is—before step (a) or as part of step (a)—obtained by conducting unsupervised machine learning, preferably unsupervised cluster analysis on the parameters of the samples. Thus, a group of samples can be analysed for identifying parameters that differentiate the at least first and second subgroup of samples, thereby identifying distinguishing parameters that determine, preferably classify, the samples as being a sample of the at least first or second subgroup of samples without requiring prior knowledge about number and nature of subgroups and sample assignment to a subgroup, respectively. Hence, subgroups can be identified based on differing parameter values in the absence of a priori available information. This is especially advantageous for initial clustering of parameter values obtained from a group of samples with unknown and/or hidden structure and thus from a group of samples with unknown and/or hidden subgroups. Thus, obtained information is preferably then used for the determination of distinguishing parameters in step (b).

### Analytical Determination Approach

In the context of the present invention, the term “parameter” refers to a variable that is used as an input for conducting analytical determination and that may serve as a biomarker in case of being a “distinguishing parameter” like a parameter that is capable of differentiating between conditions such as healthy and diseased. It is particularly preferred that a parameter represents measurable and/or quantifiable biological information, preferably wherein a value of said parameter is measured and/or quantified ex vivo in a sample. For illustration, a parameter may be the frequency of cells of a specific cell population, for example the frequency of CD4+ T_{reg} cells characterized by expression of FoxP3 besides other markers, with the values of said parameter being the respective cell frequency as measured ex vivo in a group of samples. As another example, a parameter may be the amount of a soluble and cellular blood protein, for example of LTA4H or MYOC.

The term “analytical determination” used herein refers to the analysis of parameters, obtained from a group of samples, wherein said group of samples comprises at least a first subgroup of samples and second subgroup of samples, to determine parameters that differentiate the at least first and second subgroup of samples. Hence, by applying the analytical determination approach of the present invention, distinguishing parameters are identified that determine, preferably classify, a sample as being a sample of the at least first or second subgroup of samples. It is particularly preferred that the analytical determination conducted in step (b) is an analytical classification. Hence, “conducting analytical determination” may be understood as referring to an analysis of parameters obtained from a group of samples to determine parameters that differentiate the at least first and second subgroup of samples thereby identifying distinguishing parameters that determine, preferably classify, the samples as being a sample of the at least first or second subgroup of samples.

In a preferred embodiment, step (b) of the present invention comprises decision making, preferably automated decision making. Herein automated decision making refers to a process of making a decision by automated means without manual human involvement. It is advantageous that the analytical determination approach comprises decision making as decision making facilitates determination of samples and determination of a subset of parameters of higher impact on the determination compared to the remaining parameters obtained from the samples.

In a preferred embodiment, step (b) of the present invention comprises automated decision making using machine learning. Machine learning refers to a subfield of artificial intelligence, with artificial intelligence being based on the capability of a machine to imitate human decision making and learning. More specifically, machine learning relates to the use of algorithms applied on input data, the underlying patterns, dependencies and hidden structures of which are used to generate a mathematical model that can then be applied to one or more independent data sets. Thus, conducting an analytical determination approach comprising automated decision making using machine learning is advantageous to perform complex tasks in an easy, fast and accurate way without the requirements of manual human involvement. This is especially advantageous, when larger sets of parameters, e.g. comprising fifty parameters or more, are analysed to determine distinguishing parameters and/or to identify a subtype of a neuro-inflammatory disease in a sample of a subject.

In a preferred embodiment, the analytical determination approach of the present invention comprises automated decision making using supervised machine learning. Herein, supervised machine learning refers to machine learning, wherein the input data comprise at least partially information on the subgroup a sample belongs to with the determination, preferably classification, representing the target variable. Thus, a determination model, preferably a classification model, can be obtained based on provided input data. Hence, supervised machine learning is especially advantageous for determination of samples, especially for classification of samples.

It is further preferred that in case of supervised machine learning, the step of conducting analytical determination according to the present invention further comprises the steps of
(i) Providing information about sample subgroup assignment for a first subset of the group of samples and information about sample subgroup assignment for a second subset of the group of samples;
(ii) Obtaining a determination model, preferably a classification model, based on the parameters obtained from the samples of said first subset and provided information about sample subgroup assignment of said first subset;
(iii) Obtain sample subgroup assignments for a second subset based on the obtained determination model, preferably based on the obtained classification model, and parameters obtained from the second subset of samples;
(iv) Compare provided and obtained sample subgroup assignments for the second subset.

Thus, samples are, preferably randomly, divided into two subsets, wherein the first subset can also be understood as training set and the second subset as validation set. This is advantageous as the comparison of the provided and obtained sample subgroup assignments for the second subset provides an indication on how well samples of the second subset are classified by the obtained determination model, preferably classification model, using the parameters obtained from the second subset of samples.

Thus, the present invention also relates to a neuro-inflammatory disease, preferably MS, preferably early stage MS, determination model, preferably classification model, obtainable or obtained by the method for determining distinguishing parameters of said disease according to the present invention and/or its embodiments disclosed herein.

Steps (i) to (iv) may be performed at least four times, even more preferably at least ten times, wherein the first and the second subsets differ between each round of conducting steps (i) to (iv). Such an approach may also be referred to as cross-validation. This has the advantage that the prediction accuracy of the determination model, preferably of the classification model, can be robustly determined. Thus, in a further preferred embodiment, the step of conducting analytical determination according to the present invention further comprises the step of determining the prediction accuracy, preferably using cross-validation, preferably nested cross-validation, more preferably nested and stratified cross-validation. In particular, it is preferred that said cross-validation is a at least a four-fold, preferably a at least ten-fold cross-validation.

It is particularly preferred that the analytical determination conducted in step (b) and/or the distinguishing parameters determined in step (b) of the method of the present invention has a prediction accuracy of at least 60%, preferably of at least 65%, even more preferably of at least 70%, even more preferably of at least 75%, even more preferably of at least 80%, even more preferably at least 85%, even more preferably at least 90%, even more preferably at least 95%.

Furthermore, it is preferred that in case of an analytical determination approach comprising supervised machine learning said machine learning comprises at least one or more, preferably all, of the group consisting of
a scaler,
class imbalance correction, preferably using Synthetic Minority Oversampling Technique (SMOTE) algorithm,
at least one, preferably at least two, more at least preferably three, even more preferably at least four, classifier preferably selected from the group consisting of random forest, AdaBoost classifier, linear support vector machine and radial basis function support vector machine,
cross-validation, preferably nested cross-validation, more preferably nested and stratified cross-validation, and
model performance optimization using a correlation coefficient, preferably Matthews correlation coefficient.

This is advantageous for ensuring robustness of the analytical determination approach. For example, by using within the analytical determination approach according to the present invention four different classifier, the accuracy of which may be determined using cross-validation, the best performing classifier can be chosen for the determination model, preferably the classification model. Thus, preferably at least two, more preferably at least four, classifier and cross-validation, preferably at least a four-fold, more preferably at least a ten-fold cross-validation, are used within the analytical determination approach.

Thus, in a particularly preferred embodiment, machine learning comprises
a scaler,
class imbalance correction using Synthetic Minority Oversampling Technique (SMOTE) algorithm,
four classifier selected from the group consisting of random forest, AdaBoost classifier, linear support vector machine and radial basis function support vector machine,
10-fold nested and stratified cross-validation, and
model performance optimization using Matthews correlation coefficient.

It is understood by the skilled person that one or more modifications of the analytical determination approach according to the present invention are envisioned, for example comprising artificial intelligence, unsupervised learning, ridge regression etc., under the restriction that prediction accuracies are obtained of at least 60%, preferably of at least 65%, more preferably of at least 70%, even more preferably of at least 75%, preferably of at least 80%, more preferably at least 85%, even more preferably at least 90%, even more preferably at least 95%.

According to preferred embodiments of the method according to the invention, at least partially available information on the subgroup a sample belongs to is required for a preferred analytical determination approach comprising supervised machine learning. It is thus further understood by the skilled person in the art that if said information is not available as prior knowledge, for example in form of a medical record, said information can be obtained for example, completely or partially, by conducting unsupervised machine learning, preferably unsupervised cluster analysis on the parameters of the respective samples. Information on sample assignment to identified clusters may then be used as input for supervised machine learning as described elsewhere herein in more detail.

### Determination of Distinguishing Parameters

By applying the analytical determination approach according to the present invention, parameters obtained from a group of samples are investigated in view of their potential to classify samples into one of at least two subgroups of samples. The inventors surprisingly found that a set of parameters is sufficient to classify samples into one of the at least two subgroups of samples. This is highly advantageous as it reduces time, cost and efforts associated with determination of samples and thus, subjects using parameters obtained preferably from blood samples of said subjects. The inventors determined subsets of parameters that were able to robustly classify subjects with high prediction accuracy, and thus, a prediction accuracy of at least 60%.

In the context of the present invention, the term “distinguishing parameter” thus refers to a subset of parameters that determine, preferably classify, samples as being a sample of one out of at least two subgroups of samples. Hence, distinguishing parameters can also be understood as biomarkers for one or more subgroups, preferably when comparing said one or more subgroups. Accordingly, a set of distinguishing parameters can be seen, e.g., as a biomarker profile the value pattern of which is preferably specific for a given subgroup of samples and/or which can preferably classify a sample as belonging to one of at least two subgroups. Preferably, the distinguishing parameters classify the subgroup of samples out of at least two subgroups of samples a sample belongs to, with a predictive accuracy of at least 60%, preferably of at least 65%, even more preferably of at least 70%, even more preferably of at least 75%, preferably of at least 80%, more preferably at least 85%, even more preferably at least 90%, even more preferably at least 95%. More specifically, the distinguishing parameters preferably have a prediction accuracy of at least 60%, preferably of at least 65%, even more preferably of at least 70%, even more preferably of at least 75%, preferably of at least 80%, more preferably at least 85%, even more preferably at least 90%, even more preferably at least 95%. Also a set of distinguishing parameters as defined herein has preferably a prediction accuracy of at least 60%, preferably of at least 65%, even more preferably of at least 70%, even more preferably of at least 75%, preferably of at least 80%, more preferably at least 85%, even more preferably at least 90%, even more preferably at least 95%. Prediction accuracy may preferably be determined using cross-validation.

In a preferred embodiment, an obtained parameter of step (a) is determined as distinguishing parameters in step (b) if the obtained parameter of step (a) fulfils one or more of the following:

In a preferred embodiment, the step of determining distinguishing parameters further comprises conducting a statistical analysis, preferably a hypothesis test, even more preferably a t-test. Thus, the impact of the parameters on the determination, preferably on the classification, of samples is preferably evaluated using a statistical analysis like a hypothesis test, preferably using a t-test. Based on the obtained significance results, obtained parameter of step (a) can be ranked and are preferably determined as distinguishing parameter in step (b) if the statistical analysis significance, preferably the t-test significance, of the parameter is within the top 25%, preferably top 15%, more preferably the top 10%, even more preferably the top 5%, even more preferably the top 1%. This is especially advantageous in case of distinguishing parameters being determined based on cellular parameters.

In a particularly preferred embodiment, the statistical analysis significance, preferably the t-test significance, of a parameter that is determined as distinguishing parameter is within the top x, preferably the top 10% of significances obtained for a subset, preferably of all, even more preferably of all, cellular parameters. Preferably, cellular parameters with a significance within the top 10% of t-tests significance values of all cellular parameters are determined as distinguishing parameters.

Optionally or alternatively, the top 100, preferably top 75, more preferably top 50, even more preferably top 25, even more preferably top 10 of, preferably soluble, parameters are selected as distinguishing parameters based on their respective statistical analysis significance. In a particularly preferred embodiment, the top 25 soluble parameters are determined as distinguishing parameters based on their respective statistical analysis significance. Thus, based on obtained significance results, obtained parameter of step (a) can be ranked and are preferably determined as distinguishing parameter in step (b) if the statistical analysis significance of the parameter is within the top 25 of soluble parameters after significance ranking.

In another preferred embodiment, the step of determining distinguishing parameters further comprises conducting a regression analysis, preferably LASSO. In a particularly preferred embodiment, LASSO significant parameters are selected as distinguishing parameters. In case a regression analysis like LASSO is used for determining distinguishing parameters it is furthermore preferred that cross-validation is used for tuning the penalty term lambda, preferably using at least four-fold, preferably at least ten-fold cross-validation. As “LASSO significant parameters” are referred herein parameters that have been obtained by LASSO in at least half of the cross-validations, preferably in at least 75%, even more preferably in at least 90% of the cross-validations, for example in 9 out of ten cross-validations in case of a ten-fold cross-validation. This is advantageous as a combination of regression and a minimal number of cross-validations within a given x-fold cross-validation setting traces the prediction accuracy and thus, results in a highly robust set of distinguishing parameters. Thus, an obtained parameter of step (a) is preferably determined as distinguishing parameter in step (b) if the obtained parameter of step (a) is significant according to the conducted regression analysis, preferably LASSO.

In yet another preferred embodiment, the step of determining distinguishing parameters further comprises conducting parameter clustering for determining distinguishing parameters. Accordingly, a complexity reduction approach may be applied to reduce parameter complexity. For example, instead of individual parameters obtained from a sample, a parameter profile may be obtained based on frequencies of specific parameter combinations observed in said sample. Thus, as distinguishing parameters respective parameter profiles can be obtained, based on, preferably cellular, parameters using parameter clustering for complexity reduction. This is advantageous as it provides a distinguishing profile of parameters that is smaller than sets obtained using a hypothesis test or a regression analysis, while maintaining high prediction accuracy. Thus, an obtained parameter of step (a) is preferably determined as distinguishing parameter in step (b) if the obtained parameter of step (a) is comprised in a parameter profile obtained from, preferably cellular, parameter clustering.

Herein, significance relates to a p-value obtained for example from a statistical analysis and/or a regression analysis, wherein the p-value is 0.05 or less, preferably 0.01 or less, even more preferably 0.001 or less.

### Group of Samples and Subgroups Comprised Therein

Herein, the term “sample” refers to a biological sample of a subject. A sample may be obtained from a subject for determination, preferably classification, directly or indirectly via a database. Herein, the term “subject” encompasses both a healthy individual and an individual diagnosed with a disease or suffering from a disease. Herein, a subject diagnosed with a disease or suffering from a disease is also referred to as “patient” herein, more specifically also as “patient diagnosed with a disease” or as “patient suffering from a disease”.

According to the method of the invention, a group of samples is analysed using an analytical determination approach. Preferably, the samples of said group of samples are biologically different. In particular, said samples are preferably obtained from subjects, wherein preferably no more than one sample is obtained from an individual subject. Thus, the samples of the group of samples are preferably not biologically identical and/or no subject may be represented by more than one sample in the group of samples.

According to the method of the invention, the group of samples comprises at least a first group of samples and a second group of samples.

In a preferred embodiment, the first subgroup of samples is obtained from healthy subjects, and the second subgroup of samples is obtained from subjects suffering from a neuro-inflammatory disease. Accordingly, distinguishing parameters determined by the method according to the invention are preferably parameters that classify a subject, based on its sample, as a healthy subject or as a subject suffering from a neuro-inflammatory disease.

In another preferred embodiment, the first subgroup of samples is obtained from healthy subjects, and the second subgroup of samples is obtained from subjects suffering from a subtype of a neuro-inflammatory disease, wherein said subtype is preferably an endophenotype of said neuro-inflammatory disease. In this case, distinguishing parameters determined by the method according to the invention are parameters that classify a patient based on its sample as a healthy subject or as a subject suffering from a subtype of a neuro-inflammatory disease. As the inventors surprisingly found out using the method of the invention that MS as a prototype of a neuro-inflammatory disease comprises distinct subtypes, the method of the present invention is advantageous for determining distinguishing parameters that classify a subject based on its sample as a healthy subject or as subject suffering from an endophenotype of a neuro-inflammatory disease like MS. Thus, distinguishing parameters determined by the method according to the invention are in this case preferably parameters that classify a subject based on its sample as a healthy subject or as a subject having a neuro-inflammatory disease or suffering from a neuro-inflammatory disease.

Thus, an “endophenotype” of a neuro-inflammatory disease refers herein to a distinct subtype of said neuro-inflammatory disease that can be distinguished from (an) other subtype(s) of said disease based on different characteristics of the disease. Such a characteristic may be one or more variation, alteration, mutation, deletion on the genetic level or at the protein expression level. Further, such a characteristic may be determined by distinct clinical symptoms or para-clinical parameters indicative for pathological neurological processes, their extent, or their development during the disease course comprising in particular neurological and/or immunological symptoms. The different characteristics may be determined comprising one or more of physicochemical, biochemical, proteomic, genetical and/or (para-) clinical parameters, preferably using distinguishing parameters according to the present invention.

According to the invention, the term “suffering from a disease” may be understood as a subject having symptoms of a disease, wherein the symptoms may be clinical manifested symptoms thereby leading to some kind of impairments for a subject, or wherein the symptoms are not or less pronounced or very mild such that there are no impairments or difficulties in everyday life for a subject.

Thus, in another preferred embodiment, the first subgroup of samples is obtained from subjects suffering from a first subtype of a neuro-inflammatory disease, and the second subgroup of samples is obtained from subjects suffering from a second subtype of said neuro-inflammatory disease. Alternatively or optionally, the first subgroup of samples is obtained from subjects suffering from a first endophenotype of a neuro-inflammatory disease, and the second subgroup of samples is obtained from subjects suffering from a second endophenotype of said neuro-inflammatory disease. Accordingly, distinguishing parameters determined by the method according to the invention are preferably parameters that determine or classify a subject based on its sample as a subject suffering from a first subtype of a neuro-inflammatory disease, preferably from a first endophenotype of a neuro-inflammatory disease, or as a subject suffering from a second subtype of said neuro-inflammatory disease, preferably from a second endophenotype of said neuro-inflammatory disease. More generally, distinguishing parameters determined by the method according to the invention are in this case preferably parameters that classify a subject based on its sample as a subject suffering from one of at least two subtypes of a neuro-inflammatory disease, preferably from one of at least two endophenotypes of said neuro-inflammatory disease. Preferably, the neuro-inflammatory disease is multiple sclerosis (MS) and the first and second endophenotype of said neuro-inflammatory disease are a first and a second MS endophenotype, respectively.

It is understood by the person skilled in the art that the first and the second subgroup of samples referred to herein are to be understood as illustrative example for clarification purposes only. Hence, the skilled person is aware that it is also preferred that the methods of the present invention are performed for more than two subgroups of samples, preferably three subgroups of samples, more preferably four subgroups of samples, even more preferably five subgroups of samples, even more six or more subgroups of samples. It is furthermore well understood by the skilled person that the number of subgroups may vary, e.g., depending on neuro-inflammatory disease, disease stage, selection of subjects, sample sizes, as well as number and type of parameters. Without being bound to theory, it may be hypothesized that for example in case of a neuro-inflammatory disease characterized by a high heterogeneity of clinical manifestations the number of groups that are initially assumed as well as the number identified by the analytical determination approach disclosed herein will increase with increasing sample sizes and parameter numbers.

Thus, in another preferred embodiment, the group of samples comprises a first subgroup of samples obtained from subjects suffering from a first subtype of a neuro-inflammatory disease, like E1, a second subgroup of samples obtained from subjects suffering from a second subtype of the neuro-inflammatory disease, like E2, and a further (herein also sometimes referred to as “fifth”) subgroup of samples obtained from healthy subjects. Preferably, the neuro-inflammatory disease is multiple sclerosis and the first and the second endophenotype of said neuro-inflammatory disease are a first and a second MS endophenotype, respectively.

Thus, in another preferred embodiment, the group of samples comprises a first subgroup of samples obtained from subjects suffering from a first subtype of a neuro-inflammatory disease, like E1, a second subgroup of samples obtained from subjects suffering from a second subtype of the neuro-inflammatory disease, like E2, a third subgroup of samples obtained from subjects suffering from a third subtype of the neuro-inflammatory disease, like E3, and a further (herein also sometimes referred to as “fifth”) subgroup of samples obtained from healthy subjects. Alternatively or additionally, the group of samples comprises at least a first subgroup of samples obtained from subjects suffering from a first endophenotype of a neuro-inflammatory disease, a second subgroup of samples obtained from subjects suffering from a second endophenotype of the neuro-inflammatory disease, a third subgroup of samples obtained from subjects suffering from a third endophenotype of the neuro-inflammatory disease, and a further (herein also sometimes referred to as “fifth”) subgroup of samples obtained from healthy subjects. This is especially advantageous to obtain distinguishing parameters that classify a subject based on its respective parameters as suffering from a first endophenotype, suffering from a second endophenotype, or suffering from a third endophenotype, and optionally as a healthy subject. Preferably, the neuro-inflammatory disease is MS and the first, second and third endophenotype of said neuro-inflammatory disease are a first, second and third MS endophenotype, respectively.

Thus, in another preferred embodiment, the group of samples comprises at least a first subgroup of samples obtained from subjects suffering from a first subtype of a neuro-inflammatory disease, preferably E1, a second subgroup of samples obtained from subjects suffering from a second subtype of the neuro-inflammatory disease, preferably E2, a third subgroup of samples obtained from subjects suffering from a third subtype of the neuro-inflammatory disease, preferably E3, a fourth subgroup of samples obtained from subjects suffering from a fourth subtype of the neuro-inflammatory disease, preferably E4, and optionally a fifth subgroup of samples obtained from healthy subjects. Alternatively or optionally, the group of samples comprises at least a first subgroup of samples obtained from subjects suffering from a first endophenotype of a neuro-inflammatory disease, a second subgroup of samples obtained from subjects suffering from a second endophenotype of the neuro-inflammatory disease, a third subgroup of samples obtained from subjects suffering from a third endophenotype of the neuro-inflammatory disease, a fourth subgroup of samples obtained from subjects suffering from a fourth endophenotype of the neuro-inflammatory disease, and optionally a fifth subgroup of samples obtained from healthy subjects. This is especially advantageous to obtain distinguishing parameters that classify a subject based on its respective parameters as suffering from a first endophenotype, suffering from a second endophenotype, suffering from a third endophenotype, or fourth endophenotype and optionally as a healthy subject. Preferably, the neuro-inflammatory disease is multiple sclerosis and the first, second, third and fourth endophenotype of said neuro-inflammatory disease are a first, second, third, and fourth MS endophenotype, respectively.

### Sample Sizes

According to the method for determining distinguishing parameters of a neuro-inflammatory disease of the invention, a group of samples is analysed using an analytical determination approach. Said group has preferably a minimal size as defined further herein to robustly identify and use distinguishing parameters according to the present invention.

Thus, said group of samples preferably comprises at least 25 samples, more preferably at least 50 samples, even more preferably at least 75 samples, even more preferably at least 100, even more preferably at least 150 samples, even more preferably at least 200, even more preferably at least 250 samples, even more preferably at least 300, even more preferably at least 370 samples.

Alternatively or optionally, in case of a subgroup being a subgroup of samples obtained from healthy subjects, said subgroup of samples is preferably obtained from at least 25, preferably from at least 40 healthy subjects.

Alternatively or optionally, in case of a subgroup being a subgroup of samples obtained from subjects suffering from a neuro-inflammatory disease, said subgroup of samples is preferably obtained from at least 50, preferably from at least 100, more preferably from at least 150 subjects suffering from said neuro-inflammatory disease.

Alternatively or optionally, in case of a subgroup being a subgroup of samples obtained from subjects suffering from a subtype of a neuro-inflammatory disease, said subgroup of samples is preferably obtained from at least 5, preferably at least 10, more preferably at least 25 subjects suffering from said sub-type of said neuro-inflammatory disease.

Alternatively or optionally, in case of a subgroup being a subgroup of samples obtained from subjects suffering from a first subtype of a neuro-inflammatory disease, said subgroup of samples is preferably obtained from at least 5, preferably at least 10, more preferably at least 25 subjects suffering from said first sub-type of said neuro-inflammatory disease.

Alternatively or optionally, in case of a subgroup being a subgroup of samples obtained from subjects suffering from a second subtype of a neuro-inflammatory disease, said subgroup of samples is preferably obtained from at least 5, preferably at least 10, more preferably at least 25 subjects suffering from said second sub-type of said neuro-inflammatory disease.

Alternatively or optionally, in case of a subgroup being a subgroup of samples obtained from subjects suffering from a third subtype of a neuro-inflammatory disease, said subgroup of samples is preferably obtained from at least 5, preferably at least 10, more preferably at least 25 subjects suffering from said third sub-type of said neuro-inflammatory disease.

Alternatively or optionally, in case of a subgroup being a subgroup of samples obtained from subjects suffering from a fourth subtype of a neuro-inflammatory disease, said subgroup of samples is preferably obtained from at least 5, preferably at least 10, more preferably at least 25 subjects suffering from said fourth sub-type of said neuro-inflammatory disease.

### Obtained Parameters

Herein, “soluble parameters” preferably refer to parameters that are obtained from soluble (blood) serum and/or (blood) plasma proteins. Herein, “cellular parameters” preferably refer to parameters that are obtained from blood cells, preferably from peripheral blood mononuclear cells.

In a preferred embodiment, the parameters are soluble parameters and/or cellular parameters, wherein the soluble parameters are preferably obtained and/or derived from soluble serum and/or plasma proteins, and wherein the cellular parameters are preferably obtained and/or derived from blood cells, preferably from peripheral blood mononuclear cells (PBMC). Any suitable material is encompassed according to the present invention, such as whole blood, fresh blood, buffy coat, or other material comprising soluble, cellular parameters.

According to the present invention, parameters are obtained from a sample of a subject. The sample is obtained by a physician, nurse or similarly qualified person preferably by venous puncture and aspiration of blood into a suitable container. In a preferred embodiment, the sample is selected from one or more of the group consisting of blood, serum and plasma, preferably wherein the sample is selected from one or more of the group consisting of fresh serum, cryopreserved serum, preserved serum, plasma, and blood.

In a preferred embodiment of the invention, the method for determining distinguishing parameters of a neuro-inflammatory disease foresees that the soluble parameters are obtained from soluble proteins and step (a) comprises a proteomics analysis, preferably mass spectrometry, and/or protein quantification, preferably comprising a labelling technique, more preferably a proximity extension assay (PEA-NGS), enzyme-linked immunosorbent assay (ELISA), next generation ELISA (ELLA), flow cytometry-based multiplex assays (e.g. Luminex), chemiluminescent enzyme immunoassay analysis (CLEIA) or single molecule array (SIMOA).

Further preferred, it is foreseen that the cellular parameters are derived from blood cells and step (a) comprises functional immune phenotyping, preferably comprising flow cytometry, single cell RNA sequencing (scRNAseq) mass cytometry or CiteSeq, more preferably flow cytometry. In accordance with the present invention, it has to be understood that a skilled person will be aware that any possible method may be used which allows to provide the cellular parameters.

### Distinguishing Parameters

In another aspect, the present invention relates to a set of distinguishing parameters of a neuro-inflammatory autoimmune disease, preferably MS, obtainable or obtained by the method for determining distinguishing parameters of a neuro-inflammatory disease according to the present invention and/or one if the respective embodiments disclosed herein.

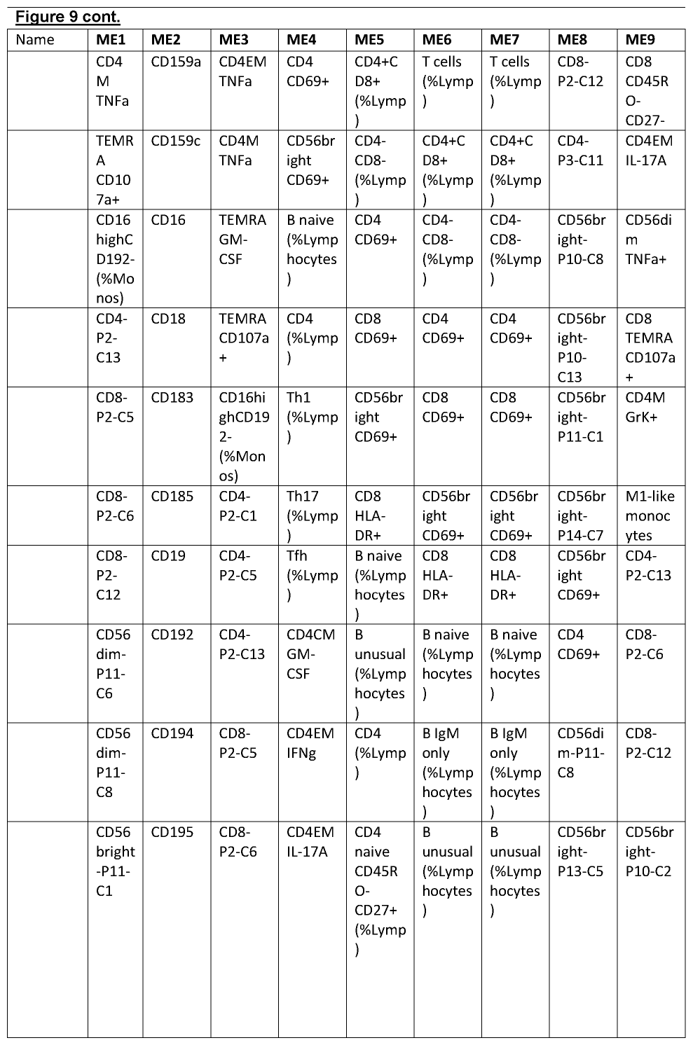

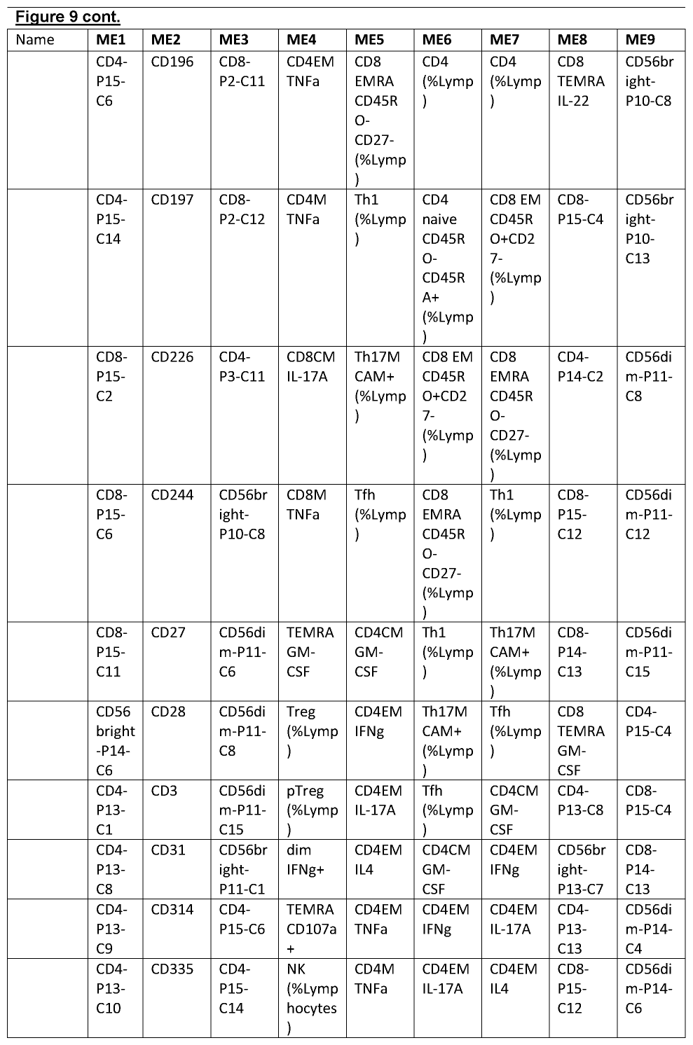

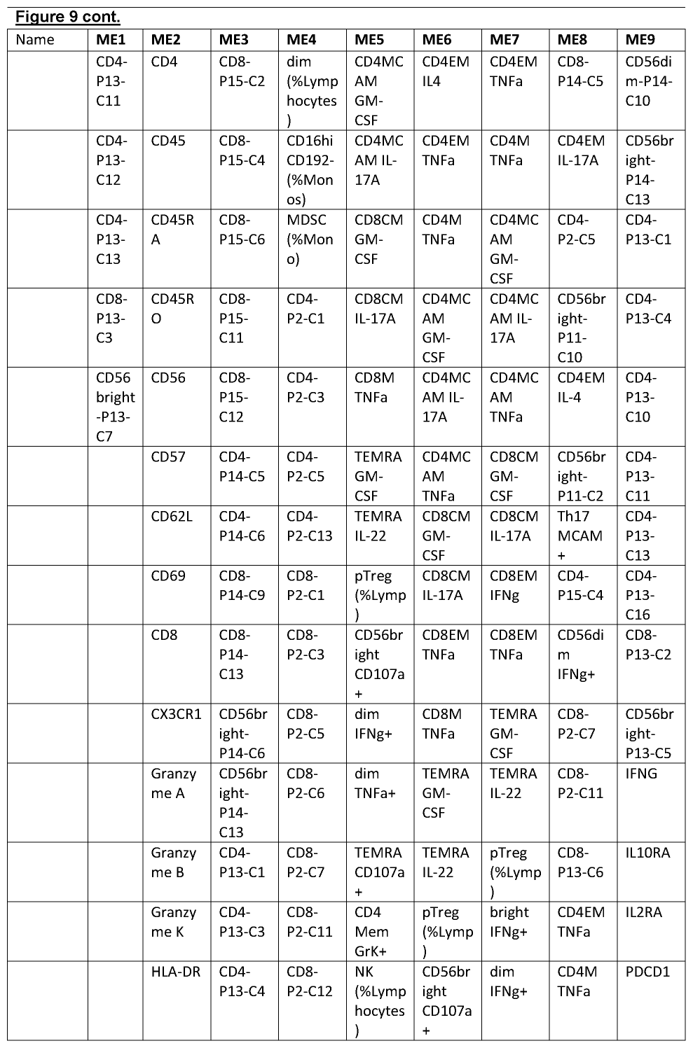

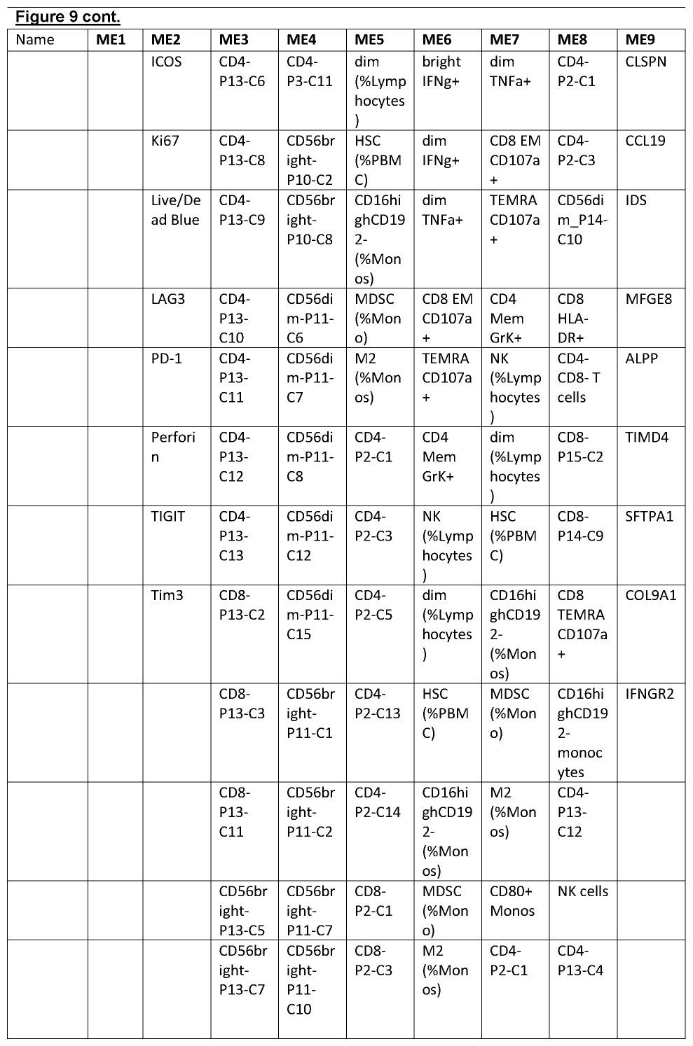

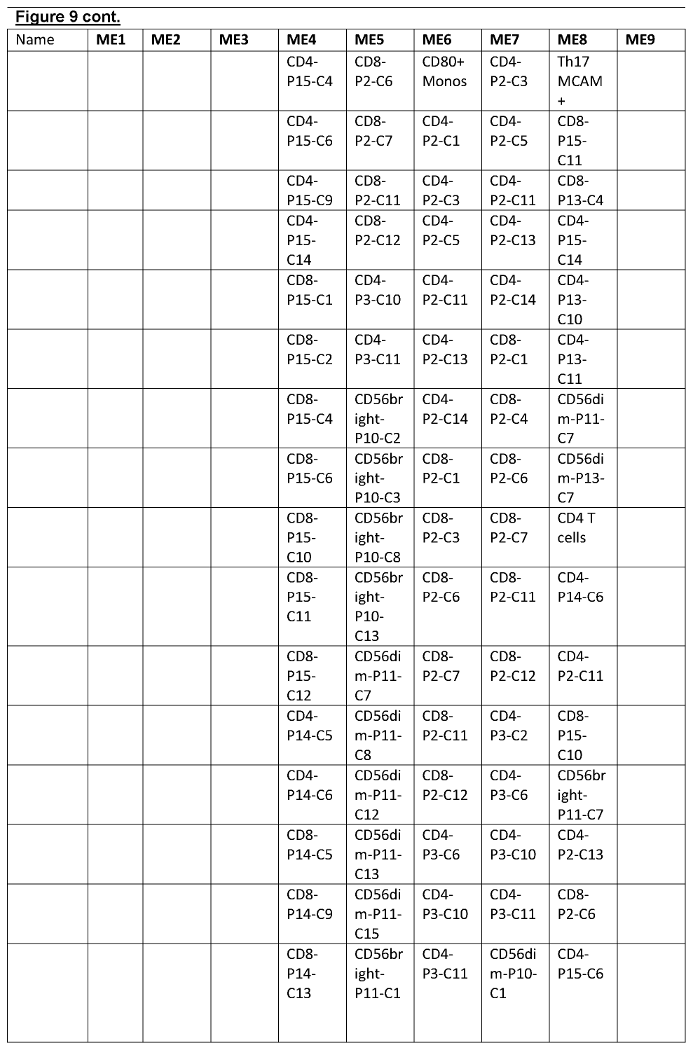

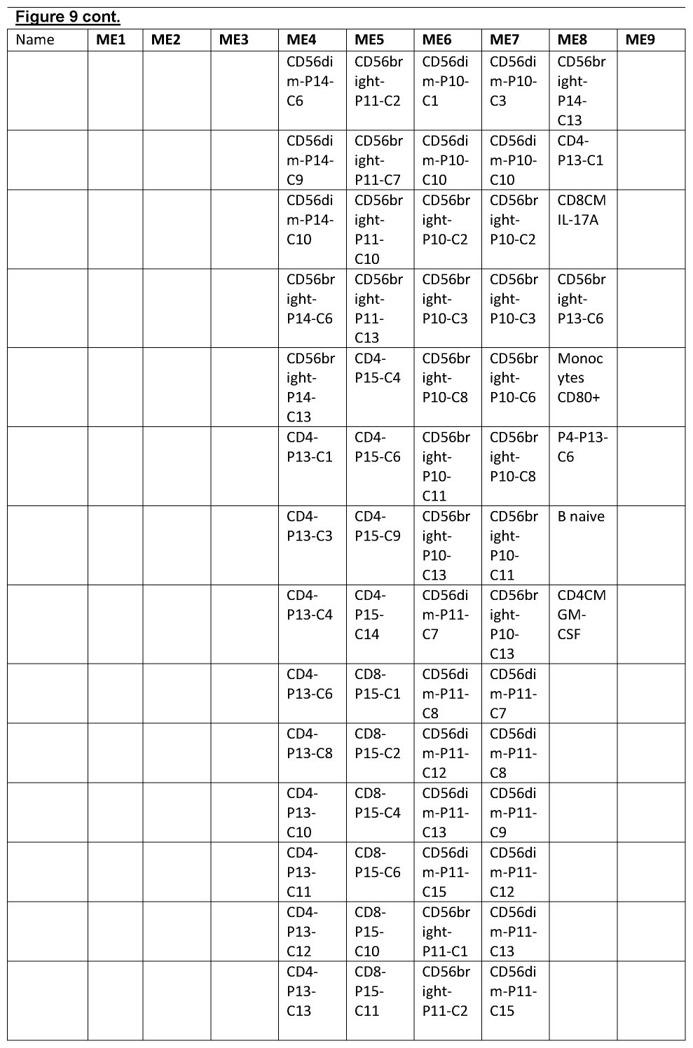

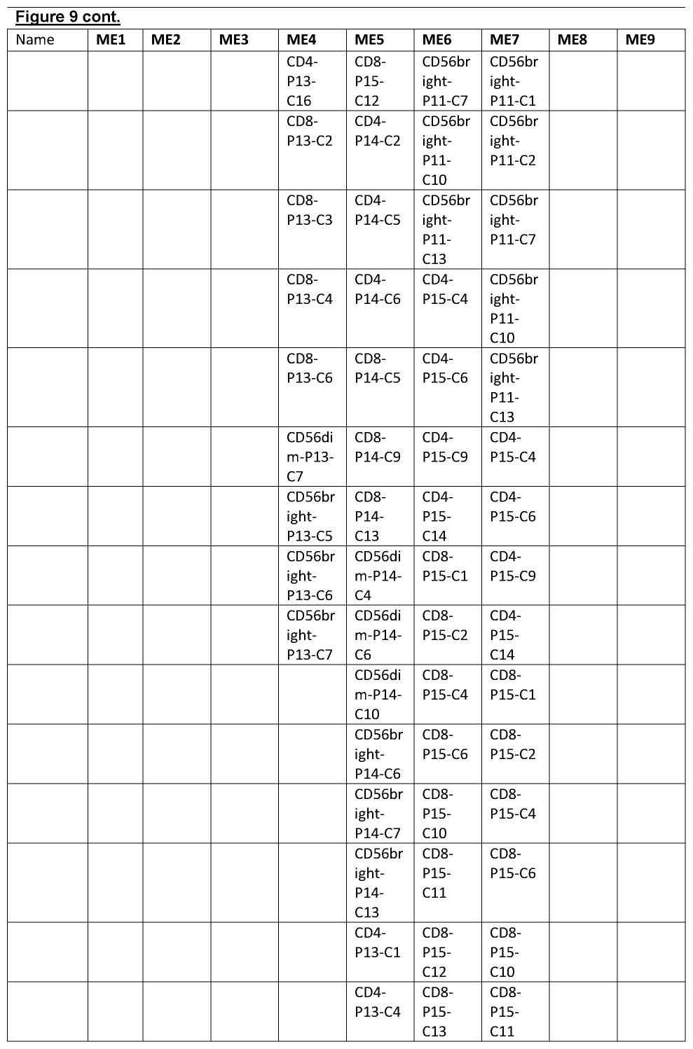

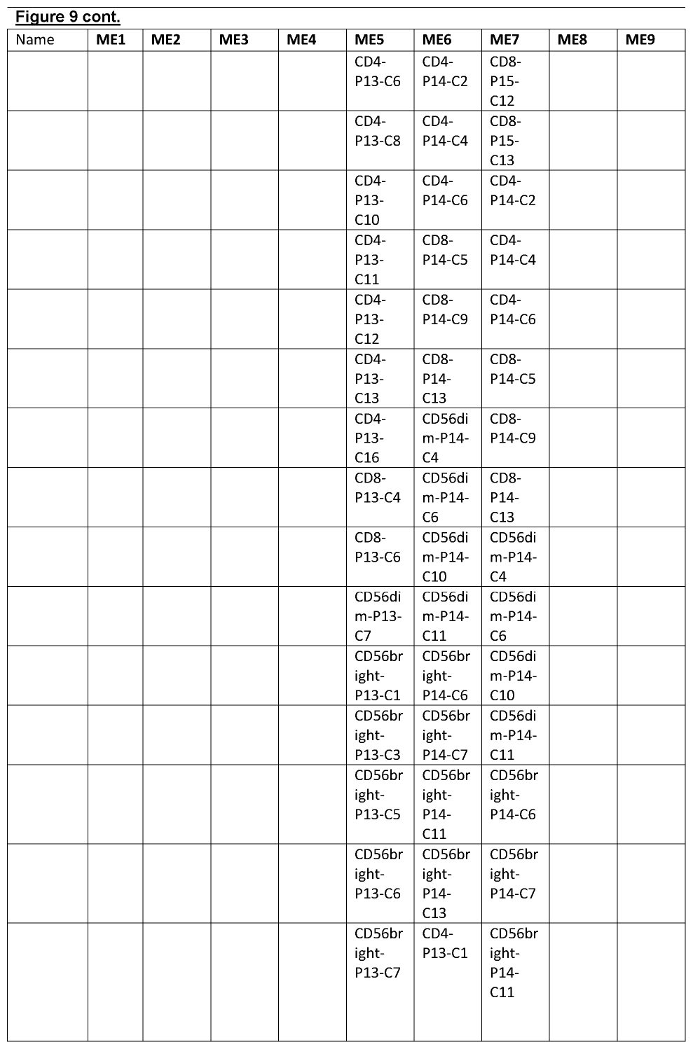

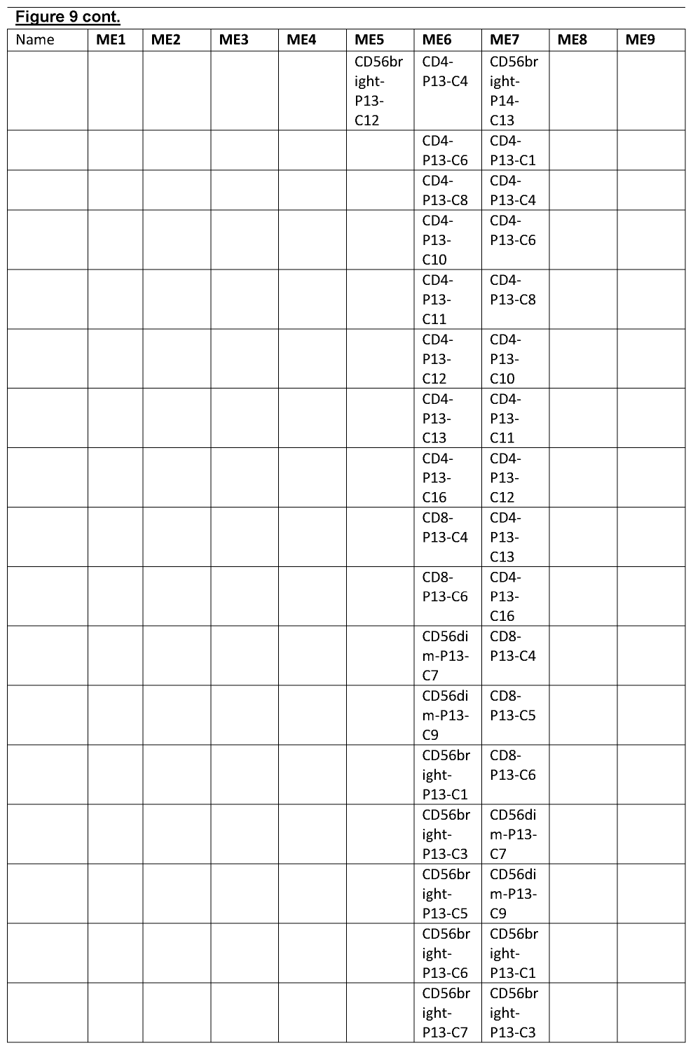

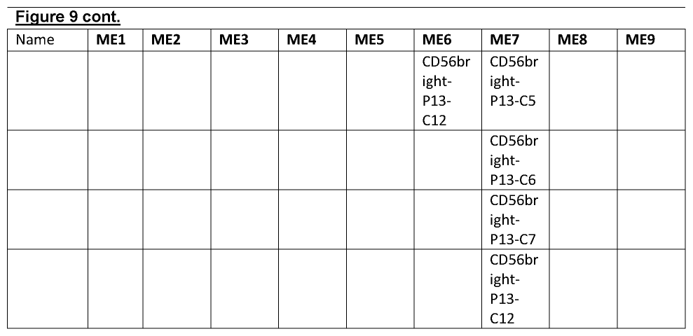

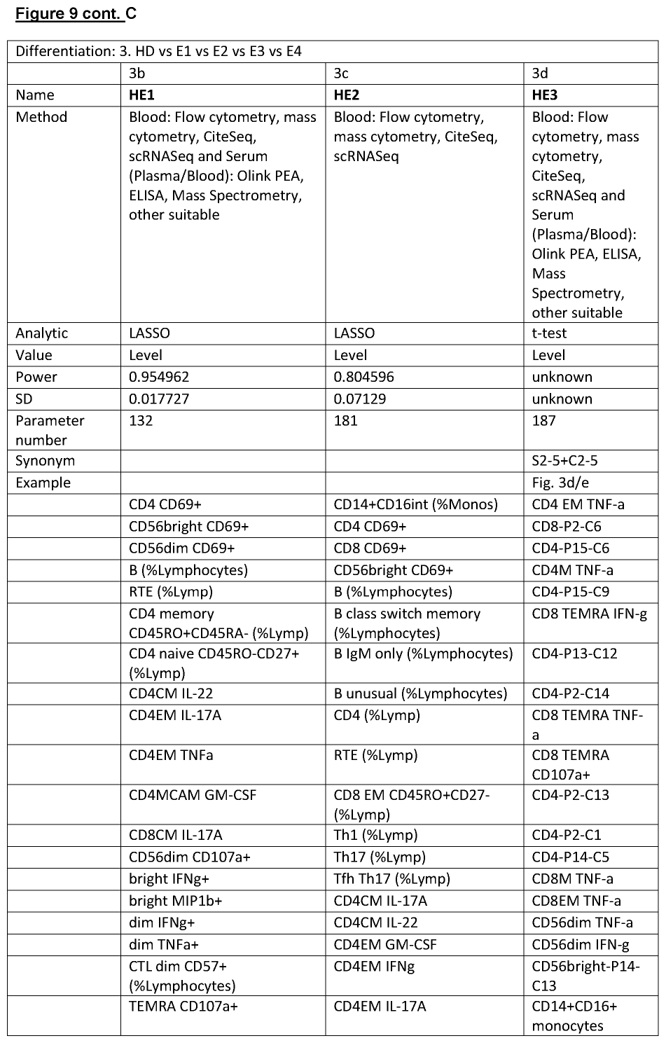

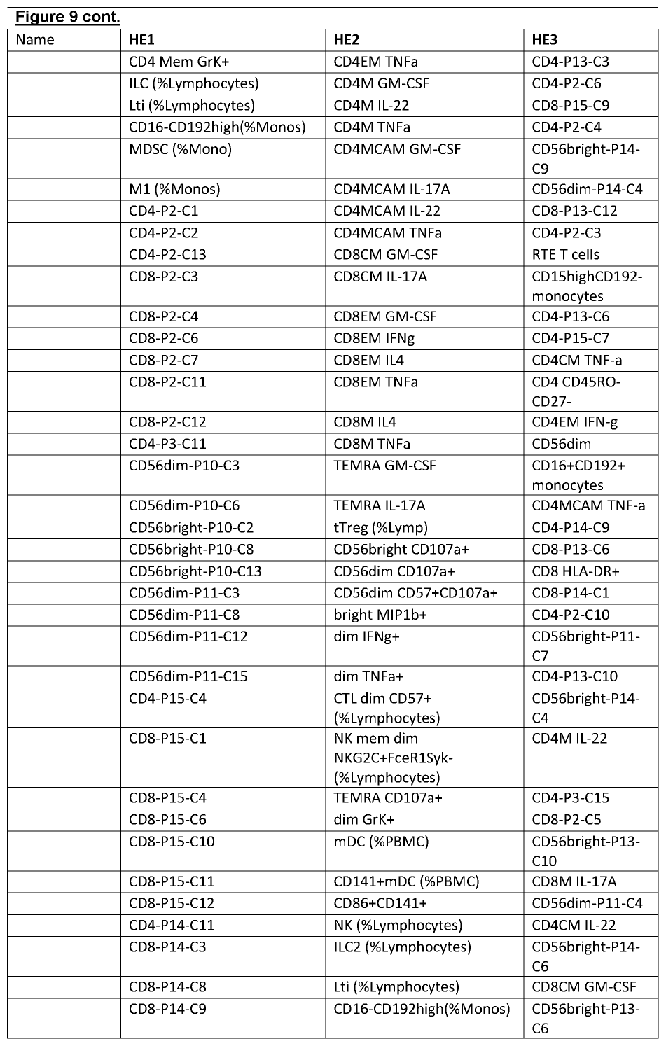

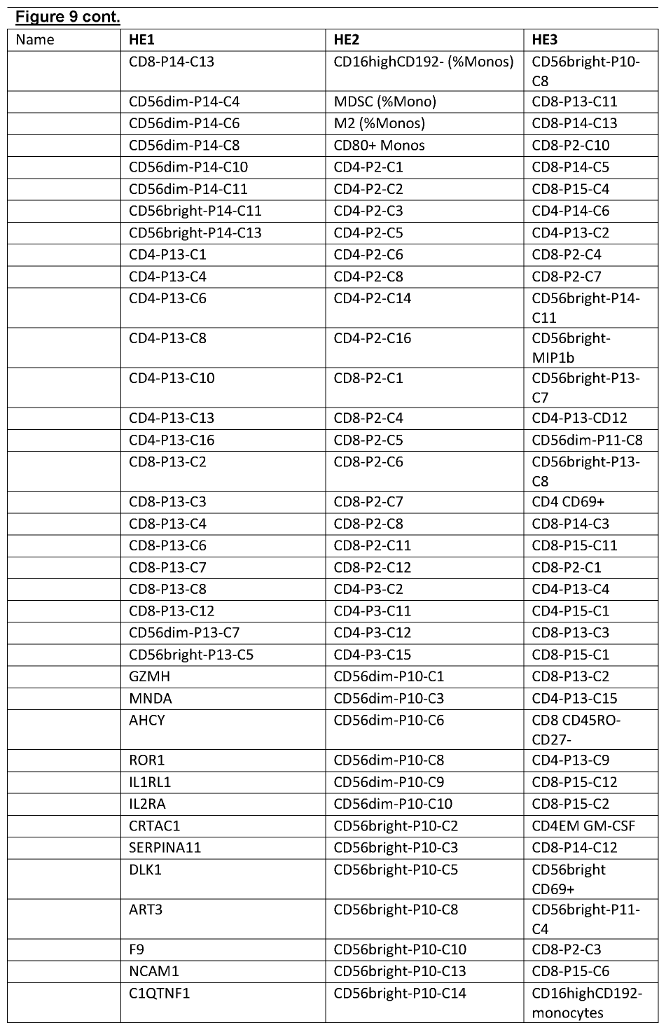

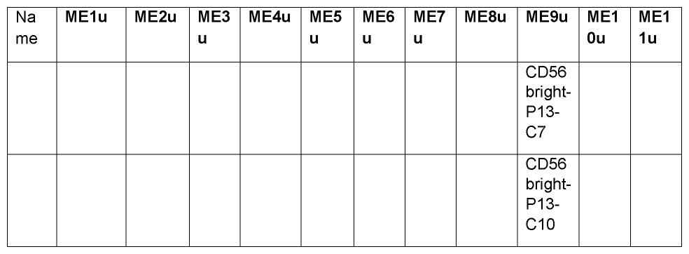

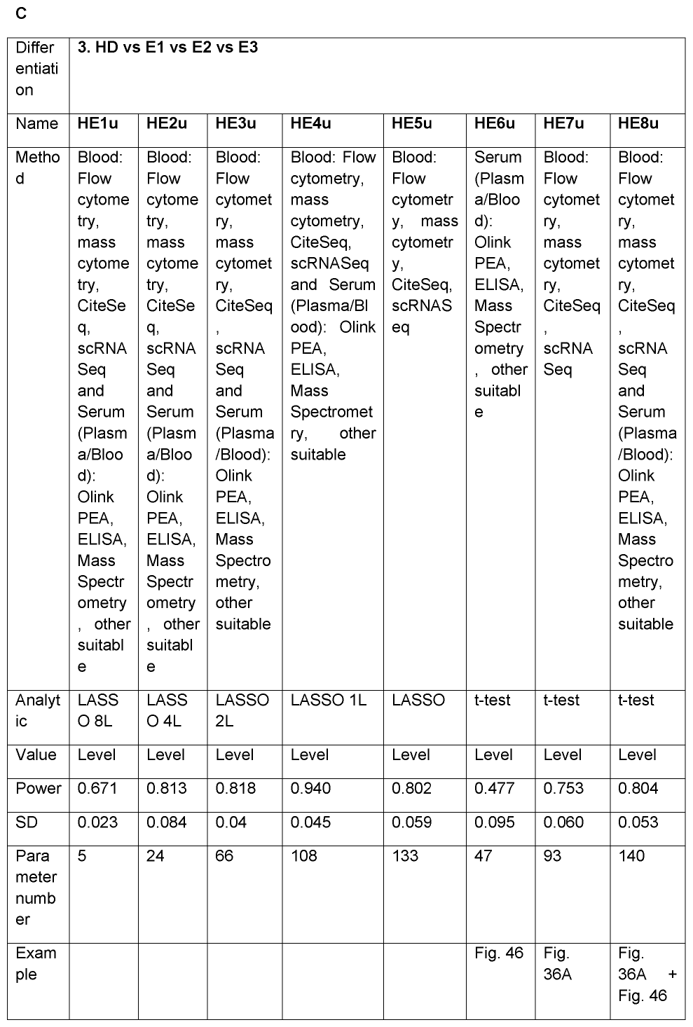

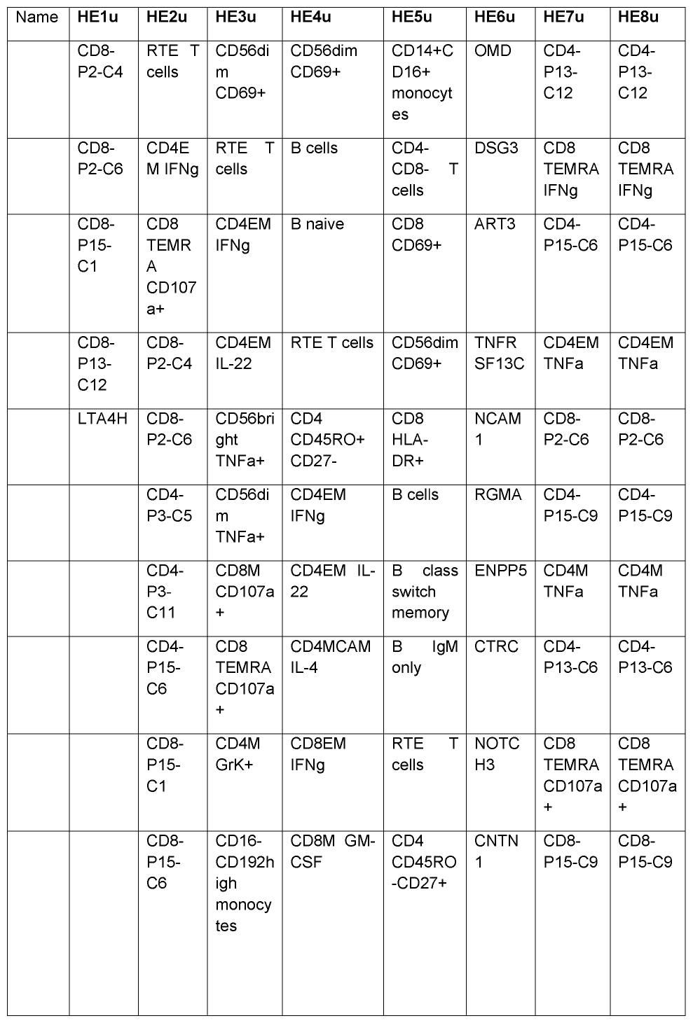

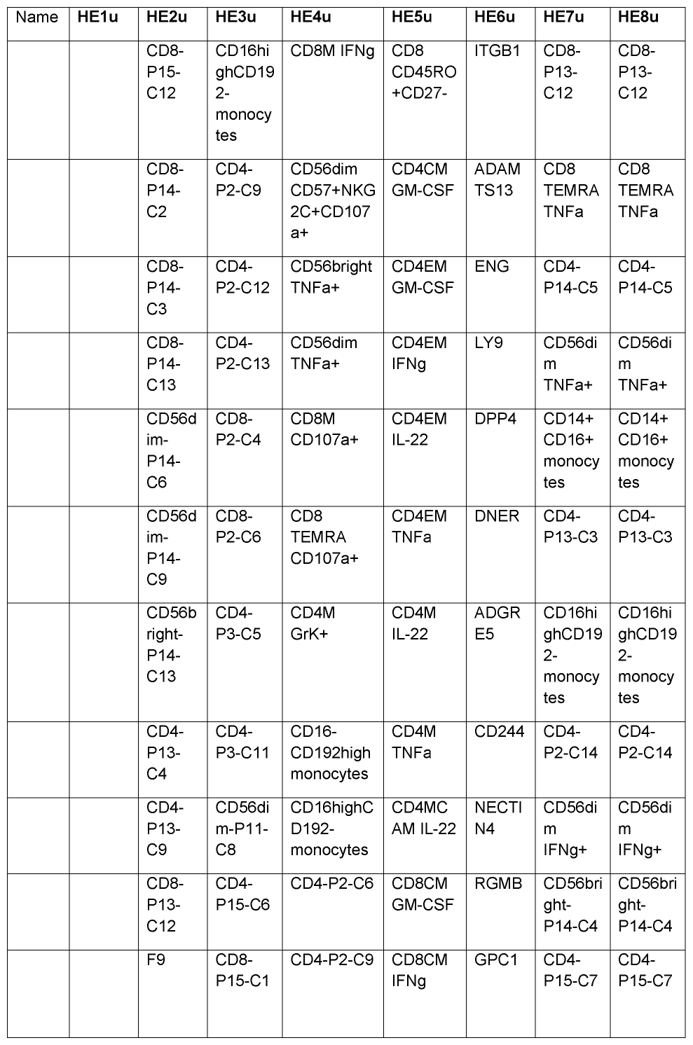

In a particularly preferred embodiment, said set of distinguishing parameters is a set of distinguishing parameters of MS, preferably of early stage MS, wherein the set comprises at least 85%, preferably 90%, more preferably 95%, even more preferably 100% of one of the sets listed in FIG. 9 (k=70, 4 endophenotypes) and in FIG. 41 (k=190, 3 endophenotypes), respectively, preferably wherein the set is identical to one of the sets listed in FIG. 9 (k=70, 4 endophenotypes) and/or in FIG. 41 (k=190, 3 endophenotypes).

In a preferred embodiment of the present invention, the set of distinguishing parameters is for use in determining whether or not a subject suffers from said neuro-inflammatory autoimmune disease, wherein the neuro-inflammatory autoimmune disease is preferably MS.

In a further preferred embodiment of the present invention, the set of distinguishing parameters is for use in determining a subtype of a neuro-inflammatory autoimmune disease in a subject, wherein said subtype is preferably an endophenotype of said neuro-inflammatory disease, wherein the neuro-inflammatory autoimmune disease is preferably MS.

Preferably, the set of distinguishing parameters is for use as described above or below, wherein the neuro-inflammatory autoimmune disease is MS and the endophenotype is, in case of k=70 (4 endophenotypes), selected from the group consisting of E1, E2, E3, and E4 or, in case of k=190 (3 endophenotypes), from the group consisting of E1, E2, and E3. The hyperparameter k is described in more detail below.

In accordance with the present invention, it has to be understood that the parameter and/or parameter sets may be comprised as one or more or all in the set of distinguishing parameters. Thus, the invention relates to a set of distinguishing parameters and encompasses a set of distinguishing parameters as defined above and below, wherein the set of distinguishing parameters may comprise any possible combination of parameters or set of parameters of the parameters as described within the application, in particular as described above and below, in the figures and in the claims.

Further preferred, the set of distinguishing parameters is for use as described above, wherein the neuro-inflammatory autoimmune disease is MS and wherein the set comprises at least 85%, preferably 90%, more preferably 95%, even more preferably 100% of one of the sets listed, in case of k=70 (4 endophenotypes), in the Table in FIG. 9 or, in case of k=190 (3 endophenotypes), in the Table in FIG. 41, preferably wherein the set is identical to one of the sets listed, in case of k=70 (4 endophenotypes), in the Table in FIG. 9 or, in case of k=190 (3 endophenotypes), in the Table in FIG. 41.

Preferably, the MS endophenotypes E1, E2 and E3 may be determined using the distinguishing parameter ME10u, which is able to determine the endophenotypes E1, E2 and E3 with about 91.9% accuracy. Also preferred, the MS endophenotypes E1, E2 and E3 may be determined using the distinguishing parameter HE7u, which is able to determine the endophenotypes E1, E2 and E3 with about 75.3% accuracy.

In a preferred embodiment of the invention, a set of distinguishing parameters is HM1 according to FIG. 9 in case of k=70 (4 endophenotypes). The set of distinguishing parameters according to HM1 comprises, or consists of, the parameters ART3, F9, NEFL, TCL1A, MYOC, MOG, and LTA4H.

In a preferred embodiment of the invention, a set of distinguishing parameters is HM2 according to FIG. 9 in case of k=70 (4 endophenotypes). The set of distinguishing parameters according to HM2 comprises, or consists of, the parameters LTA4H, MYOC, NEFL, F9, PRDX6, SERPINA9, AHCY, SEMA4C, MB, TCL1A, MOG, CASP8, GPC1, CPE, CA6, SEPTIN9, CA14, CNTN1, ENPP5, DPP4, RGMB, RGMA, IL6, CDNF, and LEP.

In a preferred embodiment of the invention, a set of distinguishing parameters is HM3 according to FIG. 9 in case of k=70 (4 endophenotypes). The set of distinguishing parameters according to HM3 comprises, or consists of, the parameters MNDA, AHCY, DLK1, ART3, F9, NCAM1, CHI3L1, CCL26, TREM2, NEFL, TCL1A, AHSP, MYOC, STXBP3, SERPINA9, MOG, PRDX6, SPINK6, LTA4H, FURIN

In a preferred embodiment of the invention, a set of distinguishing parameters is HM4 according to FIG. 9 in case of k=70 (4 endophenotypes). The set of distinguishing parameters according to HM4 comprises, or consists of, the parameters MNDA, AHCY, PRSS27, DLK1, CNTN1, ART3, F9, NCAM1, CHI3L1, IL20RA, LILRB4, RGS8, SLAMF1, TRAF2, CCL26, CLEC7A, FLT3LG, TREM2, NEFL, OBP2B, TCL1A, AHSP, CD177, MYOC, RUVBL1, STXBP3, SERPINA9, MOG, SPINK6, LTA4H, KDR, and FURIN.

In a preferred embodiment of the invention, a set of distinguishing parameters is HM5 according to FIG. 9 in case of k=70 (4 endophenotypes). The set of distinguishing parameters according to HM5 comprises, or consists of, the parameters B class switch memory (% Lymphocytes), B IgM only (% Lymphocytes), CD4CM IL-22, CD4EM GM-CSF, CD4MCAM IL-22, CD8EM TNFa, bright MIP1b+, dim TNFa+, TEMRA CD107a+, dim (% Lymphocytes), Lti (% Lymphocytes), CD4-P2-C8, CD8-P2-C1, CD8-P2-C4, CD8-P2-C8, CD4-P3-C12, CD4-P3-C15, CD56dim-P10-C6, CD56bright-P10-C1, CD56bright-P10-C5, CD56bright-P10-C8, CD56dim-P11-C4, CD56dim-P11-C7, CD56dim-P11-C14, CD56dim-P11-C15, CD56bright-P11-C2, CD4-P15-C1, CD4-P15-C9, CD8-P14-C3, CD56dim-P14-C1, CD56dim-P14-C5, CD56bright-P14-C4, CD56bright-P14-C9, CD56bright-P14-C11, CD56bright-P14-C13, CD4-P13-C6, CD4-P13-C12, CD4-P13-C15, CD8-P13-C6, CD8-P13-C12, CD56bright-P13-C10.

In a preferred embodiment of the invention, a set of distinguishing parameters is HM6 according to FIG. 9 in case of k=70 (4 endophenotypes). The set of distinguishing parameters according to HM6 comprises, or consists of, the parameters CD4+CD8+ (% Lymp), B class switch memory (% Lymphocytes), B IgM only (% Lymphocytes), CD4 naive CD45RO−CD27+ (% Lymp), Th1 (% Lymp), Memory MCAM (% Lymp), CD4CM IL-22, CD4CM IL4, CD4EM GM-CSF, CD4M IL-22, CD4MCAM IL-22, CD8EM IL4, CD8EM TNFa, tTreg (% Lymp), CD56bright CD107a+, CD56dim CD57+CD107a+, bright MIP1b+, dim TNFa+, TEMRA CD107a+, NK (% Lymphocytes), dim (% Lymphocytes), Lti (% Lymphocytes), CD80+ Monos, CD4-P2-C8, CD4-P2-C16, CD8-P2-C1, CD8-P2-C4, CD8-P2-C8, CD4-P3-C12, CD4-P3-C15, CD56dim-P10-C1, CD56dim-P10-C6, CD56dim-P10-C9, CD56bright-P10-C1, CD56bright-P10-C5, CD56bright-P10-C14, CD56dim-P11-C3, CD56dim-P11-C7, CD56dim-P11-C8, CD56dim-P11-C14, CD56dim-P11-C15, CD56bright-P11-C2, CD56bright-P11-C4, CD4-P15-C1, CD4-P15-C5, CD4-P15-C8, CD4-P15-C9, CD8-P15-C1, CD4-P14-C7, CD8-P14-C3, CD56dim-P14-C1, CD56dim-P14-C5, CD56dim-P14-C11, CD56bright-P14-C2, CD56bright-P14-C4, CD56bright-P14-C5, CD56bright-P14-C6, CD56bright-P14-C9, CD56bright-P14-C10, CD56bright-P14-C11, CD56bright-P14-C13, CD4-P13-C15, CD8-P13-C6, CD8-P13-C12, CD8-P13-C13, CD56dim-P13-C3, CD56dim-P13-C6, CD56dim-P13-C9, CD56bright-P13-C3, CD56bright-P13-C8, CD56bright-P13-C10, and CD56bright-P13-C11.

In a preferred embodiment of the invention, a set of distinguishing parameters is HM7 according to FIG. 9 in case of k=70 (4 endophenotypes). The set of distinguishing parameters according to HM7 comprises, or consists of, the parameters CD8-P2-C4, CD4-P13-C12, CD4-P15-C9, CD4-P2-C8, CD56bright-P14-C4, CD56bright-P14-C9, CD8 TEMRA CD107a+, CD4 memory MCAM+, CD8-P13-C12, CD4-P15-C6, CD4CM IL-22, CD56bright-P14-C11, CD4-P13-C6, CD56dim-P11-C14, CD56dim-P14-C5, CD8-P2-C7, CD4-P13-C3, CD56bright MIP1b+, CD4M IL-22, CD56bright-P13-C8, CD4-P13-C10, CD56bright-P10-C8, CD8 TEMRA IFN-g, CD16highCD192− (% Monos), CD56dim, CD4-P15-C3, B regulatory, CD4-P15-C1, CD56bright-P13-C10, CD4-P14-C7, CD56bright-P11-C1, B IgM only, CD4EM GM-CSF, CD56dim-P10-C9, CD4-P13-C7, CD8-P15-C9, CD56bright-P14-C6, and CD4-P15-C11.

In a preferred embodiment of the invention, a set of distinguishing parameters is ME1 according to FIG. 9 in case of k=70 (4 endophenotypes). The set of distinguishing parameters according to ME1 comprises, or consists of, the parameters CD4 CD69+, CD4EM TNFa, CD4M TNFa, TEMRA CD107a+, CD16highCD192− (% Monos), CD4-P2-C13, CD8-P2-C5, CD8-P2-C6, CD8-P2-C12, CD56dim-P11-C6, CD56dim-P11-C8, CD56bright-P11-C1, CD4-P15-C6, CD4-P15-C14, CD8-P15-C2, CD8-P15-C6, CD8-P15-C11, CD56bright-P14-C6, CD4-P13-C1, CD4-P13-C8, CD4-P13-C9, CD4-P13-C10, CD4-P13-C11, CD4-P13-C12, CD4-P13-C13, CD8-P13-C3, and CD56bright-P13-C7.

In a preferred embodiment of the invention, a set of distinguishing parameters is ME2 according to FIG. 9 in case of k=70 (4 endophenotypes). The set of distinguishing parameters according to ME2 comprises, or consists of, the parameters CD127, CD14, CD159a, CD159c, CD16, CD18, CD183, CD185, CD19, CD192, CD194, CD195, CD196, CD197, CD226, CD244, CD27, CD28, CD3, CD31, CD314, CD335, CD4, CD45, CD45RA, CD45RO, CD56, CD57, CD62L, CD69, CD8, CX3CR1, Granzyme A, Granzyme B, Granzyme K, HLA-DR, ICOS, Ki67, Live/Dead Blue, LAG3, PD-1, Perforin, TIGIT, and Tim3.

In a preferred embodiment of the invention, a set of distinguishing parameters is ME3 according to FIG. 9 in case of k=70 (4 endophenotypes). The set of distinguishing parameters according to ME3 comprises, or consists of, the parameters CD4 CD69+, CD4EM IL-17A, CD4EM TNFa, CD4M TNFa, TEMRA GM-CSF, TEMRA CD107a+, CD16highCD192− (% Monos), CD4-P2-C1, CD4-P2-C5, CD4-P2-C13, CD8-P2-C5, CD8-P2-C6, CD8-P2-C11, CD8-P2-C12, CD4-P3-C11, CD56bright-P10-C8, CD56dim-P11-C6, CD56dim-P11-C8, CD56dim-P11-C15, CD56bright-P11-C1, CD4-P15-C6, CD4-P15-C14, CD8-P15-C2, CD8-P15-C4, CD8-P15-C6, CD8-P15-C11, CD8-P15-C12, CD4-P14-C5, CD4-P14-C6, CD8-P14-C9, CD8-P14-C13, CD56bright-P14-C6, CD56bright-P14-C13, CD4-P13-C1, CD4-P13-C3, CD4-P13-C4, CD4-P13-C6, CD4-P13-C8, CD4-P13-C9, CD4-P13-C10, CD4-P13-C11, CD4-P13-C12, CD4-P13-C13, CD8-P13-C2, CD8-P13-C3, CD8-P13-C11, CD56bright-P13-C5, and CD56bright-P13-C7.

In a preferred embodiment of the invention, a set of distinguishing parameters is ME4 according to FIG. 9 in case of k=70 (4 endophenotypes). The set of distinguishing parameters according to ME4 comprises, or consists of, the parameters CD14+CD16+ (% Monos), CD4+CD8+ (% Lymp), CD4 CD69+, CD56bright CD69+, B naive (% Lymphocytes), CD4 (% Lymp), Th1 (% Lymp), Th17 (% Lymp), Tfh (% Lymp), CD4CM GM-CSF, CD4EM IFNg, CD4EM IL-17A, CD4EM TNFa, CD4M TNFa, CD8CM IL-17A, CD8M TNFa, TEMRA GM-CSF, Treg (% Lymp), pTreg (% Lymp), dim IFNg+, TEMRA CD107a+, NK (% Lymphocytes), dim (% Lymphocytes), CD16highCD192− (% Monos), MDSC (% Mono), CD4-P2-C1, CD4-P2-C3, CD4-P2-C5, CD4-P2-C13, CD8-P2-C1, CD8-P2-C3, CD8-P2-C5, CD8-P2-C6, CD8-P2-C7, CD8-P2-C11, CD8-P2-C12, CD4-P3-C11, CD56bright-P10-C2, CD56bright-P10-C8, CD56dim-P11-C6, CD56dim-P11-C7, CD56dim-P11-C8, CD56dim-P11-C12, CD56dim-P11-C15, CD56bright-P11-C1, CD56bright-P11-C2, CD56bright-P11-C7, CD56bright-P11-C10, CD4-P15-C4, CD4-P15-C6, CD4-P15-C9, CD4-P15-C14, CD8-P15-C1, CD8-P15-C2, CD8-P15-C4, CD8-P15-C6, CD8-P15-C10, CD8-P15-C11, CD8-P15-C12, CD4-P14-C5, CD4-P14-C6, CD8-P14-C5, CD8-P14-C9, CD8-P14-C13, CD56dim-P14-C6, CD56dim-P14-C9, CD56dim-P14-C10, CD56bright-P14-C6, CD56bright-P14-C13, CD4-P13-C1, CD4-P13-C3, CD4-P13-C4, CD4-P13-C6, CD4-P13-C8, CD4-P13-C10, CD4-P13-C11, CD4-P13-C12, CD4-P13-C13, CD4-P13-C16, CD8-P13-C2, CD8-P13-C3, CD8-P13-C4, CD8-P13-C6, CD56dim-P13-C7, CD56bright-P13-C5, CD56bright-P13-C6, and CD56bright-P13-C7.

In a preferred embodiment of the invention, a set of distinguishing parameters is ME5 according to FIG. 9 in case of k=70 (4 endophenotypes). The set of distinguishing parameters according to ME5 comprises, or consists of, the parameters CD14+CD16int (% Monos), CD14+CD16+ (% Monos), CD4+CD8+ (% Lymp), CD4−CD8− (% Lymp), CD4 CD69+, CD8 CD69+, CD56bright CD69+, CD8 HLA-DR+, B naive (% Lymphocytes), B unusual (% Lymphocytes), CD4 (% Lymp), CD4 naive CD45RO−CD27+ (% Lymp), CD8 EMRA CD45RO−CD27− (% Lymp), Th1 (% Lymp), Th17MCAM+ (% Lymp), Tfh (% Lymp), CD4CM GM-CSF, CD4EM IFNg, CD4EM IL-17A, CD4EM IL4, CD4EM TNFa, CD4M TNFa, CD4MCAM GM-CSF, CD4MCAM IL-17A, CD8CM GM-CSF, CD8CM IL-17A, CD8M TNFa, TEMRA GM-CSF, TEMRA IL-22, pTreg (% Lymp), CD56bright CD107a+, dim IFNg+, dim TNFa+, TEMRA CD107a+, CD4 Mem GrK+, NK (% Lymphocytes), dim (% Lymphocytes), HSC (% PBMC), CD16highCD192− (% Monos), MDSC (% Mono), M2 (% Monos), CD4-P2-C1, CD4-P2-C3, CD4-P2-C5, CD4-P2-C13, CD4-P2-C14, CD8-P2-C1, CD8-P2-C3, CD8-P2-C6, CD8-P2-C7, CD8-P2-C11, CD8-P2-C12, CD4-P3-C10, CD4-P3-C11, CD56bright-P10-C2, CD56bright-P10-C3, CD56bright-P10-C8, CD56bright-P10-C13, CD56dim-P11-C7, CD56dim-P11-C8, CD56dim-P11-C12, CD56dim-P11-C13, CD56dim-P11-C15, CD56bright-P11-C1, CD56bright-P11-C2, CD56bright-P11-C7, CD56bright-P11-C10, CD56bright-P11-C13, CD4-P15-C4, CD4-P15-C6, CD4-P15-C9, CD4-P15-C14, CD8-P15-C1, CD8-P15-C2, CD8-P15-C4, CD8-P15-C6, CD8-P15-C10, CD8-P15-C11, CD8-P15-C12, CD4-P14-C2, CD4-P14-C5, CD4-P14-C6, CD8-P14-C5, CD8-P14-C9, CD8-P14-C13, CD56dim-P14-C4, CD56dim-P14-C6, CD56dim-P14-C10, CD56bright-P14-C6, CD56bright-P14-C7, CD56bright-P14-C13, CD4-P13-C1, CD4-P13-C4, CD4-P13-C6, CD4-P13-C8, CD4-P13-C10, CD4-P13-C11, CD4-P13-C12, CD4-P13-C13, CD4-P13-C16, CD8-P13-C4, CD8-P13-C6, CD56dim-P13-C7, CD56bright-P13-C1, CD56bright-P13-C3, CD56bright-P13-C5, CD56bright-P13-C6, CD56bright-P13-C7, and CD56bright-P13-C12.

In a preferred embodiment of the invention, a set of distinguishing parameters is ME6 according to FIG. 9 in case of k=70 (4 endophenotypes). The set of distinguishing parameters according to ME6 comprises, or consists of, the parameters Lymphocytes (% PBMC), CD14+CD16int (% Monos), T cells (% Lymp), CD4+CD8+ (% Lymp), CD4−CD8− (% Lymp), CD4 CD69+, CD8 CD69+, CD56bright CD69+, CD8 HLA-DR+, B naive (% Lymphocytes), B IgM only (% Lymphocytes), B unusual (% Lymphocytes), CD4 (% Lymp), CD4 naive CD45RO−CD45RA+ (% Lymp), CD8 EM CD45RO+CD27− (% Lymp), CD8 EMRA CD45RO−CD27− (% Lymp), Th1 (% Lymp), Th17MCAM+ (% Lymp), Tfh (% Lymp), CD4CM GM-CSF, CD4EM IFNg, CD4EM IL-17A, CD4EM IL4, CD4EM TNFa, CD4M TNFa, CD4MCAM GM-CSF, CD4MCAM IL-17A, CD4MCAM TNFa, CD8CM GM-CSF, CD8CM IL-17A, CD8EM TNFa, CD8M TNFa, TEMRA GM-CSF, TEMRA IL-22, pTreg (% Lymp), CD56bright CD107a+, bright IFNg+, dim IFNg+, dim TNFa+, CD8 EM CD107a+, TEMRA CD107a+, CD4 Mem GrK+, NK (% Lymphocytes), dim (% Lymphocytes), HSC (% PBMC), CD16highCD192− (% Monos), MDSC (% Mono), M2 (% Monos), CD80+ Monos, CD4-P2-C1, CD4-P2-C3, CD4-P2-C5, CD4-P2-C11, CD4-P2-C13, CD4-P2-C14, CD8-P2-C1, CD8-P2-C3, CD8-P2-C6, CD8-P2-C7, CD8-P2-C11, CD8-P2-C12, CD4-P3-C6, CD4-P3-C10, CD4-P3-C11, CD56dim-P10-C1, CD56dim-P10-C10, CD56bright-P10-C2, CD56bright-P10-C3, CD56bright-P10-C8, CD56bright-P10-C11, CD56bright-P10-C13, CD56dim-P11-C7, CD56dim-P11-C8, CD56dim-P11-C12, CD56dim-P11-C13, CD56dim-P11-C15, CD56bright-P11-C1, CD56bright-P11-C2, CD56bright-P11-C7, CD56bright-P11-C10, CD56bright-P11-C13, CD4-P15-C4, CD4-P15-C6, CD4-P15-C9, CD4-P15-C14, CD8-P15-C1, CD8-P15-C2, CD8-P15-C4, CD8-P15-C6, CD8-P15-C10, CD8-P15-C11, CD8-P15-C12, CD8-P15-C13, CD4-P14-C2, CD4-P14-C4, CD4-P14-C6, CD8-P14-C5, CD8-P14-C9, CD8-P14-C13, CD56dim-P14-C4, CD56dim-P14-C6, CD56dim-P14-C10, CD56dim-P14-C11, CD56bright-P14-C6, CD56bright-P14-C7, CD56bright-P14-C11, CD56bright-P14-C13, CD4-P13-C1, CD4-P13-C4, CD4-P13-C6, CD4-P13-C8, CD4-P13-C10, CD4-P13-C11, CD4-P13-C12, CD4-P13-C13, CD4-P13-C16, CD8-P13-C4, CD8-P13-C6, CD56dim-P13-C7, CD56dim-P13-C9, CD56bright-P13-C1, CD56bright-P13-C3, CD56bright-P13-C5, CD56bright-P13-C6, CD56bright-P13-C7, and CD56bright-P13-C12.

In a preferred embodiment of the invention, a set of distinguishing parameters is ME7 according to FIG. 9 in case of k=70 (4 endophenotypes). The set of distinguishing parameters according to ME7 comprises, or consists of, the parameters Monocytes (% PBMC), CD14+CD16int (% Monos), T cells (% Lymp), CD4+CD8+ (% Lymp), CD4−CD8− (% Lymp), CD4 CD69+, CD8 CD69+, CD56bright CD69+, CD8 HLA-DR+, B naive (% Lymphocytes), B IgM only (% Lymphocytes), B unusual (% Lymphocytes), CD4 (% Lymp), CD8 EM CD45RO+CD27− (% Lymp), CD8 EMRA CD45RO−CD27− (% Lymp), Th1 (% Lymp), Th17MCAM+ (% Lymp), Tfh (% Lymp), CD4CM GM-CSF, CD4EM IFNg, CD4EM IL-17A, CD4EM IL4, CD4EM TNFa, CD4M TNFa, CD4MCAM GM-CSF, CD4MCAM IL-17A, CD4MCAM TNFa, CD8CM GM-CSF, CD8CM IL-17A, CD8EM IFNg, CD8EM TNFa, TEMRA GM-CSF, TEMRA IL-22, pTreg (% Lymp), bright IFNg+, dim IFNg+, dim TNFa+, CD8 EM CD107a+, TEMRA CD107a+, CD4 Mem GrK+, NK (% Lymphocytes), dim (% Lymphocytes), HSC (% PBMC), CD16highCD192− (% Monos), MDSC (% Mono), M2 (% Monos), CD80+ Monos, CD4-P2-C1, CD4-P2-C3, CD4-P2-C5, CD4-P2-C11, CD4-P2-C13, CD4-P2-C14, CD8-P2-C1, CD8-P2-C4, CD8-P2-C6, CD8-P2-C7, CD8-P2-C11, CD8-P2-C12, CD4-P3-C2, CD4-P3-C6, CD4-P3-C10, CD4-P3-C11, CD56dim-P10-C1, CD56dim-P10-C3, CD56dim-P10-C10, CD56bright-P10-C2, CD56bright-P10-C3, CD56bright-P10-C6, CD56bright-P10-C8, CD56bright-P10-C11, CD56bright-P10-C13, CD56dim-P11-C7, CD56dim-P11-C8, CD56dim-P11-C9, CD56dim-P11-C12, CD56dim-P11-C13, CD56dim-P11-C15, CD56bright-P11-C1, CD56bright-P11-C2, CD56bright-P11-C7, CD56bright-P11-C10, CD56bright-P11-C13, CD4-P15-C4, CD4-P15-C6, CD4-P15-C9, CD4-P15-C14, CD8-P15-C1, CD8-P15-C2, CD8-P15-C4, CD8-P15-C6, CD8-P15-C10, CD8-P15-C11, CD8-P15-C12, CD8-P15-C13, CD4-P14-C2, CD4-P14-C4, CD4-P14-C6, CD8-P14-C5, CD8-P14-C9, CD8-P14-C13, CD56dim-P14-C4, CD56dim-P14-C6, CD56dim-P14-C10, CD56dim-P14-C11, CD56bright-P14-C6, CD56bright-P14-C7, CD56bright-P14-C11, CD56bright-P14-C13, CD4-P13-C1, CD4-P13-C4, CD4-P13-C6, CD4-P13-C8, CD4-P13-C10, CD4-P13-C11, CD4-P13-C12, CD4-P13-C13, CD4-P13-C16, CD8-P13-C4, CD8-P13-C5, CD8-P13-C6, CD56dim-P13-C7, CD56dim-P13-C9, CD56bright-P13-C1, CD56bright-P13-C3, CD56bright-P13-C5, CD56bright-P13-C6, CD56bright-P13-C7, and CD56bright-P13-C12.

In a preferred embodiment of the invention, a set of distinguishing parameters is ME8 according to FIG. 9 in case of k=70 (4 endophenotypes). The set of distinguishing parameters according to ME8 comprises, or consists of, the parameters CD4MCAM IL-17A, M2-like monocytes, CD8-P2-C12, CD4-P3-C11, CD56bright-P10-C8, CD56bright-P10-C13, CD56bright-P11-C1, CD56bright-P14-C7, CD56bright CD69+, CD4 CD69+, CD56dim-P11-C8, CD56bright-P13-C5, CD8 TEMRA IL-22, CD8-P15-C4, CD4-P14-C2, CD8-P15-C12, CD8-P14-C13, CD8 TEMRA GM-CSF, CD4-P13-C8, CD56bright-P13-C7, CD4-P13-C13, CD8-P15-C12, CD8-P14-C5, CD4EM IL-17A, CD4-P2-C5, CD56bright-P11-C10, CD4EM IL-4, CD56bright-P11-C2, Th17 MCAM+, CD4-P15-C4, CD56dim IFNg+, CD8-P2-C7, CD8-P2-C11, CD8-P13-C6, CD4EM TNFa, CD4M TNFa, CD4-P2-C1, CD4-P2-C3, CD56dim_P14-C10, CD8 HLA-DR+, CD4−CD8− T cells, CD8-P15-C2, CD8-P14-C9, CD8 TEMRA CD107a+, CD16highCD192− monocytes, CD4-P13-C12, NK cells, CD4-P13-C4, Th17 MCAM+, CD8-P15-C11, CD8-P13-C4, CD4-P15-C14, CD4-P13-C10, CD4-P13-C11, CD56dim-P11-C7, CD56dim-P13-C7, CD4 T cells, CD4-P14-C6, CD4-P2-C11, CD8-P15-C10, CD56bright-P11-C7, CD4-P2-C113, CD8-P2-C6, CD4-P15-C6, CD56bright-P14-C13, CD4-P13-C1, CD8CM IL-17A, CD56bright-P13-C6, Monocytes CD80+, P4-P13-C6, B naïve, and CD4CM GM-CSF.

In a preferred embodiment of the invention, a set of distinguishing parameters is ME9 according to FIG. 9 in case of k=70 (4 endophenotypes). The set of distinguishing parameters according to ME9 comprises, or consists of, the parameters CD4 CD69+, CD56bright CD69+, CD8 CD45RO−CD27−, CD4EM IL-17A, CD56dim TNFa+, CD8 TEMRA CD107a+, CD4M GrK+, M1-like monocytes, CD4-P2-C13, CD8-P2-C6, CD8-P2-C12, CD56bright-P10-C2, CD56bright-P10-C8, CD56bright-P10-C13, CD56dim-P11-C8, CD56dim-P11-C12, CD56dim-P11-C15, CD4-P15-C4, CD8-P15-C4, CD8-P14-C13, CD56dim-P14-C4, CD56dim-P14-C6, CD56dim-P14-C10, CD56bright-P14-C13, CD4-P13-C1, CD4-P13-C4, CD4-P13-C10, CD4-P13-C11, CD4-P13-C13, CD4-P13-C16, CD8-P13-C2, CD56bright-P13-C5, IFNG, IL10RA, IL2RA, PDCD1, CLSPN, CCL19, IDS, MFGE8, ALPP, TIMD4, SFTPA1, COL9A1, and IFNGR2.

In a preferred embodiment of the invention, a set of distinguishing parameters is HE1 according to FIG. 9 in case of k=70 (4 endophenotypes). The set of distinguishing parameters according to HE1 comprises the parameters CD4 CD69+, CD56bright CD69+, CD56dim CD69+, B (% Lymphocytes), RTE (% Lymp), CD4 memory CD45RO+CD45RA− (% Lymp), CD4 naive CD45RO−CD27+ (% Lymp), CD4CM IL-22, CD4EM IL-17A, CD4EM TNFa, CD4MCAM GM-CSF, CD8CM IL-17A, CD56dim CD107a+, bright IFNg+, bright MIP1b+, dim IFNg+, dim TNFa+, CTL dim CD57+ (% Lymphocytes), TEMRA CD107a+, CD4 Mem GrK+, ILC (% Lymphocytes), Lti (% Lymphocytes), CD16-CD192high (% Monos), MDSC (% Mono), M1 (% Monos), CD4-P2-C1, CD4-P2-C2, CD4-P2-C13, CD8-P2-C3, CD8-P2-C4, CD8-P2-C6, CD8-P2-C7, CD8-P2-C11, CD8-P2-C12, CD4-P3-C11, CD56dim-P10-C3, CD56dim-P10-C6, CD56bright-P10-C2, CD56bright-P10-C8, CD56bright-P10-C13, CD56dim-P11-C3, CD56dim-P11-C8, CD56dim-P11-C12, CD56dim-P11-C15, CD4-P15-C4, CD8-P15-C1, CD8-P15-C4, CD8-P15-C6, CD8-P15-C10, CD8-P15-C11, CD8-P15-C12, CD4-P14-C11, CD8-P14-C3, CD8-P14-C8, CD8-P14-C9, CD8-P14-C13, CD56dim-P14-C4, CD56dim-P14-C6, CD56dim-P14-C8, CD56dim-P14-C10, CD56dim-P14-C11, CD56bright-P14-C11, CD56bright-P14-C13, CD4-P13-C1, CD4-P13-C4, CD4-P13-C6, CD4-P13-C8, CD4-P13-C10, CD4-P13-C13, CD4-P13-C16, CD8-P13-C2, CD8-P13-C3, CD8-P13-C4, CD8-P13-C6, CD8-P13-C7, CD8-P13-C8, CD8-P13-C12, CD56dim-P13-C7, CD56bright-P13-C5, GZMH, MNDA, AHCY, ROR1, IL1RL1, IL2RA, CRTAC1, SERPINA11, DLK1, ART3, F9, NCAM1, C1QTNF1, TIMD4, CHI3L1, IL10RA, IL20RA, IFNG, TRAF2, CCL26, COL9A1, FLT3LG, CCL11, CCL4, TREM2, SLC16A1, CLSPN, NEFL, IFNGR2, TCL1A, PSME2, SMPD1, CCL19, AHSP, CA6, MYOC, MFGE8, SFTPA1, PDP1, RANGAP1, RUVBL1, PSMA1, AGR3, AKR1B1, LHB, SERPINA9, ALPP, MOG, PDCD1, PRDX6, LTA4H, KLK1, and FURIN.

In a preferred embodiment of the invention, a set of distinguishing parameters is HE2 according to FIG. 9 in case of k=70 (4 endophenotypes). The set of distinguishing parameters according to HE2 comprises, or consists of, the parameters CD14+CD16int (% Monos), CD4 CD69+, CD8 CD69+, CD56bright CD69+, B (% Lymphocytes), B class switch memory (% Lymphocytes), B IgM only (% Lymphocytes), B unusual (% Lymphocytes), CD4 (% Lymp), RTE (% Lymp), CD8 EM CD45RO+CD27− (% Lymp), Th1 (% Lymp), Th17 (% Lymp), Tfh Th17 (% Lymp), CD4CM IL-17A, CD4CM IL-22, CD4EM GM-CSF, CD4EM IFNg, CD4EM IL-17A, CD4EM TNFa, CD4M GM-CSF, CD4M IL-22, CD4M TNFa, CD4MCAM GM-CSF, CD4MCAM IL-17A, CD4MCAM IL-22, CD4MCAM TNFa, CD8CM GM-CSF, CD8CM IL-17A, CD8EM GM-CSF, CD8EM IFNg, CD8EM IL4, CD8EM TNFa, CD8M IL4, CD8M TNFa, TEMRA GM-CSF, TEMRA IL-17A, tTreg (% Lymp), CD56bright CD107a+, CD56dim CD107a+, CD56dim CD57+CD107a+, bright MIP1b+, dim IFNg+, dim TNFa+, CTL dim CD57+ (% Lymphocytes), NK mem dim NKG2C+FceR1Syk− (% Lymphocytes), TEMRA CD107a+, dim GrK+, mDC (% PBMC), CD141+mDC (% PBMC), CD86+CD141+, NK (% Lymphocytes), ILC2 (% Lymphocytes), Lti (% Lymphocytes), CD16-CD192high (% Monos), CD16highCD192− (% Monos), MDSC (% Mono), M2 (% Monos), CD80+ Monos, CD4-P2-C1, CD4-P2-C2, CD4-P2-C3, CD4-P2-C5, CD4-P2-C6, CD4-P2-C8, CD4-P2-C14, CD4-P2-C16, CD8-P2-C1, CD8-P2-C4, CD8-P2-C5, CD8-P2-C6, CD8-P2-C7, CD8-P2-C8, CD8-P2-C11, CD8-P2-C12, CD4-P3-C2, CD4-P3-C11, CD4-P3-C12, CD4-P3-C15, CD56dim-P10-C1, CD56dim-P10-C3, CD56dim-P10-C6, CD56dim-P10-C8, CD56dim-P10-C9, CD56dim-P10-C10, CD56bright-P10-C2, CD56bright-P10-C3, CD56bright-P10-C5, CD56bright-P10-C8, CD56bright-P10-C10, CD56bright-P10-C13, CD56bright-P10-C14, CD56dim-P11-C3, CD56dim-P11-C4, CD56dim-P11-C6, CD56dim-P11-C7, CD56dim-P11-C8, CD56dim-P11-C9, CD56dim-P11-C12, CD56dim-P11-C14, CD56dim-P11-C15, CD56bright-P11-C1, CD56bright-P11-C2, CD56bright-P11-C4, CD56bright-P11-C7, CD56bright-P11-C8, CD56bright-P11-C10, CD56bright-P11-C13, CD4-P15-C1, CD4-P15-C4, CD4-P15-C6, CD4-P15-C7, CD4-P15-C9, CD8-P15-C1, CD8-P15-C2, CD8-P15-C3, CD8-P15-C4, CD8-P15-C6, CD8-P15-C10, CD8-P15-C12, CD4-P14-C1, CD4-P14-C2, CD4-P14-C4, CD4-P14-C6, CD4-P14-C7, CD4-P14-C9, CD4-P14-C10, CD8-P14-C1, CD8-P14-C3, CD8-P14-C5, CD8-P14-C9, CD8-P14-C13, CD56dim-P14-C1, CD56dim-P14-C4, CD56dim-P14-C5, CD56dim-P14-C6, CD56dim-P14-C8, CD56dim-P14-C9, CD56dim-P14-C10, CD56dim-P14-C11, CD56bright-P14-C2, CD56bright-P14-C4, CD56bright-P14-C5, CD56bright-P14-C6, CD56bright-P14-C9, CD56bright-P14-C10, CD56bright-P14-C11, CD56bright-P14-C13, CD4-P13-C1, CD4-P13-C2, CD4-P13-C3, CD4-P13-C4, CD4-P13-C6, CD4-P13-C7, CD4-P13-C8, CD4-P13-C9, CD4-P13-C10, CD4-P13-C11, CD4-P13-C12, CD4-P13-C13, CD4-P13-C15, CD4-P13-C16, CD8-P13-C3, CD8-P13-C4, CD8-P13-C6, CD8-P13-C7, CD8-P13-C9, CD8-P13-C11, CD8-P13-C13, CD56dim-P13-C3, CD56dim-P13-C7, CD56dim-P13-C8, CD56dim-P13-C9, CD56bright-P13-C1, CD56bright-P13-C3, CD56bright-P13-C5, CD56bright-P13-C6, CD56bright-P13-C7, CD56bright-P13-C8, CD56bright-P13-C10, and CD56bright-P13-C11.

In a preferred embodiment of the invention, a set of distinguishing parameters is HE3 according to FIG. 9 in case of k=70 (4 endophenotypes). The set of distinguishing parameters according to HE3 comprises, or consists of, the parameters CD4 EM TNF-a, CD8-P2-C6, CD4-P15-C6, CD4M TNF-a, CD4-P15-C9, CD8 TEMRA IFN-g, CD4-P13-C12, CD4-P2-C14, CD8 TEMRA TNF-a, CD8 TEMRA CD107a+, CD4-P2-C13, CD4-P2-C1, CD4-P14-C5, CD8M TNF-a, CD8EM TNF-a, CD56dim TNF-a, CD56dim IFN-g, CD56bright-P14-C13, CD14+CD16+ monocytes, CD4-P13-C3, CD4-P2-C6, CD8-P15-C9, CD4-P2-C4, CD56bright-P14-C9, CD56dim-P14-C4, CD8-P13-C12, CD4-P2-C3, RTE T cells, CD15highCD192− monocytes, CD4-P13-C6, CD4-P15-C7, CD4CM TNF-a, CD4 CD45RO-CD27−, CD4EM IFN-g, CD56dim, CD16+CD192+ monocytes, CD4MCAM TNF-a, CD4-P14-C9, CD8-P13-C6, CD8 HLA-DR+, CD8-P14-C1, CD4-P2-C10, CD56bright-P11-C7, CD4-P13-C10CD56bright-P14-C4, CD4M IL-22, CD4-P3-C15, CD8-P2-C5, CD56bright-P13-C10, CD8M IL-17A, CD56dim-P11-C4, CD4CM IL-22, CD56bright-P14-C6, CD8CM GM-CSF, CD56bright-P13-C6, CD56bright-P10-C8, CD8-P13-C11, CD8-P14-C13, CD8-P2-C10, CD8-P14-C5, CD8-P15-C4, CD4-P14-C6, CD4-P13-C2, CD8-P2-C4, CD8-P2-C7, CD56bright-P14-C11, CD56bright-MIP1b, CD56bright-P13-C7, CD4-P13-CD12, CD56dim-P11-C8, CD56bright-P13-C8, CD4 CD69+, CD8-P14-C3, CD8-P15-C11, CD8-P2-C1, CD4-P13-C4, CD4-P15-C1, CD8-P13-C3, CD8-P15-C1, CD8-P13-C2, CD4-P13-C15, CD8 CD45RO-CD27−, CD4-P13-C9, CD8-P15-C12, CD8-P15-C2, CD4EM GM-CSF, CD8-P14-C12, CD56bright CD69+, CD56bright-P11-C4, CD8-P2-C3, CD8-P15-C6, CD16highCD192− monocytes, CD4-P13-C13, CD8-P15-C10, CD4-P2-C2, CD56dim CD69+, CD4-P13-C7, CD8 TEMRA IL-17A, CD56dim-P14-C5, CD56bright-P11-C1, CD56dim-MIP1b, CD4-P2-C9, CD4-P2-C8, CD4 CD45RO+CD27−, CD4-P13-C16, CD56bright-P13-C3, CD4-P2-C12, CD56dim-P14-C1, CD4-P2-C15, CD56dim-P11-C15, CD8-P2-C12, CD56dim-P11-C5, CD56dim-P11-C7, CD4-P3-C11, CD56dim-P10-C11, CD56bright-P10-C13, CD56dim-P11-C6, CD56dim-P10-C10, CD4-P13-C8, NEFL, LTA4H, MYOC, F9, AHCY, PRDX6, PSME2, RANGAP1, CASP8, BLVRB, SERPINA9, CNTN1, TCL1A, RARRES2, FAP, CA14, MB, ITGB1, DPP4, CD244, CTRC, CCL27, LEP, CCL7, COL9A1, PSPN, MOG, CD6, CDNF, SEMA4C, PDCD1, GPC1, GZMA, CPE, LTBP2, CR2, KLRB1, MMP12, CLSTN2, LY9, ENPP5, NECTIN4, LAT, IL6, CD160, OBP2B, RGMB, CCL26, SH2D1A, NTRK3, ART3, TNFRSF13C, ADGRG2, GCG, TSHB, TREM2, DEFB4A_DEFB4B, CA6, SEPTIN9, CHI3L1, FURIN, ALPP, APOH, RGMA, PIGR, IL1RN, PTS, and SFRP1.

In a preferred embodiment of the invention, a set of distinguishing parameters is HM1u according to FIG. 41 in case of k=190 (3 endophenotypes). HM1u is identical to HM1.

In a preferred embodiment of the invention, a set of distinguishing parameters is HM2u according to FIG. 41 in case of k=190 (3 endophenotypes). The set of distinguishing parameters according to HM2u comprises, or consists of, the parameters of LTA4H, MYOC, NEFL, PRDX6, TCL1A, WFIKKN2, NCAM1, RGMA, MOG, CCL26, AHCY, MB, GPC1, F9, SERPINA9, CR2, RGMB, CA6, ART3, SEMA4C, DNER, CNTN1, DPP4, ENPP5, and DLK1.

In a preferred embodiment of the invention, a set of distinguishing parameters is HM3u according to FIG. 41 in case of k=190 (3 endophenotypes). HM3u is identical to HM3.

In a preferred embodiment of the invention, a set of distinguishing parameters is HM4u according to FIG. 41 in case of k=190 (3 endophenotypes). HM4u is identical to HM4.

In a preferred embodiment of the invention, a set of distinguishing parameters is HM5u according to FIG. 41 in case of k=190 (3 endophenotypes). HM5u is identical to HM5.

In a preferred embodiment of the invention, a set of distinguishing parameters is HM6u according to FIG. 41 in case of k=190 (3 endophenotypes). HM6u is identical to HM6.

In a preferred embodiment of the invention, a set of distinguishing parameters is HM7u according to FIG. 41 in case of k=190 (3 endophenotypes). HM7u is identical to HM7.

In a preferred embodiment of the invention, a set of distinguishing parameters is ME1u according to FIG. 41 in case of k=190 (3 endophenotypes). The set of distinguishing parameters according to ME1u comprises, or consists of, the parameters of CD8 TEMRA CD107a+, CD16highCD192− monocytes, CD14+CD16+ monocytes, CD8 TEMRA IFNg, CD4EM TNFa, CD56bright TNFa+, CD8-P13-C6, CD56dim-P14-C5, CD4-P15-C7, CD56dim-P14-C10, CD8-P15-C1, CD8-P14-C13, CD8-P2-C3, CD8-P14-C3, CD8-P15-C6, CD4-P14-C1, CD4-P14-C6, CD4-P13-C9, CD56bright-P10-C3, CD56bright-P13-C7, CD4-P13-C6, B IgM only, CD8-P14-C1, CD4-P3-C10, B cells, CD56dim-P11-C13, CD8-P15-C9, CD4-P15-C6, CD56dim-P13-C10, CD4-P2-C6, CD4CM GM-CSF, CD8M IL-17A, CD56dim-P11-C15, CD8-P15-C10, CD56bright-P14-C8, CD56dim-P11-C5, CD4-P13-C3, CD56bright-P10-C13, CD56dim-P14-C8, CD4MCAM IL-4, CD56bright-P10-C5, CD8-P2-C12, CD4−CD8-T cells, CD4-P2-C9, CD4-P3-C5, CD8EM IL-4, CD56bright-P13-C3, CD4M GrK+, CD56brightP13-C10, CD4-P13-C12, CD8 CD69+, CD8EM IFNg, CD4-P13-C11, CD56dim CD57+, CD4-P2-C12, CD56dim-P14-C6, CD4EM IL-4, CD4-P3-C11, and CD56dim-P13-C6.

In a preferred embodiment of the invention, a set of distinguishing parameters is ME2u according to FIG. 41 in case of k=190 (3 endophenotypes). The set of distinguishing parameters according to ME2u comprises, or consists of, the parameters of CD45RA, L/D, CD127/IL-7Ra, CX3CR1, CD314/NKG2D, CD196/CCR6, CD56, CD8, CD69, CD226/DNAM-1, CD278/ICOS, CD57, CD19, CD192/CCR2, CD27, CD183/CXCR3, CD3, CD195/CCR5, CD45RO, CD14, CD62L, HLA-DR, TIGIT, CD335/NKp46, Granzyme K, Granzyme A, CD4, CD31, CD244/2B4, Perforin, CD197/CCR7, PD-1, Tim3, CD16, CD159c/NKG2C, CD194/CCR4, Granzyme B, CD185/CXCR5, CD18, CD159a/NKG2A, and CD45.

In a preferred embodiment of the invention, a set of distinguishing parameters is ME3u according to FIG. 41 in case of k=190 (3 endophenotypes). The set of distinguishing parameters according to ME3u comprises, or consists of, the parameters of CD8-P2-C6, CD8-P15-C1, and CD4-P13-C6.

In a preferred embodiment of the invention, a set of distinguishing parameters is ME4u according to FIG. 41 in case of k=190 (3 endophenotypes). The set of distinguishing parameters according to ME4u comprises, or consists of, the parameters of CD4EM IFNg, CD4EM TNFa, CD4M TNFa, CD8 TEMRA IFNg, CD8 TEMRA CD107a+, CD4-P2-C13, CD8-P2-C6, CD4-P15-C6, CD8-P15-C1, CD4-P13-C3, CD4-P13-C6, CD4-P13-C9, CD4-P13-C10, CD4-P13-C12, and CD56bright-P13-C7.

In a preferred embodiment of the invention, a set of distinguishing parameters is ME5u according to FIG. 41 in case of k=190 (3 endophenotypes). The set of distinguishing parameters according to ME5u comprises, or consists of, the parameters of CD8 CD69+, CD4EM IFNg, CD4EM TNFa, CD4M TNFa, CD8 TEMRA IFNg, CD56bright TNFa+, CD8 TEMRA CD107a+, CD4M GrK+, CD16highCD192− monocytes, CD4-P2-C9, CD4-P2-C11, CD4-P2-C13, CD8-P2-C3, CD8-P2-C6, CD4-P3-C11, CD56dim-P11-C8, CD4-P15-C6, CD4-P15-C7, CD8-P15-C1, CD8-P15-C6, CD8-P15-C9, CD8-P15-C12, CD4-P14-C5, CD8-P14-C3, CD56dim-P14-C6, CD56dim-P14-C9, CD4-P13-C3, CD4-P13-C6, CD4-P13-C9, CD4-P13-C11, CD4-P13-C12, CD8-P13-C7, and CD56bright-P13-C7.

In a preferred embodiment of the invention, a set of distinguishing parameters is ME6u according to FIG. 41 in case of k=190 (3 endophenotypes). The set of distinguishing parameters according to ME6u comprises, or consists of, the parameters of CD14+CD16+ monocytes, CD8 CD69+, CD56dim CD69+, B cells, RTE T cells, CD4EM IFNg, CD4EM IL-4, CD4EM TNFa, CD4MCAM IL-4, CD8EM IFNg, CD8M IL-17A, CD8 TEMRA IFNg, CD56bright CD107a+, CD56bright TNFa+, CD56dim TNFa+, CD56dim CD57+, CD8 TEMRA CD107a+, CD4M GrK+, CD16highCD192− monocytes, CD4-P2-C3, CD4-P2-C6, CD4-P2-C9, CD4-P2-C11, CD4-P2-C12, CD4-P2-C13, CD8-P2-C3, CD8-P2-C6, CD8-P2-C12, CD4-P3-C5, CD4-P3-C10, CD4-P3-C11, CD56bright-P10-C4, CD56dim-P11-C5, CD56dim-P11-C8, CD56dim-P11-C15, CD56bright-P11-C16, CD4-P15-C6, CD4-P15-C7, CD4-P15-C14, CD8-P15-C1, CD8-P15-C6, CD8-P15-C9, CD8-P15-C10, CD8-P15-C12, CD4-P14-C5, CD4-P14-C6, CD8-P14-C2, CD8-P14-C3, CD8-P14-C13, CD56dim-P14-C5, CD56dim-P14-C6, CD56dim-P14-C9, CD56dim-P14-C10, CD56bright-P14-C8, CD56bright-P14-C13, CD4-P13-C3, CD4-P13-C6, CD4-P13-C9, CD4-P13-C11, CD4-P13-C12, CD8-P13-C3, CD8-P13-C4, CD8-P13-C12, CD56dim-P13-C10, CD56bright-P13-C3, CD56bright-P13-C7.

In a preferred embodiment of the invention, a set of distinguishing parameters is ME7u according to FIG. 41 in case of k=190 (3 endophenotypes). The set of distinguishing parameters according to ME7u comprises, or consists of, the parameters of CD14+CD16+ monocytes, CD4−CD8− T cells, CD8 CD69+, CD56dim CD69+, B cells, B class switch memory, B IgM only, RTE T cells, Th17, CD4CM GM-CSF, CD4EM IFNg, CD4EM IL-22, CD4EM IL-4, CD4EM TNFa, CD4MCAM IL-4, CD4MCAM TNFa, CD8EM IFNg, CD8EM IL-22, CD8EM IL-4, CD8M IL-17A, CD8 TEMRA IFNg, CD8 TEMRA IL-4, CD56bright CD107a+, CD56bright TNFa+, CD56dim TNFa+, CD56dim CD57+, CD8 TEMRA CD107a+, CD4M GrK+, pDC, CD16+CD192+ monocytes, CD16highCD192− monocytes, CD4-P2-C1, CD4-P2-C3, CD4-P2-C6, CD4-P2-C9, CD4-P2-C11, CD4-P2-C12, CD4-P2-C13, CD8-P2-C3, CD8-P2-C5, CD8-P2-C6, CD8-P2-C12, CD4-P3-C5, CD4-P3-C10, CD4-P3-C11, CD56bright-P10-C2, CD56bright-P10-C3, CD56bright-P10-C4, CD56bright-P10-C5, CD56bright-P10-C11, CD56bright-P10-C13, CD56dim-P11-C5, CD56dim-P11-C8, CD56dim-P11-C15, CD56bright-P11-C10, CD56bright-P11-C16, CD4-P15-C6, CD4-P15-C7, CD4-P15-C9, CD8-P15-C1, CD8-P15-C4, CD8-P15-C6, CD8-P15-C9, CD8-P15-C10, CD8-P15-C12, CD4-P14-C1, CD4-P14-C2, CD4-P14-C5, CD4-P14-C6, CD8-P14-C1, CD8-P14-C2, CD8-P14-C3, CD8-P14-C13, CD56dim-P14-C4, CD56dim-P14-C5, CD56dim-P14-C6, CD56dim-P14-C8, CD56dim-P14-C9, CD56dim-P14-C10, CD56bright-P14-C2, CD56bright-P14-C8, CD56bright-P14-C13, CD4-P13-C3, CD4-P13-C6, CD4-P13-C9, CD4-P13-C11, CD4-P13-C12, CD8-P13-C3, CD8-P13-C4, CD8-P13-C6, CD8-P13-C9, CD8-P13-C12, CD56dim-P13-C6, CD56dim-P13-C10, CD56bright-P13-C3, CD56bright-P13-C4, CD56bright-P13-C7, CD56bright-P13-C10.

In a preferred embodiment of the invention, a set of distinguishing parameters is ME8u according to FIG. 41 in case of k=190 (3 endophenotypes). The set of distinguishing parameters according to ME8u comprises, or consists of, the parameters of CD14+CD16+ monocytes, CD4−CD8− T cells, CD8 CD69+, CD56dim CD69+, B cells, B class switch memory, B IgM only, Th17, CD4CM GM-CSF, CD4EM IL-22, CD4EM IL-4, CD4EM TNFa, CD4MCAM IL-4, CD4MCAM TNFa, CD8EM IFNg, CD8EM IL-22, CD8EM IL-4, CD8M IL-17A, CD8 TEMRA IFNg, CD8 TEMRA IL-4, CD56bright CD107a+, CD56bright TNFa+, CD56dim TNFa+, CD56dim CD57+, CD8M CD107a+, CD8 TEMRA CD107a+, CD4M GrK+, pDC, CD56bright, HSC, CD16+CD192+ monocytes, CD16highCD192− monocytes, CD4-P2-C1, CD4-P2-C3, CD4-P2-C6, CD4-P2-C9, CD4-P2-C11, CD4-P2-C12, CD4-P2-C13, CD8-P2-C3, CD8-P2-C5, CD8-P2-C6, CD8-P2-C12, CD4-P3-C5, CD4-P3-C10, CD4-P3-C11, CD56dim-P10-C1, CD56bright-P10-C3, CD56bright-P10-C4, CD56bright-P10-C5, CD56bright-P10-C6, CD56bright-P10-C11, CD56bright-P10-C13, CD56dim-P11-C1, CD56dim-P11-C5, CD56dim-P11-C8, CD56dim-P11-C13, CD56dim-P11-C15, CD56bright-P11-C10, CD56bright-P11-C16, CD4-P15-C4, CD4-P15-C6, CD4-P15-C7, CD4-P15-C9, CD8-P15-C1, CD8-P15-C4, CD8-P15-C6, CD8-P15-C9, CD8-P15-C10, CD8-P15-C12, CD4-P14-C1, CD4-P14-C2, CD4-P14-C5, CD4-P14-C6, CD4-P14-C7, CD8-P14-C1, CD8-P14-C3, CD8-P14-C13, CD56dim-P14-C4, CD56dim-P14-C5, CD56dim-P14-C6, CD56dim-P14-C8, CD56dim-P14-C9, CD56dim-P14-C10, CD56dim-P14-C11, CD56bright-P14-C2, CD56bright-P14-C4, CD56bright-P14-C8, CD56bright-P14-C11, CD56bright-P14-C12, CD56bright-P14-C13, CD4-P13-C3, CD4-P13-C6, CD4-P13-C9, CD4-P13-C11, CD4-P13-C12, CD8-P13-C3, CD8-P13-C4, CD8-P13-C6, CD8-P13-C9, CD8-P13-C12, CD56dim-P13-C6, CD56dim-P13-C10, CD56bright-P13-C3, CD56bright-P13-C4, CD56bright-P13-C7, CD56bright-P13-C10.

In a preferred embodiment of the invention, a set of distinguishing parameters is ME9u according to FIG. 41 in case of k=190 (3 endophenotypes). The set of distinguishing parameters according to ME9u comprises, or consists of, the parameters of CD14+CD16+ monocytes, CD4−CD8− T cells, CD8 CD69+, CD56dim CD69+, CD8 HLA-DR+, B cells, B naive, B class switch memory, B IgM only, Th17, CD4CM GM-CSF, CD4EM IL-22, CD4EM IL-4, CD4EM TNFa, CD4MCAM IL-4, CD4MCAM TNFa, CD8EM IFNg, CD8EM IL-17A, CD8EM IL-22, CD8EM IL-4, CD8M IL-17A, CD8 TEMRA IFNg, CD8 TEMRA IL-4, CD56bright CD107a+, CD56bright TNFa+, CD56dim IFNg+, CD56dim TNFa+, CD56dim CD57+, CD8M CD107a+, CD8 TEMRA CD107a+, CD4M GrK+, pDC, CD56bright, HSC, CD16+CD192+ monocytes, CD16highCD192− monocytes, MDSC-like, Monocytes CD86+, CD4-P2-C1, CD4-P2-C3, CD4-P2-C6, CD4-P2-C9, CD4-P2-C11, CD4-P2-C12, CD4-P2-C13, CD8-P2-C3, CD8-P2-C5, CD8-P2-C6, CD8-P2-C12, CD4-P3-C5, CD4-P3-C10, CD4-P3-C11, CD56dim-P10-C1, CD56bright-P10-C3, CD56bright-P10-C4, CD56bright-P10-C5, CD56bright-P10-C6, CD56bright-P10-C11, CD56bright-P10-C13, CD56bright-P10-C15, CD56dim-P11-C1, CD56dim-P11-C5, CD56dim-P11-C13, CD56dim-P11-C15, CD56bright-P11-C10, CD56bright-P11-C16, CD4-P15-C4, CD4-P15-C6, CD4-P15-C7, CD4-P15-C8, CD4-P15-C9, CD8-P15-C1, CD8-P15-C4, CD8-P15-C6, CD8-P15-C9, CD8-P15-C10, CD8-P15-C12, CD4-P14-C1, CD4-P14-C2, CD4-P14-C5, CD4-P14-C6, CD4-P14-C7, CD8-P14-C1, CD8-P14-C3, CD8-P14-C13, CD56dim-P14-C4, CD56dim-P14-C5, CD56dim-P14-C6, CD56dim-P14-C8, CD56dim-P14-C9, CD56dim-P14-C10, CD56dim-P14-C11, CD56bright-P14-C2, CD56bright-P14-C4, CD56bright-P14-C8, CD56bright-P14-C11, CD56bright-P14-C12, CD56bright-P14-C13, CD4-P13-C3, CD4-P13-C6, CD4-P13-C9, CD4-P13-C11, CD4-P13-C12, CD8-P13-C3, CD8-P13-C4, CD8-P13-C6, CD8-P13-C9, CD8-P13-C11, CD8-P13-C12, CD56dim-P13-C6, CD56dim-P13-C10, CD56bright-P13-C3, CD56bright-P13-C4, CD56bright-P13-C7, CD56bright-P13-C10.

In a preferred embodiment of the invention, a set of distinguishing parameters is ME10u according to FIG. 41 in case of k=190 (3 endophenotypes). The set of distinguishing parameters according to ME10u comprises, or consists of, the parameters of CD4EM IFNg, CD4EM IL-22, CD56bright TNFa+, CD8 TEMRA CD107a+, CD4M GrK+, CD4-P2-C1, CD4-P2-C9, CD4-P2-C13, CD4-P3-C5, CD4-P3-C10, CD56bright-P10-C3, CD4-P15-C6, CD8-P15-C1, CD8-P15-C6, CD8-P15-C12, CD4-P14-C8, CD8-P14-C3, CD8-P14-C13, CD56dim-P14-C3, CD56dim-P14-C8, CD56dim-P14-C10, CD56bright-P14-C2, CD56bright-P14-C8, CD4-P13-C4, CD4-P13-C6, CD4-P13-C9, CD8-P13-C2, CD8-P13-C12, and CD56bright-P13-C3.

In a preferred embodiment of the invention, a set of distinguishing parameters is ME11u according to FIG. 41 in case of k=190 (3 endophenotypes). The set of distinguishing parameters according to ME11u comprises, or consists of, the parameters of RTE T cells, CD4EM IFNg, CD4EM IL-22, CD56bright TNFa+, CD8 TEMRA CD107a+, CD4M GrK+, CD4-P2-C9, CD4-P2-C13, CD4-P3-C5, CD4-P3-C11, CD4-P15-C6, CD8-P15-C1, CD8-P15-C6, CD8-P14-C2, CD8-P14-C3, CD56dim-P14-C6, CD56bright-P14-C2, CD4-P13-C4, CD4-P13-C6, CD4-P13-C9, CD8-P13-C2, CD8-P13-C12, CD56bright-P13-C3, CELA3A, LTO1, and CD33.

In a preferred embodiment of the invention, a set of distinguishing parameters is HE1u according to FIG. 41 in case of k=190 (3 endophenotypes). The set of distinguishing parameters according to HE1u comprises, or consists of, the parameters of CD8-P2-C4, CD8-P2-C6, CD8-P15-C1, CD8-P13-C12, and LTA4H.

In a preferred embodiment of the invention, a set of distinguishing parameters is HE2u according to FIG. 41 in case of k=190 (3 endophenotypes). The set of distinguishing parameters according to HE2u comprises, or consists of, the parameters of RTE T cells, CD4EM IFNg, CD8 TEMRA CD107a+, CD8-P2-C4, CD8-P2-C6, CD4-P3-C5, CD4-P3-C11, CD4-P15-C6, CD8-P15-C1, CD8-P15-C6, CD8-P15-C12, CD8-P14-C2, CD8-P14-C3, CD8-P14-C13, CD56dim-P14-C6, CD56dim-P14-C9, CD56bright-P14-C13, CD4-P13-C4, CD4-P13-C9, CD8-P13-C12, F9, NEFL, MYOC, and LTA4H.

In a preferred embodiment of the invention, a set of distinguishing parameters is HE3u according to FIG. 41 in case of k=190 (3 endophenotypes). The set of distinguishing parameters according to HE3u comprises, or consists of, the parameters of CD56dim CD69+, RTE T cells, CD4EM IFNg, CD4EM IL-22, CD56bright TNFa+, CD56dim TNFa+, CD8M CD107a+, CD8 TEMRA CD107a+, CD4M GrK+, CD16-CD192high monocytes, CD16highCD192− monocytes, CD4-P2-C9, CD4-P2-C12, CD4-P2-C13, CD8-P2-C4, CD8-P2-C6, CD4-P3-C5, CD4-P3-C11, CD56dim-P11-C8, CD4-P15-C6, CD8-P15-C1, CD8-P15-C6, CD8-P15-C12, CD4-P14-C11, CD8-P14-C2, CD8-P14-C3, CD8-P14-C13, CD56dim-P14-C3, CD56dim-P14-C4, CD56dim-P14-C6, CD56dim-P14-C8, CD56dim-P14-C9, CD56dim-P14-C11, CD56dim-P14-C12, CD56bright-P14-C8, CD56bright-P14-C13, CD4-P13-C4, CD4-P13-C6, CD4-P13-C8, CD4-P13-C9, CD4-P13-C16, CD8-P13-C2, CD8-P13-C12, CD56bright-P13-C3, CD56bright-P13-C7, IL6, LEP, AHCY, ART3, F9, LTO1, IL24, PTX3, LTA, ADAM23, TREM2, NEFL, OBP2B, TCL1A, MYOC, LHB, MOG, PRDX6, LTA4H, CD33, and FURIN.

In a preferred embodiment of the invention, a set of distinguishing parameters is HE4u according to FIG. 41 in case of k=190 (3 endophenotypes). The set of distinguishing parameters according to HE4u comprises, or consists of, the parameters of CD56dim CD69+, B cells, B naive, RTE T cells, CD4 CD45RO+CD27−, CD4EM IFNg, CD4EM IL-22, CD4MCAM IL-4, CD8EM IFNg, CD8M GM-CSF, CD8M IFNg, CD56dim CD57+NKG2C+CD107a+, CD56bright TNFa+, CD56dim TNFa+, CD8M CD107a+, CD8 TEMRA CD107a+, CD4M GrK+, CD16-CD192high monocytes, CD16highCD192− monocytes, CD4-P2-C6, CD4-P2-C9, CD4-P2-C13, CD8-P2-C4, CD8-P2-C6, CD4-P3-C5, CD4-P3-C10, CD4-P3-C11, CD56dim-P11-C8, CD4-P15-C6, CD8-P15-C1, CD8-P15-C6, CD8-P15-C12, CD4-P14-C8, CD4-P14-C11, CD8-P14-C2, CD8-P14-C3, CD8-P14-C13, CD56dim-P14-C3, CD56dim-P14-C4, CD56dim-P14-C6, CD56dim-P14-C8, CD56dim-P14-C9, CD56dim-P14-C10, CD56dim-P14-C11, CD56dim-P14-C12, CD56bright-P14-C4, CD56bright-P14-C8, CD56bright-P14-C11, CD56bright-P14-C13, CD4-P13-C4, CD4-P13-C6, CD4-P13-C8, CD4-P13-C9, CD4-P13-C16, CD8-P13-C2, CD8-P13-C4, CD8-P13-C6, CD8-P13-C12, CD56dim-P13-C8, CD56bright-P13-C7, HK2, CEP43, IL6, MNDA, OLR1, AHCY, UMOD, CES1, CELA3A, ART3, F9, NCAM1, CHI3L1, IL20RA, IL4, LTO1, IL24, IL1A, CCL26, SELPLG, LTA, VEGFA, ADAM23, CTSC, TREM2, CTRC, NEFL, AFP, OBP2B, TCL1A, AHSP, PIGR, MYOC, MAGED1, RANGAP1, RUVBL1, STXBP3, LHB, SERPINA9, VMO1, MOG, PRDX6, HBQ1, LTA4H, CD33, KDR, KLK1, and FURIN.

In a preferred embodiment of the invention, a set of distinguishing parameters is HE5u according to FIG. 41 in case of k=190 (3 endophenotypes). The set of distinguishing parameters according to HE5u comprises, or consists of, the parameters of CD14+CD16+ monocytes, CD4−CD8− T cells, CD8 CD69+, CD56dim CD69+, CD8 HLA-DR+, B cells, B class switch memory, B IgM only, RTE T cells, CD4 CD45RO−CD27+, CD8 CD45RO+CD27−, CD4CM GM-CSF, CD4EM GM-CSF, CD4EM IFNg, CD4EM IL-22, CD4EM TNFa, CD4M IL-22, CD4M TNFa, CD4MCAM IL-22, CD8CM GM-CSF, CD8CM IFNg, CD8CM IL-17A, CD8EM IFNg, CD8EM IL-4, CD8EM TNFa, CD8M IL-17A, CD8M IL-22, CD8M IL-4, CD8 TEMRA IFNg, CD8 TEMRA IL-4, CD56bright CD107a+, CD56bright MIP1b+, CD56bright TNFa+, CD56dim TNFa+, CD56dim CD57+, CD56dim NKG2C+FceR1Syk−, CD8 EM CD107a+, CD8 TEMRA CD107a+, CD4M GrK+, mDC2, mDC2 CD86+, ILC, NK cells, ILC2, LTi, CD16-CD192high monocytes, CD16+CD192+ monocytes, CD4-P2-C1, CD4-P2-C3, CD4-P2-C6, CD4-P2-C8, CD4-P2-C9, CD4-P2-C11, CD4-P2-C12, CD8-P2-C1, CD8-P2-C4, CD8-P2-C6, CD8-P2-C8, CD4-P3-C5, CD4-P3-C10, CD4-P3-C11, CD4-P3-C12, CD56dim-P10-C6, CD56dim-P10-C9, CD56bright-P10-C5, CD56bright-P10-C6, CD56bright-P10-C8, CD56bright-P10-C11, CD56dim-P11-C5, CD56dim-P11-C7, CD56dim-P11-C8, CD56dim-P11-C14, CD56dim-P11-C15, CD56bright-P11-C2, CD56bright-P11-C4, CD56bright-P11-C13, CD4-P15-C1, CD4-P15-C4, CD4-P15-C6, CD4-P15-C7, CD4-P15-C8, CD4-P15-C9, CD4-P15-C11, CD8-P15-C1, CD8-P15-C2, CD8-P15-C6, CD8-P15-C8, CD8-P15-C9, CD8-P15-C10, CD8-P15-C12, CD4-P14-C1, CD4-P14-C4, CD4-P14-C7, CD8-P14-C1, CD8-P14-C3, CD8-P14-C13, CD56dim-P14-C1, CD56dim-P14-C4, CD56dim-P14-C5, CD56dim-P14-C6, CD56dim-P14-C8, CD56dim-P14-C10, CD56dim-P14-C11, CD56bright-P14-C2, CD56bright-P14-C4, CD56bright-P14-C5, CD56bright-P14-C6, CD56bright-P14-C8, CD56bright-P14-C9, CD56bright-P14-C10, CD56bright-P14-C11, CD56bright-P14-C12, CD56bright-P14-C13, CD4-P13-C1, CD4-P13-C2, CD4-P13-C3, CD4-P13-C4, CD4-P13-C6, CD4-P13-C9, CD4-P13-C11, CD4-P13-C12, CD4-P13-C15, CD8-P13-C6, CD8-P13-C9, CD8-P13-C12, CD56dim-P13-C3, CD56dim-P13-C6, CD56dim-P13-C10, CD56bright-P13-C3, CD56bright-P13-C7, CD56bright-P13-C8, CD56bright-P13-C9, and CD56bright-P13-C10.

In a preferred embodiment of the invention, a set of distinguishing parameters is HE6u according to FIG. 41 in case of k=190 (3 endophenotypes). The set of distinguishing parameters according to HE6u comprises, or consists of, the parameters of OMD, DSG3, ART3, TNFRSF13C, NCAM1, RGMA, ENPP5, CTRC, NOTCH3, CNTN1, ITGB1, ADAMTS13, ENG, LY9, DPP4, DNER, ADGRE5, CD244, NECTIN4, RGMB, GPC1, ADGRG2, ANGPTL7, FCRL1, LTA, MYOC, AGR2, TCL1A, SERPINA9, CCL26, CST5, IL1RL1, CA6, CR2, WFIKKN2, DLK1, MB, LTA4H, AHCY, F9, PRDX6, SEMA4C, NAMPT, MOG, LEP, NEFL, and CASP8.

In a preferred embodiment of the invention, a set of distinguishing parameters is HE7u according to FIG. 41 in case of k=190 (3 endophenotypes). The set of distinguishing parameters according to HE7u comprises, or consists of, the parameters of CD4-P13-C12, CD8 TEMRA IFNg, CD4-P15-C6, CD4EM TNFa, CD8-P2-C6, CD4-P15-C9, CD4M TNFa, CD4-P13-C6, CD8 TEMRA CD107a+, CD8-P15-C9, CD8-P13-C12, CD8 TEMRA TNFa, CD4-P14-C5, CD56dim TNFa+, CD14+CD16+ monocytes, CD4-P13-C3, CD16highCD192− monocytes, CD4-P2-C14, CD56dim IFNg+, CD56bright-P14-C4, CD4-P15-C7, CD4-P13-C10, CD4 CD45RO−CD27−, CD56dim-P14-C4, CD8-P13-C7, CD4-P2-C13, CD56bright-P14-C13, CD8M TNFa, CD56dim, CD4-P2-C4, CD16+CD192+ monocytes, CD56bright-P14-C9, CD4-P2-C1, CD56bright-P10-C4, CD4-P14-C9, CD4-P13-C4, CD8-P2-C4, CD4-P2-C3, CD56bright-P13-C8, CD56bright-P10-C8, CD56bright-P11-C1, CD4-P15-C1, CD4-P13-C9, CD8-P2-C7, CD4-P2-C12, CD4-P2-C10, CD56dim-P14-C1, CD56bright-P13-C3, CD56bright-P13-C7, CD56bright-P14-C11, CD56bright-P14-C6, CD4MCAM IFNg, CD56dim-P11-C8, CD56dim-P11-C6, CD4 CD45RO+CD27−, CD4-P2-C2, CD4-P3-C11, CD4-P2-C6, CD56bright MIP1b+, CD56bright-P10-C13, CD4 CD69+, CD56dim-P14-C5, CD8CM GM-CSF, CD56dim-P11-C7, CD4-P13-C13, CD56dim-P10-C11, CD8-P13-C6, CD4-P15-C11, CD4-P2-C9, CD56bright-P13-C10, CD8-P15-C1, CD8-P2-C1, CD4-P13-C15, CD8-P14-C3, CD8-P2-C5, CD8-P2-C3, CD8-P15-C12, CD8-P13-C2, CD8 CD45RO−CD27−, CD8-P15-C11, CD8-P13-C3, CD8-P14-C13, CD8-P14-C12, CD8 CD45RO+CD27−, CD8-P15-C10, CD8-P14-C5, CD4-P13-C2, CD8-P13-C4, CD4EM IL-22, CD56dim-P10-C9, CD8-P13-C11, CD56dim CD57+, and CD8-P15-C6.

In a preferred embodiment of the invention, a set of distinguishing parameters is HE8u according to FIG. 41 in case of k=190 (3 endophenotypes). The set of distinguishing parameters according to HE8u comprises, or consists of, the parameters of CD4-P13-C12, CD8 TEMRA IFNg, CD4-P15-C6, CD4EM TNFa, CD8-P2-C6, CD4-P15-C9, CD4M TNFa, CD4-P13-C6, CD8 TEMRA CD107a+, CD8-P15-C9, CD8-P13-C12, CD8 TEMRA TNFa, CD4-P14-C5, CD56dim TNFa+, CD14+CD16+ monocytes, CD4-P13-C3, CD16highCD192− monocytes, CD4-P2-C14, CD56dim IFNg+, CD56bright-P14-C4, CD4-P15-C7, CD4-P13-C10, CD4 CD45RO−CD27−, CD56dim-P14-C4, CD8-P13-C7, CD4-P2-C13, CD56bright-P14-C13, CD8M TNFa, CD56dim, CD4-P2-C4, CD16+CD192+ monocytes, CD56bright-P14-C9, CD4-P2-C1, CD56bright-P10-C4, CD4-P14-C9, CD4-P13-C4, CD8-P2-C4, CD4-P2-C3, CD56bright-P13-C8, CD56bright-P10-C8, CD56bright-P11-C1, CD4-P15-C1, CD4-P13-C9, CD8-P2-C7, CD4-P2-C12, CD4-P2-C10, CD56dim-P14-C1, CD56bright-P13-C3, CD56bright-P13-C7, CD56bright-P14-C11, CD56bright-P14-C6, CD4MCAM IFNg, CD56dim-P11-C8, CD56dim-P11-C6, CD4 CD45RO+CD27−, CD4-P2-C2, CD4-P3-C11, CD4-P2-C6, CD56bright MIP1b+, CD56bright-P10-C13, CD4 CD69+, CD56dim-P14-C5, CD8CM GM-CSF, CD56dim-P11-C7, CD4-P13-C13, CD56dim-P10-C11, CD8-P13-C6, CD4-P15-C11, CD4-P2-C9, CD56bright-P13-C10, CD8-P15-C1, CD8-P2-C1, CD4-P13-C15, CD8-P14-C3, CD8-P2-C5, CD8-P2-C3, CD8-P15-C12, CD8-P13-C2, CD8 CD45RO−CD27−, CD8-P15-C11, CD8-P13-C3, CD8-P14-C13, CD8-P14-C12, CD8 CD45RO+CD27−, CD8-P15-C10, CD8-P14-C5, CD4-P13-C2, CD8-P13-C4, CD4EM IL-22, CD56dim-P10-C9, CD8-P13-C11, CD56dim CD57+, CD8-P15-C6, OMD, DSG3, ART3, TNFRSF13C, NCAM1, RGMA, ENPP5, CTRC, NOTCH3, CNTN1, ITGB1, ADAMTS13, ENG, LY9, DPP4, DNER, ADGRE5, CD244, NECTIN4, RGMB, GPC1, ADGRG2, ANGPTL7, FCRL1, LTA, MYOC, AGR2, TCL1A, SERPINA9, CCL26, CST5, IL1RL1, CA6, CR2, WFIKKN2, DLK1, MB, LTA4H, AHCY, F9, PRDX6, SEMA4C, NAMPT, MOG, LEP, NEFL, and CASP8.

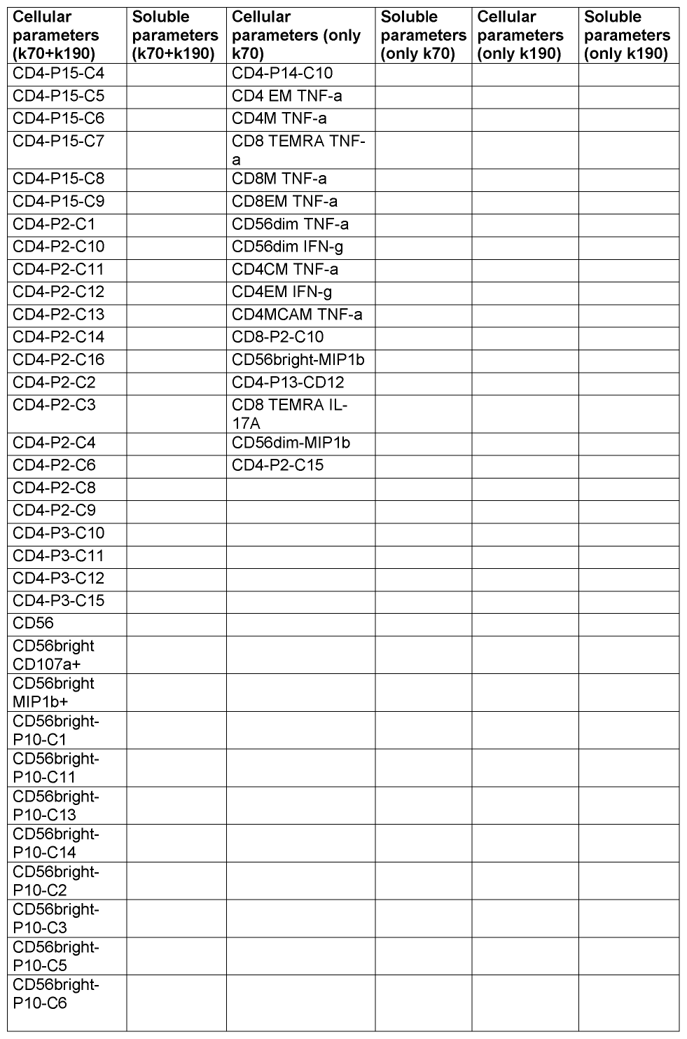

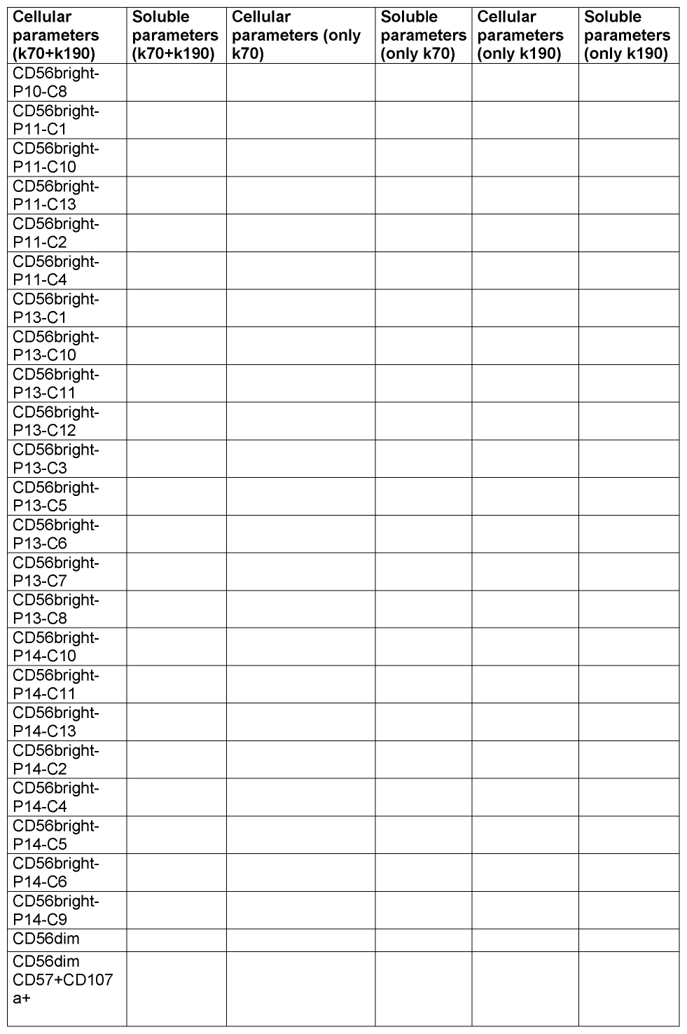

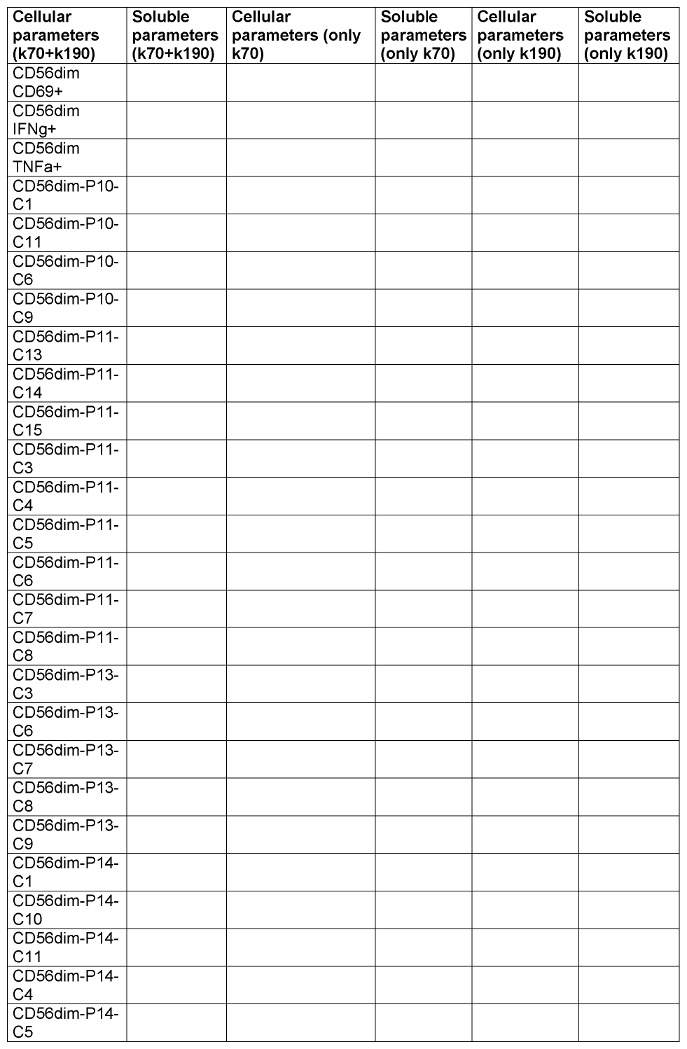

In a preferred embodiment of the invention, a set of distinguishing parameters is a set of distinguishing parameters that has been identified in both analyses using k=70 (4 endophenotypes) and k=190 (3 endophenotypes), respectively, as cellular distinguishing parameters (“Cellular parameters (k70+k190)” according to FIG. 48). This set of distinguishing parameters comprises, or consists of, the parameters of B class switch memory (% Lymphocytes), B IgM only, B IgM only (% Lymphocytes), B naive, B regulatory, bright MIP1b+, CD127, CD14, CD14+CD16+ monocytes, CD159a, CD159c, CD16, CD16+CD192+ monocytes, CD16highCD192− (% Monos), CD16highCD192− monocytes, CD18, CD183, CD185, CD19, CD192, CD194, CD195, CD196, CD197, CD226, CD244, CD27, CD3, CD31, CD314, CD335, CD4, CD4 CD45RO+CD27−, CD4 CD45RO−CD27−, CD4 CD69+, CD4 memory MCAM+, CD4 naive CD45RO−CD27+ (% Lymp), CD4+CD8+ (% Lymp), CD45, CD45RA, CD45RO, CD4−CD8− T cells, CD4CM GM-CSF, CD4CM IL-22, CD4CM IL4, CD4EM GM-CSF, CD4EM IFNg, CD4EM IL-4, CD4EM TNFa, CD4M GrK+, CD4M IL-22, CD4M TNFa, CD4MCAM IL-22, CD4MCAM TNFa, CD4-P13-C1, CD4-P13-C10, CD4-P13-C11, CD4-P13-C12, CD4-P13-C13, CD4-P13-C15, CD4-P13-C16, CD4-P13-C2, CD4-P13-C3, CD4-P13-C4, CD4-P13-C6, CD4-P13-C7, CD4-P13-C8, CD4-P13-C9, CD4-P14-C1, CD4-P14-C11, CD4-P14-C2, CD4-P14-C4, CD4-P14-C5, CD4-P14-C6, CD4-P14-C7, CD4-P14-C9, CD4-P15-C1, CD4-P15-C11, CD4-P15-C14, CD4-P15-C3, CD4-P15-C4, CD4-P15-C5, CD4-P15-C6, CD4-P15-C7, CD4-P15-C8, CD4-P15-C9, CD4-P2-C1, CD4-P2-C10, CD4-P2-C11, CD4-P2-C12, CD4-P2-C13, CD4-P2-C14, CD4-P2-C16, CD4-P2-C2, CD4-P2-C3, CD4-P2-C4, CD4-P2-C6, CD4-P2-C8, CD4-P2-C9, CD4-P3-C10, CD4-P3-C11, CD4-P3-C12, CD4-P3-C15, CD56, CD56bright CD107a+, CD56bright MIP1b+, CD56bright-P10-C1, CD56bright-P10-C11, CD56bright-P10-C13, CD56bright-P10-C14, CD56bright-P10-C2, CD56bright-P10-C3, CD56bright-P10-C5, CD56bright-P10-C6, CD56bright-P10-C8, CD56bright-P11-C1, CD56bright-P11-C10, CD56bright-P11-C13, CD56bright-P11-C2, CD56bright-P11-C4, CD56bright-P13-C1, CD56bright-P13-C10, CD56bright-P13-C11, CD56bright-P13-C12, CD56bright-P13-C3, CD56bright-P13-C5, CD56bright-P13-C6, CD56bright-P13-C7, CD56bright-P13-C8, CD56bright-P14-C10, CD56bright-P14-C11, CD56bright-P14-C13, CD56bright-P14-C2, CD56bright-P14-C4, CD56bright-P14-C5, CD56bright-P14-C6, CD56bright-P14-C9, CD56dim, CD56dim CD57+CD107a+, CD56dim CD69+, CD56dim IFNg+, CD56dim TNFa+, CD56dim-P10-C1, CD56dim-P10-C11, CD56dim-P10-C6, CD56dim-P10-C9, CD56dim-P11-C13, CD56dim-P11-C14, CD56dim-P11-C15, CD56dim-P11-C3, CD56dim-P11-C4, CD56dim-P11-C5, CD56dim-P11-C6, CD56dim-P11-C7, CD56dim-P11-C8, CD56dim-P13-C3, CD56dim-P13-C6, CD56dim-P13-C7, CD56dim-P13-C8, CD56dim-P13-C9, CD56dim-P14-C1, CD56dim-P14-C10, CD56dim-P14-C11, CD56dim-P14-C4, CD56dim-P14-C5, CD56dim-P14-C6, CD56dim-P14-C8, CD56dim-P14-C9, CD57, CD62L, CD69, CD8, CD8 CD45RO−CD27−, CD8 CD69+, CD8 EM CD107a+, CD8 HLA-DR+, CD8 TEMRA CD107a+, CD8 TEMRA IFN-g, CD80+ Monos, CD8CM GM-CSF, CD8CM IL-17A, CD8EM IFNg, CD8EM IL4, CD8EM TNFa, CD8M IL-17A, CD8M TNFa, CD8-P13-C11, CD8-P13-C12, CD8-P13-C13, CD8-P13-C2, CD8-P13-C3, CD8-P13-C4, CD8-P13-C5, CD8-P13-C6, CD8-P13-C7, CD8-P13-C9, CD8-P14-C1, CD8-P14-C12, CD8-P14-C13, CD8-P14-C3, CD8-P14-C5, CD8-P15-C1, CD8-P15-C10, CD8-P15-C11, CD8-P15-C12, CD8-P15-C2, CD8-P15-C4, CD8-P15-C6, CD8-P15-C9, CD8-P2-C1, CD8-P2-C12, CD8-P2-C3, CD8-P2-C4, CD8-P2-C5, CD8-P2-C6, CD8-P2-C7, CD8-P2-C8, CX3CR1, dim (% Lymphocytes), dim TNFa+, Granzyme A, Granzyme B, Granzyme K, HLA-DR, ICOS, Live/Dead Blue, Lti (% Lymphocytes), Memory MCAM (% Lymp), NK (% Lymphocytes), NK cells, PD-1, Perforin, RTE T cells, TEMRA CD107a+, Th1 (% Lymp), Th17 MCAM+, TIGIT, Tim3, and tTreg (% Lymp).

In a preferred embodiment of the invention, a set of distinguishing parameters is a set of distinguishing parameters that has been identified in both analyses using k=70 (4 endophenotypes) and k=190 (3 endophenotypes), respectively, as soluble distinguishing parameters (“Soluble parameters (k70+k190)” according to FIG. 48). This set of distinguishing parameters comprises, or consists of, the parameters of ADGRG2, AHCY, ART3, CA6, CASP8, CCL26, CD177, CHI3L1, CLEC7A, CNTN1, CR2, CTRC, DLK1, DPP4, ENPP5, F9, FLT3LG, FURIN, GPC1, IL1RL1, IL20RA, IL6, ITGB1, KDR, KLK1, LEP, LHB, LILRB4, LTA4H, LY9, MB, MNDA, MOG, MYOC, NCAM1, NECTIN4, NEFL, OBP2B, PIGR, PRDX6, PRSS27, RANGAP1, RGMA, RGMB, RGS8, RUVBL1, SEMA4C, SERPINA9, SLAMF1, SPINK6, STXBP3, TCL1A, TNFRSF13C, TRAF2, and TREM2.

Another set of distinguishing parameters is a set of distinguishing parameters that has been identified only in case of k=70 (4 endophenotypes) as cellular distinguishing parameters (“Cellular parameters (only k70)” according to FIG. 48). This set of distinguishing parameters comprises, or consists of, the parameters of CD28, Ki67, LAG3, CD4EM IL-17A, TEMRA GM-CSF, CD4-P2-C5, CD8-P2-C11, CD8-P14-C9, CD14+CD16+ (% Monos), CD56bright CD69+, B naive (% Lymphocytes), CD4 (% Lymp), Th17 (% Lymp), Tfh (% Lymp), Treg (% Lymp), pTreg (% Lymp), dim IFNg+, MDSC (% Mono), CD56dim-P11-C12, CD56bright-P11-C7, CD14+CD16int (% Monos), CD4−CD8− (% Lymp), B B unusual (% Lymphocytes), CD8 EMRA CD45RO−CD27− (% Lymp), Th17MCAM+ (% Lymp), CD4EM IL4, CD4MCAM GM-CSF, CD4MCAM IL-17A, TEMRA IL-22, CD4 Mem GrK+, HSC (% PBMC), M2 (% Monos), CD8-P2-C7, CD56bright-P14-C7, Lymphocytes (% PBMC), T cells (% Lymp), CD4 naive CD45RO−CD45RA+ (% Lymp), CD8 EM CD45RO+CD27− (% Lymp), bright IFNg+, CD4-P3-C6, CD56dim-P10-C10, CD8-P15-C13, Monocytes (% PBMC), CD4-P3-C2, CD56dim-P10-C3, CD56dim-P11-C9, M2-like monocytes, CD8 TEMRA IL-22, CD8 TEMRA GM-CSF, CD56dim_P14-C10, CD4 T cells, CD4-P2-C113, Monocytes CD80+, M1-like monocytes, B (% Lymphocytes), RTE (% Lymp), CD4 memory CD45RO+CD45RA− (% Lymp), CD56dim CD107a+, CTL dim CD57+ (% Lymphocytes), ILC (% Lymphocytes), CD16-CD192high (% Monos), M1 (% Monos), CD8-P14-C8, CD8-P13-C8, Tfh Th17 (% Lymp), CD4CM IL-17A, CD4M GM-CSF, CD8EM GM-CSF, CD8M IL4, TEMRA IL-17A, NK mem dim NKG2C+FceR1Syk− (% Lymphocytes), dim GrK+, mDC (% PBMC), CD141+mDC (% PBMC), CD86+CD141+, ILC2 (% Lymphocytes), CD56dim-P10-C8, CD56bright-P10-C10, CD56bright-P11-C8, CD8-P15-C3, CD4-P14-C10, CD4 EM TNF-a, CD4M TNF-a, CD8 TEMRA TNF-a, CD8M TNF-a, CD8EM TNF-a, CD56dim TNF-a, CD56dim IFN-g, CD4CM TNF-a, CD4EM IFN-g, CD4MCAM TNF-a, CD8-P2-C10, CD56bright-MIP1b, CD4-P13-CD12, CD8 TEMRA IL-17A, CD56dim-MIP1b, and CD4-P2-C15.

Another set of distinguishing parameters is a set of distinguishing parameters that has been identified only in case of k=70 (4 endophenotypes) as soluble distinguishing parameters (“Soluble parameters (only k70)” according to FIG. 48). This set of distinguishing parameters comprises, or consists of, the parameters of CPE, SEPTIN9, CA14, CDNF, IFNG, IL10RA, IL2RA, PDCD1, CLSPN, CCL19, IDS, MFGE8, ALPP, TIMD4, SFTPA1, COL9A1, IFNGR2, GZMH, ROR1, CRTAC1, SERPINA11, C1QTNF1, CCL11, CCL4, SLC16A1, PSME2, SMPD1, PDP1, PSMA1, AGR3, AKR1B1, BLVRB, RARRES2, FAP, CCL27, CCL7, PSPN, CD6, GZMA, LTBP2, KLRB1, MMP12, CLSTN2, LAT, CD160, SH2D1A, NTRK3, GCG, TSHB, DEFB4A_DEFB4B, APOH, IL1RN, PTS, and SFRP1.

Another set of distinguishing parameters is a set of distinguishing parameters that has been identified only in case of k=190 (3 endophenotypes) as cellular distinguishing parameters (“Cellular parameters (only k190)” according to FIG. 48). This set of distinguishing parameters comprises, or consists of, the parameters of CD8 TEMRA IFNg, CD56bright TNFa+, B cells, B class switch memory, CD4EM IL-22, Th17, CD4MCAM IL-4, CD4-P3-C5, CD8-P14-C2, CD4-P14-C8, CD56dim CD57+, CD8EM IL-22, CD8EM IL-4, CD56dim-P13-C10, CD8 TEMRA IL-4, CD4-P3-C5, CD56bright-P10-C4, CD56bright-P11-C16, CD56bright-P14-C8, MDSC-like, Monocytes CD86+, pDC, CD56bright, HSC, CD56brightP13-C10, CD56dim-P11-C1, CD56bright-P10-C15, CD56bright-P14-C12, CD56bright-P13-C4, CD56dim-P14-C3, CD8EM IL-17A, CD8M CD107a+, CD16-CD192high monocytes, CD8M GM-CSF, CD8M IFNg, CD56dim CD57+NKG2C+CD107a+, CD4 CD45RO−CD27+, CD8 CD45RO+CD27−, CD8CM IFNg, CD8M IL-22, CD8M IL-4, CD56dim-P14-C12, CD56dim NKG2C+FceR1Syk−, mDC2, mDC2 CD86+, ILC, ILC2, LTi, CD4MCAM IFNg, CD8 TEMRA TNFa, CD56bright-P13-C9, and CD8-P15-C8.

Another set of distinguishing parameters is a set of distinguishing parameters that has been identified only in case of k=190 (3 endophenotypes) as soluble distinguishing parameters (“Soluble parameters (only k190)” according to FIG. 48). This set of distinguishing parameters comprises, or consists of, the parameters of CELA3A, LTO1, CD33, WFIKKN2, DNER, OMD, DSG3, ADGRE5, AGR2, IL24, PTX3, LTA, ADAM23, NAMPT, HK2, CEP43, UMOD, CES1, ANGPTL7, FCRL1, CST5, NOTCH3, VMO1, HBQ1, ADAMTS13, ENG, OLR1, IL4, IL1A, SELPLG, VEGFA, CTSC, AFP, MAGED1, and RANGAP1.

In a further preferred embodiment, the set of distinguishing parameters according to the invention is for use in selecting a subject for a distinct treatment depending on the subtype, preferably an endophenotype, of said neuro-inflammatory disease, and wherein the subject is preferably suffering from a neuro-inflammatory autoimmune disease, preferably from MS.

Preferably, the set of distinguishing parameters according to the invention is for use in predicting disease progression and/or treatment response to a therapeutic or preventive treatment in a subject, wherein the therapeutic or preventive treatment is preferably selected from the group consisting of immune-modulation, immune-trafficking and/or immune cell-depletion, and wherein subject is preferably suffering from a neuro-inflammatory autoimmune disease, preferably from MS.

The inventors were able to identify and validate three or four discrete immunological endophenotypes of multiple sclerosis that provide insights into different pathological mechanisms of the disease. The inventors uncovered endophenotypes exhibiting patterns of inflammatory versus degenerative diseases features and, strikingly, revealed discrete endophenotype-specific treatment responses to commonly applied disease modifying treatments (DMTs). This approach demonstrates a vast potential of individual endophenotyping for precisely tailored treatment selection in multiple sclerosis.

According to the set of distinguishing parameters of the present invention, it is possible to determine three or four distinct endophenotypes of MS in a subject. As also be shown in the examples, one of three or four determined endophenotypes, termed “degenerative endophenotype”—E1, k=70/k=190—, was associated with a high degree of early structural damage and disability progression, while the termed “inflammatory endophenotype”, also called “IFN non responder”, —E4, k=70 and E3, k=190 respectively—, was associated with a high degree of relapse activity. The other one or two endophenotypes exhibited mixed patterns of degenerative vs. inflammatory markers. The endophenotype E2, k=70/k=190, is termed “brain atrophy”, and the endophenotype E3, k=70 is termed “converter”. The E3 endophenotype relates to a group of patients with clinical isolated syndromes—CIS—which may have an elevated risk to develop severe MS symptoms in a short time. On the background of said three or four endophenotypes, it is further possible to individually select an exact treatment for a subject suffering from MS. Moreover, it is also possible on the background of said three or four endophenotypes to predict disease progression and/or the response to a distinct therapeutic or preventive treatment in a subject.

Therefore, the new and inventive method for determining of distinguishing parameters and of course the set of distinguishing parameters per se according to the invention allows a more precise diagnosing of patients suffering from MS in form of the four distinct endophenotypes E1, E2, E3 and E4 (in case of k=70) and the three distinct endophenotypes E1, E2, and E3 (in case of k=190), respectively. Moreover, this allows in a further step the provision of an optimized treatment selection or prognosis of a treatment success of a patient. Thereby, the present invention provides an advantageous improvement of the diagnosis on the one side, and of the treatment and prevention of MS or symptoms of MS on the other side.

### Disease (Subtype) Identification Using Method According to the Invention

Distinguishing parameters and optionally or alternatively the determination model obtained by the method for determining distinguishing parameters according to the present invention can be used to classify samples, the subgroup they belong is unknown. Thus, the determination model and/or the distinguishing parameters according to the present invention are advantageous for determining a neuro-inflammatory disease, in particular a subtype of a neuro-inflammatory disease, preferably an endophenotype of a neuro-inflammatory disease, such as MS, in a subject. Hence, said subject can be efficiently and precisely stratified for eligibility to a therapeutic or preventive treatment, preferably in case of MS.

Thus, in yet another aspect, the present invention relates to a method for determining a subtype of a neuro-inflammatory autoimmune disease in a subject, the method comprising (a) comparing values of distinguishing parameters for said neuro-inflammatory autoimmune disease obtained from a sample of said subject with values of the sets of distinguishing parameters according to the invention and/or with values of distinguishing parameters as determined according to a method according to the invention, and (b) determining the subtype of said neuro-inflammatory autoimmune disease based on said comparison, preferably by conducting analytical determination, wherein said subtype is preferably an endophenotype of said neuro-inflammatory disease, preferably an endophenotype of MS selected from the group consisting of i) E1, E2, E3, and E4 or ii) E1, E2, and E3. Preferably, the neuro-inflammatory autoimmune disease is multiple sclerosis (MS) and/or the subtype is preferably a MS endophenotype.

Values of distinguishing parameters as determined according to a method for determining distinguishing parameters according to the invention described herein may be seen as reference values of said distinguishing parameters. Accordingly, it is preferred that the method is a method for determining a subtype of MS, wherein the subtype is preferably a MS endophenotype, the method comprising (a) comparing values of distinguishing parameters obtained from a sample with reference values of said distinguishing parameters, and (b) determining the subtype based on said comparison.

Preferably, the present invention relates to a method for determining whether or not a subject is suffering from a neuro-inflammatory autoimmune disease comprising
conducting the method for determining distinguishing parameters of a neuro-inflammatory disease according to the present invention and/or one of its embodiments,
wherein said group of samples comprises a sample of said subject,
wherein first subgroup of samples is obtained from healthy subjects, and
wherein the second subgroup of samples is obtained from subjects suffering from the neuro-inflammatory disease.

In yet another aspect, the present invention relates to a method for determining whether or not a subject is suffering from a subtype of a neuro-inflammatory autoimmune disease comprising
conducting the method for determining distinguishing parameters of a neuro-inflammatory disease according to the present invention and/or one of its embodiments,
wherein said group of samples comprises a sample of said subject,
wherein the first subgroup of samples is obtained from subjects suffering from a first subtype of said neuro-inflammatory disease, and
wherein the second subgroup of samples is obtained from subjects suffering from a second subtype of said neuro-inflammatory disease,
wherein said first and said second subtypes are preferably a first and a second endophenotype of said neuro-inflammatory disease.

In yet another aspect, the present invention relates to a method for determining whether or not a subject is suffering from a subtype of a neuro-inflammatory autoimmune disease comprising
conducting the method for determining distinguishing parameters of a neuro-inflammatory disease according to the present invention and/or one of its embodiments,
wherein said group of samples comprises a sample of said subject,
wherein said group of samples comprises at least
   a first subgroup of samples, wherein the first subgroup of samples is obtained from subjects suffering from a first subtype of said neuro-inflammatory disease,
   a second subgroup of samples, wherein the second subgroup of samples is obtained from subjects suffering from a second subtype of said neuro-inflammatory disease,
   a third subgroup of samples, wherein the third subgroup of samples is obtained from subjects suffering from a third subtype of said neuro-inflammatory disease,
   optionally a further (herein sometimes also referred to as “fifth”) subgroup of samples, wherein said subgroup of samples is obtained from healthy subjects, and
wherein said first, second, and third subtypes are preferably a first, second, and third endophenotype of said neuro-inflammatory disease.

In yet another aspect, the present invention relates to a method for determining whether or not a subject is suffering from a subtype of a neuro-inflammatory autoimmune disease comprising
conducting the method for determining distinguishing parameters of a neuro-inflammatory disease according to the present invention and/or one of its embodiments,
wherein said group of samples comprises a sample of said subject,
wherein said group of samples comprises at least
   a first subgroup of samples, wherein the first subgroup of samples is obtained from subjects suffering from a first subtype of said neuro-inflammatory disease,
   a second subgroup of samples, wherein the second subgroup of samples is obtained from subjects suffering from a second subtype of said neuro-inflammatory disease,
   a third subgroup of samples, wherein the third subgroup of samples is obtained from subjects suffering from a third subtype of said neuro-inflammatory disease,
   a fourth subgroup of samples, wherein the fourth subgroup of samples is obtained from subjects suffering from a fourth subtype of said neuro-inflammatory disease, and
   optionally fifth subgroup of samples, wherein the fifth subgroup of samples is obtained from healthy subjects, and
wherein said first, second, third, and fourth subtypes are preferably a first, second, third, and fourth endophenotype of said neuro-inflammatory disease.

In yet another aspect, the present invention relates to a method for determining whether or not a subject is suffering from a subtype of a neuro-inflammatory autoimmune disease comprising
determining the subject as suffering from said subtype of a neuro-inflammatory autoimmune disease based on
   (i) parameters obtained from a sample of said subject, and
   (ii) a determination model, preferably a classification model, obtained from an analytical determination approach described herein, and
   (iii) preferably further a set of distinguishing parameters obtainable or obtained by the method for determining distinguishing parameters of a neuro-inflammatory disease according to the present invention and/or one if the respective embodiments disclosed herein.

In a particularly preferred embodiment, the neuro-inflammatory disease is MS and said set of distinguishing parameters is a set of distinguishing parameters of MS, preferably of early stage MS, wherein the set comprises at least 85%, preferably 90%, more preferably 95%, even more preferably 100% of one of the sets HM1, HM2, HM3, HM4, HM5, HM6, HM7, ME1, ME2, ME3, ME4, ME5, ME6, ME7, ME8, ME9, HE1, HE2, and HE3 listed in the Table in FIG. 9, preferably wherein the set is identical to one of the sets HM1, HM2, HM3, HM4, HM5, HM6, HM7, ME1, ME2, ME3, ME4, ME5, ME6, ME7, ME8, ME9, HE1, HE2, and HE3 listed in Table of FIG. 9. Additionally or alternatively, it is particularly preferred that the neuro-inflammatory disease is MS and that the set of distinguishing parameters is a set of distinguishing parameters of MS, preferably of early stage MS, wherein the set comprises at least 85%, preferably 90%, more preferably 95%, even more preferably 100% of one of the sets HM1u, HM2u, HM3u, HM4u, HM5u, HM6u, HM7u, ME1u, ME2u, ME3u, ME4u, ME5u, ME6u, ME7u, ME8u, ME9u, ME10u, ME11u, HE1u, HE2u, HE3u, HE4u, HE5u, HE6u, HE7u, and HE8u listed in the Table in FIG. 41. Additionally or alternatively, it is particularly preferred that the neuro-inflammatory disease is MS and that the set of distinguishing parameters is a set of distinguishing parameters of MS, preferably of early stage MS, wherein the set comprises at least 85%, preferably 90%, more preferably 95%, even more preferably 100% of one of the sets “Cellular parameters (k70+k190)”, “Soluble parameters (k70+k190)”, “Cellular parameters (only k70)”, “Soluble parameters (only k70)”, “Cellular parameters (only k190)”, and “Soluble parameters (only k190)” listed in the Table in FIG. 48.

In a particularly preferred embodiment of the present invention, the neuro-inflammatory disease is MS and the set of distinguishing parameters is selected from the group consisting of HM1, HM2, HM3, HM4, HM5, HM6, HM7, ME1, ME2, ME3, ME4, ME5, ME6, ME7, ME8, ME9, HE1, HE2, and HE3 as described in detail above and as shown in FIG. 9. In a preferred embodiment of the invention, one or more or all of the sets of distinguishing parameters HM1, HM2, HM3, HM4, HM5, HM6, HM7, ME1, ME2, ME3, ME4, ME5, ME6, ME7, ME8, ME9, HE1, HE2, and HE3 as described in detail above and as shown in FIG. 9 are selected. Additionally or alternatively, it is particularly preferred that the neuro-inflammatory disease is MS and that the set of distinguishing parameters is selected from the group consisting of HM1u, HM2u, HM3u, HM4u, HM5u, HM6u, HM7u, ME1u, ME2u, ME3u, ME4u, ME5u, ME6u, ME7u, ME8u, ME9u, ME10u, ME11u, HE1u, HE2u, HE3u, HE4u, HE5u, HE6u, HE7u, and HE8u as described in detail above and as shown in FIG. 41. In a preferred embodiment of the invention, one or more or all of the sets of distinguishing parameters HM1u, HM2u, HM3u, HM4u, HM5u, HM6u, HM7u, ME1u, ME2u, ME3u, ME4u, ME5u, ME6u, ME7u, ME8u, ME9u, ME10u, ME11u, HE1u, HE2u, HE3u, HE4u, HE5u, HE6u, HE7u, and HE8u as described in detail above and as shown in FIG. 41 are selected. Additionally or alternatively, it is particularly preferred that the neuro-inflammatory disease is MS and that the set of distinguishing parameters is selected from the group consisting of “Cellular parameters (k70+k190)”, “Soluble parameters (k70+k190)”, “Cellular parameters (only k70)”, “Soluble parameters (only k70)”, “Cellular parameters (only k190)”, and “Soluble parameters (only k190)” as described in detail above and as shown in FIG. 48. In a preferred embodiment of the invention, one or more or all of the sets of distinguishing parameters “Cellular parameters (k70+k190)”, “Soluble parameters (k70+k190)”, “Cellular parameters (only k70)”, “Soluble parameters (only k70)”, “Cellular parameters (only k190)”, and “Soluble parameters (only k190)” as described in detail above and as shown in FIG. 48 are selected.

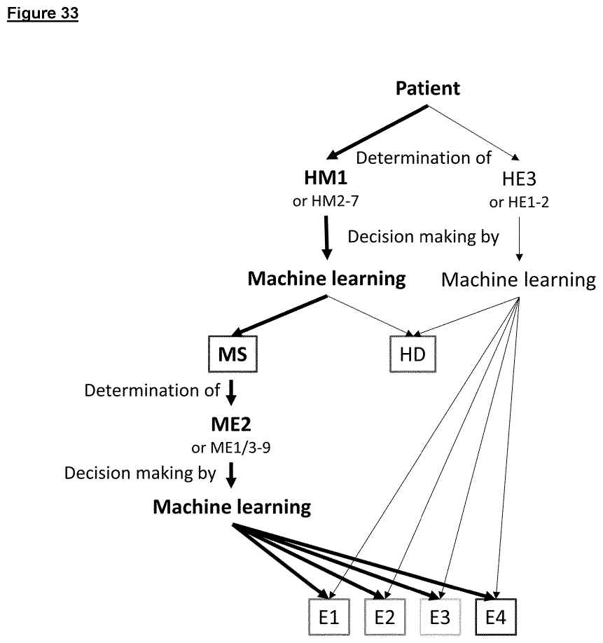

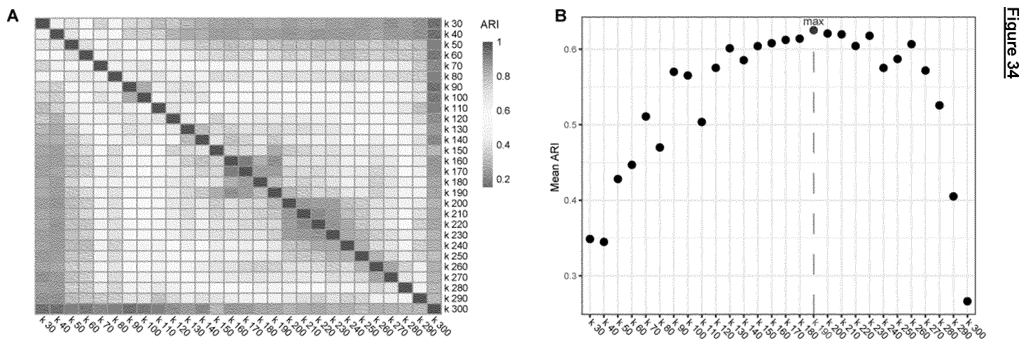

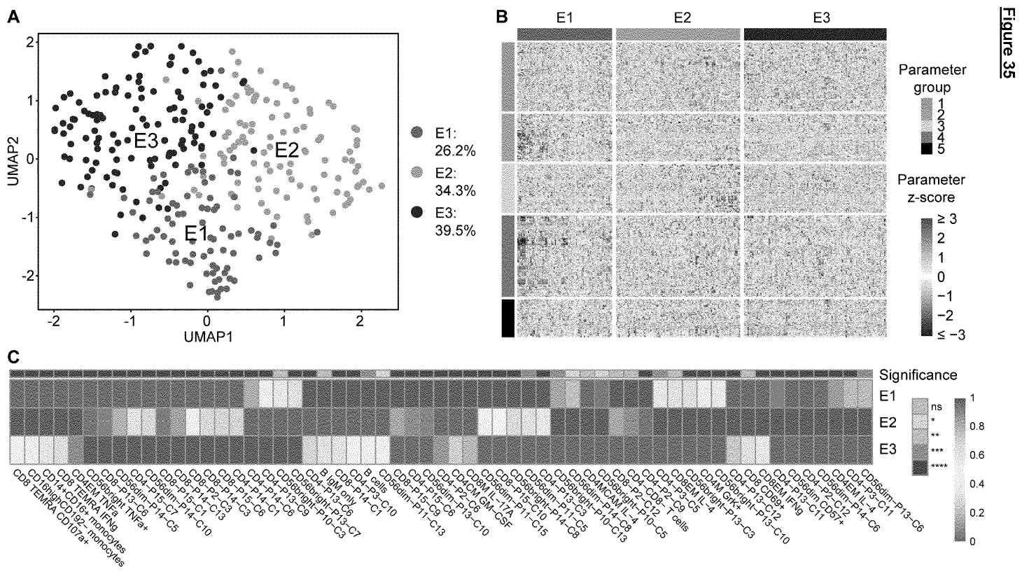

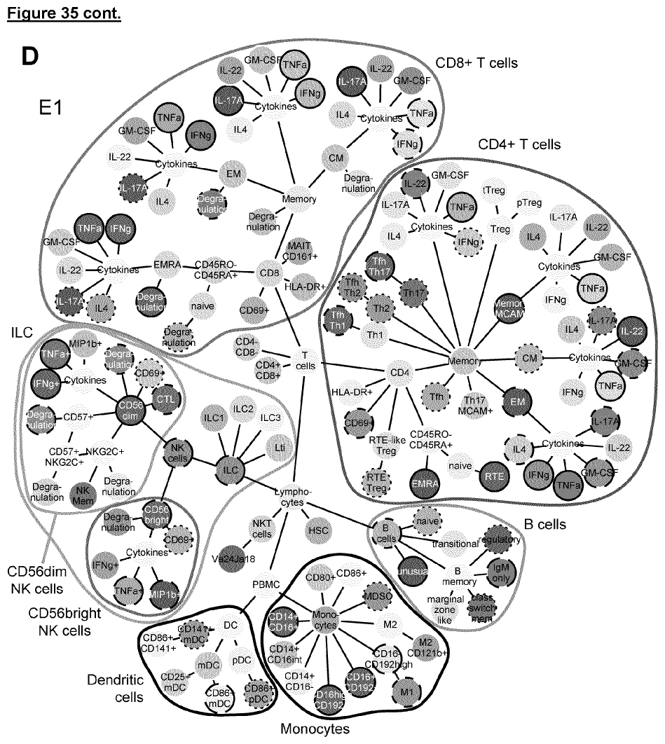

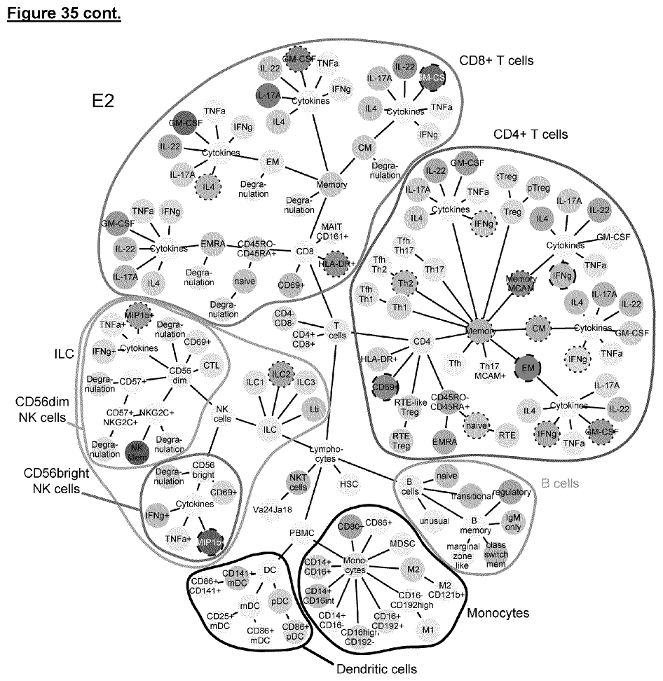

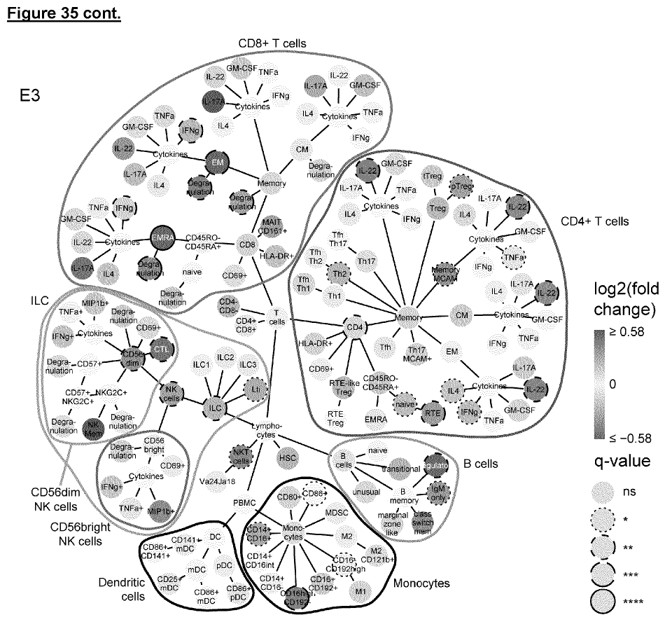

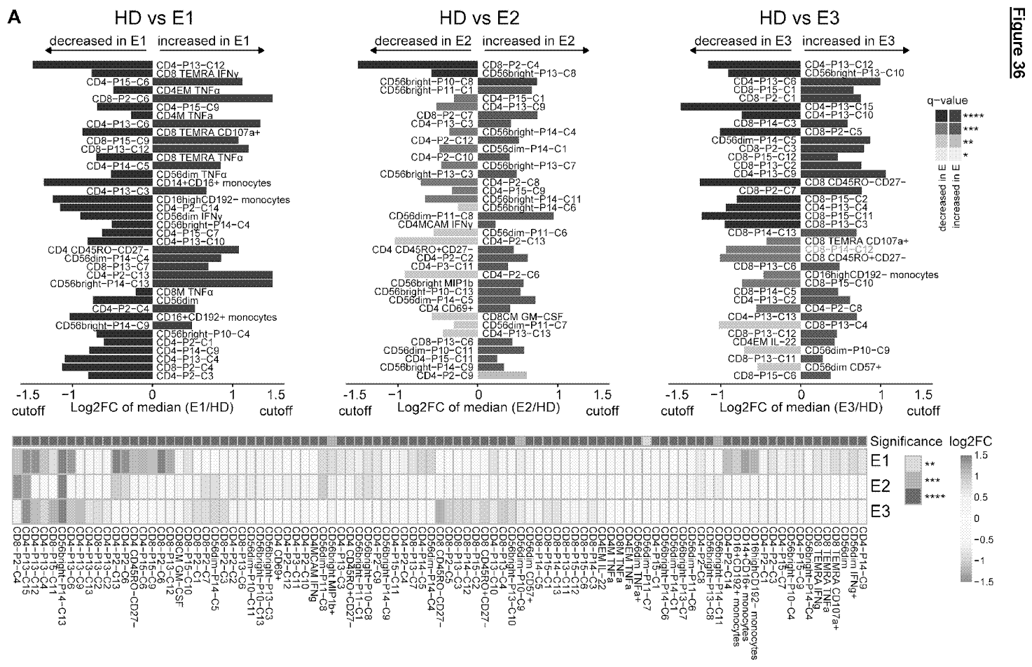

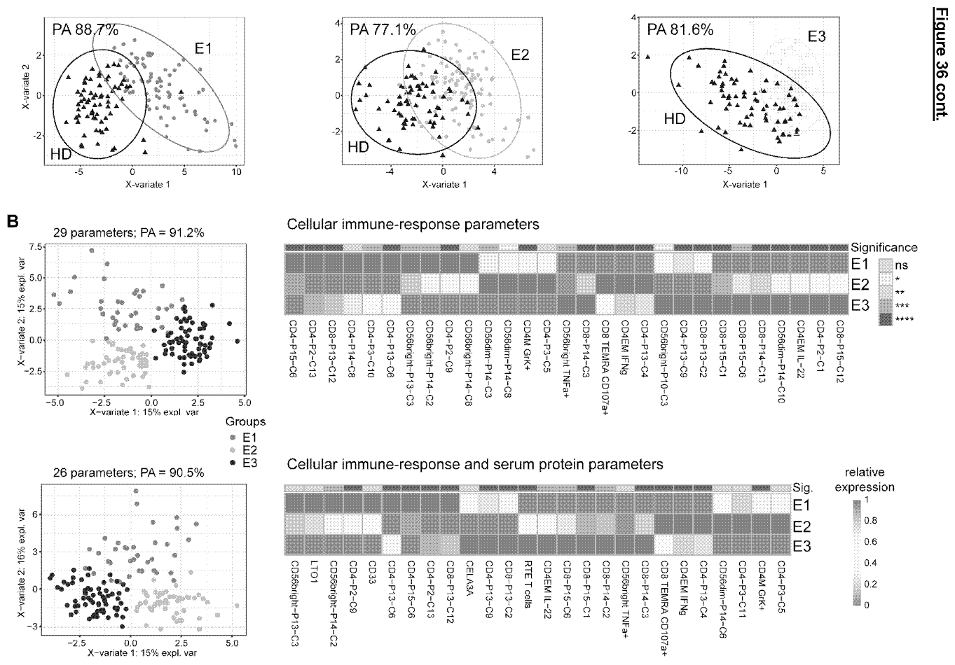

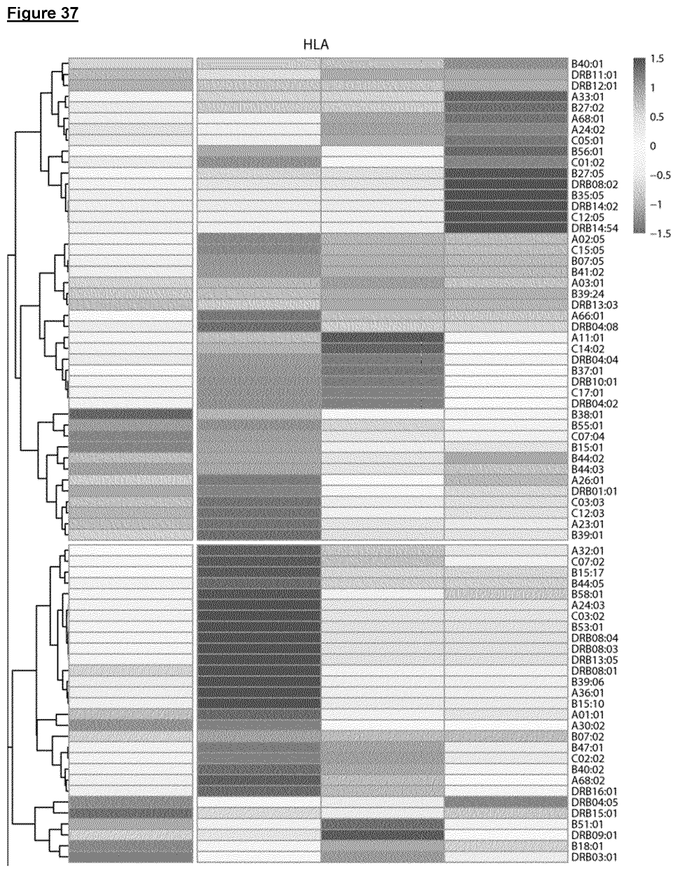

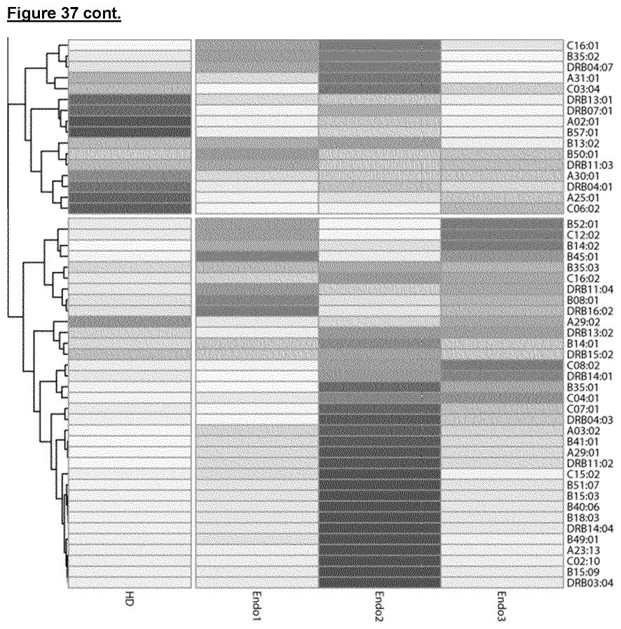

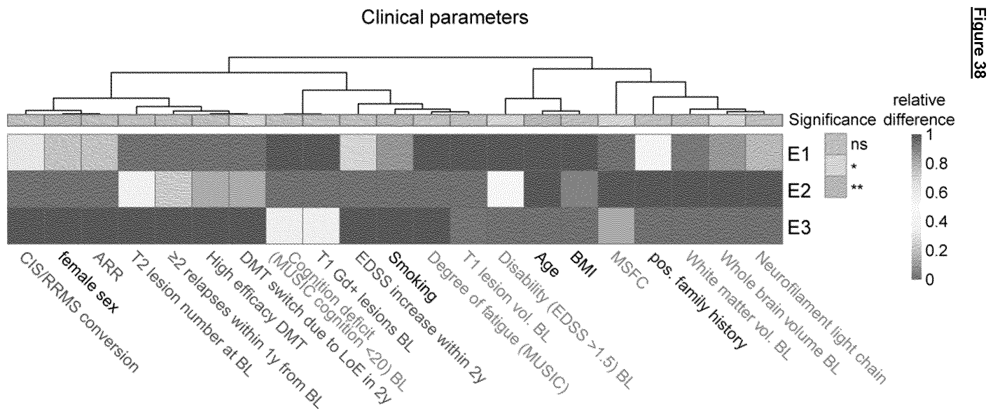

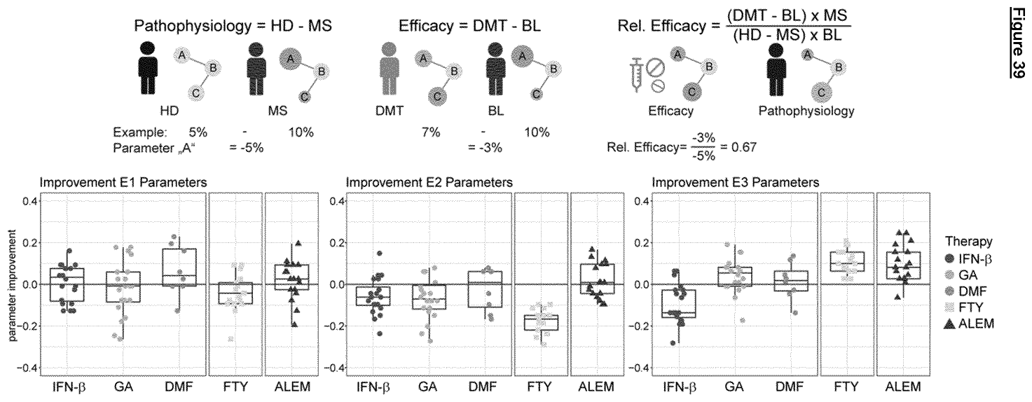

Further, in case of k=70 (4 endophenotypes), a particularly preferred embodiment of the present invention foresees the set of distinguishing parameters HM1, HM2, HM3, HM4, HM5, HM6, HM7, ME1, ME2, ME3, ME4, ME5, ME6, ME7, ME8, ME9, HE1, HE2, and HE3 as described in detail above and as shown in FIG. 9 for use in determining whether or not a subject suffers from MS, and/or optionally or additionally for use in determining MS and an endophenotype selected from the group of E1, E2, E3, E4. Said particularly preferred embodiment of the present invention is shown in the overview of FIG. 33. In a preferred embodiment of the invention, one or more or all of the sets of distinguishing parameters HM1, HM2, HM3, HM4, HM5, HM6, HM7, ME1, ME2, ME3, ME4, ME5, ME6, ME7, ME8, ME9, HE1, HE2, and HE3 as described in detail above and as shown in FIG. 9 are selected.

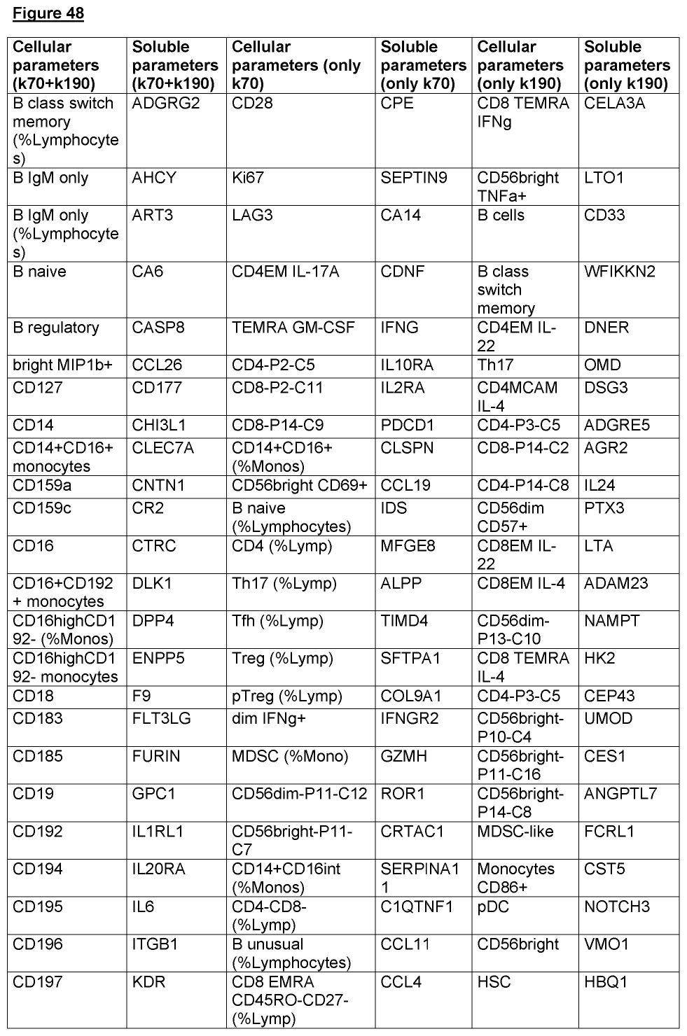

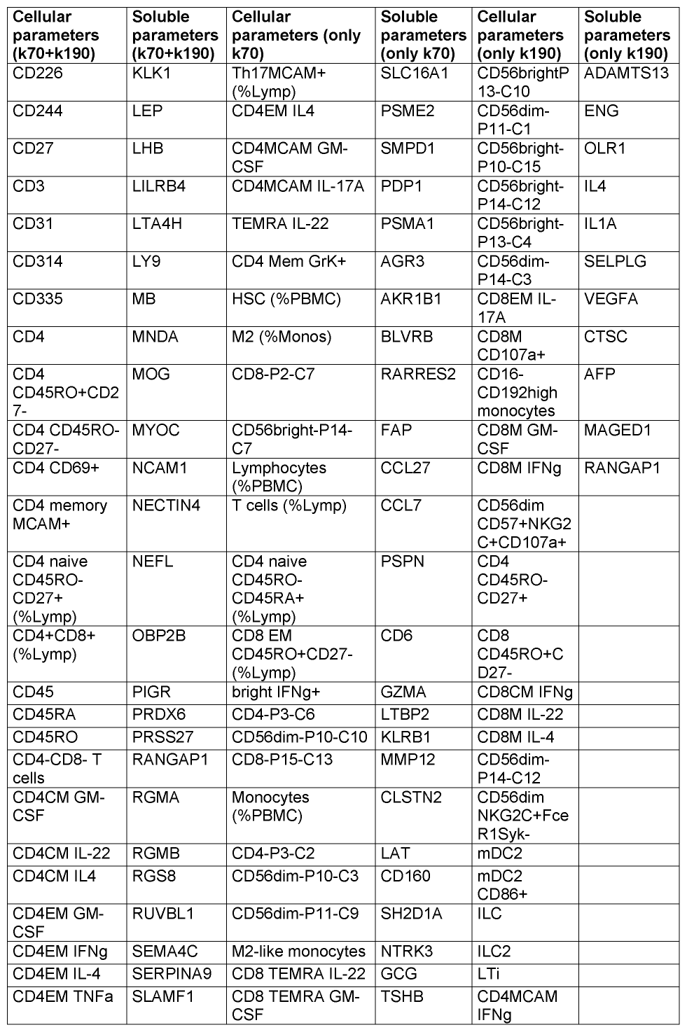

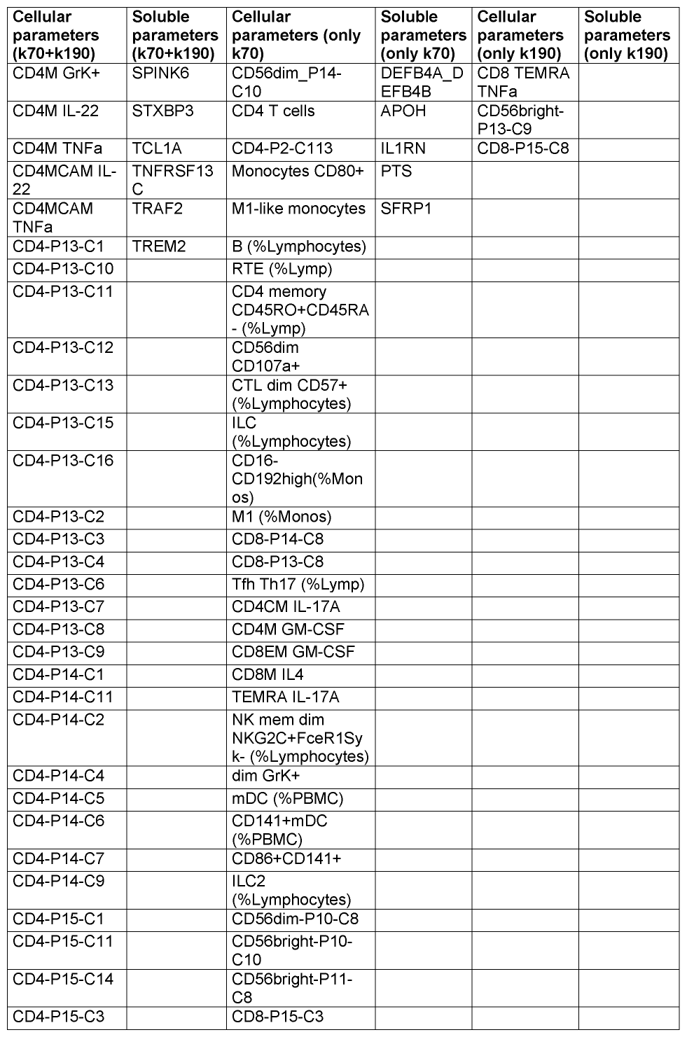

Additionally or alternatively, in case of k=190 (3 endophenotypes), a particularly preferred embodiment of the present invention foresees the set of distinguishing parameters HM1u, HM2u, HM3u, HM4u, HM5u, HM6u, HM7u, ME1u, ME2u, ME3u, ME4u, ME5u, ME6u, ME7u, ME8u, ME9u, ME10u, ME11u, HE1u, HE2u, HE3u, HE4u, HE5u, HE6u, HE7u, and HE8u as described in detail above and as shown in FIG. 41 for use in determining whether or not a subject suffers from MS, and/or optionally or additionally for use in determining MS and an endophenotype selected from the group of E1, E2, and E3. Said particularly preferred embodiment of the present invention is shown in the overview of FIG. 47. In a preferred embodiment of the invention, one or more or all of the sets of distinguishing parameters HM1u, HM2u, HM3u, HM4u, HM5u, HM6u, HM7u, ME1u, ME2u, ME3u, ME4u, ME5u, ME6u, ME7u, ME8u, ME9u, ME10u, ME11u, HE1u, HE2u, HE3u, HE4u, HE5u, HE6u, HE7u, and HE8u as described in detail above and as shown in FIG. 41 are selected.

Additionally or alternatively, a particularly preferred embodiment of the present invention foresees the set of distinguishing parameters “Cellular parameters (k70+k190)”, “Soluble parameters (k70+k190)”, “Cellular parameters (only k70)”, “Soluble parameters (only k70)”, “Cellular parameters (only k190)”, and “Soluble parameters (only k190)” as described in detail above and as shown in FIG. 48 for use in determining whether or not a subject suffers from MS, and/or optionally or additionally for use in determining MS and an endophenotype selected from the group of i) E1, E2, E3, and E4 and/or ii) E1, E2, and E3. In a preferred embodiment of the invention, one or more or all of the sets of distinguishing parameters “Cellular parameters (k70+k190)”, “Soluble parameters (k70+k190)”, “Cellular parameters (only k70)”, “Soluble parameters (only k70)”, “Cellular parameters (only k190)”, and “Soluble parameters (only k190)” as described in detail above and as shown in FIG. 48 are selected.

### Disease Subtype Identification Using Database

In yet another aspect, the present invention relates to a method for determining a subtype of a neuro-inflammatory autoimmune disease a subject is suffering from comprising
determining the subtype of said disease based on the comparison of values of a set of distinguishing parameters for said neuro-inflammatory autoimmune disease obtained from a sample of said subject with values of said set of distinguishing parameters obtained from a database,
wherein said set of distinguishing factors is obtainable or obtained by the method for determining distinguishing parameters of a neuro-inflammatory disease according to the present invention and/or one if the respective embodiments disclosed herein,
wherein preferably said set comprises at least 85%, preferably 90%, more preferably 95%, even more preferably 100% of one of the sets listed in the Table in FIG. 9 and/or in the Table in FIG. 41 and/or in the Table in FIG. 48, preferably wherein said set is identical to one of the sets listed in the Table in FIG. 9 and/or in the Table in FIG. 41 and/or in the Table in FIG. 48,
   wherein said set of distinguishing parameters is comprised in said database,
wherein preferably said database further comprises values of said distinguishing parameters obtained from samples used in said method for determining distinguishing parameters of a neuro-inflammatory disease, and
   wherein said subtype is preferably an endophenotype of said neuro-inflammatory disease, preferably an endophenotype of MS selected from the group consisting of i) E1, E2, E3, and E4 or ii) E1, E2, and E3.

Thus, when conducting the method for determining distinguishing parameters according to the present invention and/or one of its embodiments, distinguishing parameters are obtained as well as respective values for the group of samples used. Said values can be summarized and provided in form of a database, for example by providing ranges of obtained values per distinguishing parameter and per subgroup comprised in the group of samples analysed. Said database may then be used to compare values of at least a part of the distinguishing parameters comprised in said database between a sample of the subject under study and respective database entries. This is advantageous as determination, especially classification, of disease subtypes, preferably of MS endophenotypes, can be obtained fast and, optionally even without computational effort.

### Computer Implementation

It is particularly preferred that the method for determining distinguishing parameters according to the present invention and/or its embodiments is a computer-implemented method.

It is also particularly preferred that the method for determining a subtype of a neuro-inflammatory disease according to the present invention and/or its embodiments is a computer-implemented method.

In yet another aspect, the present invention relates to a computer program comprising instructions to cause a computer to execute the steps of at least one of the computer-implemented methods according to the invention and/or at least one of the respective embodiments disclosed herein.

In yet another aspect, the present invention relates to a computer-readable medium having stored thereon said computer program, optionally further at least one of the sets listed in the Table in FIG. 9 and/or in the Table in FIG. 41 and/or in the Table in FIG. 48.

### Uses

In yet another aspect, the present invention relates to the use of a set of distinguishing parameters for determining whether or not a subject suffers from multiple sclerosis (MS) and/or a MS subtype, wherein said MS subtype is preferably a MS endophenotype, and wherein said set of distinguishing parameters comprises, or consists of, “Cellular parameters (k70+k190)” as described in detail above and as shown in FIG. 48. As regards the set of distinguishing parameters, the same applies as stated herein above including features and advantages mentioned in the context of the respective embodiments described herein above (both embodiments of sets and of sets for use).

In some embodiments, the set of distinguishing parameters comprises, or consists of, “Cellular parameters (k70+k190)” as described in detail above and as shown in FIG. 48 as well as “Soluble parameters (k70+k190)” as described in detail above and as shown in FIG. 48. In some embodiments, the set of distinguishing parameters comprises, or consists of, “Cellular parameters (k70+k190)” as described in detail above and as shown in FIG. 48 as well as “Cellular parameters (only k70)” and/or “Soluble parameters (only k70)” as described in detail above and as shown in FIG. 48. In some embodiments, the set of distinguishing parameters comprises, or consists of, “Cellular parameters (k70+k190)” and “Soluble parameters (k70+k190)” as described in detail above and as shown in FIG. 48 as well as “Cellular parameters (only k70)” and/or “Soluble parameters (only k70)” as described in detail above and as shown in FIG. 48. In preferred embodiments, the set of distinguishing parameters comprises, or consists of, “Cellular parameters (k70+k190)” as described in detail above and as shown in FIG. 48 as well as “Cellular parameters (only k190)” and/or “Soluble parameters (only k190)” as described in detail above and as shown in FIG. 48. In preferred embodiments, the set of distinguishing parameters comprises, or consists of, “Cellular parameters (k70+k190)” and “Soluble parameters (k70+k190)” as described in detail above and as shown in FIG. 48 as well as “Cellular parameters (only k190)” and/or “Soluble parameters (only k190)” as described in detail above and as shown in FIG. 48. In preferred embodiments, the set of distinguishing parameters comprises, or consists of, “Cellular parameters (k70+k190)”, “Soluble parameters (k70+k190)”, “Cellular parameters (only k190)” and “Soluble parameters (only k190)” as described in detail above and as shown in FIG. 48.

In preferred embodiments, the set comprising, or consisting of, HM5, HM6 and/or HM7 is used for determining whether or not the subject suffers from MS. Additionally or alternatively, it is preferred that the set comprising, or consisting of, HM5u, HM6u and/or HM7u is used for determining whether or not the subject suffers from MS.

In preferred embodiments, the set comprising, or consisting of, ME1, ME2, ME3, ME4, ME5, ME6, ME7, ME8 and/or ME9 is used for determining the MS endophenotype. Additionally or alternatively, it is preferred that the set comprising, or consisting of, ME1u, ME2u, ME3u, ME4u, ME5u, ME7u, ME8u, ME9u, ME10u and/or ME11u is used for determining the MS endophenotype.

In preferred embodiments, the set comprising, or consisting of, HE1, HE2 and/or HE3 is used for determining whether or not a subject suffers from a MS endophenotype and for determining the same, preferably wherein the set comprises, or consists of HE1 and/or HE3. Additionally or alternatively, it is preferred that the set comprising, or consisting of, HE1u, HE2u, HE3u, HE4u, HE5u, HE6u, HE7u and/or HE8u is used for determining whether or not a subject suffers from a MS endophenotype and for determining the same, preferably wherein the set comprises, or consists of, HE1u, HE2u, HE3u, HE4u, and/or HE8u.

In yet another aspect, the present invention relates to the use of a set of distinguishing parameters for electing a subject suffering from MS for a treatment depending on MS subtype, wherein said MS subtype is preferably a MS endophenotype, and wherein said set of distinguishing parameters comprises, or consists of, “Cellular parameters (k70+k190)” as described in detail above and as shown in FIG. 48. As regards the set of distinguishing parameters, the same applies as stated herein above including features and advantages mentioned in the context of the respective embodiments described herein above (both embodiments of sets and of sets for use).

In some embodiments, the set of distinguishing parameters comprises, or consists of, “Cellular parameters (k70+k190)” as described in detail above and as shown in FIG. 48 as well as “Soluble parameters (k70+k190)” as described in detail above and as shown in FIG. 48. In some embodiments, the set of distinguishing parameters comprises, or consists of, “Cellular parameters (k70+k190)” as described in detail above and as shown in FIG. 48 as well as “Cellular parameters (only k70)” and/or “Soluble parameters (only k70)” as described in detail above and as shown in FIG. 48. In some embodiments, the set of distinguishing parameters comprises, or consists of, “Cellular parameters (k70+k190)” and “Soluble parameters (k70+k190)” as described in detail above and as shown in FIG. 48 as well as “Cellular parameters (only k70)” and/or “Soluble parameters (only k70)” as described in detail above and as shown in FIG. 48. In preferred embodiments, the set of distinguishing parameters comprises, or consists of, “Cellular parameters (k70+k190)” as described in detail above and as shown in FIG. 48 as well as “Cellular parameters (only k190)” and/or “Soluble parameters (only k190)” as described in detail above and as shown in FIG. 48. In preferred embodiments, the set of distinguishing parameters comprises, or consists of, “Cellular parameters (k70+k190)” and “Soluble parameters (k70+k190)” as described in detail above and as shown in FIG. 48 as well as “Cellular parameters (only k190)” and/or “Soluble parameters (only k190)” as described in detail above and as shown in FIG. 48. In preferred embodiments, the set of distinguishing parameters comprises, or consists of, “Cellular parameters (k70+k190)”, “Soluble parameters (k70+k190)”, “Cellular parameters (only k190)” and “Soluble parameters (only k190)” as described in detail above and as shown in FIG. 48.

In preferred embodiments, the set comprising, or consisting of, HM5, HM6 and/or HM7 is used for determining whether or not the subject suffers from MS. Additionally or alternatively, it is preferred that the set comprising, or consisting of, HM5u, HM6u and/or HM7u is used for determining whether or not the subject suffers from MS.

In preferred embodiments, the set comprising, or consisting of, ME1, ME2, ME3, ME4, ME5, ME6, ME7, ME8 and/or ME9 is used for determining the MS endophenotype. Additionally or alternatively, it is preferred that the set comprising, or consisting of, ME1u, ME2u, ME3u, ME4u, ME5u, ME7u, ME8u, ME9u, ME10u and/or ME11u is used for determining the MS endophenotype.

In preferred embodiments, the set comprising, or consisting of, HE1, HE2 and/or HE3 is used for determining whether or not a subject suffers from a MS endophenotype and for determining the same, preferably wherein the set comprises, or consists of HE1 and/or HE3. Additionally or alternatively, it is preferred that the set comprising, or consisting of, HE1u, HE2u, HE3u, HE4u, HE5u, HE6u, HE7u and/or HE8u is used for determining whether or not a subject suffers from a MS endophenotype and for determining the same, preferably wherein the set comprises, or consists of, HE1u, HE2u, HE3u, HE4u, and/or HE8u.

In yet another aspect, the present invention relates to the use of a set of distinguishing parameters for predicting disease progression and/or treatment response to a therapeutic and/or preventive treatment in a subject suffering from MS depending on MS subtype, preferably wherein the therapeutic or preventive treatment is selected from the group consisting of immune-modulation, immune-trafficking and/or immune cell-depletion, wherein said MS subtype is preferably a MS endophenotype, and wherein said set of distinguishing parameters comprises, or consists of, “Cellular parameters (k70+k190)” as described in detail above and as shown in FIG. 48. As regards the set of distinguishing parameters, the same applies as stated herein above including features and advantages mentioned in the context of the respective embodiments described herein above (both embodiments of sets and of sets for use).

In some embodiments, the set of distinguishing parameters comprises, or consists of, “Cellular parameters (k70+k190)” as described in detail above and as shown in FIG. 48 as well as “Soluble parameters (k70+k190)” as described in detail above and as shown in FIG. 48. In some embodiments, the set of distinguishing parameters comprises, or consists of, “Cellular parameters (k70+k190)” as described in detail above and as shown in FIG. 48 as well as “Cellular parameters (only k70)” and/or “Soluble parameters (only k70)” as described in detail above and as shown in FIG. 48. In some embodiments, the set of distinguishing parameters comprises, or consists of, “Cellular parameters (k70+k190)” and “Soluble parameters (k70+k190)” as described in detail above and as shown in FIG. 48 as well as “Cellular parameters (only k70)” and/or “Soluble parameters (only k70)” as described in detail above and as shown in FIG. 48. In preferred embodiments, the set of distinguishing parameters comprises, or consists of, “Cellular parameters (k70+k190)” as described in detail above and as shown in FIG. 48 as well as “Cellular parameters (only k190)” and/or “Soluble parameters (only k190)” as described in detail above and as shown in FIG. 48. In preferred embodiments, the set of distinguishing parameters comprises, or consists of, “Cellular parameters (k70+k190)” and “Soluble parameters (k70+k190)” as described in detail above and as shown in FIG. 48 as well as “Cellular parameters (only k190)” and/or “Soluble parameters (only k190)” as described in detail above and as shown in FIG. 48. In preferred embodiments, the set of distinguishing parameters comprises, or consists of, “Cellular parameters (k70+k190)”, “Soluble parameters (k70+k190)”, “Cellular parameters (only k190)” and “Soluble parameters (only k190)” as described in detail above and as shown in FIG. 48.

In preferred embodiments, the set comprising, or consisting of, HM5, HM6 and/or HM7 is used for determining whether or not the subject suffers from MS. Additionally or alternatively, it is preferred that the set comprising, or consisting of, HM5u, HM6u and/or HM7u is used for determining whether or not the subject suffers from MS.

In preferred embodiments, the set comprising, or consisting of, ME1, ME2, ME3, ME4, ME5, ME6, ME7, ME8 and/or ME9 is used for determining the MS endophenotype. Additionally or alternatively, it is preferred that the set comprising, or consisting of, ME1u, ME2u, ME3u, ME4u, ME5u, ME7u, ME8u, ME9u, ME10u and/or ME11u is used for determining the MS endophenotype.

In preferred embodiments, the set comprising, or consisting of, HE1, HE2 and/or HE3 is used for determining whether or not a subject suffers from a MS endophenotype and for determining the same, preferably wherein the set comprises, or consists of HE1 and/or HE3. Additionally or alternatively, it is preferred that the set comprising, or consisting of, HE1u, HE2u, HE3u, HE4u, HE5u, HE6u, HE7u and/or HE8u is used for determining whether or not a subject suffers from a MS endophenotype and for determining the same, preferably wherein the set comprises, or consists of, HE1u, HE2u, HE3u, HE4u, and/or HE8u.

A further aspect of the present invention relates to a set of distinguishing parameters for use in diagnosing multiple sclerosis (MS), wherein the diagnosing comprises one or more of the following: (i) determining whether or not a subject suffers from multiple sclerosis (MS) and/or determining an MS subtype; (ii) electing a subject suffering from MS for a treatment depending on MS subtype; (iii) predicting disease progression and/or treatment response to a therapeutic and/or preventive treatment in a subject suffering from MS depending on MS subtype; wherein said MS subtype is preferably a MS endophenotype.

Preferably, the set of distinguishing parameters for use according to the invention is set of distinguishing parameters comprises one or more or all of the parameters and/or parameter sets as defined above or in the claims.

Further preferred, the set of distinguishing parameters for use according to the invention is in case of i) the set of
HM1, HM2, HM3, HM4, HM5, HM6 and/or HM7, or of
HM1u, HM2u, HM3u, HM4u, HM5u, HM6u and/or HM7u,
   wherein the set is used for determining whether or not the subject suffers from MS.

Preferably, the set of distinguishing parameters for use according to the invention is the set of
ME1, ME2, ME3, ME4, ME5, ME6, ME7, ME8 and/or ME9 or of
ME1u, ME2u, ME3u, ME4u, ME5u, ME6u, ME7u, ME8u, ME9u, ME10u and/or ME11u,
   wherein is used for determining the MS endophenotype.

Further preferred, the set of distinguishing parameters for use according to the invention is the set of
HE1, HE2 and/or HE3, or of
HE1u, HE2u, HE3u, HE4u, HE5u, HE6u, HE7u and/or HE8u,
   wherein the set is used for determining whether or not a subject suffers from an MS endophenotype and for determining said MS endophenotype, preferably wherein the set of distinguishing parameters is
HE1 and/or HE3, or
HE1u, HE2u, HE3u, HE4u and/or HE8u.

Preferably, the set of distinguishing parameters for use according to the invention is such that the therapeutic or preventive treatment is selected from the group consisting of immune-modulation, immune-trafficking and/or immune cell-depletion.

### EXAMPLES OF THE INVENTION

Methods and materials are described herein for use in the present disclosure; other, suitable methods and materials known in the art can also be used. The materials, methods, and examples are illustrative only and not intended to be limiting.

### Summary

An exemplarily study was conducted to identify MS endophenotypes using high-dimensional blood immune phenotyping and computational biology based on a multicentric cohort with >1000 therapy-naïve early (≤2 years from disease onset) MS patients and a prospective comprehensive and standardized clinical data collection (NationMS cohort), paired with additional extensive cellular and non-cellular biomaterial collection in a sub-cohort of 378 MS patients. Biospecimens were subjected to a high dimensional characterization of peripheral immune-response signatures by combining multi-parameter flow cytometry and targeted proteomics, which allowed differentiation of individuals into clinically distinct subgroups (FIG. 47).

With the aim to provide individualized diagnostic and treatment for distinct patient subgroups, the inventors conducted the following experimental results based on a multicentric cohort with >1000 therapy-naïve early (≤2 years from disease onset) multiple sclerosis patients and a prospective comprehensive and standardized clinical data collection (NationMS cohort; (Ostkamp et al., 2021; von Bismarck et al., 2018)), paired with additional extensive cellular and non-cellular biomaterial collection in a sub-cohort of 378 multiple sclerosis patients. Biospecimens were subjected to a high dimensional characterization of peripheral immune-response signatures by combining multi-parameter flow cytometry and targeted proteomics. The inventors find that in early multiple sclerosis cellular immune-response signatures split into three or four distinct immunological endophenotypes. One of these three or four discovered endophenotypes, that the inventors termed “degenerative endophenotype”, was associated with a high degree of early structural damage and disability progression, while the “inflammatory endophenotype” was associated with a high degree of relapse activity. The other one or two endophenotypes exhibited mixed patterns of degenerative vs. inflammatory markers. Distinct immune-therapeutic principles differed in their capacity to reverse alterations associated with each endophenotype. Thus, stratification of multiple sclerosis patients based on peripheral immune-response signatures with the potential to guide personalized multiple sclerosis treatment decisions.

### Results

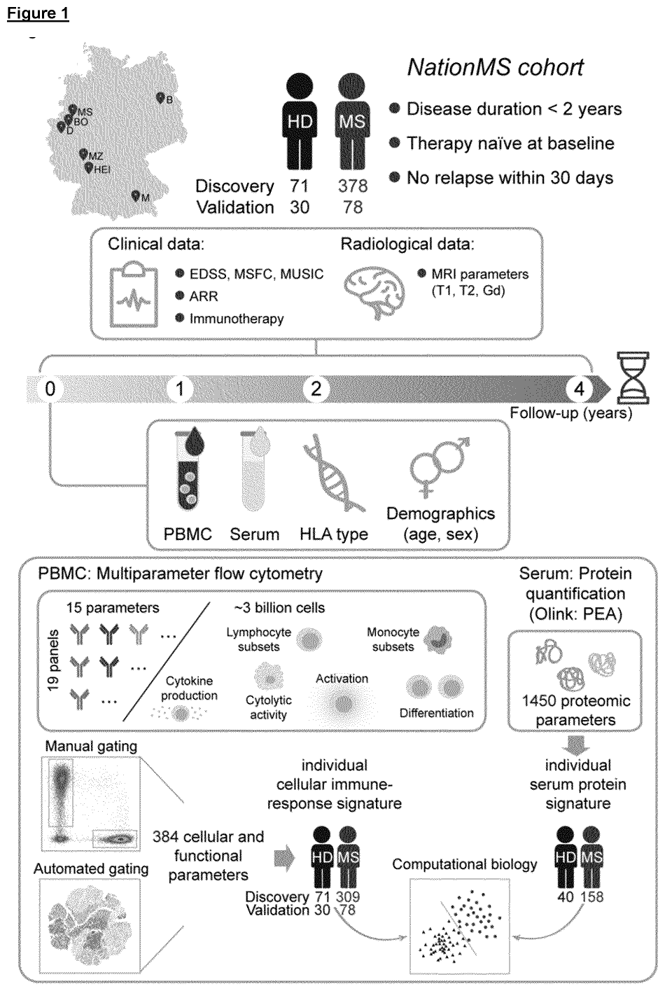

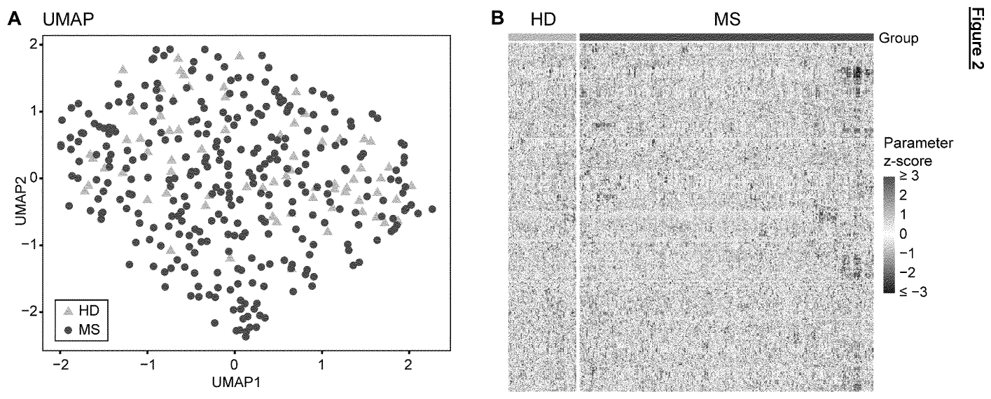

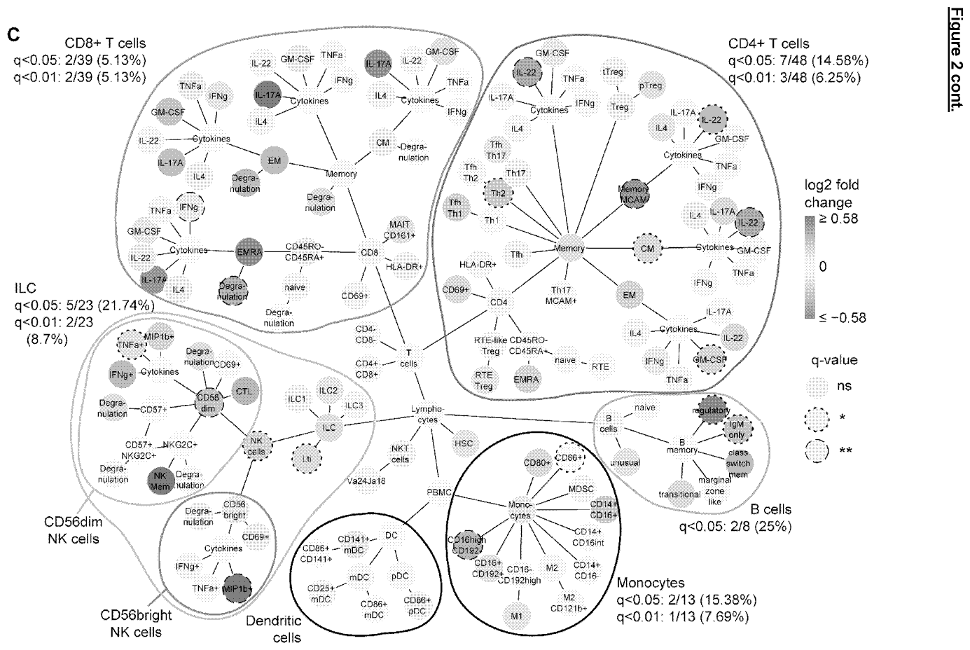

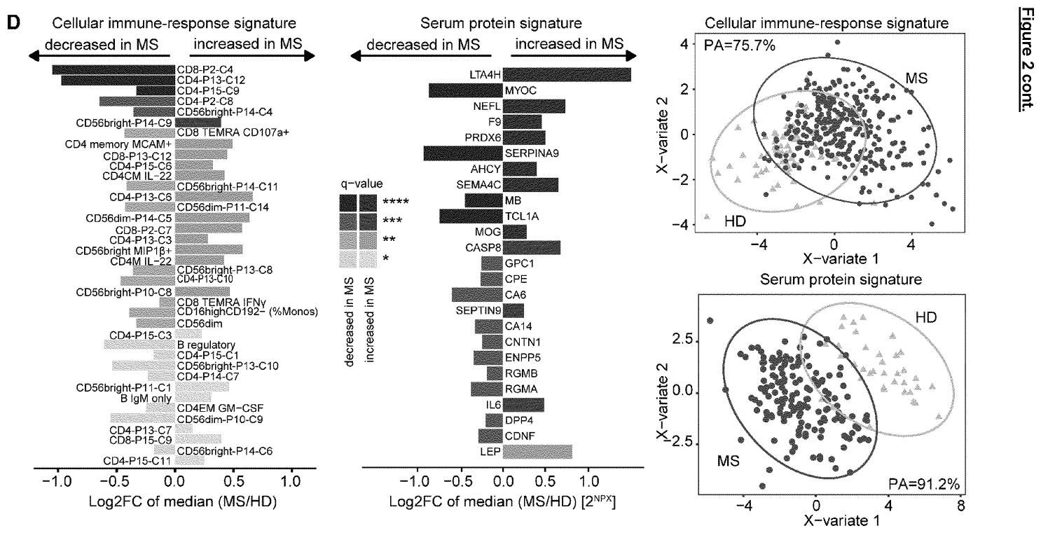

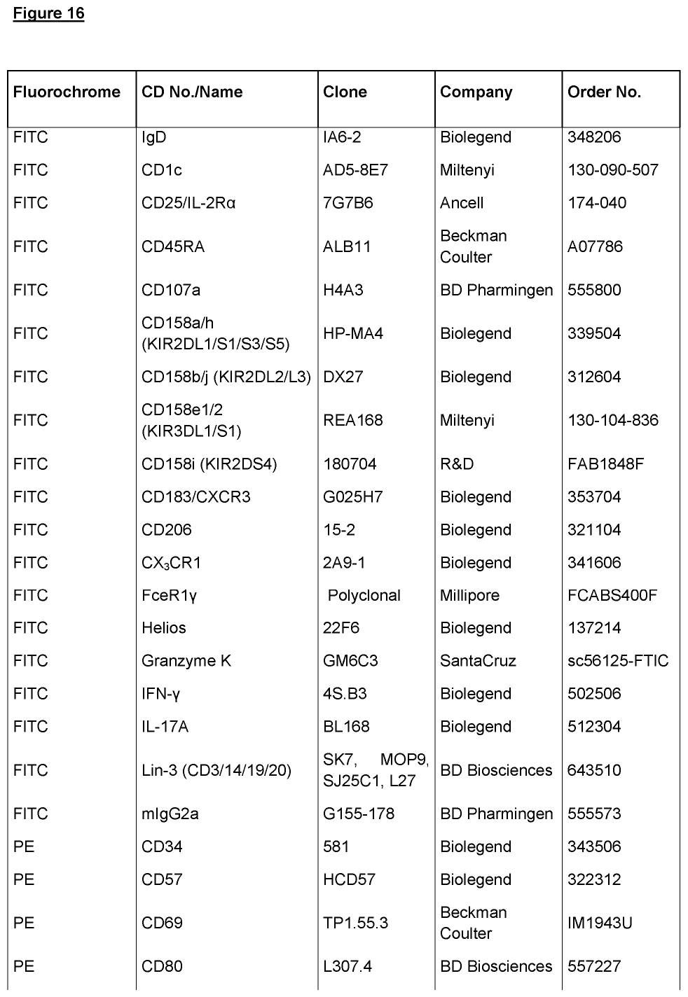

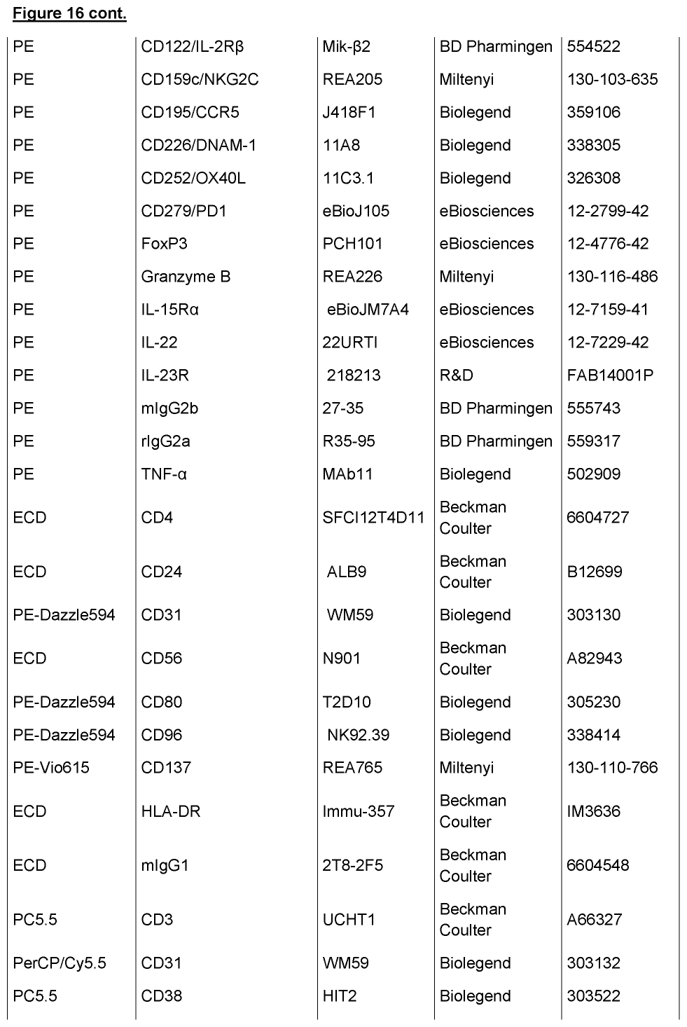

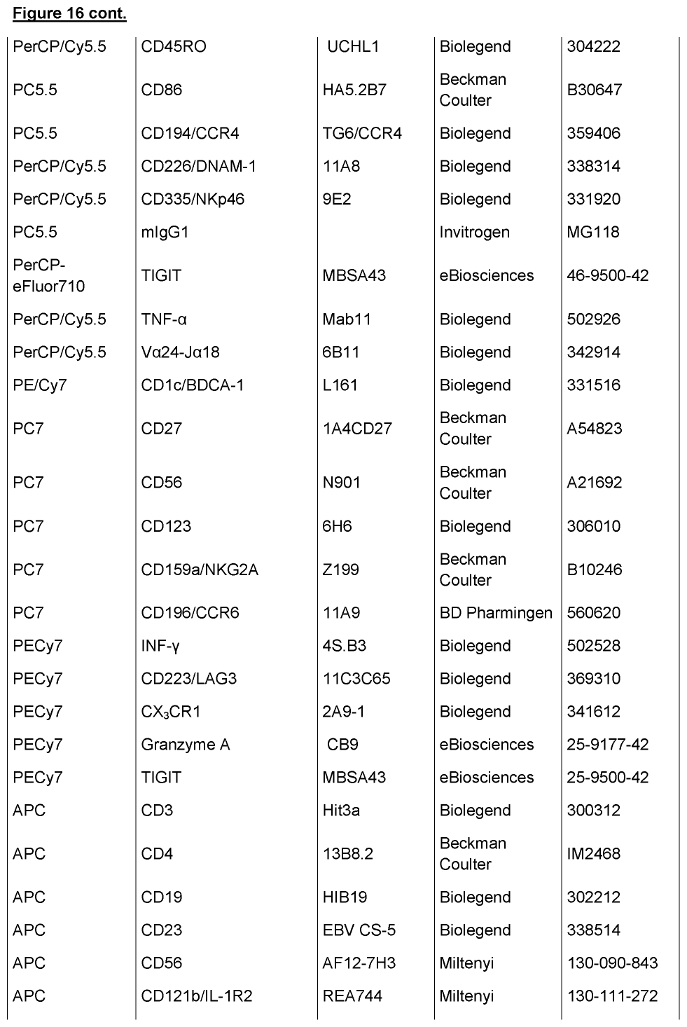

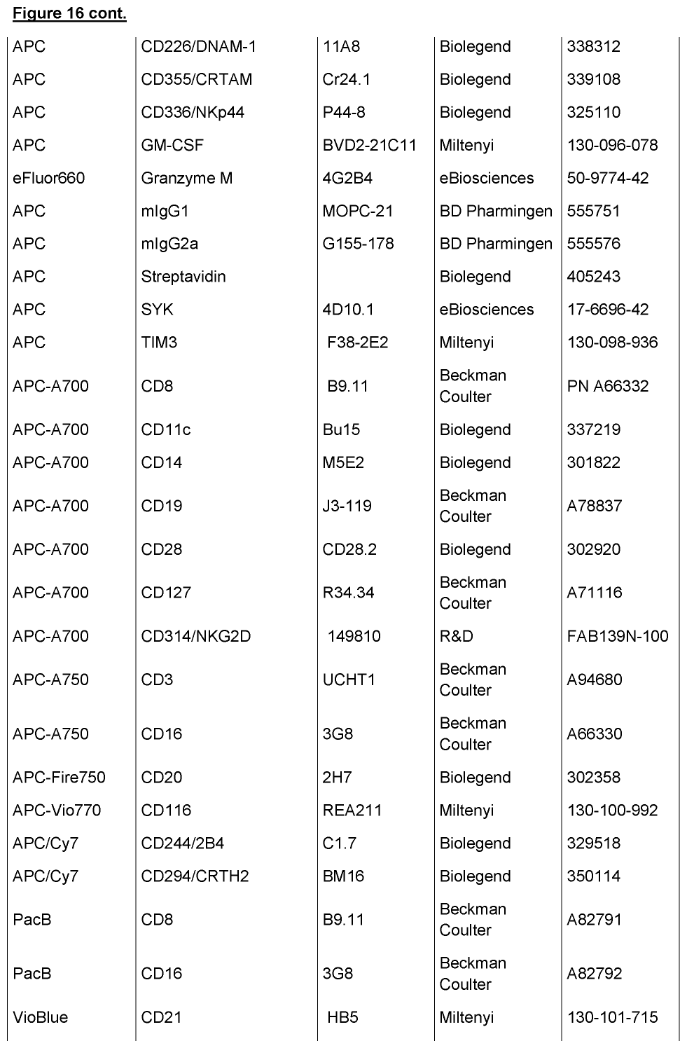

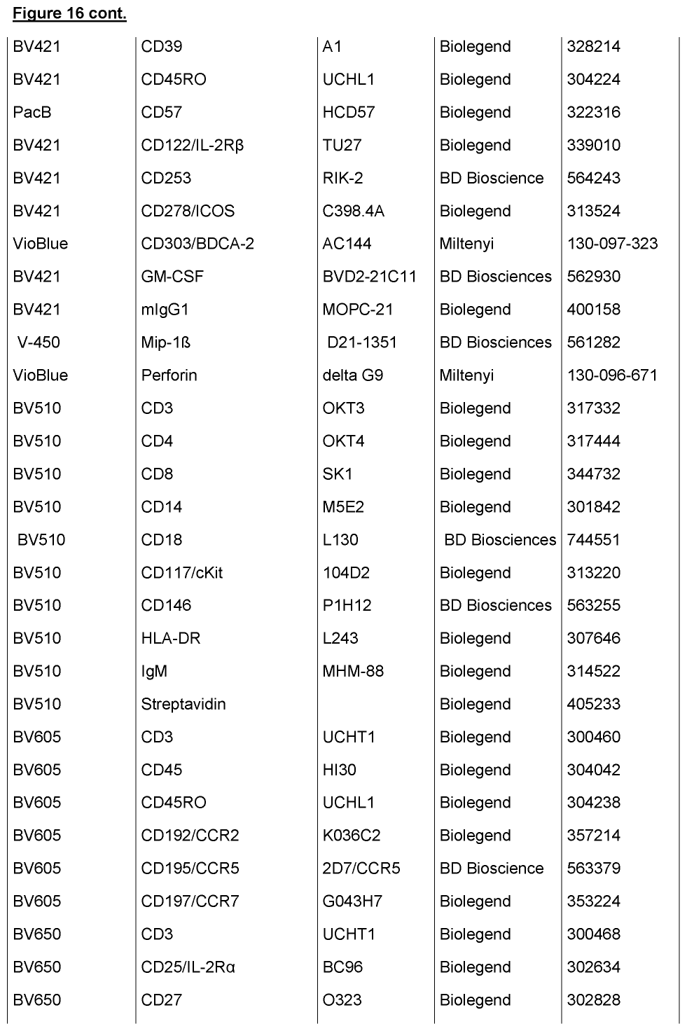

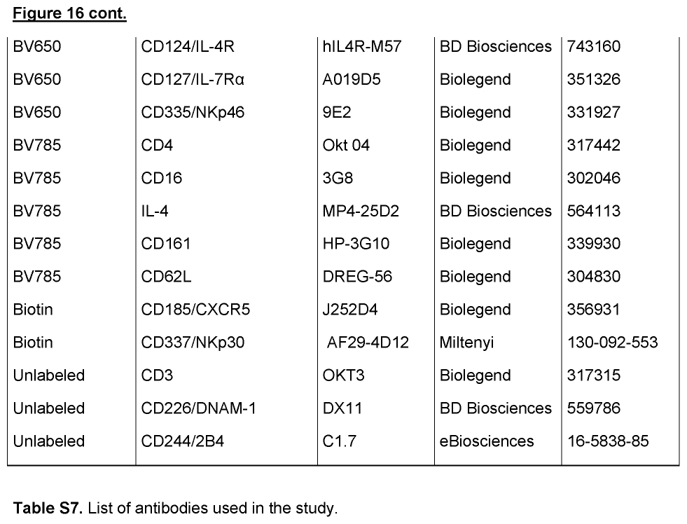

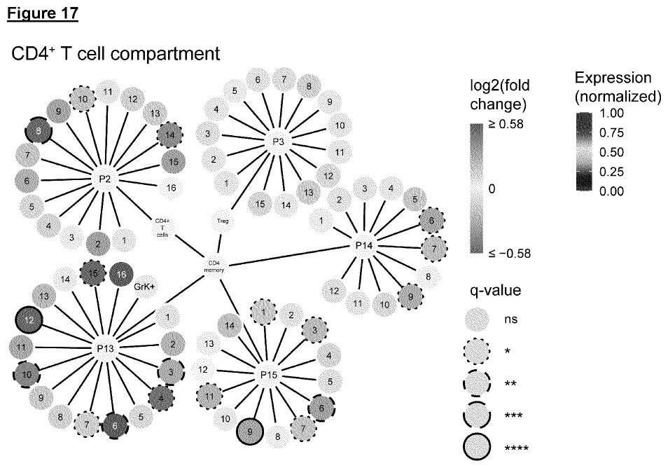

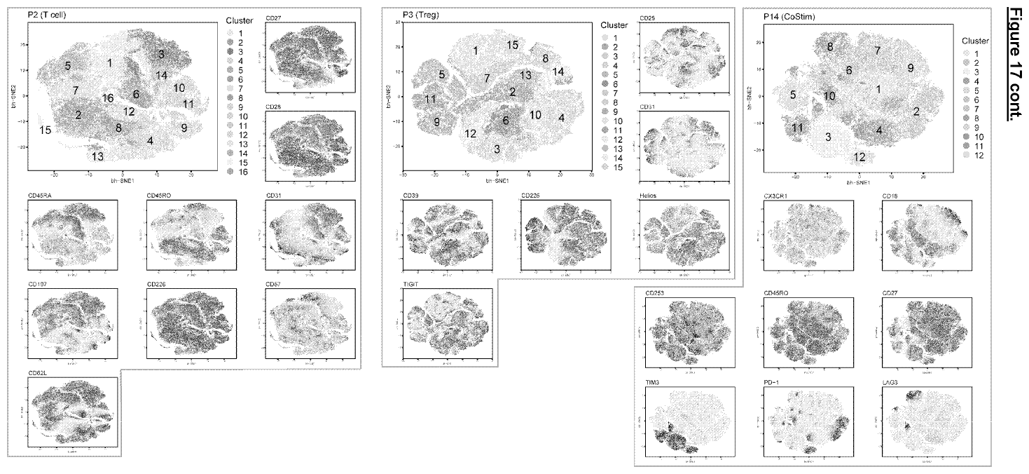

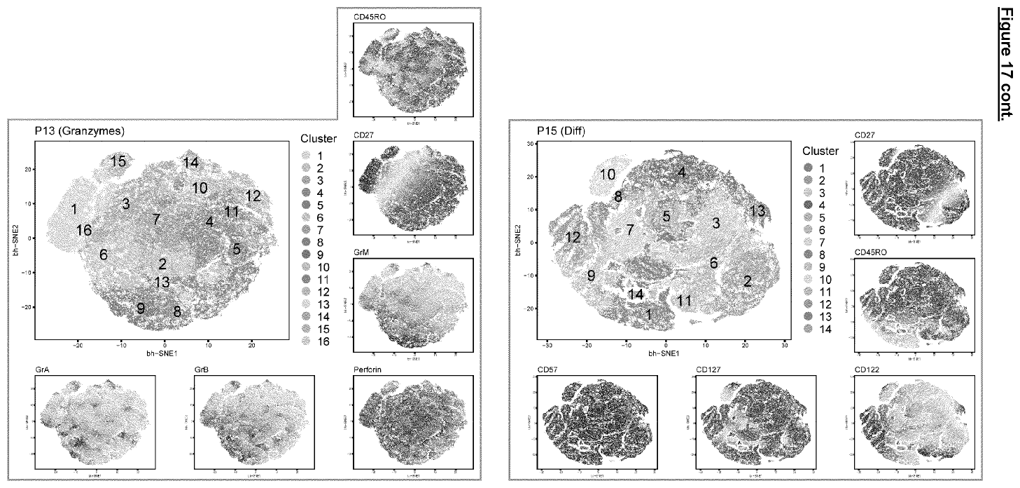

Study design for investigation of systemic immune-response signatures in early MS The inventors analyzed changes in cellular and soluble parameters in a well-curated prospectively recruited subcohorts of 378 (discovery) and 78 (validation) treatment-naïve relapsing-remitting multiple sclerosis patients (FIG. 1, FIG. 10 with Supplementary Table 1), who were recruited within the first 2 years of disease onset at 7 German centers (NationMS cohort; full cohort 1 200 patients (Ostkamp et al., 2021; von Bismarck et al., 2018)). The patients were longitudinally followed up using a standardized study protocol, including expanded disability status scale (EDSS) (Kurtzke, 1983), multiple sclerosis functional composite (MSFC) test (Fischer et al., 1999), multiple sclerosis inventor cognition (MUSIC) test (Calabrese et al., 2004), annual relapse rate (ARR), disease modifying treatment (DMT) as well as classical morphometric cMRI parameters (brain volume, numbers of T1 and T2 lesions, gadolinium enhancement). Seventy-one age- and sex-matched healthy individuals served as controls (FIG. 10 with Supplementary Table 1). Rigorous quality assessment resulted in the exclusion of 69 multiple sclerosis patients from the discovery cohort (see methods). The inventors performed multi-parameter flow cytometry of peripheral blood mononuclear cells (PBMC) and targeted proteomics of serum, and analyzed the results applying computational biology methods (FIG. 1). A combination of manual gating (FIG. 11 with Supplementary Table 2) with unsupervised dimensionality reduction and automated gating (FIGS. 18-21 with Supplementary FIG. 1-4) revealed 384 parameters defining distinct innate and adaptive immune-cell subsets and their function; thereby, resulting in individual cellular immune-response signatures of 309 multiple sclerosis patients and 71 healthy individuals (FIG. 1). Furthermore, low input proteomics (Filbin et al., 2021) in the serum of a sub-cohort of 158 out of 378 multiple sclerosis patients and 40 out of 71 healthy individuals yielded in 1 450 soluble markers defining the individual serum protein signatures. Combined cellular immune-response and serum protein signatures formed the individual immune-response signatures of each individual.

### Peripheral Immune-Regulatory Networks are Disturbed in Early Multiple Sclerosis

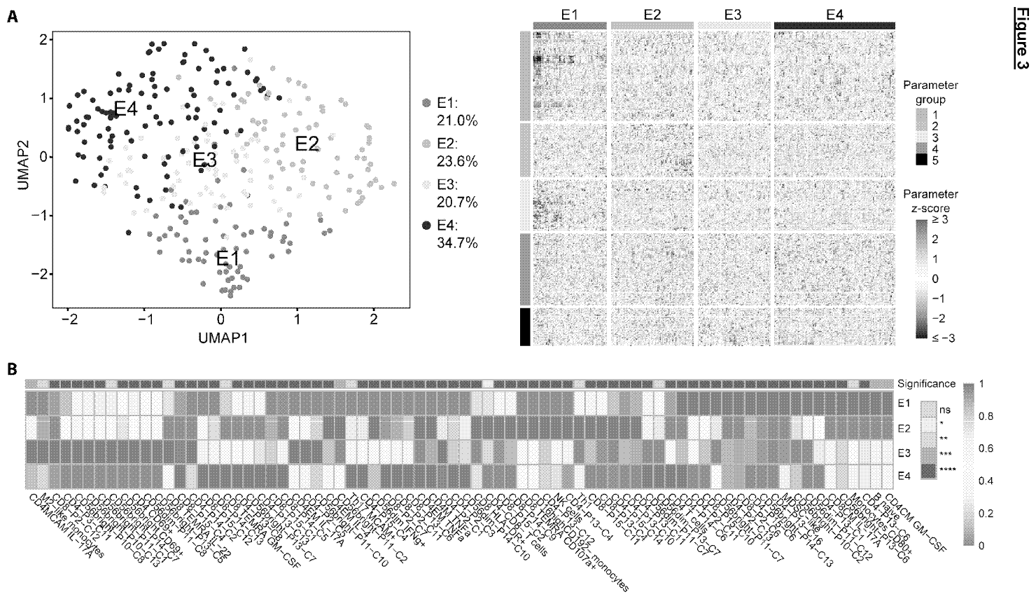

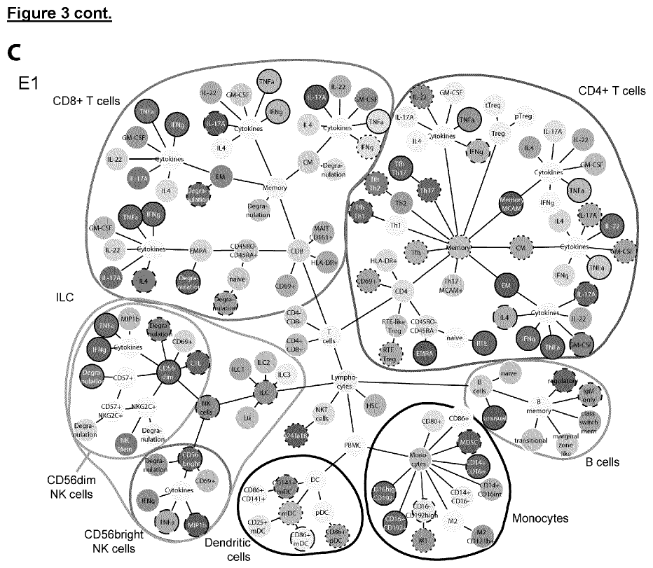

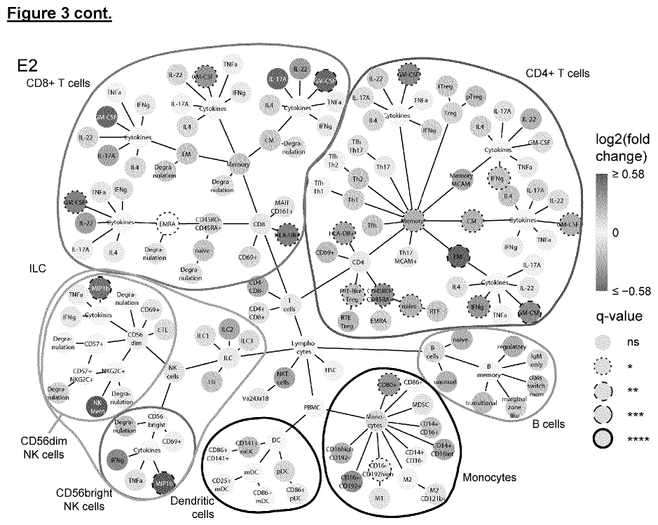

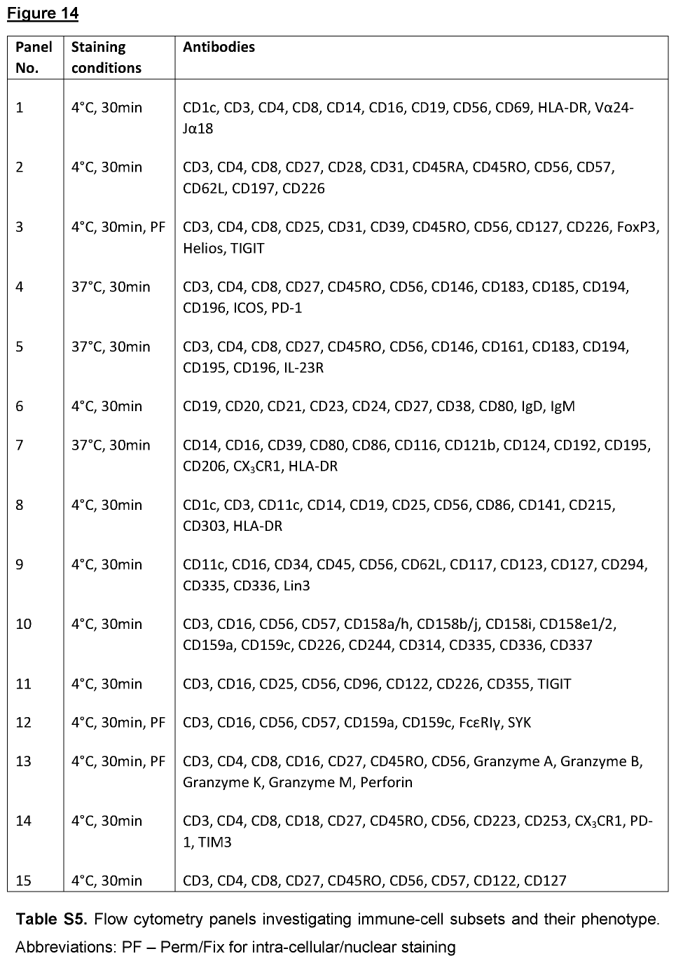

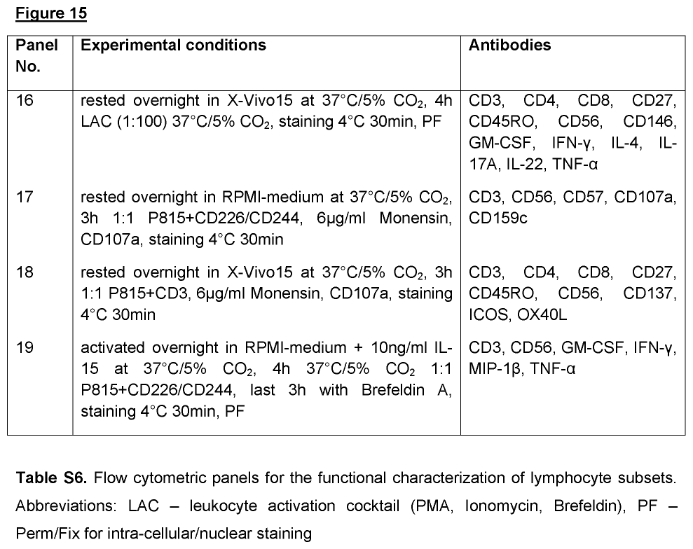

In case of k=70 and k=190, the following was observed. To identify whether and how the cellular immune-response signature differs between multiple sclerosis patients and healthy individuals, dimensionality reduction and unsupervised cluster analysis was performed. An overlap between healthy individuals and multiple sclerosis patients (FIG. 2A) suggested similar cellular immune-response signatures and reflected the heterogeneity of multiple sclerosis. Along that line, hierarchical clustering of cellular immune-response signatures demonstrated that differences between individuals dominate multiple sclerosis-specific alterations (FIG. 2B). The analysis revealed numerous marginal, but statistically significant multiple sclerosis-specific alterations in immune-cell subsets of both the lymphoid and myeloid lineage (FIG. 2C, https://osmzhlab.unimuenster.de/shiny/ms_signatures/, username: reviewer, password: review258, FIGS. 17-20 with Supplementary FIG. 1-4) indicating that multiple sclerosis has an impact on all immune-cell compartments. Accordingly, the top 10% of the most significantly altered cellular phenotypic and functional parameters associated with multiple sclerosis covered innate and adaptive immune-cells and differentiated patients from healthy individuals with a prediction accuracy of 75.7% (FIG. 2D). Next, the inventors investigated the potential of soluble parameters to differentiate multiple sclerosis patients from healthy individuals. Notably, soluble parameters differentiate multiple sclerosis patients from healthy individuals with a higher prediction accuracy of 91.2% (FIG. 2D). In addition to the already well-investigated (Barro et al., 2018a; Kuhle et al., 2016a) prognostic marker neurofilament light chain (NEFL), we discovered increased levels of leukotriene A4 hydrolase and decreased myocilin (MYOC) levels among the top candidates classifying multiple sclerosis patients (FIG. 2D, Supplementary Table 3). In summary, our high-dimensional approach demonstrated disturbances of peripheral immune-regulatory networks in early multiple sclerosis encompassing all major immune-cell subsets as well as soluble molecules.

### Early Multiple Sclerosis Forms Three or Four Distinct Immunological Endophenotypes

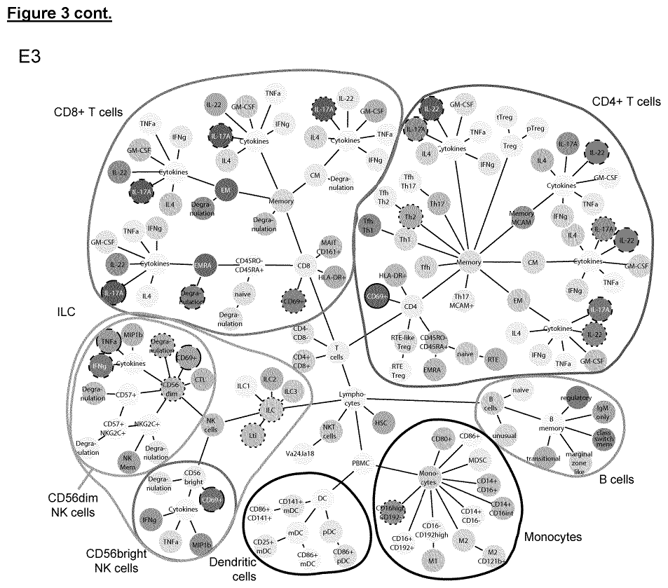

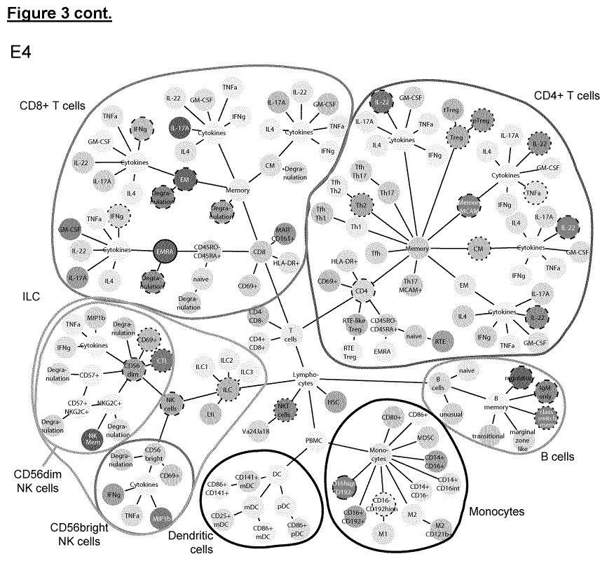

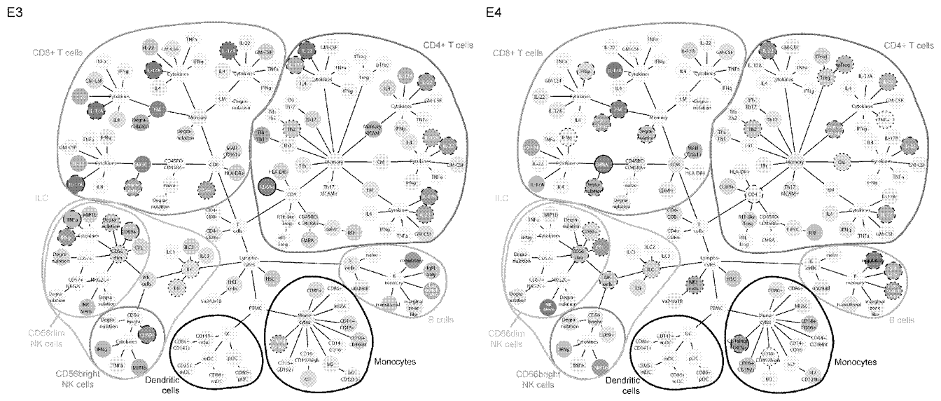

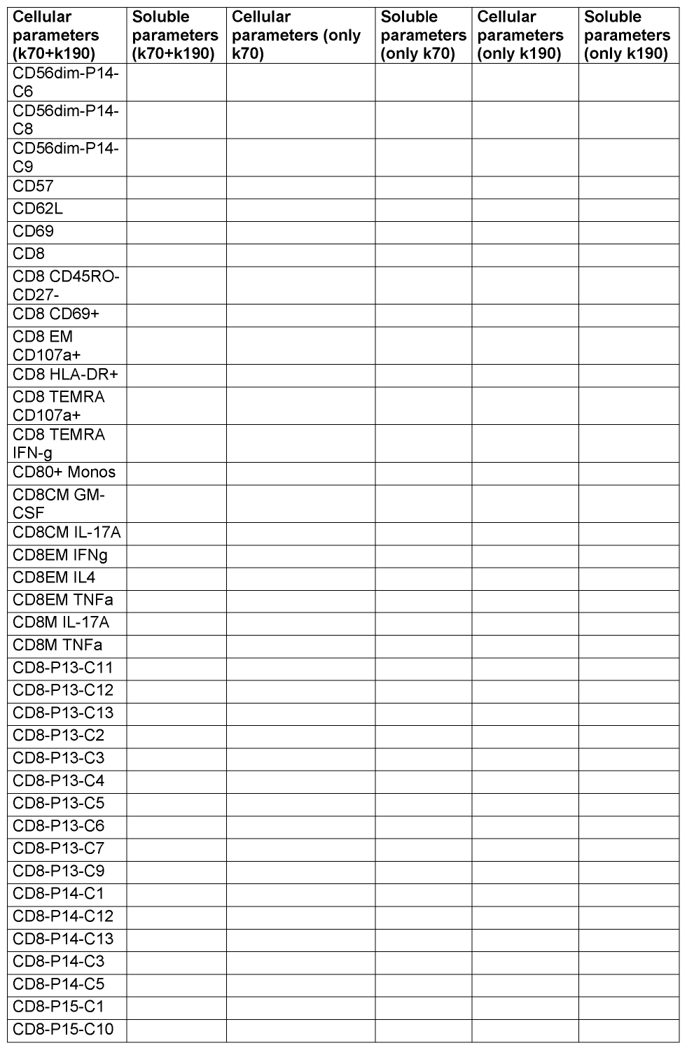

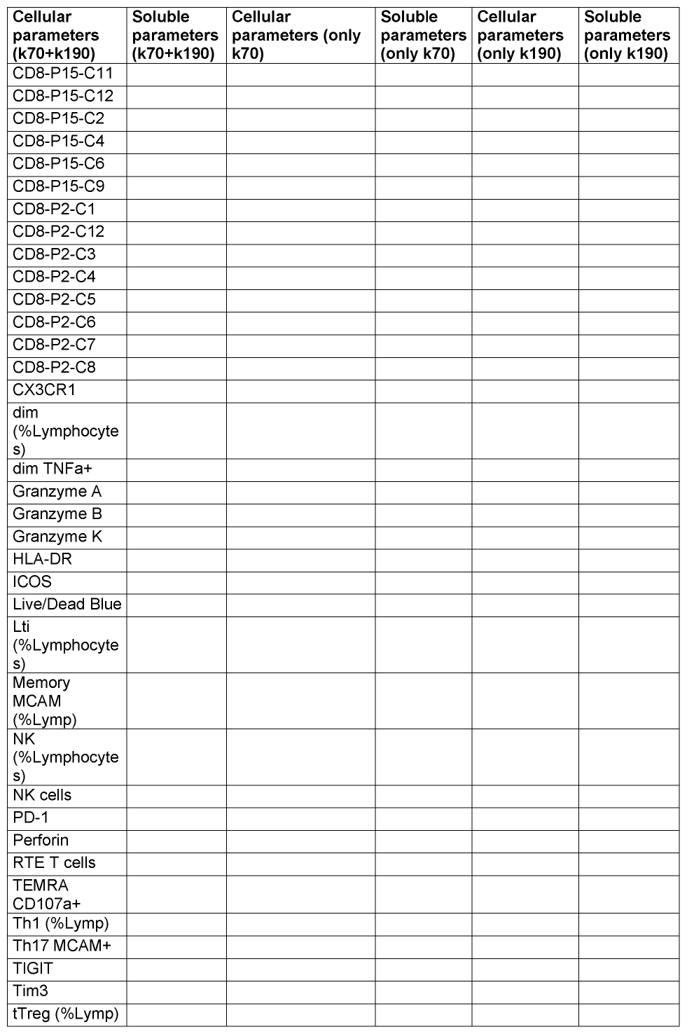

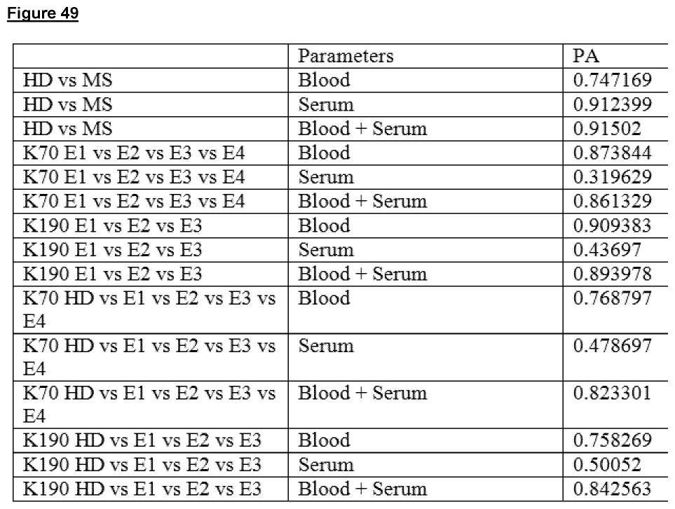

The inventors further investigated whether the observed heterogeneity in multiple sclerosis-signatures could be resolved by distinct cellular and/or soluble parameters. Intriguingly, unsupervised cluster analysis of cellular immune-response signatures revealed separation of early multiple sclerosis patients, e.g., into three or four subgroups (FIG. 35A and FIG. 3A, respectively). More specifically, unsupervised cluster analysis of cellular immune-response signatures revealed separation of early multiple sclerosis patients into subgroups (FIG. 34) depending on the tuning to the hyperparameter k for Phenograph-driven grouping. Robust outcomes were consistently observed for results with three and four endophenotypes (FIG. 34 B), with the overall most robust result for k=190 resulting in three endophenotypes (FIG. 34 B, dark line). To exemplify most relevant results, analyses are illustrated herein based on group assignment using k=70 (4 endophenotypes) and k=190 (3 endophenotypes). When comparing identified distinguishing parameter sets for k=70 and k=190, a high overlap was observed of 71% in case of distinguishing cellular parameters, 50% in case of distinguishing soluble parameters, and 65% in case of both (% are given with reference to the respective distinguishing parameter number observed for k=70). To ensure that the distinguishing parameters identified in both k=70 and k=190 analyses were key for differentiating healthy condition, MS, and/or MS endophenotypes, prediction accuracies were investigated as summarized in the table shown in FIG. 49. As it can be seen, as in case of the identified full sets of distinguishing parameter for k=70 and k=190, respectively, soluble parameters were suitable for distinguishing HD vs MS, and cellular parameters (with and without soluble parameters) were capable of distinguishing and/or differentiating between groups in any comparison investigated. Thus, the overlapping “core” parameter set of distinguishing parameters identified in case of both k=70 and k=190 were comparable to the respective full parameter sets in view of predictive accuracy in view of differentiating between subgroups investigated in a given analysis.

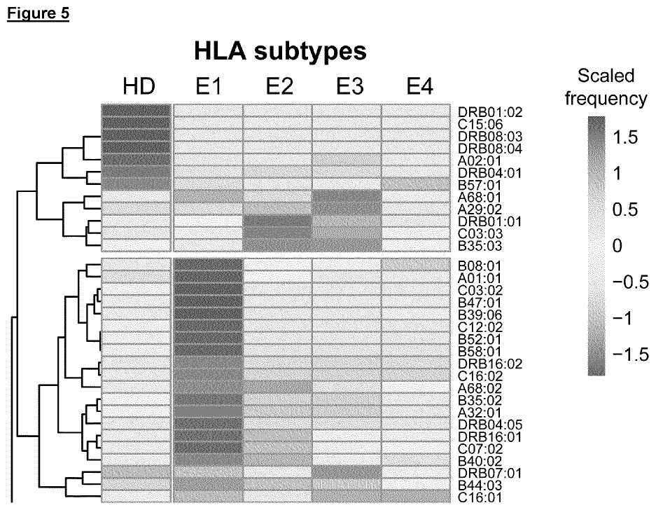

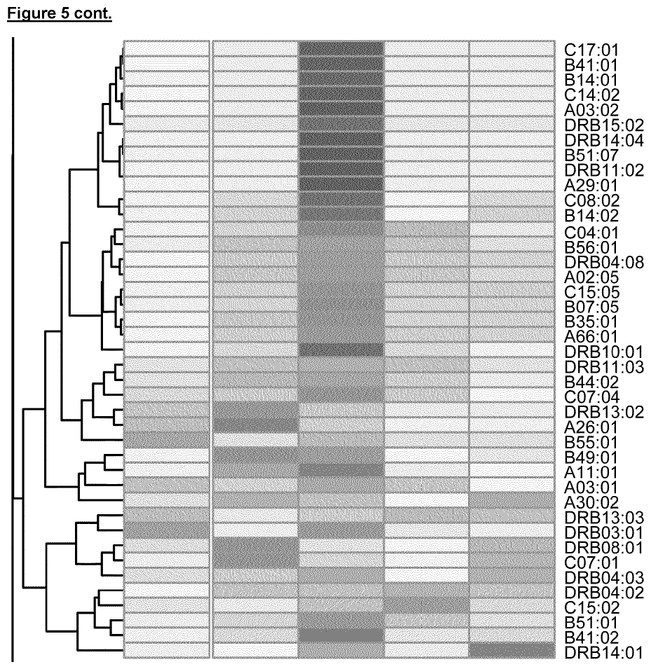

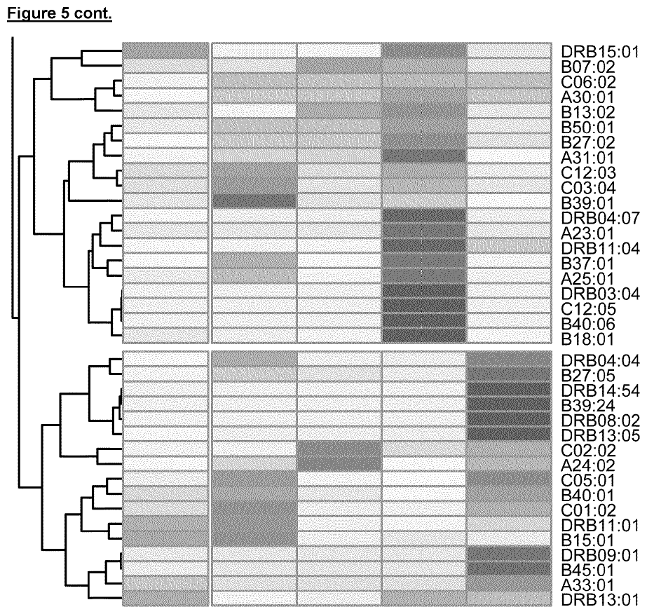

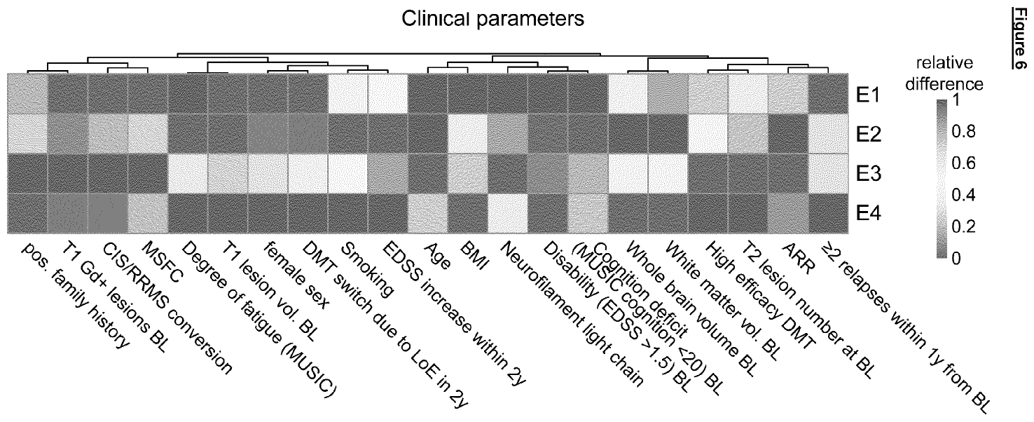

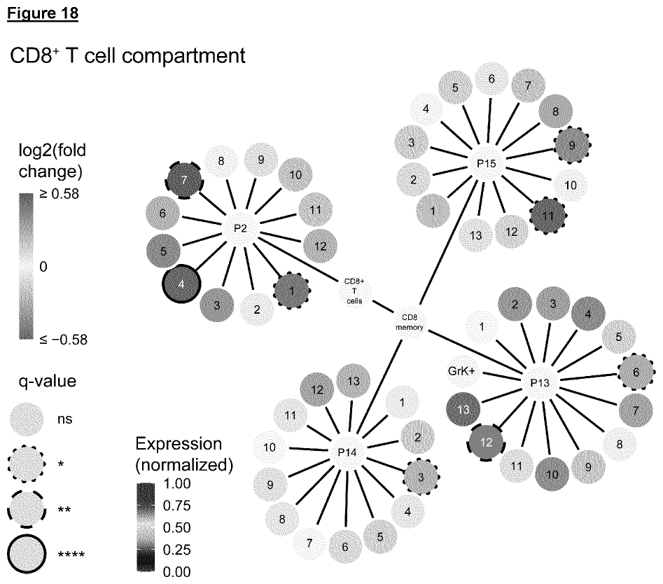

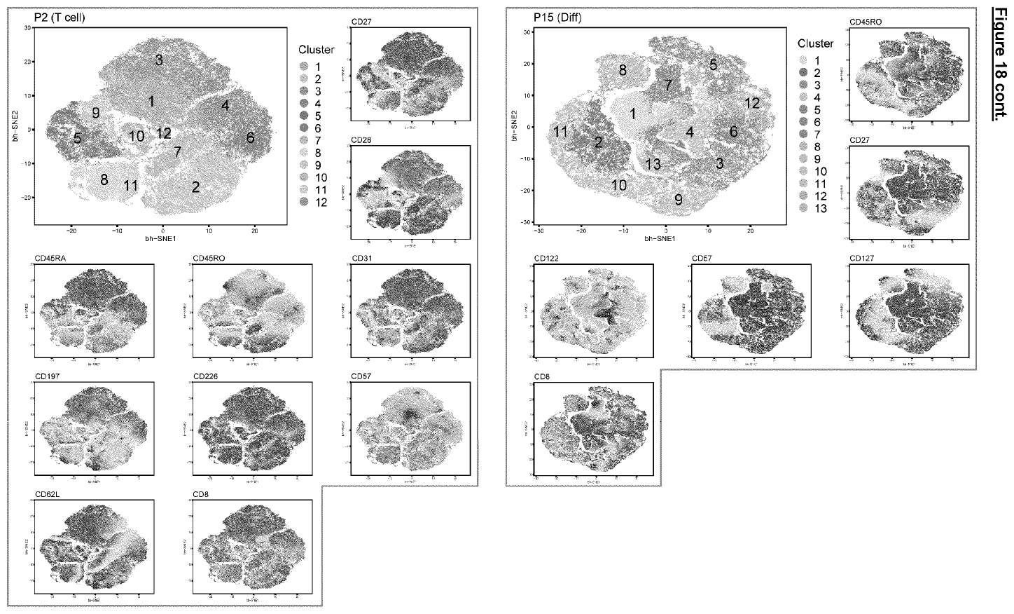

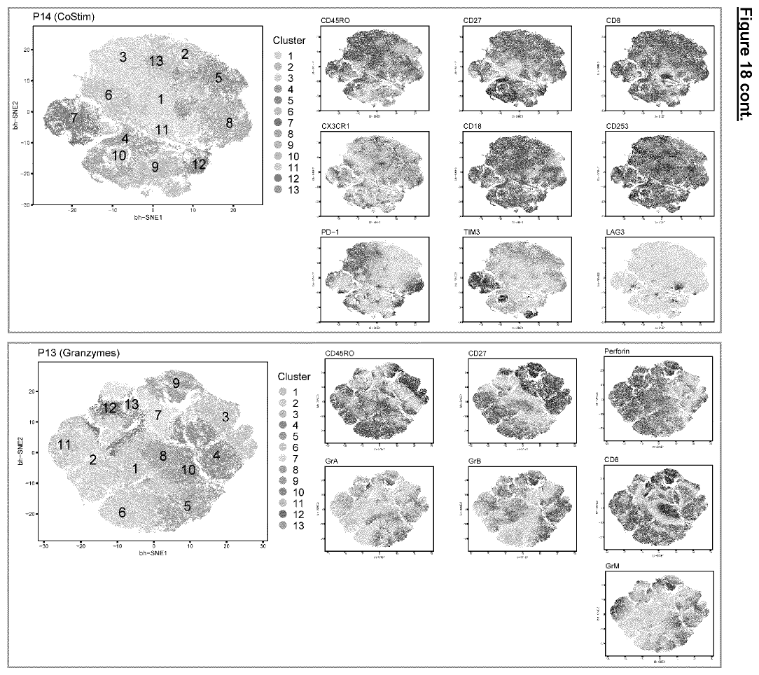

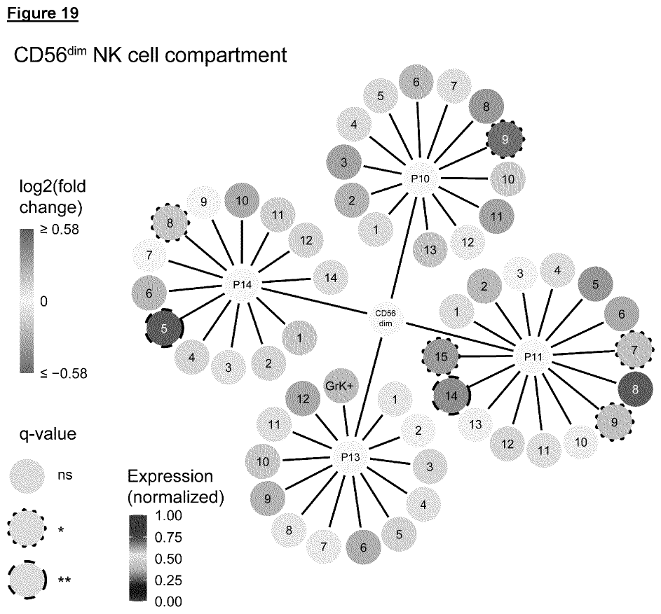

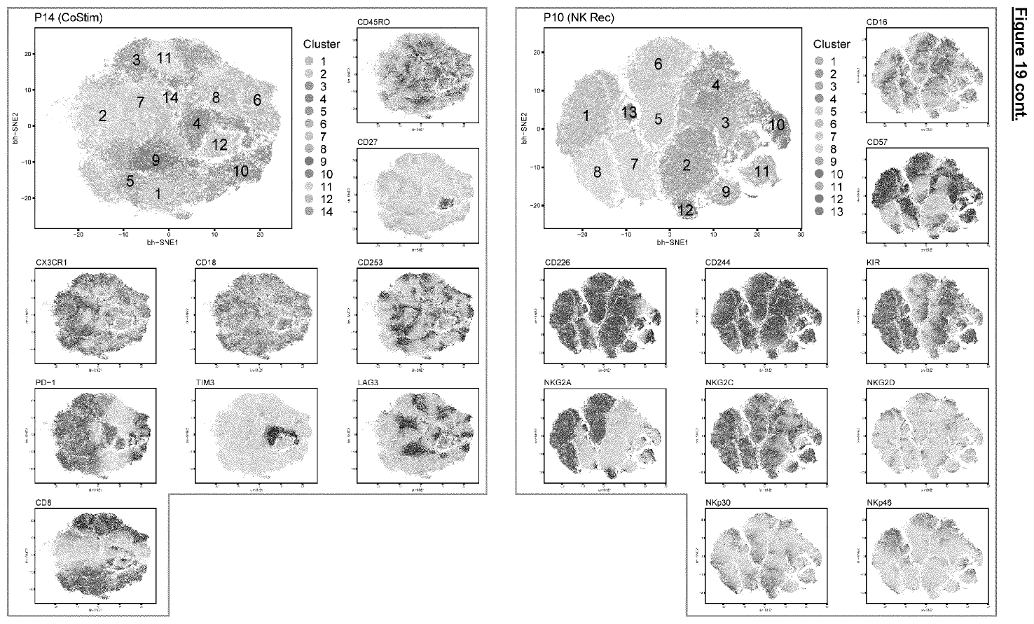

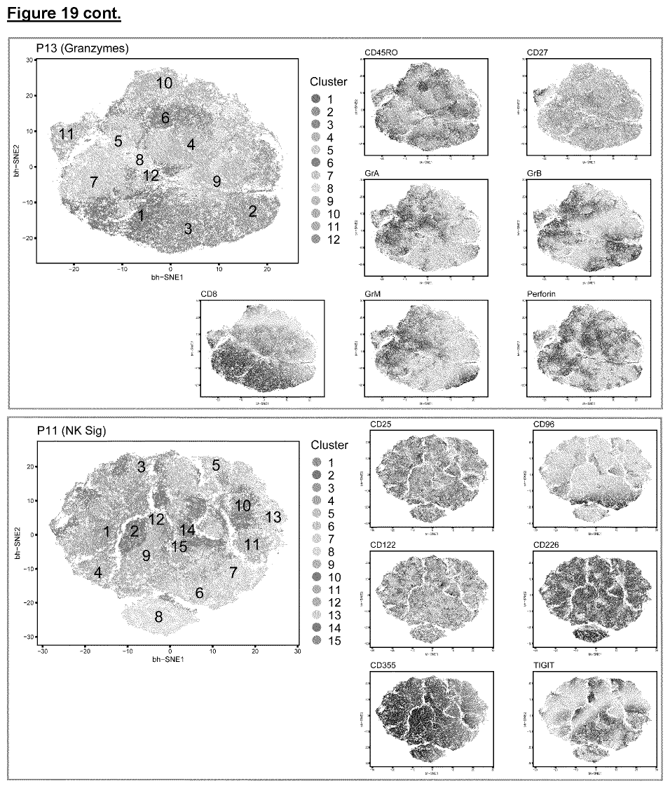

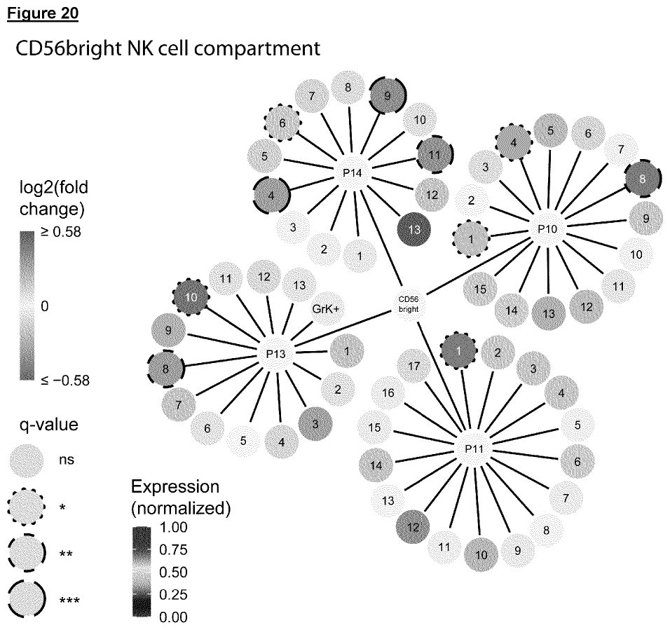

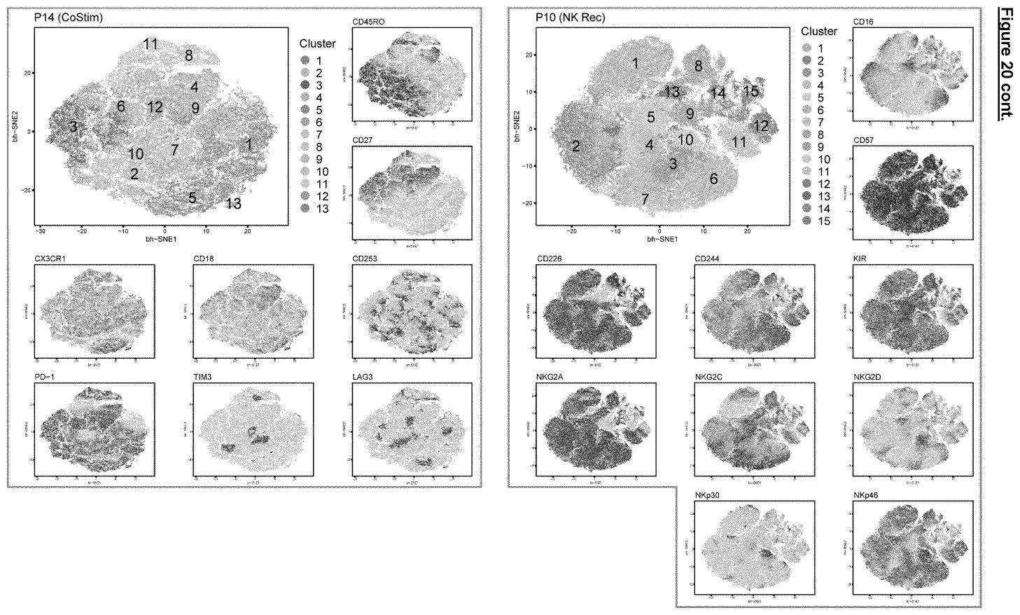

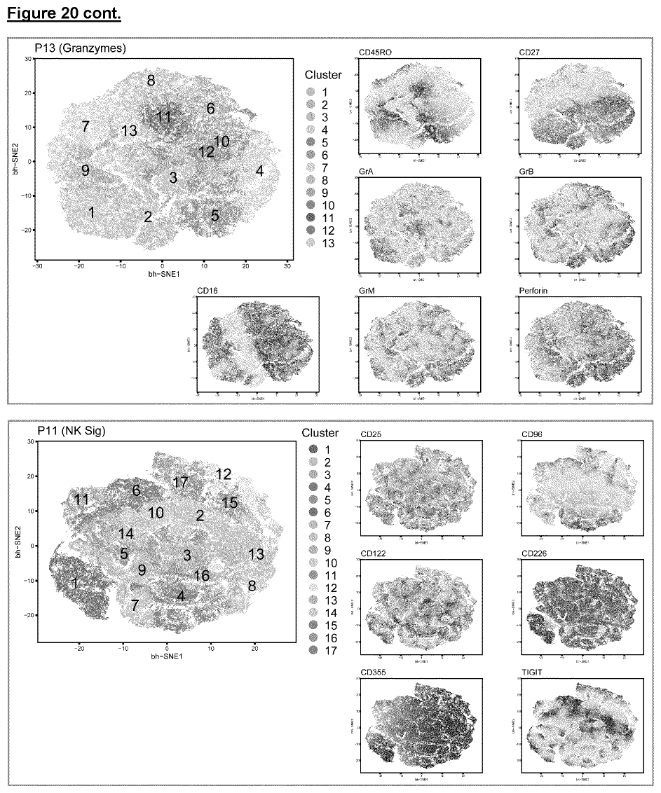

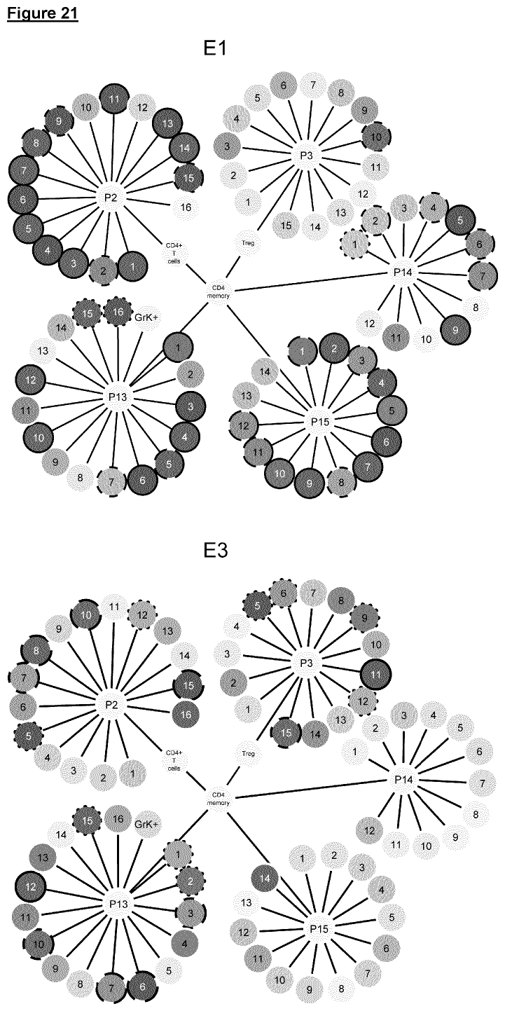

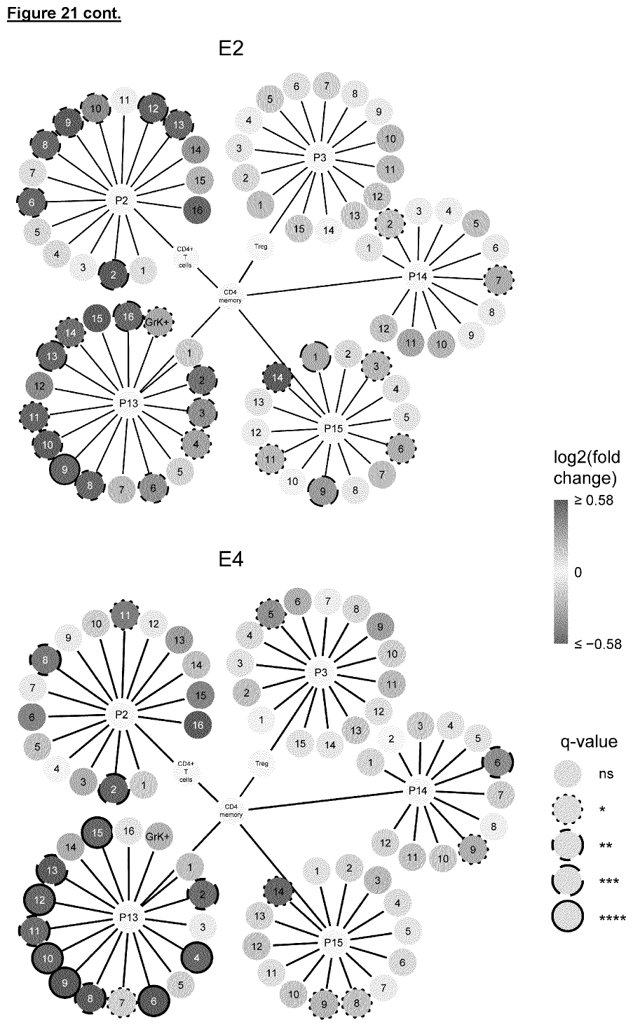

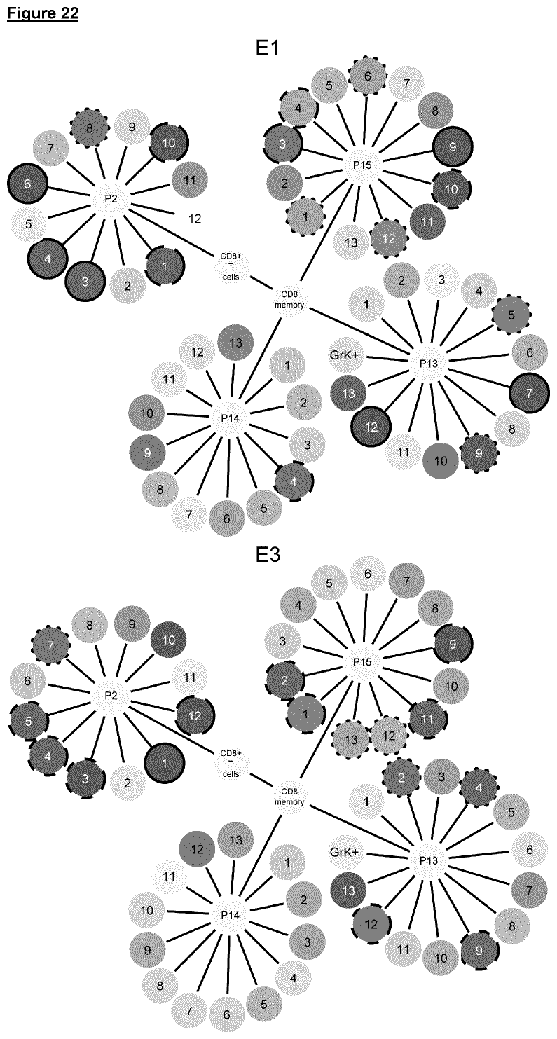

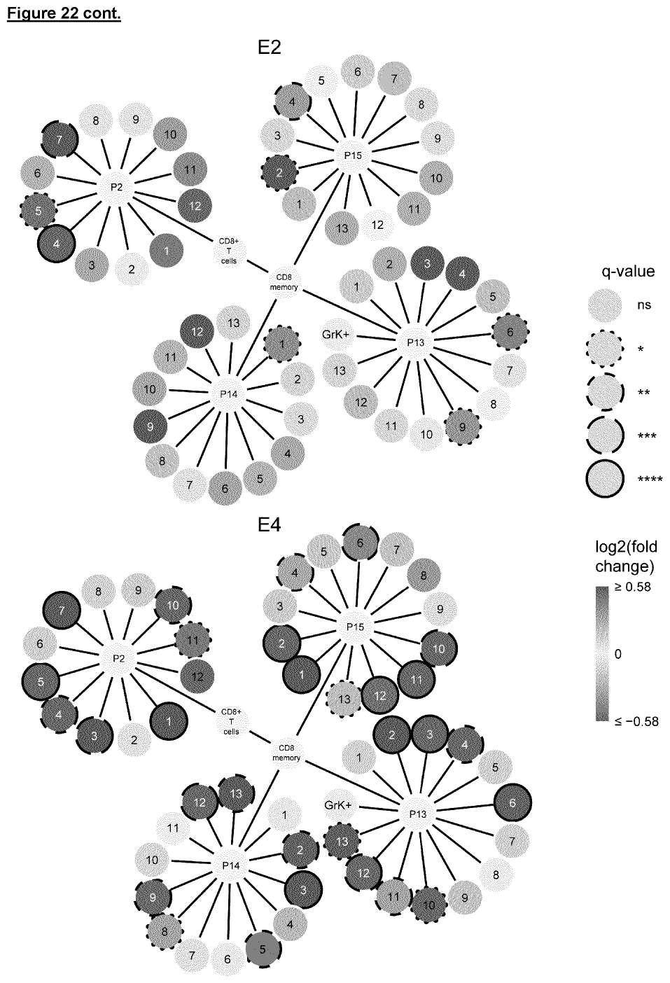

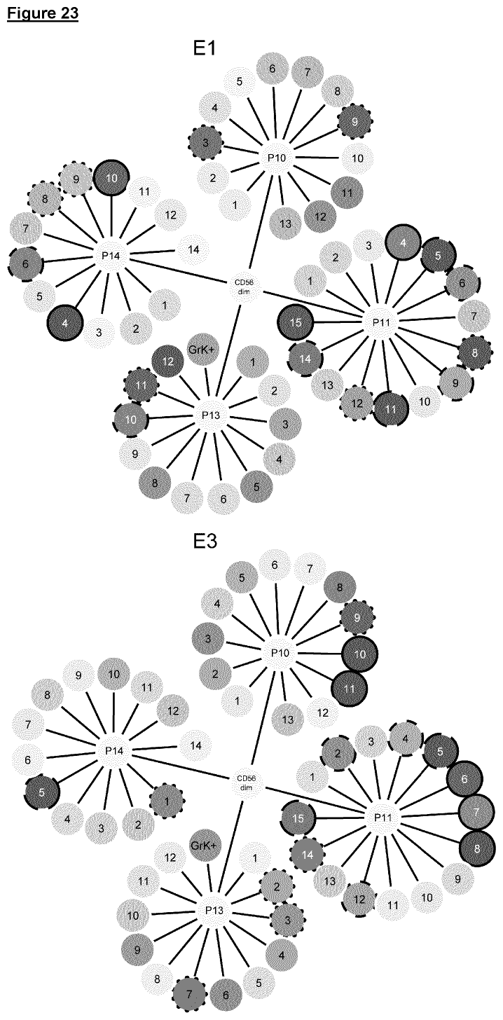

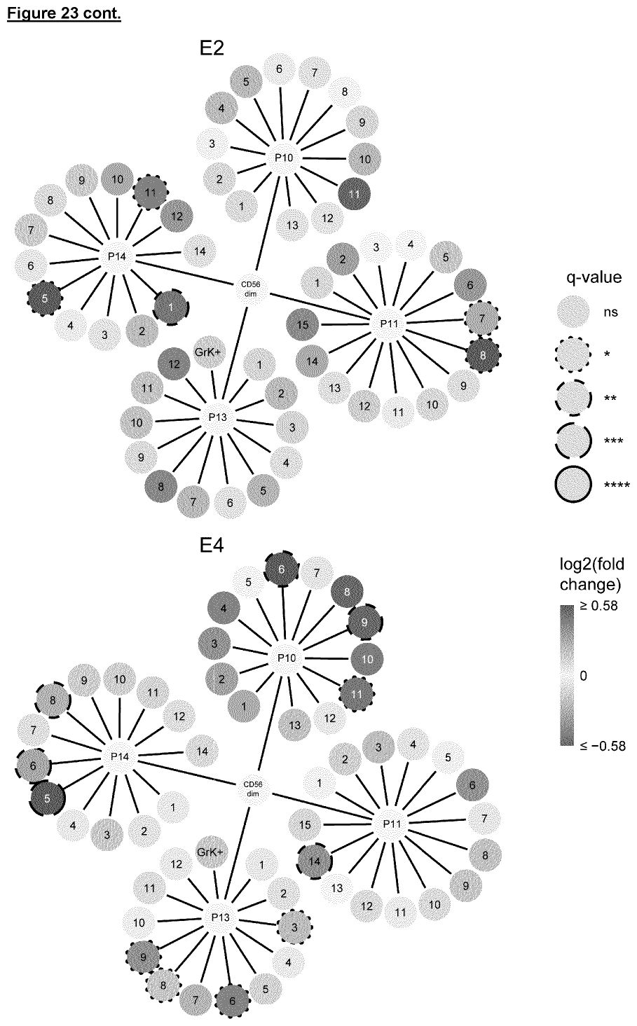

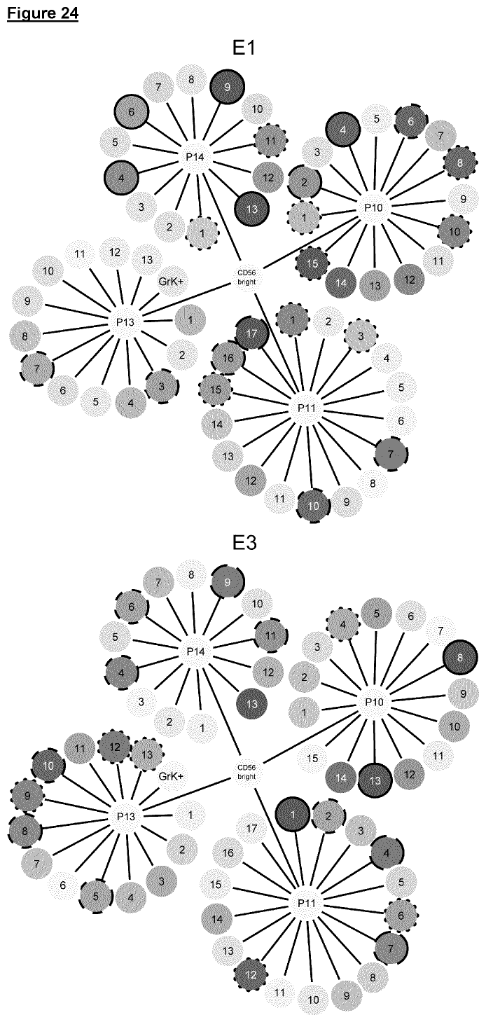

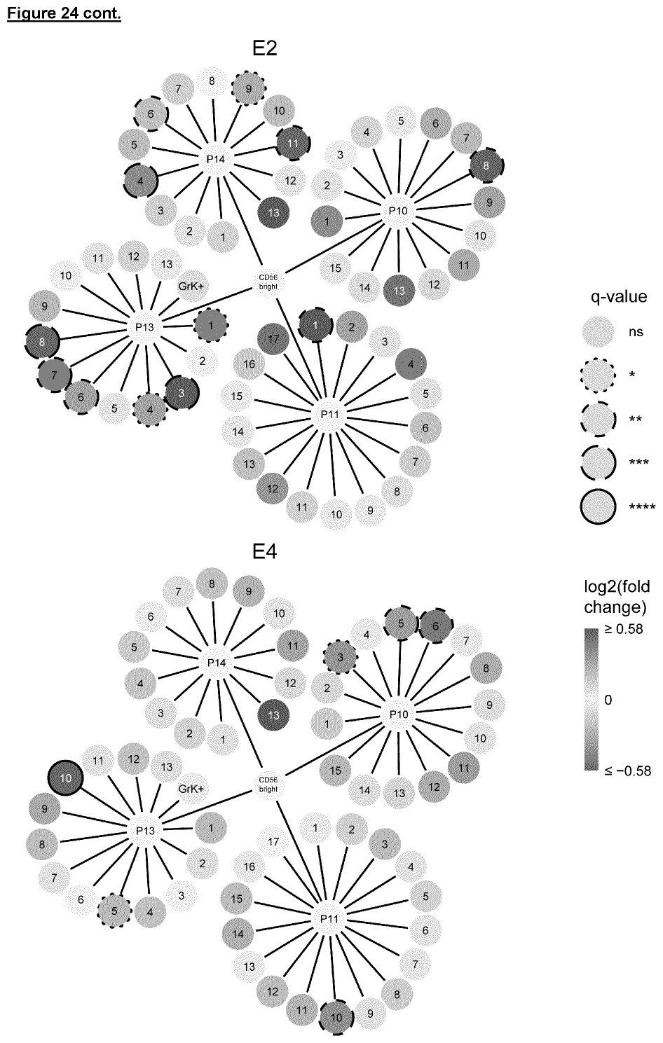

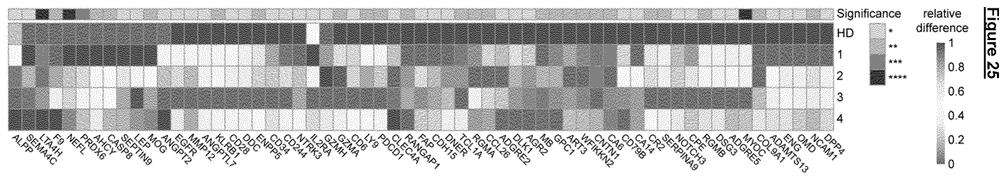

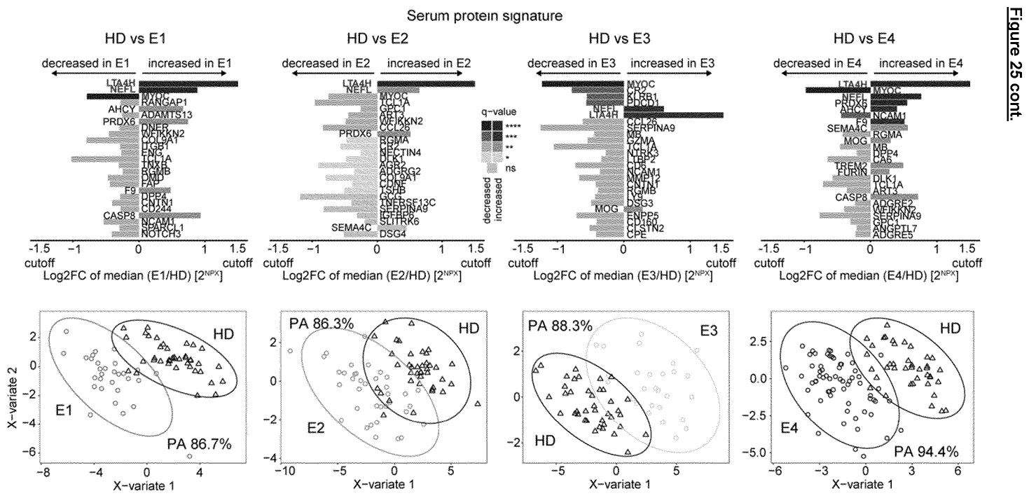

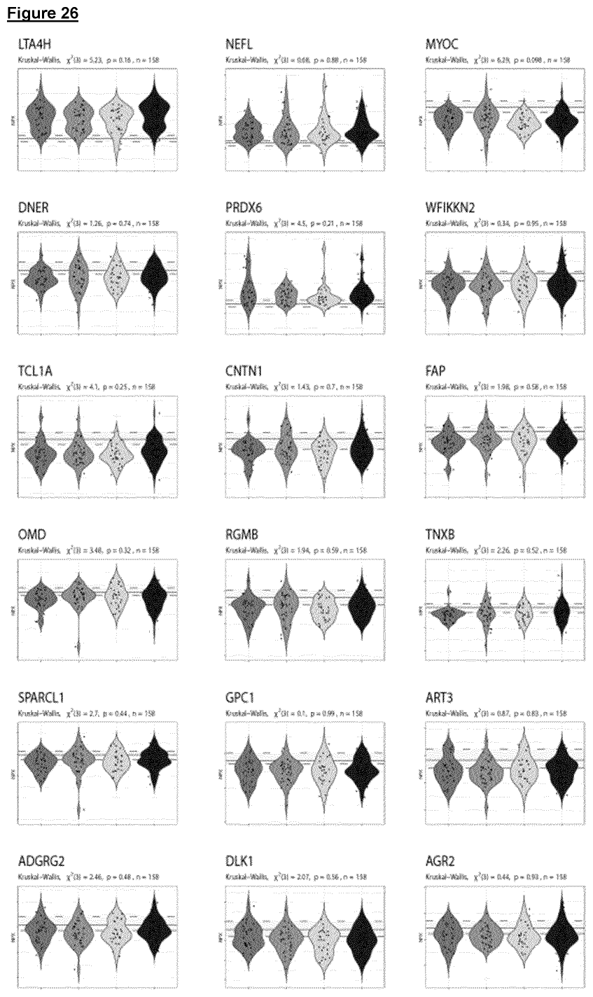

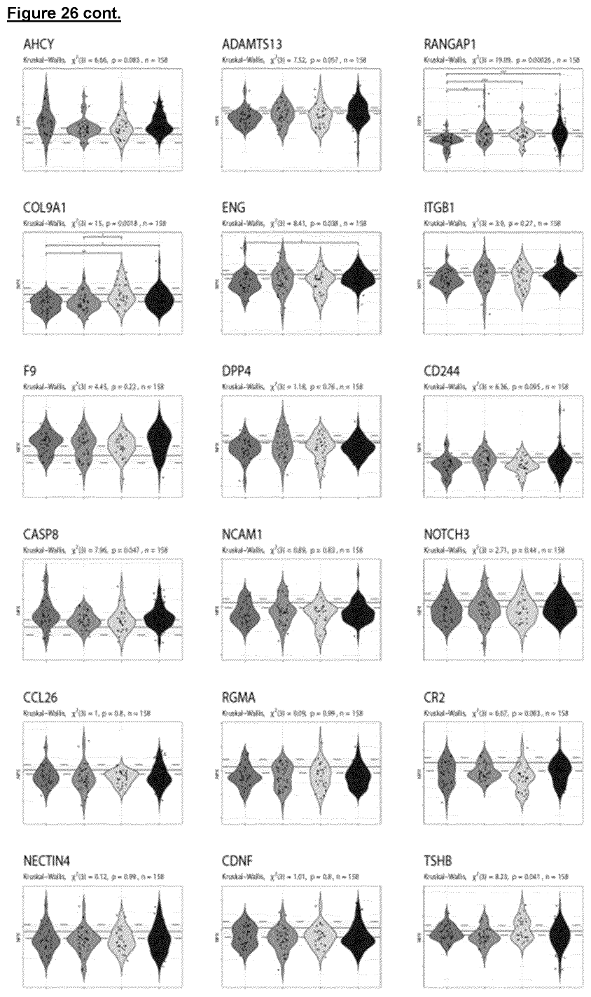

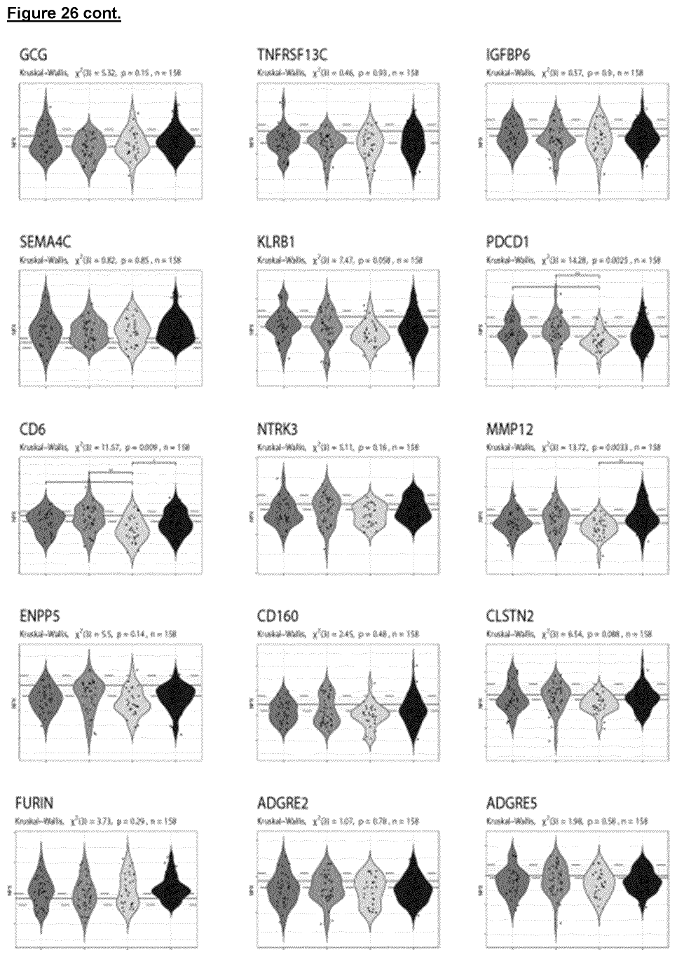

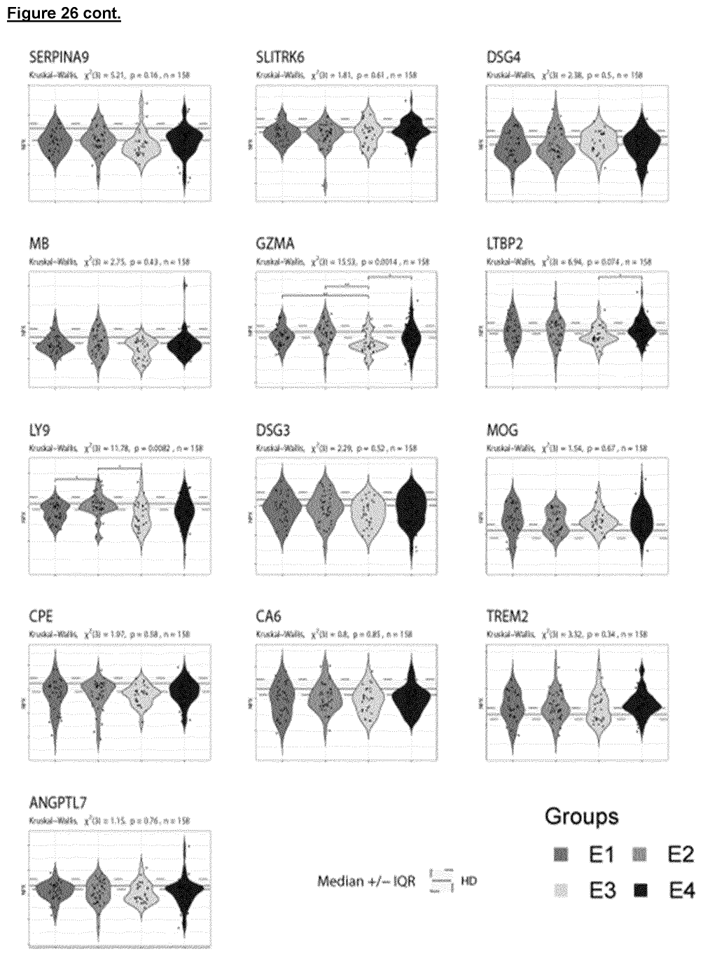

UMAP-based dimensionality reduction was used to visualize the distinctness of the four (k=70) or three (k=190) endophenotypes (FIG. 3A left and FIG. 35A, respectively), and heatmaps provided an overview of the patient-individual differences on single-parameter level (FIG. 3A right and FIG. 35B, respectively). The inventors identified 76 (FIG. 3B; cf. distinguishing parameter set ME8) and 59 (FIG. 35C; cf. distinguishing parameter set ME1u) subgroup-specific parameters, respectively, by LASSO regression characterizing discrete alterations in innate and adaptive immune-cell subsets and their function (e.g. FIG. 3B). Thus, it is tempting to speculate that subgroup-specific alterations in immunological traits result in formation of discrete immunological endophenotypes in multiple sclerosis. Each endophenotype displayed multiple sclerosis-specific characteristics (e.g. in case of k=70: FIG. 3C, https://osmzhlab.uni-muenster.de/shiny/ms_signatures/, username: reviewer, password: review258, FIGS. 21-24 with Supplementary FIG. 5-8; in case of k=190: FIG. 35D). While in case of k=70 endophenotypes 1, 2, and 4 were mainly characterized by alterations in the T-cell compartment, endophenotype 3 exhibited features of impaired immune-regulatory function. Alterations in the T-cell compartment differed between endophenotype 1, 2, and 4 such as increased proportions of CD4 memory subsets producing type 17 cytokines (IL-17A, GM-CSF, IL-22) in endophenotype 1, a shift towards CD8 TEMRA cells concomitant with an increase in granzyme B producing T cells in endophenotype 2, and disturbances in T cell differentiation, particularly of CD8 T cells, in endophenotype 4. In contrast to these T-cell dominated endophenotypes, endophenotype 3 displayed features of impaired immune-regulatory function marked by reduced DNAM-1 (CD226) expression on regulatory T- and NK cells (Gross et al., 2016; Piedavent-Salomon et al., 2015), concomitant with increased frequencies of recent thymic emigrants (RTE) as well as increased production of the type 17 cytokines by T cells. In case of k=190, endophenotype 1 was dominated by alterations in the CD4 T-cell compartment, whereas the most prominent alterations in endophenotype 2 resided in the NK-cell compartment and endophenotype 3 was characterized by shifts in the CD8 T-cell compartment (FIG. 36A). Taken together, the data demonstrate that early multiple sclerosis forms different immunological endophenotypes characterized by distinct cellular immune-response signatures, potentially reflecting differential immunological pathways involved in disease pathogenesis.

### Cellular Parameters are Superior to Soluble Parameters for Classifying Early Multiple Sclerosis

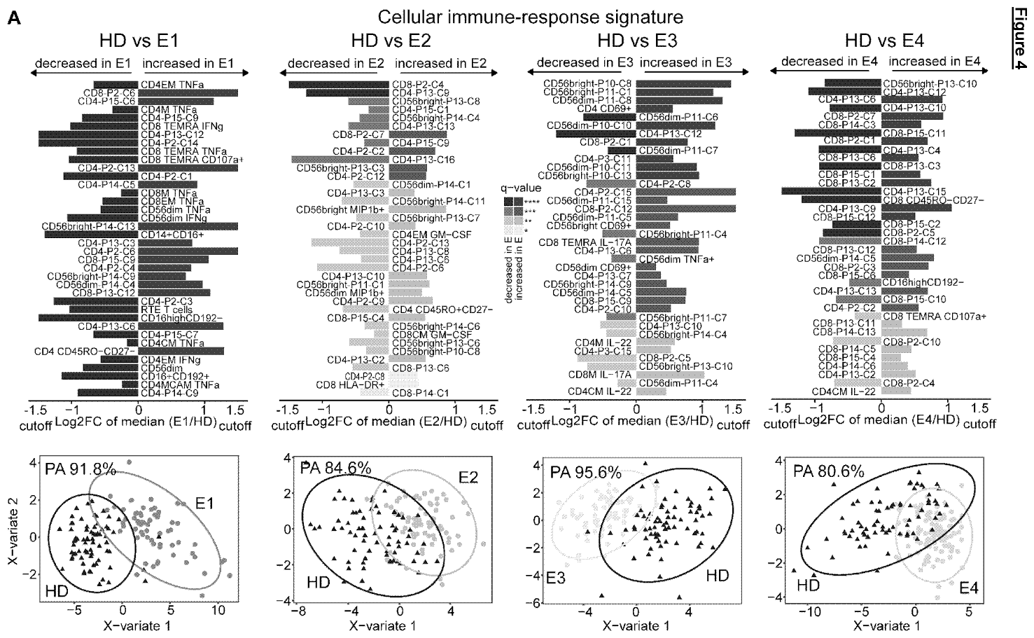

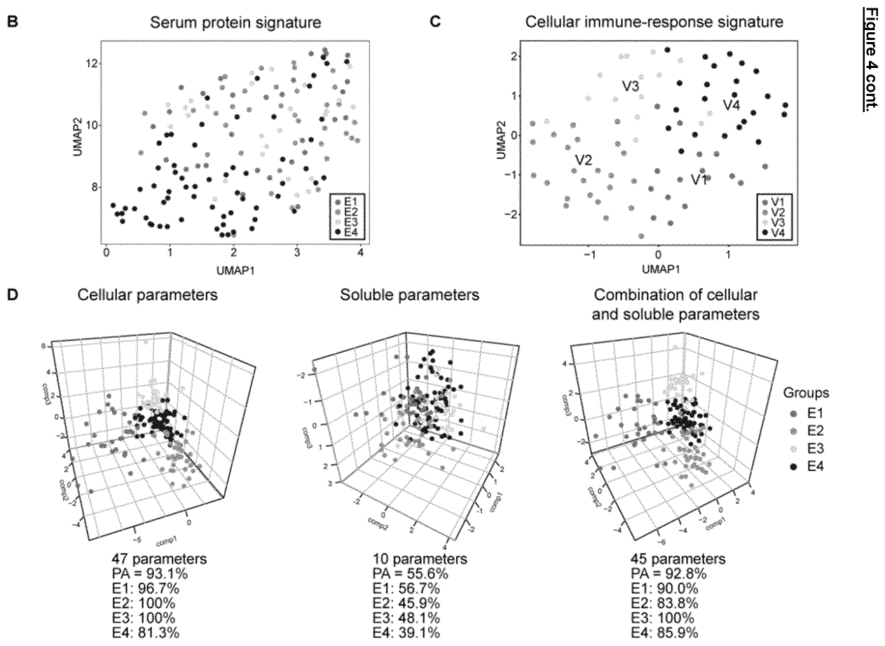

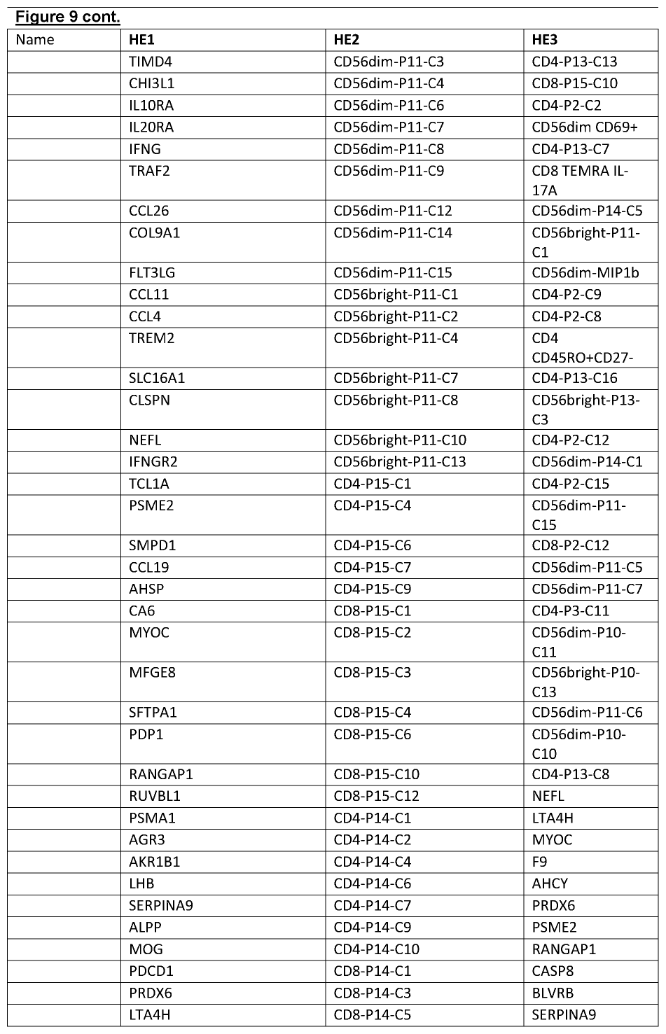

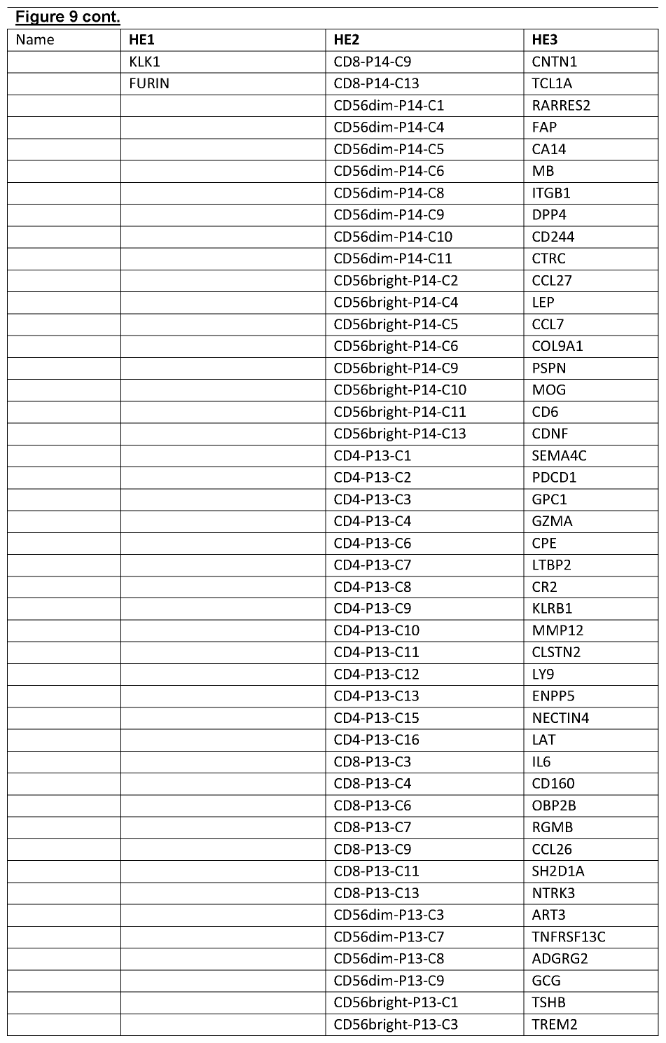

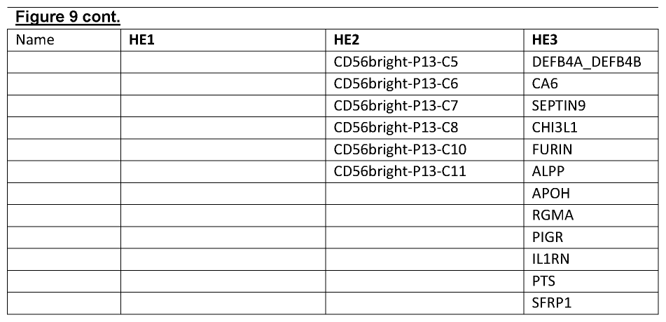

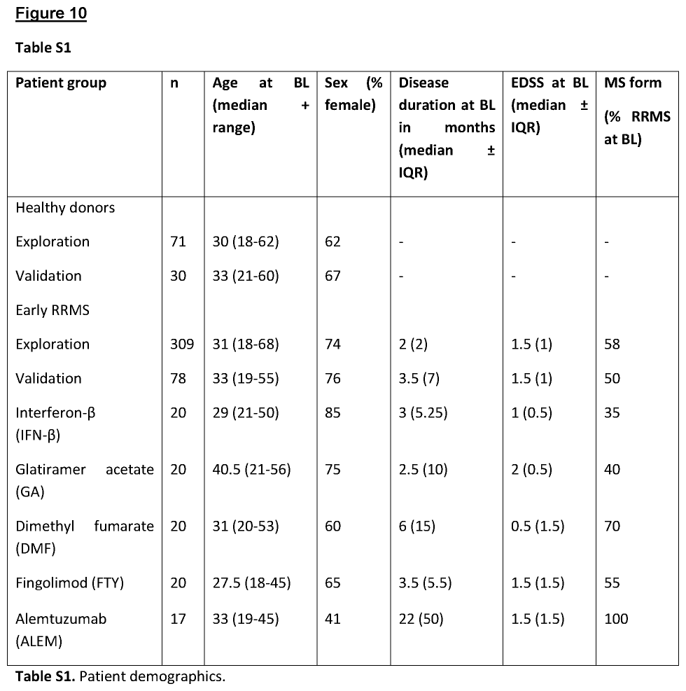

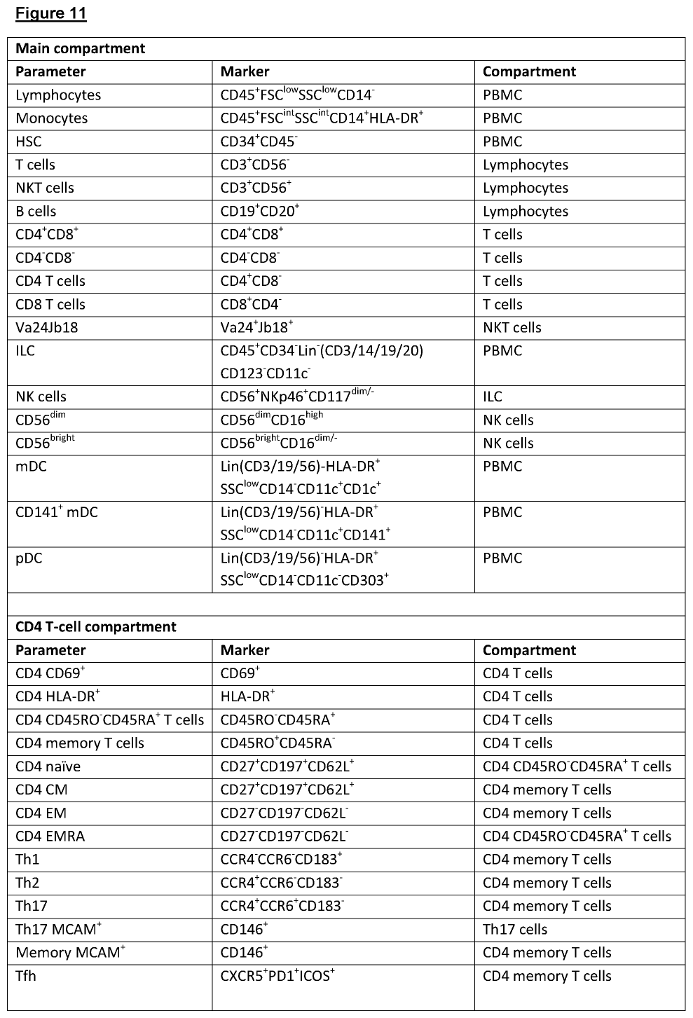

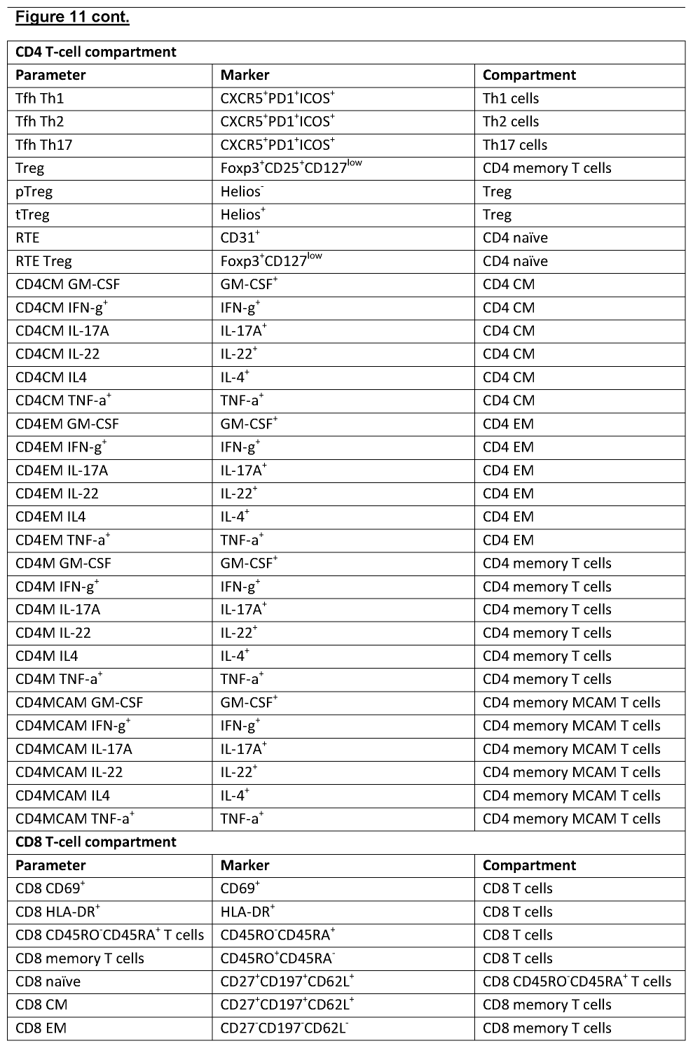

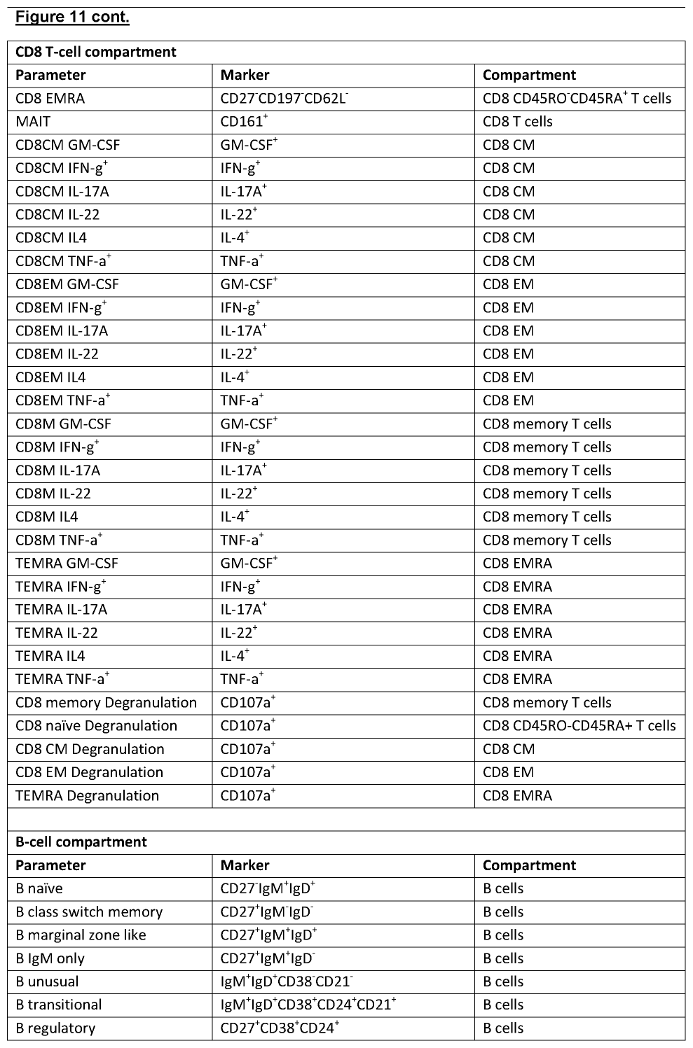

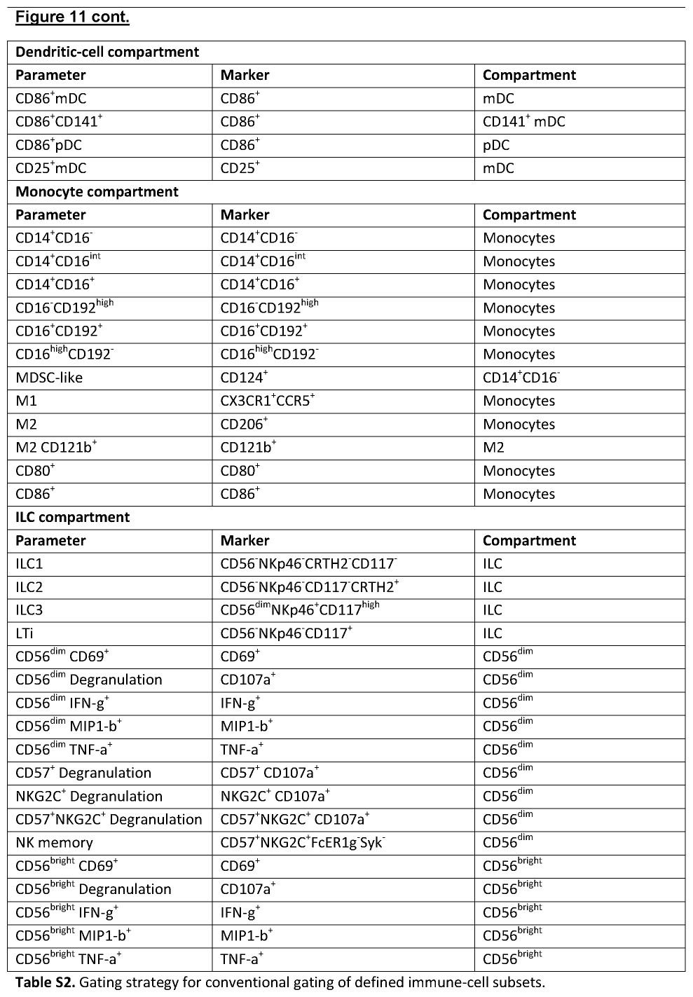

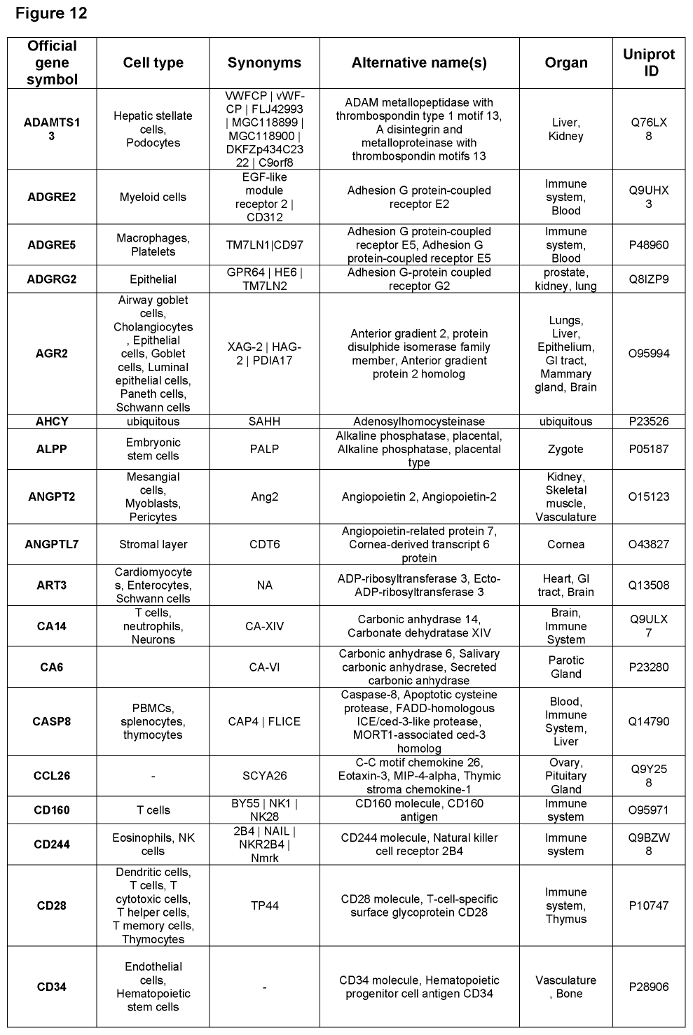

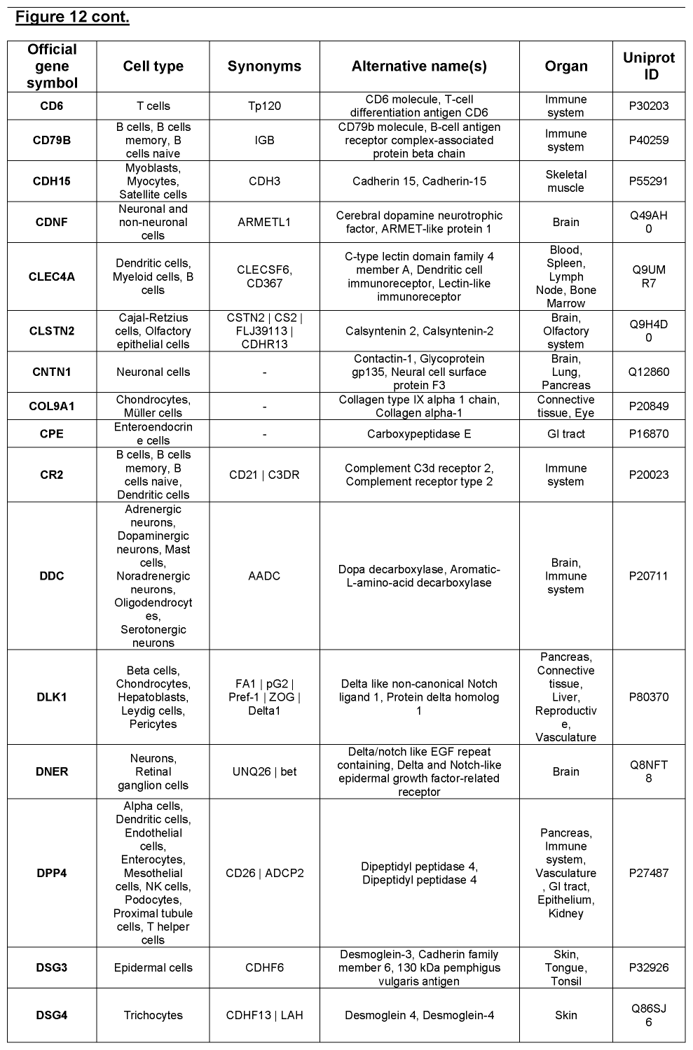

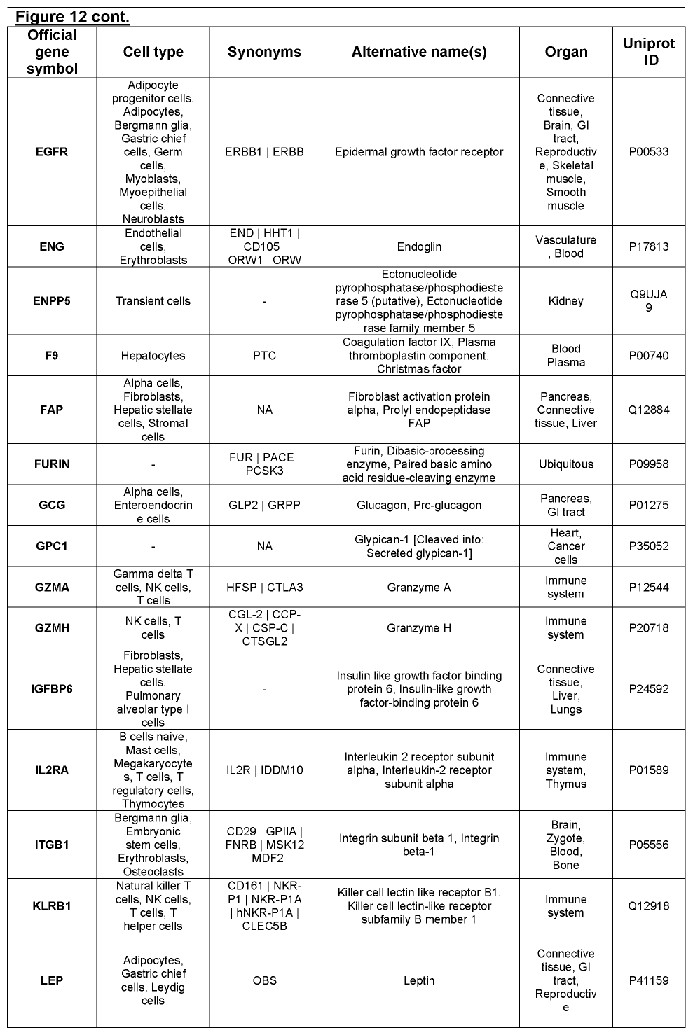

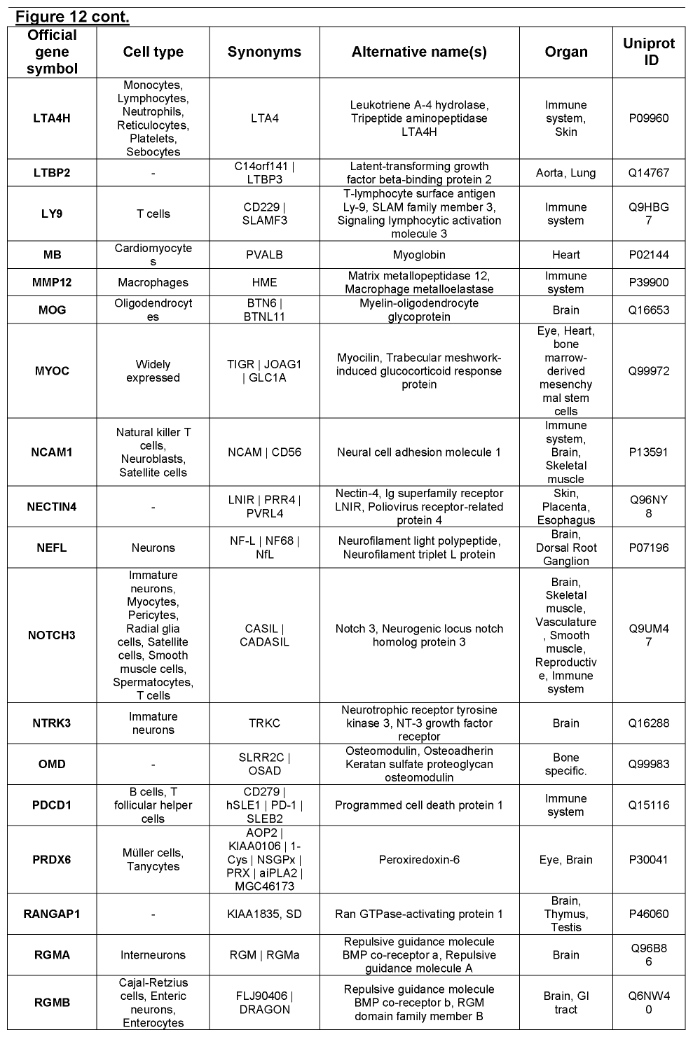

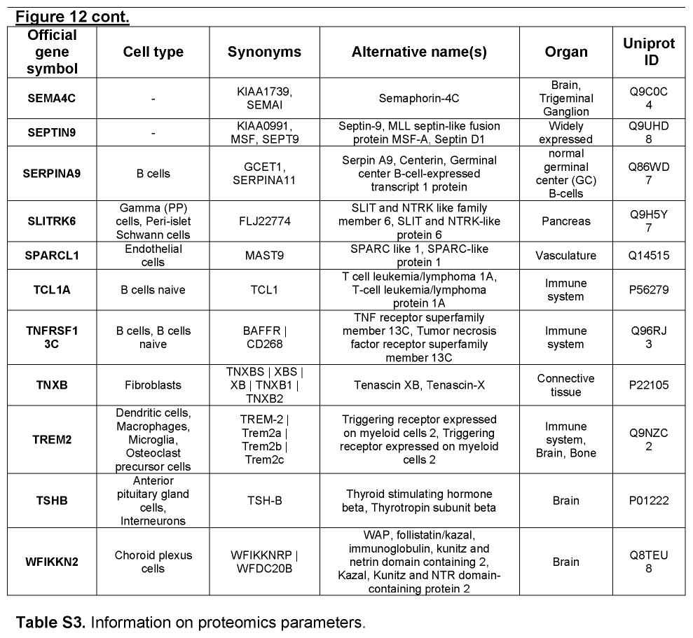

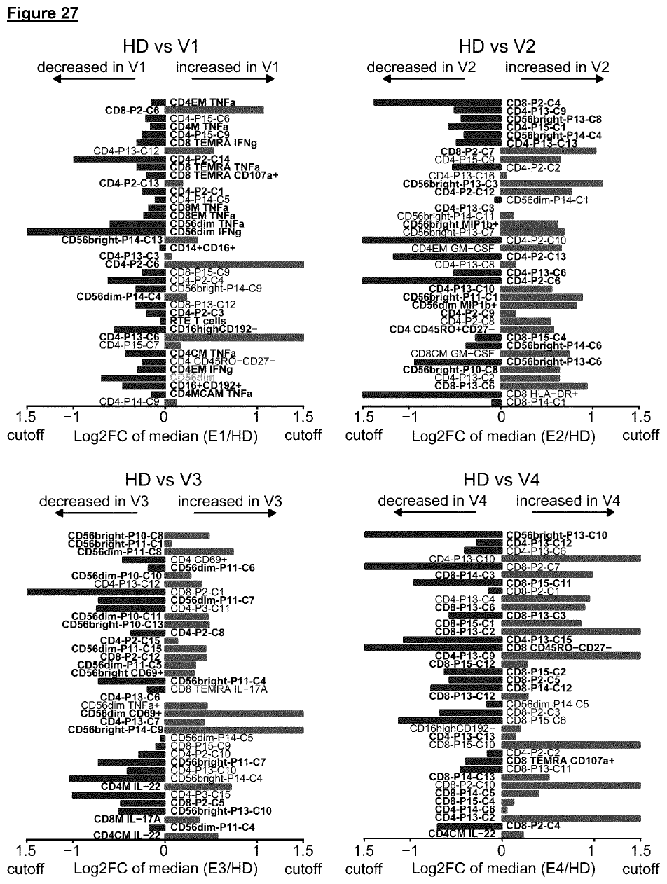

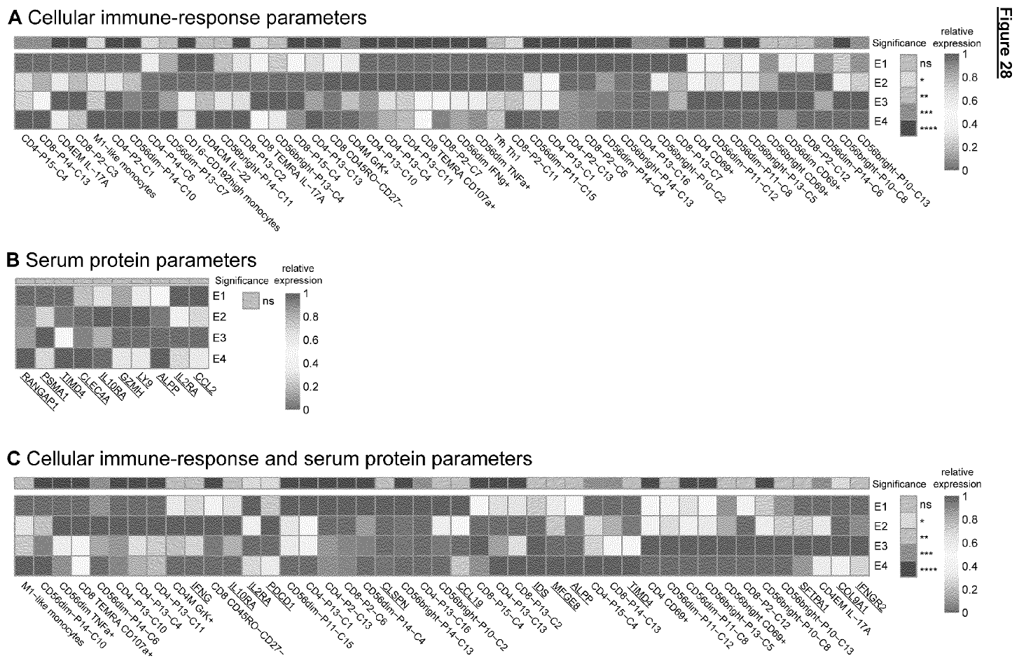

In case of k=70, the top 10% of cellular parameters defining each endophenotype distinguish multiple sclerosis patients from healthy individuals, with high prediction accuracies (E1: 91.8%, E2: 84.6%, E3: 95.6%, and E4: 80.6%; FIG. 4A). In contrast to cellular parameters, soluble parameters did not result in subsetting of multiple sclerosis patients into distinct endophenotypes (FIG. 4B) and accordingly the same set of soluble parameters distinguished each endophenotype (as pre-defined by cellular immune response signatures) from healthy individuals (E1: 86.7%, E2: 86.3%, E3: 88.3%, and E4: 94.4%) (FIGS. 25+26 with Supplementary FIG. 9+10). An independent validation cohort 78 multiple sclerosis patients and 30 healthy individuals confirmed the existence of four discrete immunological endophenotypes based on cellular immune-response signatures (FIG. 4C, FIG. 27 with Supplementary FIG. 11). The inventors next aimed to identify a minimal data set of significantly altered cellular and/or soluble parameters required for the stratification of early multiple sclerosis patients into the four identified endophenotypes. Therefore, the inventors used the complete data set of 384 cellular and 1 450 soluble parameters acquired from a sub-cohort of 158 multiple sclerosis patients (FIG. 4D). In a first step, the inventors identified 47 out of 384 cellular parameters using the LASSO regression (FIG. 28 with Supplementary FIG. 12A). These 47 cellular parameters were sufficient to classify each endophenotype with a high (total) prediction accuracy of 93.1% (E1: 96.7%, E2: 100%, E3: 100%, E4: 81.3%, FIG. 4D). In a second step, the inventors investigated 1 450 serum-derived soluble parameters. In contrast to the cellular parameters, the inventors only identified 10 differentiating soluble parameters, which classified each endophenotype with a total prediction accuracy of 55.6% (E1: 56.7%, E2: 45.9%, E3: 48.1%, E4: 39.1%, FIG. 4D, FIG. 28 with Supplementary FIG. 12B). Finally, analysis of 384 cellular and 1 450 soluble parameters together revealed that a combination of 32 cellular and 13 soluble parameters (ME9) classified each endophenotype with a total prediction accuracy of 92.8% (E1: 90.0%, E2: 83.8%, E3: 100%, E4: 85.9%, FIG. 4D, FIG. 28 with Supplementary FIG. 12C). These data indicate that cellular parameters are indispensable for identifying the different endophenotypes.

In case of k=190, the inventors identified 29 out of 384 cellular parameters using the LASSO regression (cf. distinguishing parameter data set ME10u). These cellular parameters were sufficient to classify each endophenotype with a high prediction accuracy of 91.2% (FIG. 36B). When investigating the 1 450 serum-derived soluble parameters, the inventors observed that, in contrast to the cellular parameters, soluble parameters were insufficient for the classification of endophenotypes. Analysis of 384 cellular and 1 450 soluble parameters together revealed that a combination of 23 cellular plus 3 soluble parameters (cf. distinguishing parameter data set ME11u) classified each endophenotype with a prediction accuracy of 90.5% (FIG. 36B). These data indicate that cellular parameters are indispensable for identifying the different endophenotypes.

Immunological Endophenotypes are Associated with Discrete HLA Backgrounds

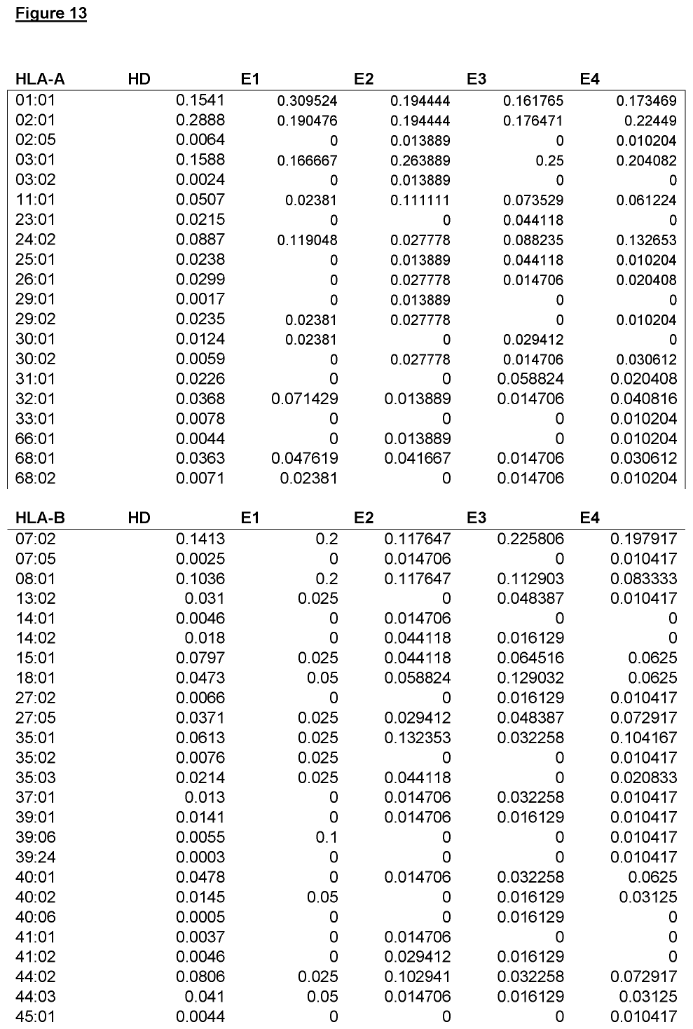

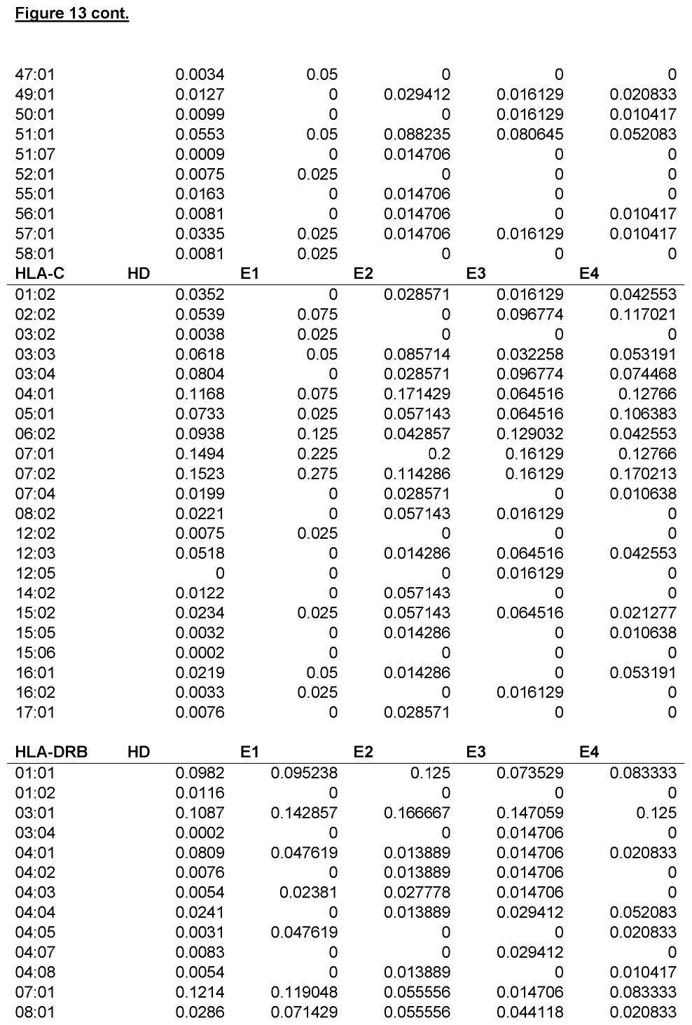

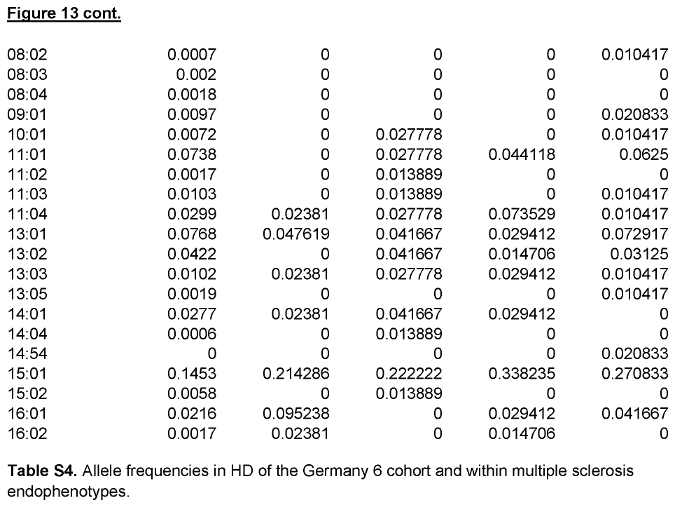

In light of the observed alterations in the adaptive immune compartment, the inventors next investigated, whether the four immunological endophenotypes are associated with a discrete HLA background. To this end, the four-digit HLA-genotype (Lank et al., 2012) for HLA-A, -B, -C, and -DR was assessed in a sub-cohort of 170 multiple sclerosis patients and compared to data from the Germany pop 6 cohort (http://www.allelefrequencies.net/hla6006a.asp?hla_population=2752) including information of 8862 controls (Schmidt et al., 2009) (in case of k=70: FIG. 5, FIG. 13 with Supplementary Table 4; in case of k=190: FIG. 37). Strikingly, hierarchic clustering revealed that the three or four immunological subtypes identified by cellular immune-response signatures correlated with distinct HLA-subtypes; henceforth, justifying the term endophenotype and further supporting the idea of discrete endophenotypes in multiple sclerosis (Gottesman and Gould, 2003; John and Lewis, 1966). Notably, among the endophenotype-specific alterations in HLA-subtypes we detected increased frequencies of the multiple sclerosis risk alleles HLA-DRB15: 01 (International Multiple Sclerosis Genetics et al., 2011) in endophenotype 3 (both in case of k=70 and k=190) and HLA-DRB03: 01 (Marrosu et al., 1997; Modin et al., 2004) and DRB13: 03 (Kwon et al., 1999) in endophenotype 2 (both in case of k=70 and k=190), whereas the protective allele HLA-DRB14: 01 (Zhang et al., 2011) was particularly decreased in endophenotype 4 (in case of k=70) and endophenotype 3 (in case of k=190). Taken together these data suggest that distinct immunological subtypes in multiple sclerosis maybe at least partially triggered by the respective HLA background.

Immunological Endophenotypes are Associated with Disease Severity

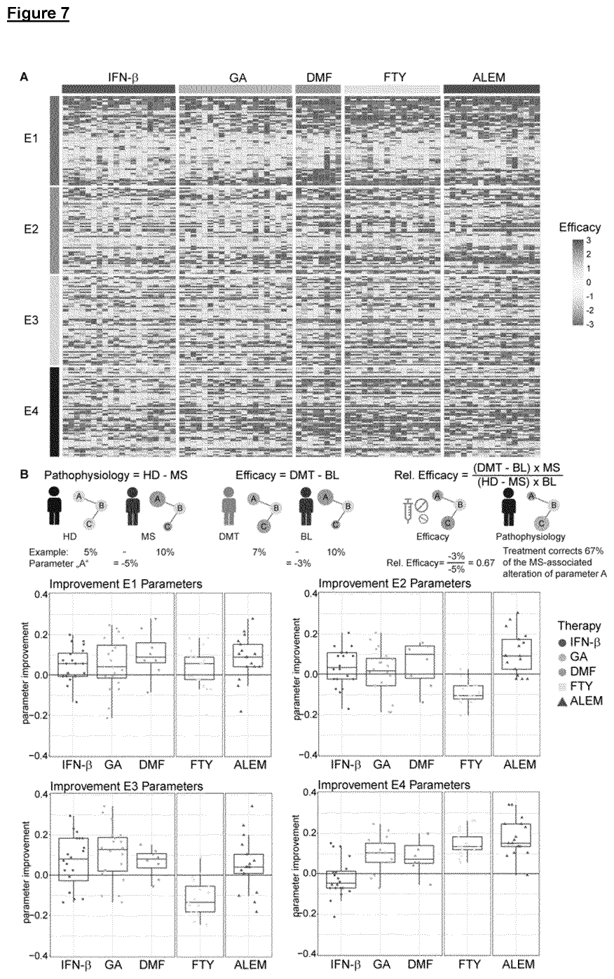

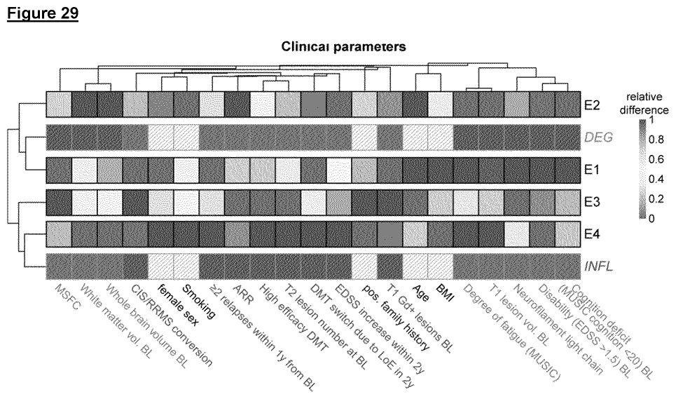

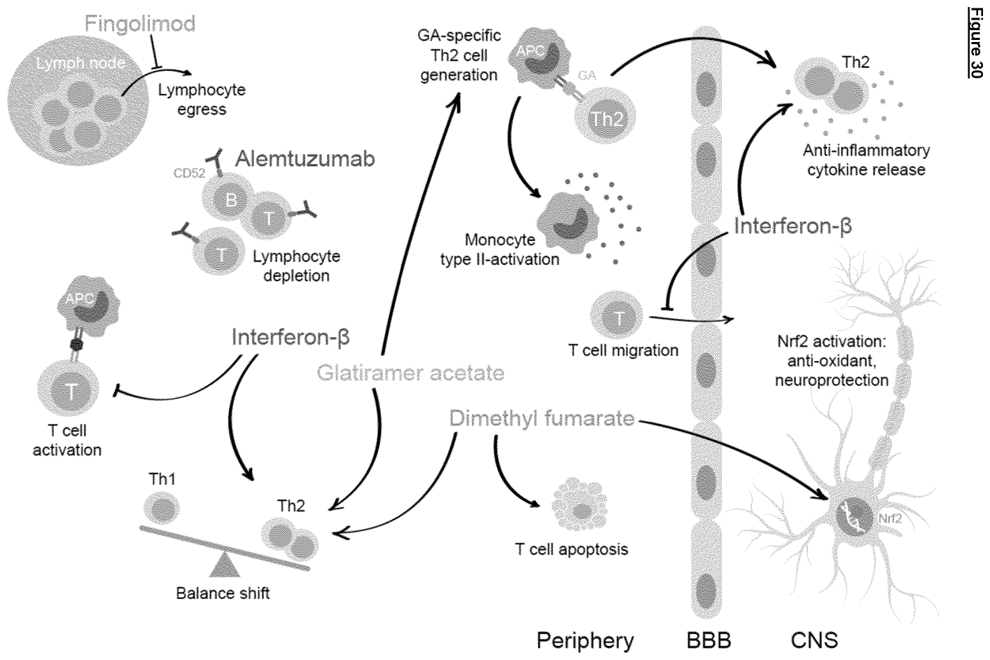

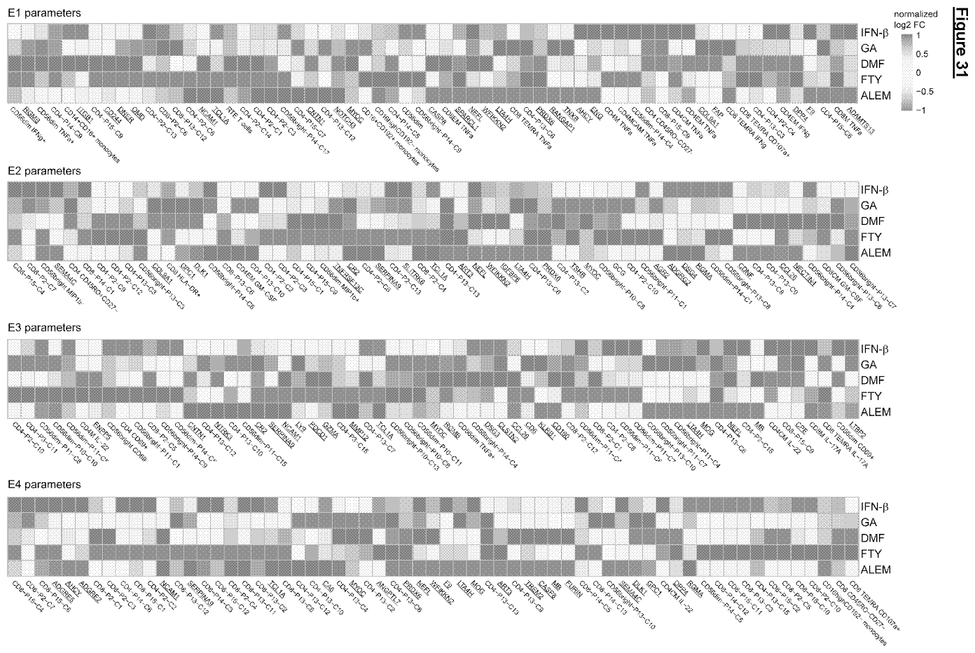

The inventors next investigated whether the three or four identified endophenotypes were associated with specific clinical and paraclinical disease trajectories. The inventors used the longitudinally acquired clinical and paraclinical data of our multicentric cohort, which were collected prospectively in a highly standardized manner (see methods) within the NationMS consortium (Ostkamp et al., 2021; von Bismarck et al., 2018) (FIG. 1). Notably, two of the three or four identified immunological endophenotypes were associated with opposite disease trajectories. Endophenotype 1 (both in case of k=70 and k=190) was associated with clinical and paraclinical markers pointing towards a higher degree of early structural brain damage and disease severity, including disability at baseline (EDSS>1.5) (Kurtzke, 1983), impaired cognition already at baseline (MUSIC cognition (Calabrese et al., 2004) <20), increased levels of the progression marker neurofilament light chain in the serum at baseline (NEFL), a higher degree of fatigue at baseline (MUSIC Test), as well as higher MRI-derived T1 lesion volume at baseline (FIG. 6 and FIG. 38). In contrast, endophenotype 4 (in case of k=70) and endophenotype 3 (in case of k=190), respectively, displayed a pattern reflecting signs of higher inflammatory disease activity, illustrated by higher relapse rate (≥2) within one year from baseline, EDSS (Kurtzke, 1983) increase within two years after baseline, numbers of gadolinium-enhancing (in case of k=70: T1 and T2, in case of k=190 only T2) esp. T2 lesions at baseline, as well as the use of highly-active DMTs already early in the disease course. According to these different disease trajectories, we therefore termed these subsets degenerative endophenotype (endophenotype 1 in case of k=70) and inflammatory endophenotype (endophenotype 4 in case of k=70, endophenotype 3 in case of k=190). The remaining other one or two endophenotypes exhibited intermediate characteristics and patterns of disease severity and activity. This assignment of endophenotypes to distinct disease trajectories could be confirmed by automated hierarchical clustering (e.g. in case of k=70: FIG. 6 and FIG. 29 with Supplementary FIG. 13, in case of k=190 FIG. 38). Overall, the data indicate that multiple sclerosis not only consists of distinct immunological endophenotypes, but that these endophenotypes might have predictive value for prognosis of inflammatory versus degenerative disease activity.

Different Capacity of Distinct Immune-Therapeutic Principles to Normalize Immune-Alterations Associated with Each Endophenotype

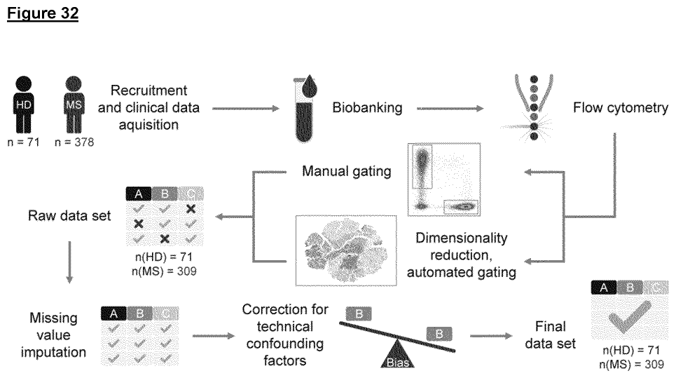

The inventors next wondered whether different approved MS treatments might exert differential effects on the three or four identified endophenotypes and whether this might correlate with different therapeutic responses. To this end, the inventors first determined the specific alterations of cellular and soluble parameters induced by treatment with different DMTs across various modes of action (FIG. 7A). The inventors analyzed samples from patients before and during treatment with interferon-beta, glatiramer acetate, and dimethyl fumarate as immune-modulatory drugs, the S1P-modulator fingolimod (immune-trafficking drug), and the monoclonal CD52-antibody alemtuzumab (immune cell-depleting agent) (FIG. 30 with Supplementary FIG. 14). Overall—and as expected—treatment with DMTs resulted in treatment-specific alterations in the immune-response signatures (FIG. 31 with Supplementary FIG. 15). While similar alterations were induced by all immune-modulatory drugs, fingolimod and alemtuzumab resulted in clearly distinct alterations of the immune-response signatures. Next, we, the inventors, calculated the capacity of each drug to normalize the immune-response signatures affected in each of the 3 and 4 endophenotypes (k=190 and k=70, respectively) to determine the effect size of each treatment in each of these endophenotypes (FIG. 7B and FIG. 39). Strikingly, immune-response signatures associated with the degenerative endophenotype 1 were normalized to a comparable extent by all DMTs investigated, whereas the two and three other endophenotypes, respectively, displayed differential treatment responses towards different therapeutical principles. This indicates that the degenerative endophenotype responds invariably to diverse treatments, while the inflammatory endophenotype shows more specific responses towards immunotherapeutic principles. Notably, fingolimod had a reduced capacity to improve immune response signatures associated with the mixed endophenotypes 2 and particularly 3 (k=70) and endophenotype 2 (k=190), respectively. In contrast, immunotherapy with alemtuzumab displayed a high capacity for immune-response signature normalization in all three or four endophenotypes investigated and particularly in the inflammatory endophenotype 4 (k=70) and 3 (k=190), respectively. In contrast, interferon-beta treatment had limited effects on signature alterations in the inflammatory endophenotype 4 (k=70) and 3 (k=190), respectively, whereas glatiramer acetate and dimethyl fumarate treatment resulted in a pronounced ‘improvement’ of these signatures.

### Treatment Responses Differ Between Endophenotypes

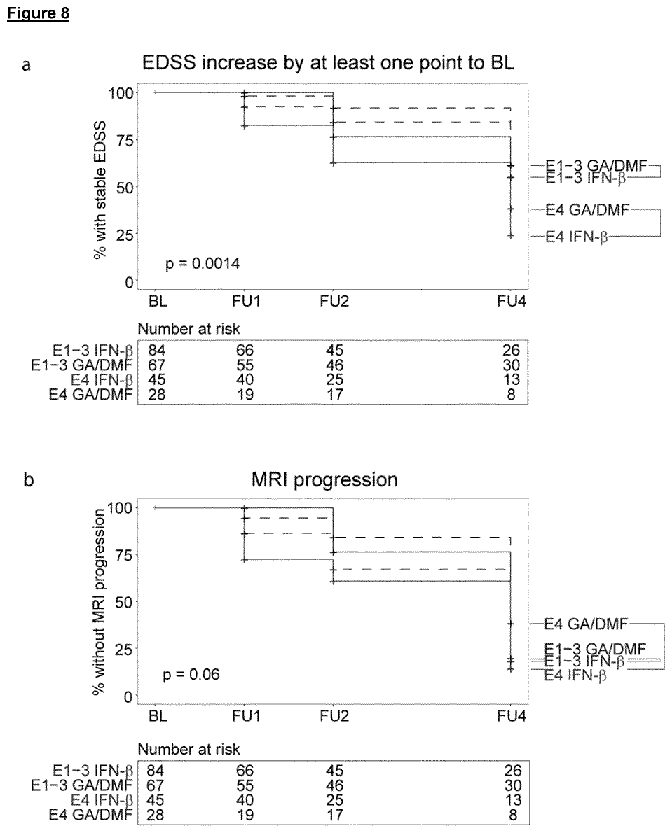

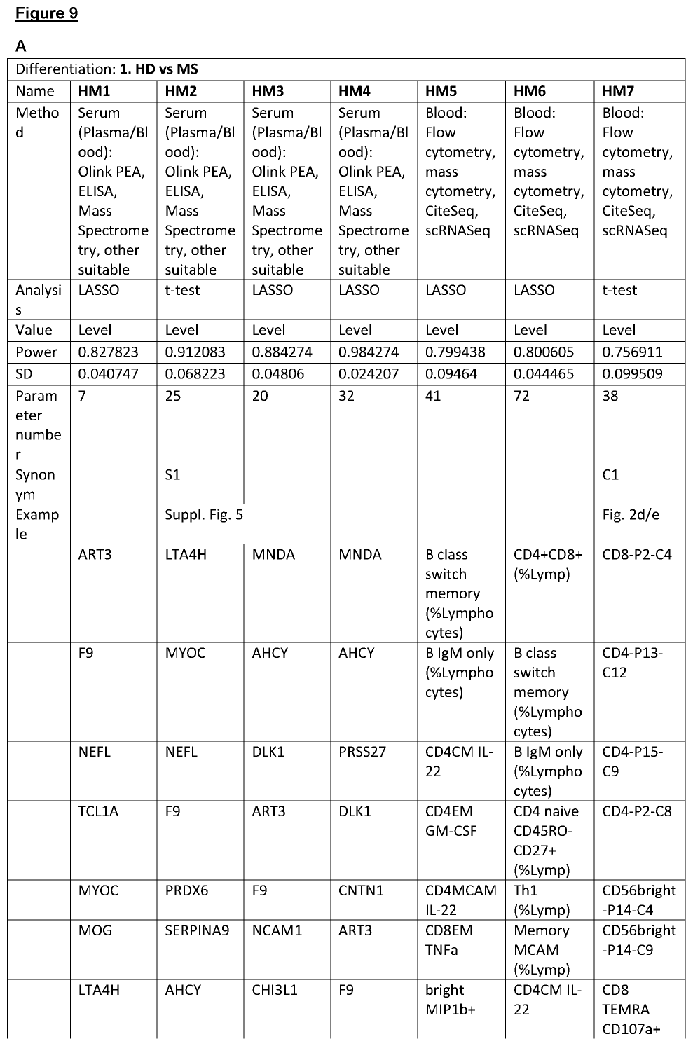

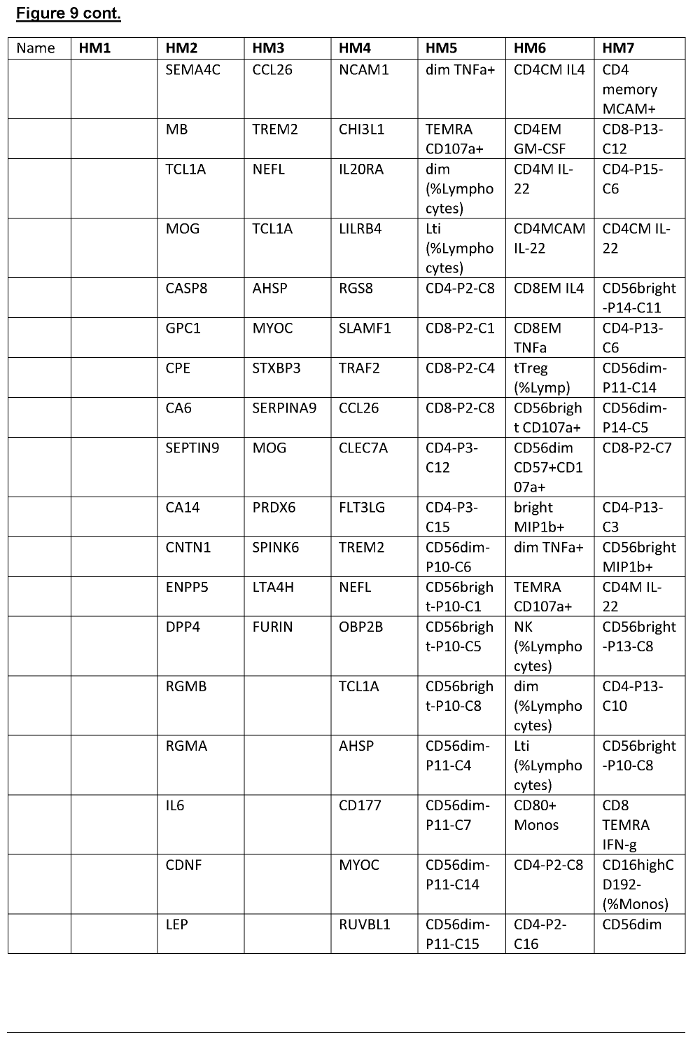

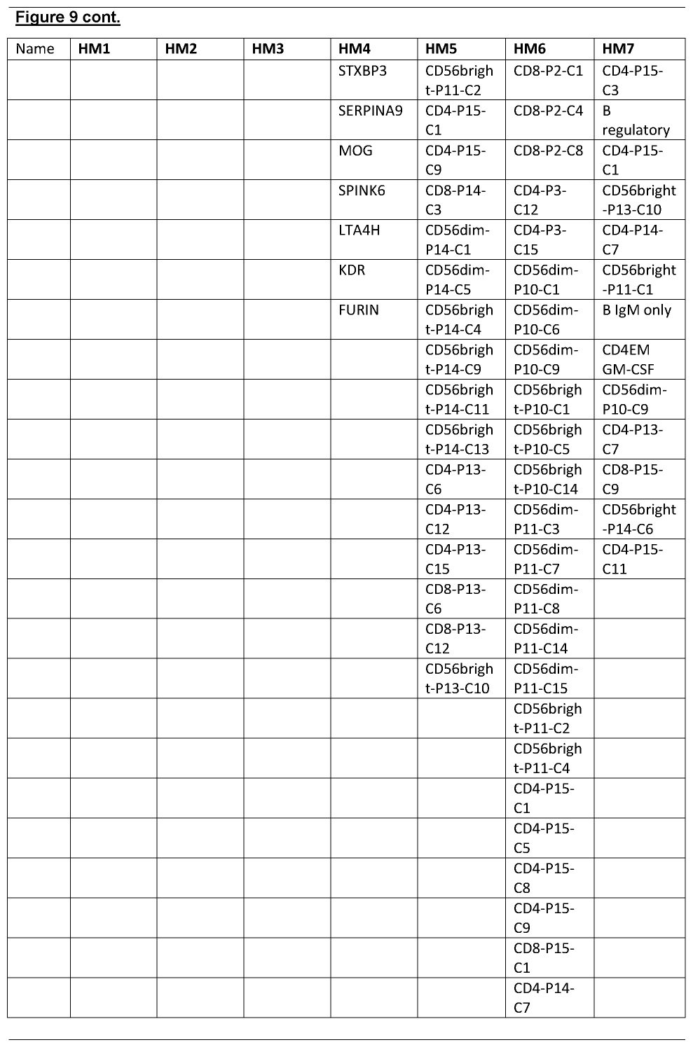

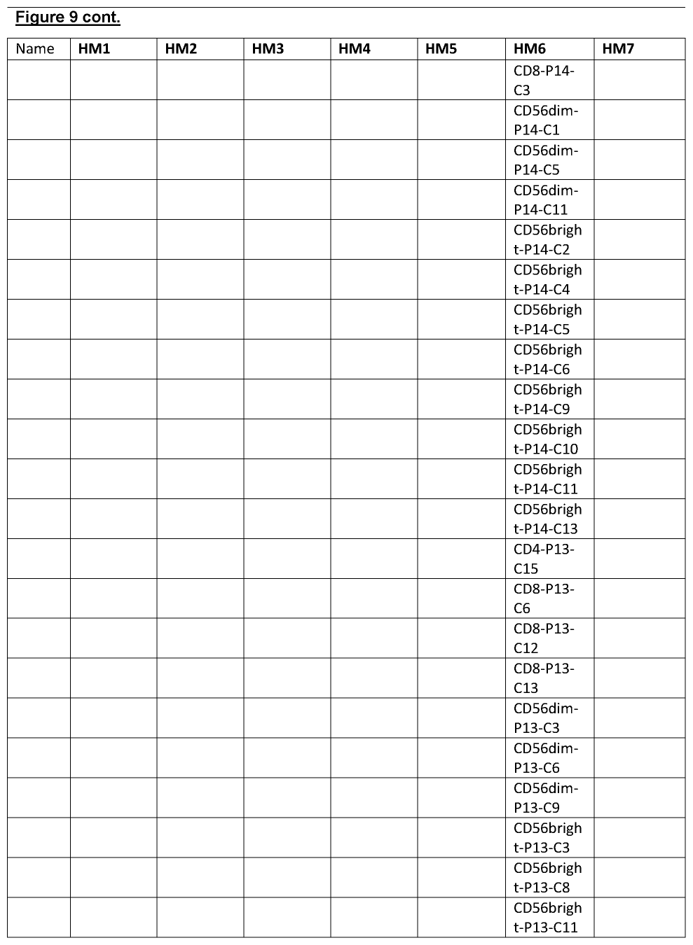

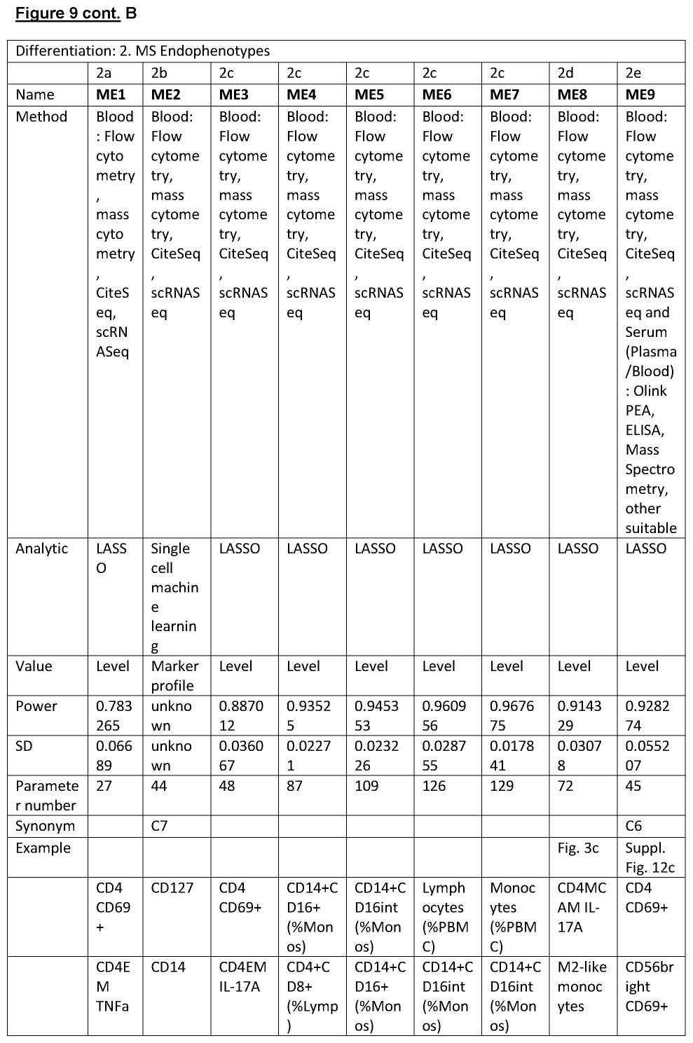

To test the clinical relevance of this concept of differential treatment responses depending on the prevailing endophenotype, the inventors compared the clinical response towards interferon beta versus other platform therapies (i.e. glatiramer acetate or dimethyl fumarate) in patients with the inflammatory endophenotype 4 (k=70) and 3 (k=190), respectively, over up to 4 years (FIG. 8 and FIG. 38). Pooled endophenotype 1-3 (k=70) and 1-2 (k=190), respectively, patients served as a control group. Strikingly, and in accordance with the lack of improvement of inflammatory endophenotype 4 (k=70) and 3 (k=190), respectively, immune response signatures by interferon-beta treatment, interferon-treated endophenotype 4 (k=70) and 3 (k=190), respectively, patients displayed a significantly enhanced increase in disability (EDSS, FIG. 8A and FIG. 38A) when compared to patients of the same endophenotype receiving other platform therapies. In support of this notion, the proportion of patients with ongoing MRI disease activity during treatment (i.e. novel back holes, brain atrophy, new or enlarging T2 lesions or Gd+ lesions, all together termed “MRI progression”) was increased in interferon-treated endophenotype 4 (k=70) and 3 (k=190), respectively, patients compared to endophenotype 4 (k=70) and 3 (k=190), respectively, patients receiving other platform therapies (FIG. 8B and FIG. 38B). Notably, differences between those treatments were less pronounced in multiple sclerosis patients of pooled remaining endophenotype 1-3 (k=70) and 1-2 (k=190), respectively, again in accordance with similar fitting accuracies as depicted in FIG. 7B. These data demonstrate that treatment responses to specific immune therapies may depend on and can be predicted by immunological endophenotypes quantified before treatment initiation. Therefore, a priori knowledge of the respective endophenotypes could help optimizing treatment decisions.

### Material and Methods

### Ethics Statement

The study was conducted in accordance with the Declaration of Helsinki. The NationMS study was approved by the ethics committee of the Ruhr-University Bochum (Registration no. 3714-10), and consecutively, by all local committees of the participating centres. Healthy individuals and additional multiple sclerosis patients of the treatment cohort were recruited based on ethics votes of the ethics committee of the University of Münster (Registration nos. 2010-378-b-S, 2010-262-f-S, 2011-665-f-S, 2012-236-f-S, 2013-350-f-S, 2014-068-f-S, 2014-398-f-S, 2016-053-f-S, 2019-712-f-S) and Ruhr University Bochum (3714-10). All patients provided written informed consent.

### Patient Cohorts

The current study was performed using the so-called NationMS cohort (FIG. 1, (Ostkamp et al., 2021; von Bismarck et al., 2018)), a multicentric cohort of >1 000 therapy-naïve early multiple sclerosis patients with prospective comprehensive and standardized clinical data collection followed up to 4 years. Patients with clinically isolated syndrome (CIS) fulfilling 3 out of 4 Barkhof criteria (Barkhof, 1997) or early relapsing remitting multiple sclerosis (RRMS) patients according to Barkhof or McDonald criteria (Polman et al., 2011) were included. At baseline, patients did not show any other neurological or psychiatric conditions or a progressive disease course, and had never received any disease modifying therapy (DMT). At baseline, EDTA blood and serum was withdrawn during the stable phase of the disease at least 30 days following relapse. Peripheral blood mononuclear cells (PBMC) were isolated. PBMC and serum were cryopreserved until further use. PBMC of a sub-cohort of 387 multiple sclerosis patients were analyzed by flow cytometry in the discovery cohort and 78 independent patients in the validation cohort (FIG. 10 with Table S1). Proteomic analysis via Olink was performed in a sub-cohort of 158 multiple sclerosis patients. Seventy-one (discovery) and 30 (validation) age- and sex-matched healthy individuals served as controls (FIG. 1, FIG. 10 with Table S1). To assess the impact of diseases modifying treatments (DMTs) PBMC and serum derived from 20 interferon-β (IFN-β), 20 glatiramer acetate (GA), 20 dimethyl fumarate (DMF), 20 fingolimod (FTY), and 17 alemtuzumab (ALEM; follow-up 12 months after the last injection cycle) multiple sclerosis patients were analyzed prior to and at least one year following treatment initiation (FIG. 10 with Table S1).

### Clinical Assessment

Multiple sclerosis patients included in the NationMS cohort underwent a thorough initial assessment of clinical, MRI, demographic, environmental, and genetic parameters and were routinely followed up for up to 4 years (FIG. 1). Clinical parameters included expanded disability status scale (EDSS) at baseline dichotomized into patients without disability (EDSS≤1.5) and with at least mild disability (EDSS>1.5). Accordingly, patients were distinguished based on the annualized relapse rate throughout the observation period as well as the occurrence of at least 2 relapses within the first year after baseline. In addition, EDSS progression of more than 1 point within the first 2 years of observations was assessed. Further clinical scores included the multiple sclerosis functional composite (MSFC) measure at baseline (Fischer et al., 1999), rating the leg, arm, and cognitive function, the multiple sclerosis inventory cognition (MUSIC) test (Calabrese et al., 2004), allowing identification of patients with cognition deficits (<20) at baseline, and rating of fatigue. Disease severity and progression were further reflected by the proportion of patients transitioning from CIS to multiple sclerosis, the initial need for highly effective DMTs (FTY, Natalizumab, or ALEM; Rituximab/Ocrelizumab were not available at baseline), and the necessity to switch DMTs due to lack of efficacy within the first two years. Whereas CNS integrity was assessed in all patients at baseline as the number of T2 lesions, a sub-cohort of patients (n=118) underwent more detailed MRI workup. This investigation included assessment of the presence of gadolinium enhancing T1 lesions, whole brain volume, cerebral white matter volume, and T1 lesion volume at baseline, which were normalized into z-scores for comparison. Demographic and environmental factors included age, sex, body mass index, smoking, and cases of multiple sclerosis within parents, siblings, or cousins.

### Isolation and Cryo-Preservation of PBMC

80 ml EDTA blood was withdrawn using 9 ml K3 EDTA S-Monovettes (Sarstedt). PBMC were isolated from EDTA blood by density gradient centrifugation using Lymphoprep (Stemcell technologies). For this purpose, 15 ml Lymphoprep were carefully overlaid with 35 ml EDTA blood diluted with PBS at a 1:1 ratio. Tubes were centrifuged at 800 g for 30 min without acceleration or brake. The interphase was carefully aspirated and transferred into a new tube before being washed twice with PBS. Resulting PBMC were counted and centrifuged at 300 g for 10 min before being resuspended in CTL-C solution (CTL Cryo ABC kit, Immunospot). Finally, CTL-AB solution (Immunospot) was slowly added at a 1:1 ratio and the cell suspension at a final concentration of 10×10⁶ cells per ml was cryo-preserved in Cryotubes (Nunc) by gradual freezing in MrFrosty cryocontainers (Nalgene) for 48h before being transferred to the vapor phase of a liquid nitrogen tank (Unutmaz et al., 2014).

### Serum Preparation and Storage

Serum samples were centrifuged at 2000 g for 10 min and 1 ml aliquots of cell-free supernatant was frozen at −80° C. until analysis.

### OLink

Cryo-preserved serum samples were sent to Olink for proteomic analysis by the Explore 1536 panel (Olink proteomics) by proximity extension assay (PEA) using next generation sequencing as recently published (Filbin et al., 2021). For this purpose, samples are incubated with specific antibodies conjugated to unique DNA oligo sequences. Since only matched DNA sequences from identical antibodies bound to the same molecule are detected, unspecific signals are reduced, thus, allowing high-dimensional multiplexing. All samples were acquired in a single run to avoid batch effects. Resulting relative expression values were used for subsequent analysis.

### Flow Cytometry

For flow cytometry, PBMC were thawed in a 37° C. water bath for 8 min. The cell suspension was transferred to a 50 ml conical tube and 9 ml pre-warmed RPMI-medium (RPMI (Sigma Aldrich), 10% FCS Gold Plus (BioSell), 1% Glutamax (Gibco), 1% Na-Pyruvate (Invitrogen)) was added prior to centrifugation at 300 g for 10 min. Supernatant was discarded and the cell pellet was resuspended in RPMI-medium. PBMC were counted and viability was assessed using a Countess II automated cell counter (Invitrogen). Subsequently, PBMC were subjected to functional deep immune phenotyping by flow cytometry. For this purpose, PBMC were directly stained with fluorochrome-conjugated antibodies directed against lineage-defining epitopes, markers of cellular differentiation, activation, and maturation as well as receptors involved in proliferation and regulation of effector functions (FIG. 16 with Table S7). In addition, intra-cellular/-nuclear epitopes were investigated by incubation of PBMC with Perm/Fix buffer (BD Biosciences) for 20 min at room temperature and subsequent staining for 30 min at 4° C. in Perm buffer (BD Biosciences) (FIG. 14 with Table S5). Finally, the functional capacity of immune-cell subsets was investigated by both, unspecific stimulation with PMA/lonomycin/Brefeldin A (leukocytes activation cocktail, LAC, BD Biosciences) and specific stimulation by re-directed cross-linking of CD3 or DNAM1 and 2B4 followed by staining for lineage markers as well as cytokines or CD107a as a marker for the directed degranulation of cytolytic vesicles (FIG. 15 with Table S6). Specificity of the staining was validated using isotype control antibodies (FIG. 16 with Table S7). Samples were acquired on a Cytoflex flow cytometer (Beckman Coulter) under daily quality control by CytoFlex Daily QC Fluorospheres (Beckman Coulter). Stability of measurements was further verified by repeated analysis of reference samples throughout the duration of the entire study. For the discovery cohort cryopreserved PBMC from 449 donors were analyzed by flow cytometry (FIG. 32 with Fig. S16). From these, 68 donors were retrospectively excluded due to low sample viability and/or quality. One patient was excluded due to unclear diagnosis. Following missing value imputation in the remaining data set and correction for technical confounding factors such as center specific variations, a final data set encompassing a total of 380 donors (FIG. 32 with Fig. S16) was investigated for differences in immune-response signatures between healthy individuals (n=71) and early multiple sclerosis (n=309) as well as within early multiple sclerosis (E1 n=65, E2 n=73, E3 n=64, E4 n=107). In addition, PBMC from 98 early multiple sclerosis patients before and after at least 12 months of treatment with distinct DMTs were investigated, from which one was discarded due to withdrawal of consent. As a result, samples from 20 IFN-β, 20 GA, 20 DMF, 20 FTY, and 17 ALEM patients were further investigated for DMT-related effects on cellular parameters altered in early multiple sclerosis.

### Manual Gating

Data resulting from flow cytometric investigations was analyzed by Kaluza 2.1 (Beckman Coulter) by manual gating on established PBMC subsets (FIG. 11 with Table S2) and on markers for degranulation of cytolytic vesicles or cytokines.

### Automated Gating

The inventors used automated gating for the investigation of complex phenotypic properties within defined lymphocyte subsets. For this purpose, flow cytometry files were loaded to FlowJo (V10.3) and manually gated for CD4 or CD8 memory T cells (FSC^{low}SSC^{low}CD3⁺CD56⁻CD4/CD8⁺CD45RO⁺) as well as CD56^{dim}CD16⁺ or CD56^{bright}CD16^{dim/-} NK cells (FSC^{low}SSC^{low}CD3⁻CD56^{dim/bright}CD16^{+/dim/-}) Information on fluorescence intensities from the respective cell subsets was exported as new fcs files, linearized by ArcSin transformation, and normalized using Rstudio. Subsequently, files were loaded to Cyt V2 (Amir et al., 2013) run in Matlab R2016a (Mathworks). Samples were randomly subsampled to equal cell numbers and bh-SNE was performed followed by PhenoGraph with differentially expressed markers. Cluster-assignment of individual cells was exported and converted to relative numbers. The expression of cluster-defining markers is depicted in FIGS. 17-20 with Fig. S1-4.

### Data Processing

Manual gating for known cell subsets and markers for cellular effector functions such as cytokines and cytolytic activity as well as automated gating for CD4 and CD8 T-cell and CD56^{dim} and CD56^{bright} NK-cell phenotypes resulted in a data set of 694 parameters in total. Several variables in the data set presented between 0.0% and 17.3% of missing values with global missingness of 1% calculated over all variables in the data set. Parameters with a high missingness were carefully removed. Remaining missing values were imputed using the bootstrap expectation-maximization algorithm provided by the function Amelia of the R package “Amelia” (Honaker et al., 2011) (1.8.0). Imputation involved estimating m values for each missing value in the data set and creating m complete data sets, where the observed values are the same but the missing values are filled using a distribution of values that reflects the uncertainty around those missing values. The inventors considered 5 imputations, and a single estimate of missing values was then obtained by selecting the median. Diagnostic analysis of the results by over-imputation plots and distribution plots of imputed values showed that the algorithm converged successfully and the imputed values were adequate as they fell within the range of the measured data.

Following imputation of missing values with Amelia, parameters were investigated for potential confounding factors based on the center (representing differences in biobanking and shipment) and day of measurement (including technical confounders like handling, flow cytometer, and antibody lots) by principal variance component analysis (PVCA) per panel using the “pvca” package (1.28.0). Data was corrected for center and time of measurement based confounding factors by ComBat procedure (Johnson et al., 2007; Leek et al., 2012) of the “sva” package (3.36.0) using an empirical Bayes framework. Elimination of respective confounding factors was verified by PVCA. Of note, data was not corrected for demographic factors since cohorts were carefully age- and sex-matched.

Due to the multi-panel nature of this study, the resulting data set contained numerous duplicate parameters. For example, the same lineage markers were present in multiple panels to identify populations of interest prior to phenotyping. To avoid duplicate data and enable direct comparison, only parameters with the highest level of definition were retained, i.e. B cells were defined as CD19⁺CD20⁺ lymphocytes from panel 6 instead of just CD19+ lymphocytes from panel 1. In addition, the inventors utilized the specific powers of manual gating and automatic gating following dimensionality reduction. Since manual gating provides the opportunity to identify even tiny well-defined populations, all information on leukocyte subset proportions as well as expression of cytokines and markers for cytolytic activity was derived thereby. In contrast, automatic gating excels in examination of complex phenotypes. Hence, information on the expression of markers of cellular differentiation, activation, and maturation as well as of receptors involved in proliferation and regulation of effector functions was derived thereof. The result of this comprehensive parameter selection was a data set of 384 unique parameters consisting of 149 parameters from manual gating describing leukocyte proportions and effector functions as well as 235 parameters from automated gating describing complex phenotypes.

### HLA-Genotyping

High-resolution HLA typing was performed at the DKMS Life Science Lab, accredited by European Federation of Immunogenetics (EFI) and American Society for Histocompatibility and Immunogenetics (ASHI). Exons 2 and 3 of HLA-A, HLA-B, HLA-C and HLA-DRB1 were sequenced by next generation sequencing. Allele frequencies of four-digit HLA types were calculated for endophenotype 1 (n=21), endophenotype 2 (n=36), endophenotype 3 (n=34), and endophenotype 4 (n=49) and compared to allele frequencies of the Germany 6 cohort (n=8862, http://www.allelefrequencies.net/hla6006a.asp?hla_population=2752 (Schmidt et al., 2009)). Allele frequencies were centered and scaled to mean 0 and standard deviation 1 individually for each HLA-allele across all compared groups (HD, i) E1, E2, E3 and E4 or ii) E1, E2, and E3).

### Statistics

Unless stated otherwise, statistical and computational analyses were performed in Rstudio (1.1.442) running R4.0.2. For continuous parameters, p-values were calculated by Mann-Whitney test or Kruskal-Wallis test, respectively. Categorical data was compared by Fisher's exact test. Resulting p-values were corrected for multiple testing by Benjamini-Hochberg method resulting in the adjusted q-values. Mono-parametric alterations were depicted as fold-change or log 2 fold-change using GraphPad Prism 6. Alternatively, differences between two groups were plotted as networks using Cytoscape (3.9.0). For this purpose, tables with nodes, edges, significance labels, and log 2 fold change values were imported and information was illustrated by distinct border lines types and fillings in grey shades.

### Efficacy Scores

The efficacy of disease modifying therapies was calculated from matched samples at baseline, i.e. without prior treatment, and after treatment with the respective drug for at least 1 year. In case of alemtuzumab as a depleting therapy, samples were taken 1 year after the last infusion directly before the next treatment cycle. Demographic and clinical data of this treatment cohort are summarized in FIG. 10 with Table S1. Absolute efficacy was calculated as the difference between under-treatment (DMT) and baseline (BL). The results were set into relation with the disease-related alteration of the respective parameters by division by the difference between median healthy individuals and multiple sclerosis levels and corrected by possible batch effects by multiplication with median multiple sclerosis levels and division by median baseline levels:

$$Efficacy = DMT - B L$$

$$Pathophysiology = HD - MS$$

$$Rel . Efficacy = \frac{( DMT - B L ) x MS}{( HD - MS ) x BL}$$

As a result, patient individual efficacy scores for each parameter were obtained, indicating deterioration (<0), partial (0-1), or full (>1) amelioration.

### Heatmaps

For the generation of heatmaps, raw data was standardized as z-scores or linearly as indicated. Cut-off for z-scores was set to ±3 to prevent a dominant effect of outliers on the grey shade scale. The hierarchic clustering of both samples and parameters was performed using the Rstudio “pheatmap” package (1.0.12), followed by manually gathering of samples into their respective groups (e.g. healthy individuals and early multiple sclerosis). For heatmaps summarizing the relative difference between distinct parameters, the inventors calculated the median (continuous parameters) or mean (categorical parameters) for each parameter and each group prior to min/max normalization of the result. Resulting data was plotted by “pheatmap”.

### UMAP and Phenograph

Dimensionality reduction technique Uniform Manifold Approximation and Projection (UMAP) (Becht et al., 2019) implemented in the R package “umap” (0.2.7.0) was used to visually summarize the multidimensional structure of the data characterizing each individual donor. Hyperparameters “n_neighbors” (size of the local neighborhood) and “min_dist” (minimum distance between points in the low dimensional representation) were tuned from 20 to 70 and 0.01 to 0.9, respectively. The PhenoGraph algorithm (Levine et al., 2015) implemented in the R package “Rphenograph” (0.99.1) was applied for automated grouping (clustering) based on high-dimensional characteristics of the respective data. Hyperparameter “k” was tuned from 20 to 80 based on homogenicity of the resulting cluster assignment.

### Kaplan-Meier Plots

Kaplan-Meier plots were generated by selecting early multiple sclerosis patients from the exploration cohort who received IFN-β, GA, or DMF as first therapy after BL. Disability (EDSS) and MRI progression (i.e. defined as either detection of novel black holes, general brain atrophy, detection of new Gd enhancing T1 lesion or general increase in T2 lesion load) were analyzed over the course of 4 years under unaltered treatment or censored in case of change of treatment. Deterioration was visualized using packages ggpubr (0.4.0) and survminer (0.4.9) in Rstudio. Statistical analysis was performed by log-rank test.

### Machine Learning Pipeline

For classification, the inventors designed, trained, optimized, and evaluated an extensive machine learning pipeline comprising a robust scaler, class imbalance correction using the Synthetic Minority Oversampling Technique (SMOTE) algorithm (Fernández et al., 2018), and four classifiers (random forest, AdaBoost classifier, as well as linear and radial basis function (rbf) support vector machines (SVC)). To ensure the generalizability of the resulting models, we used nested, stratified 10-fold cross-validation to optimize choice of classifiers and predict a participant's class (e.g. healthy control vs. patient). Model performance was optimized for Matthews correlation coefficient. All analyses were performed using the PHOTON AI software package (Leenings et al., 2021b).

### LASSO

The inventors employed a two-step Least Absolute Shrinkage and Selection Operator (LASSO) focused analysis to determine parameters robustly contributing to the differentiation of multiple sclerosis endophenotypes. Therefore, the respective dataset was split randomly into 10 subsets. The inventors performed LASSO regularisation assessed by multinomial logistic regression by “glmnet” package (4.1-3) (Friedman et al., 2010) on 9 out of 10 in each run, while tuning lambda by inner cross-validation. From the ten resulting sets of selected parameters differentiating the groups we selected only those parameters which were selected in at least nine out of ten sets of parameters, thus trading some prediction accuracy for robustness and in parallel further reducing the number of parameters needed for separation by about 55%.

### Hyperparameter k

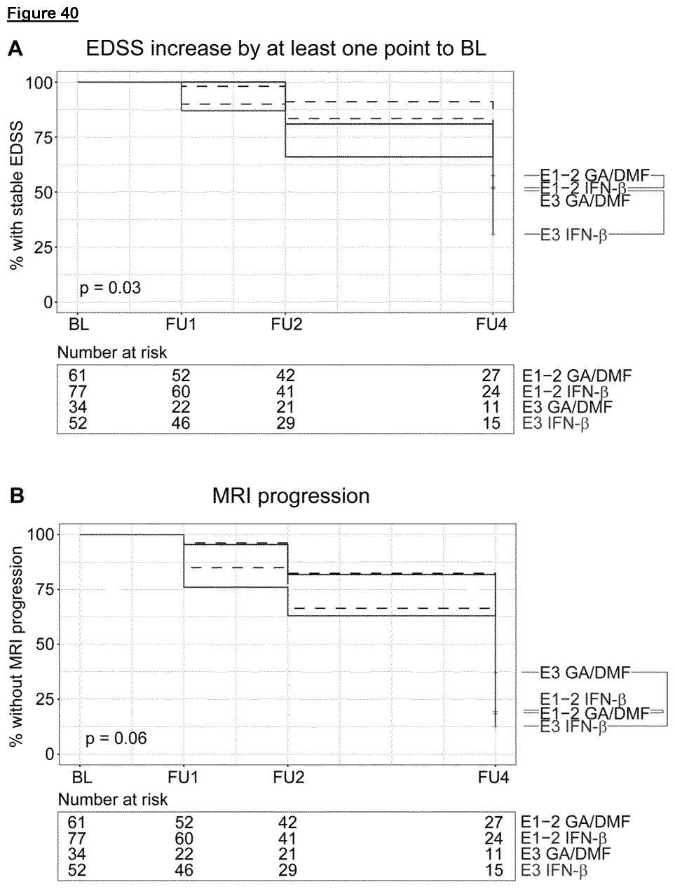

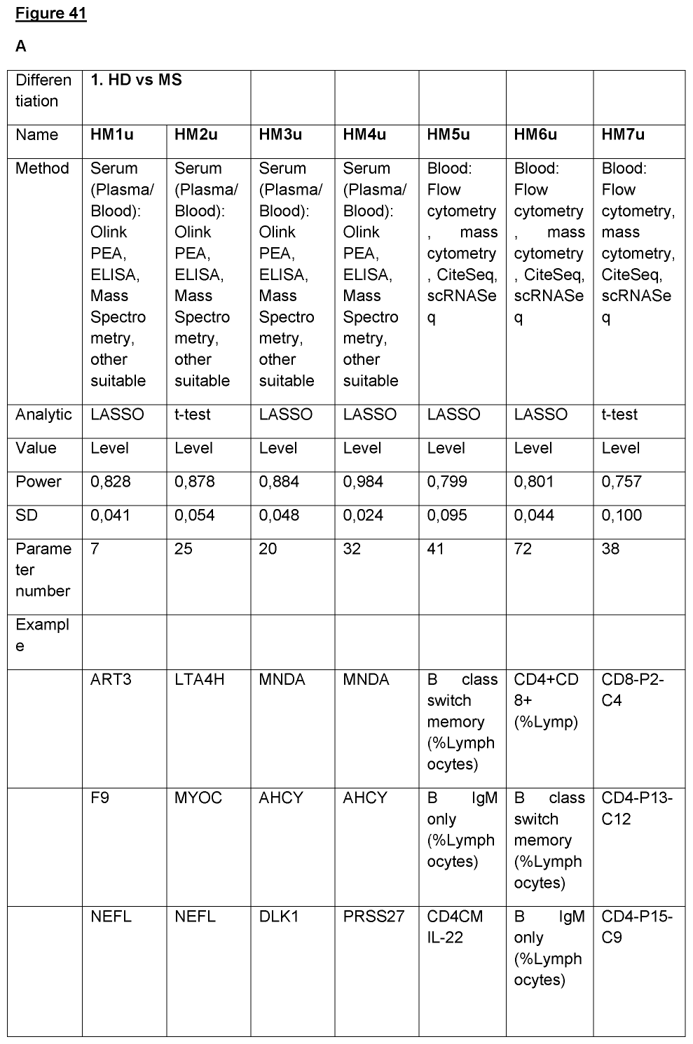

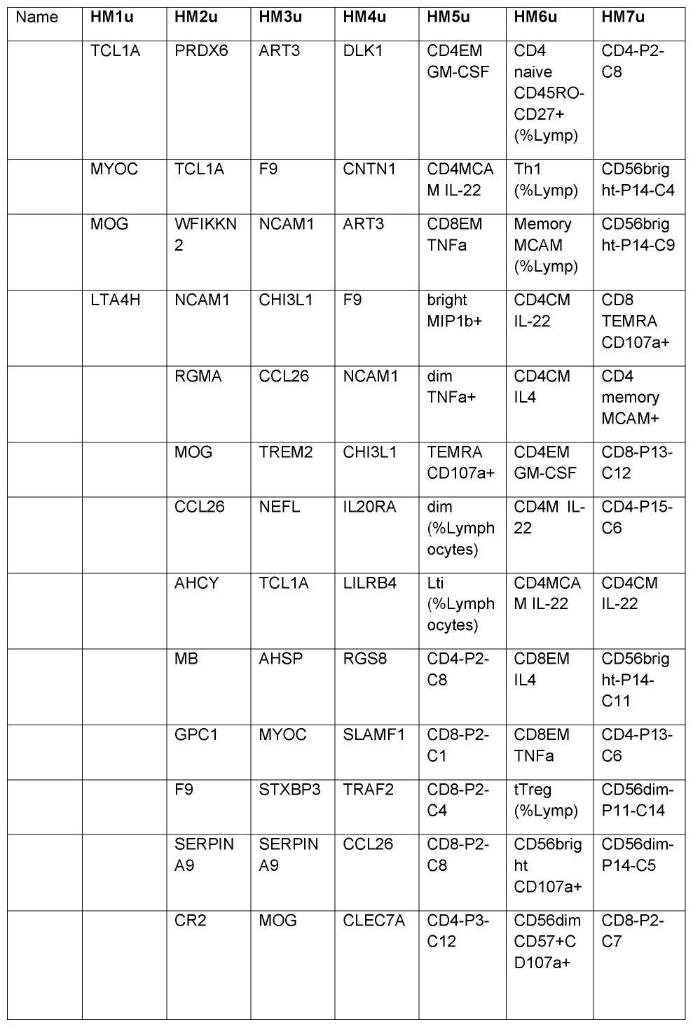

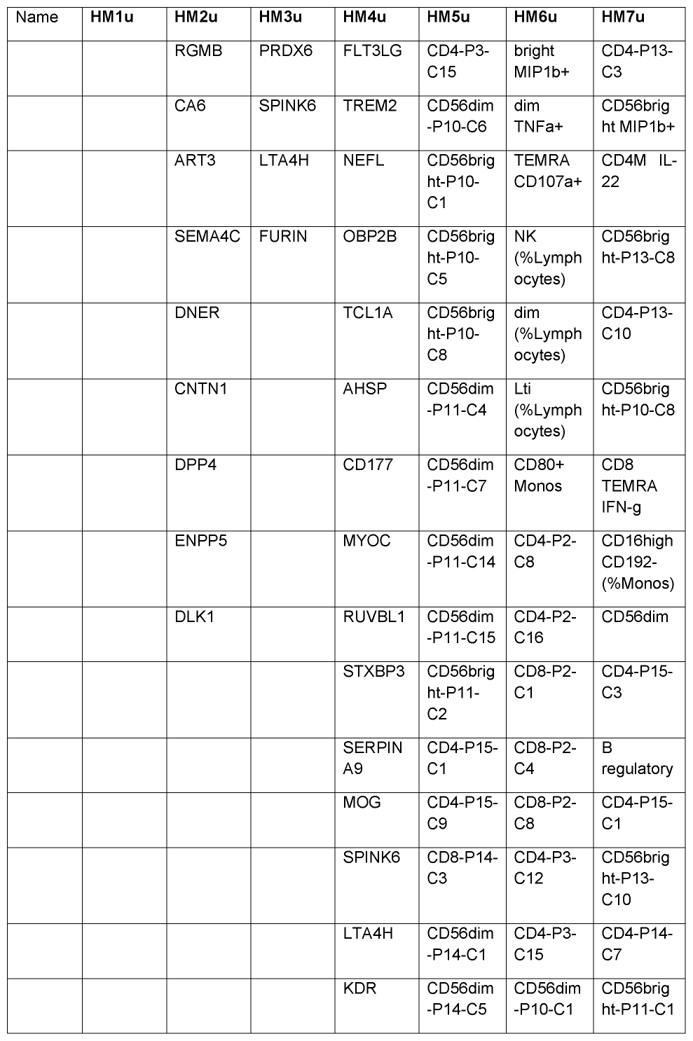

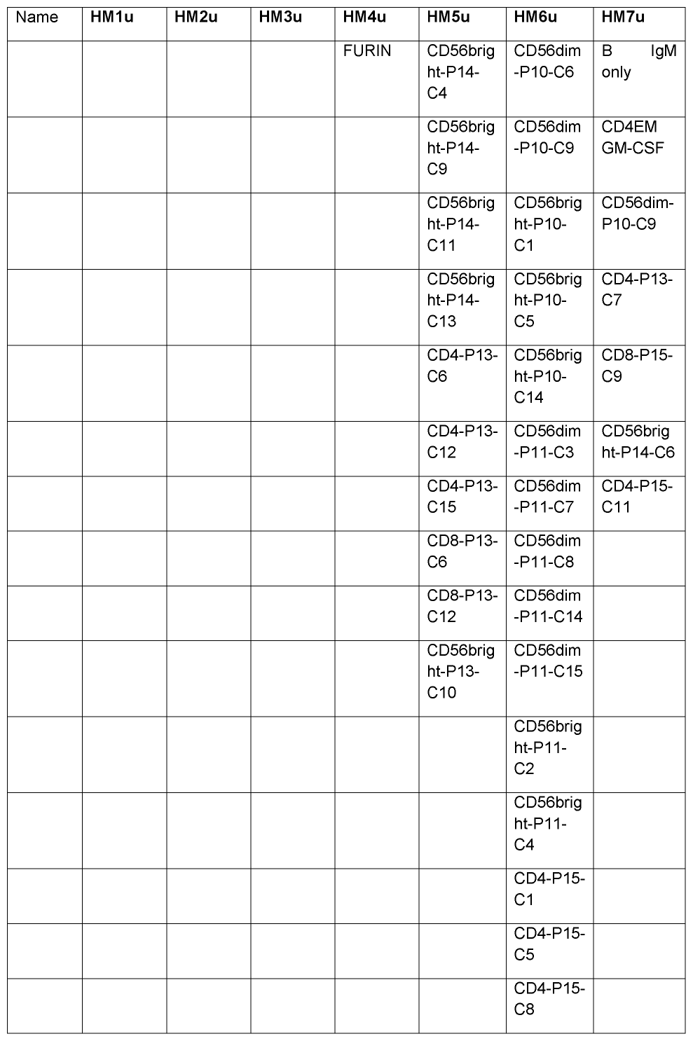

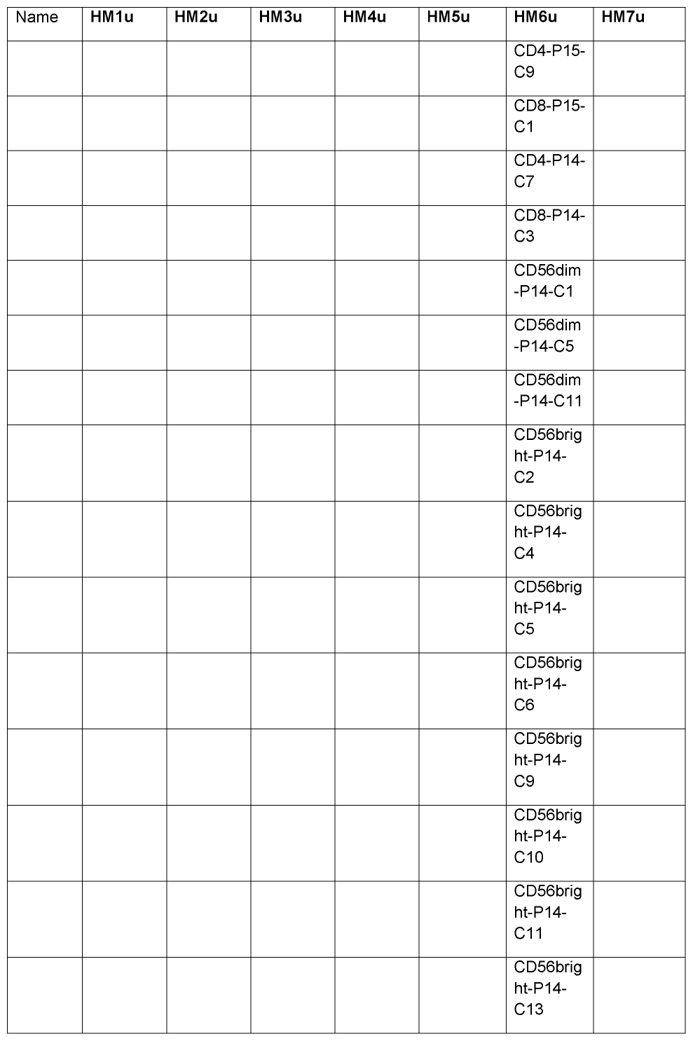

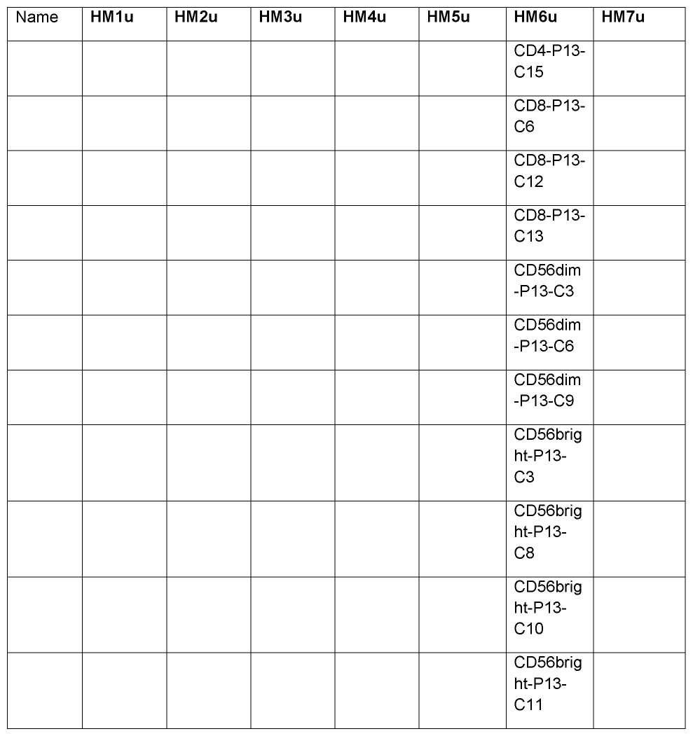

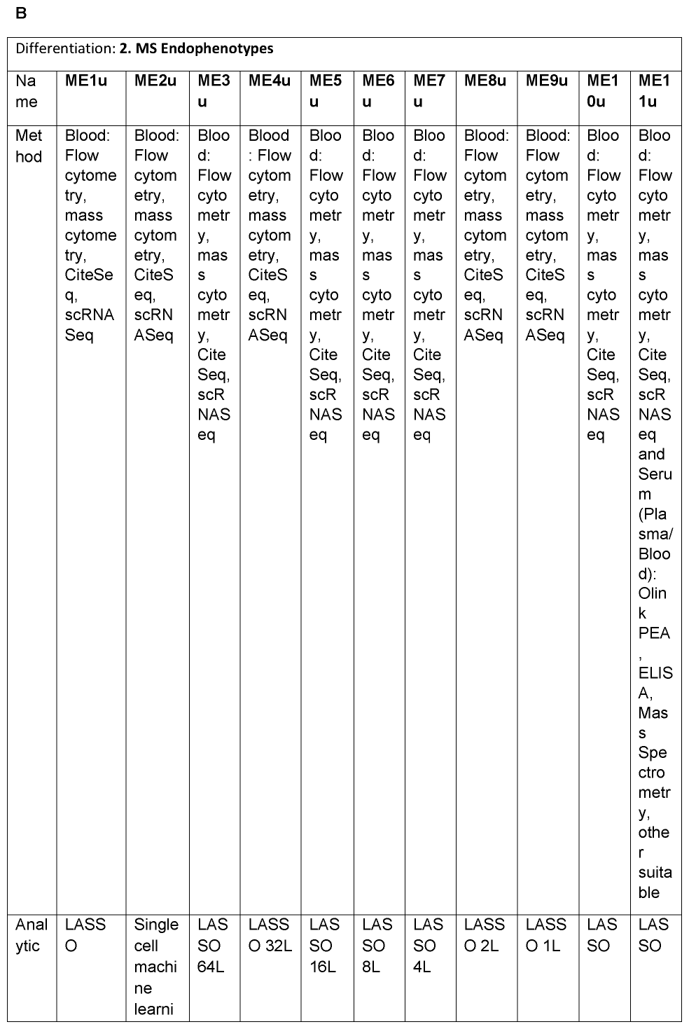

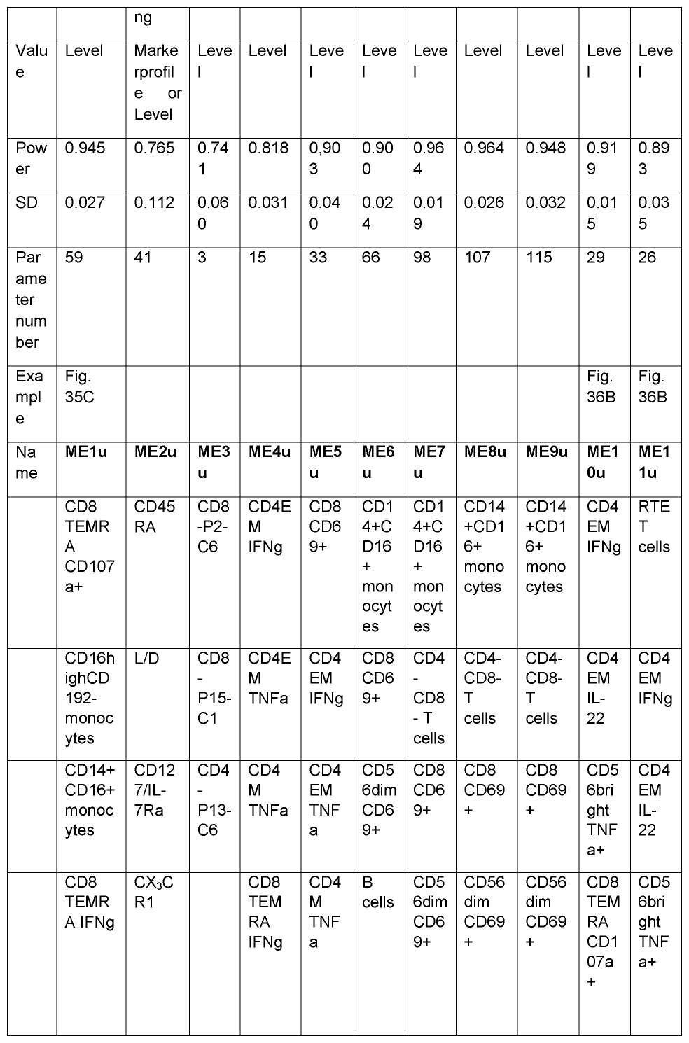

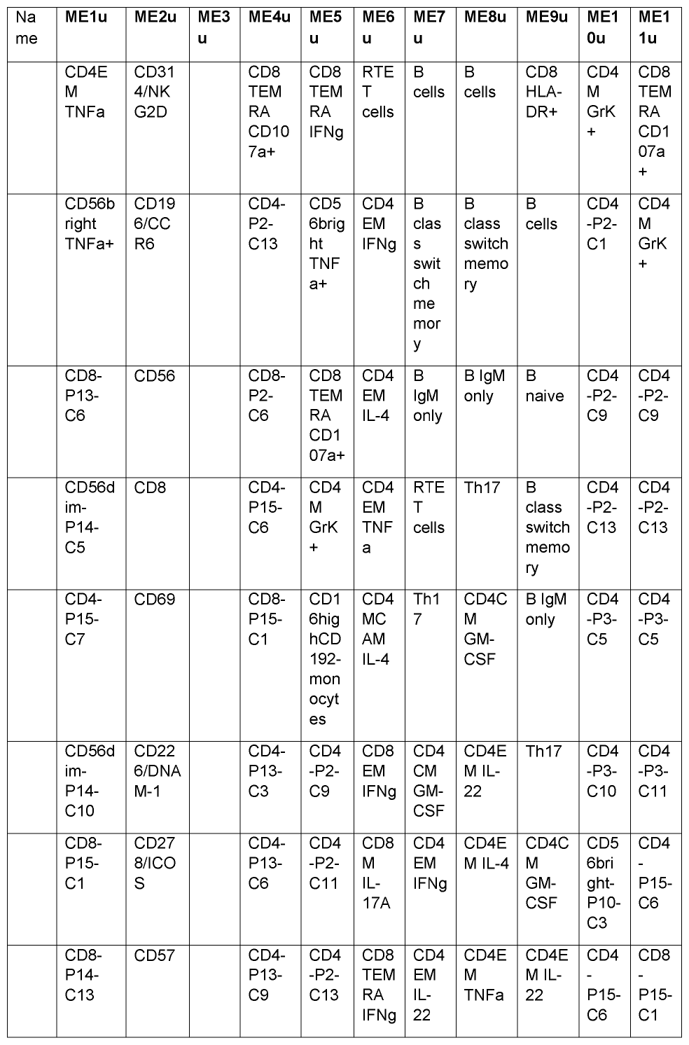

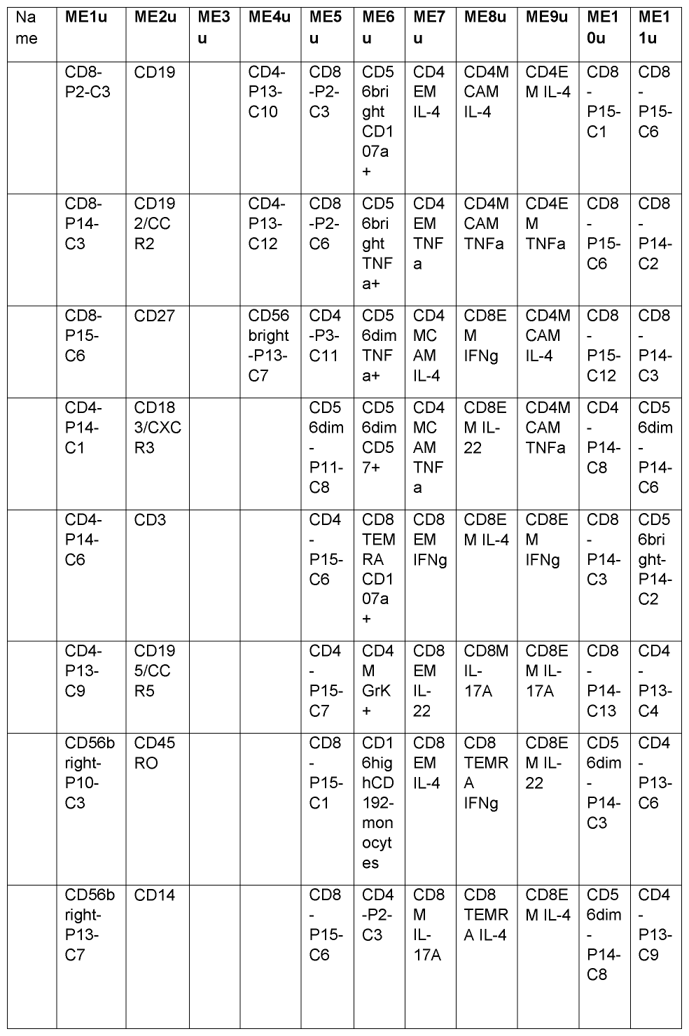

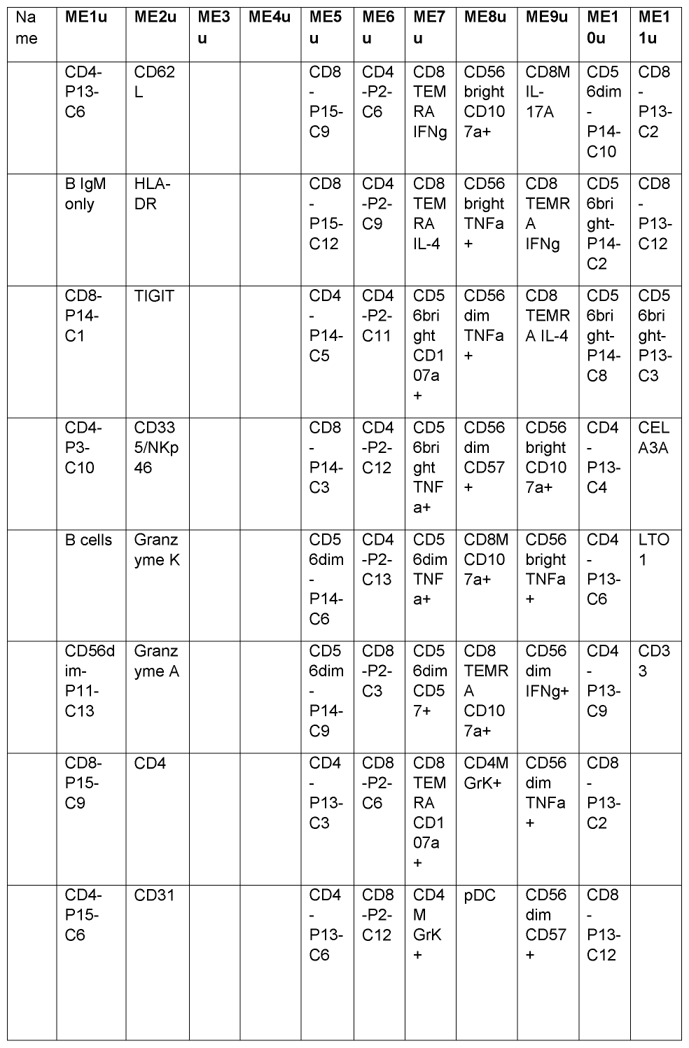

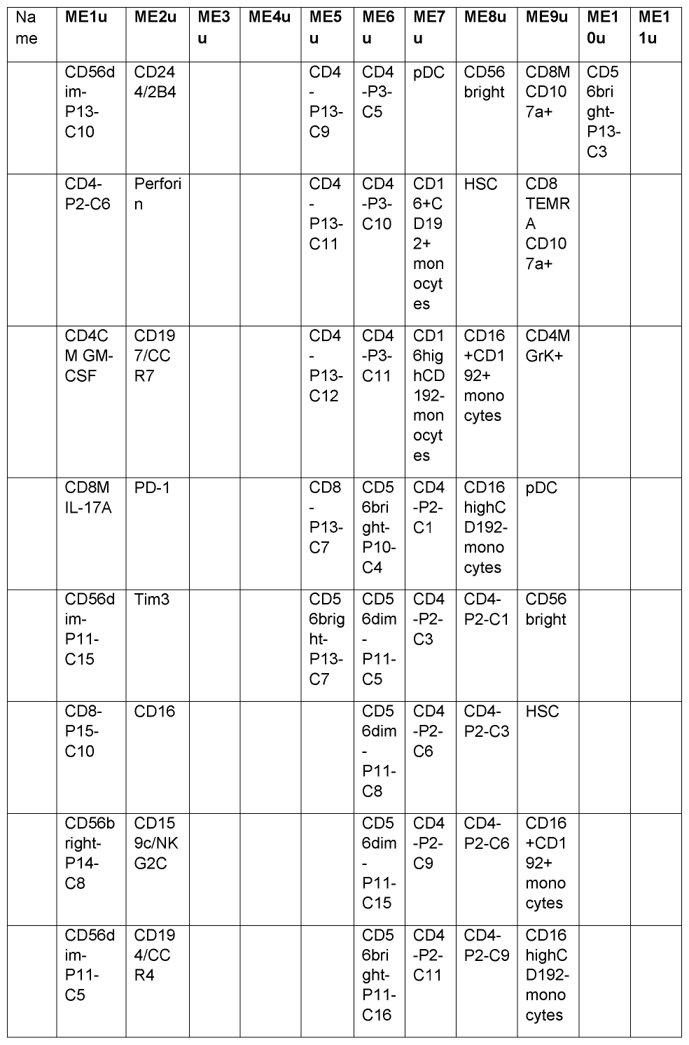

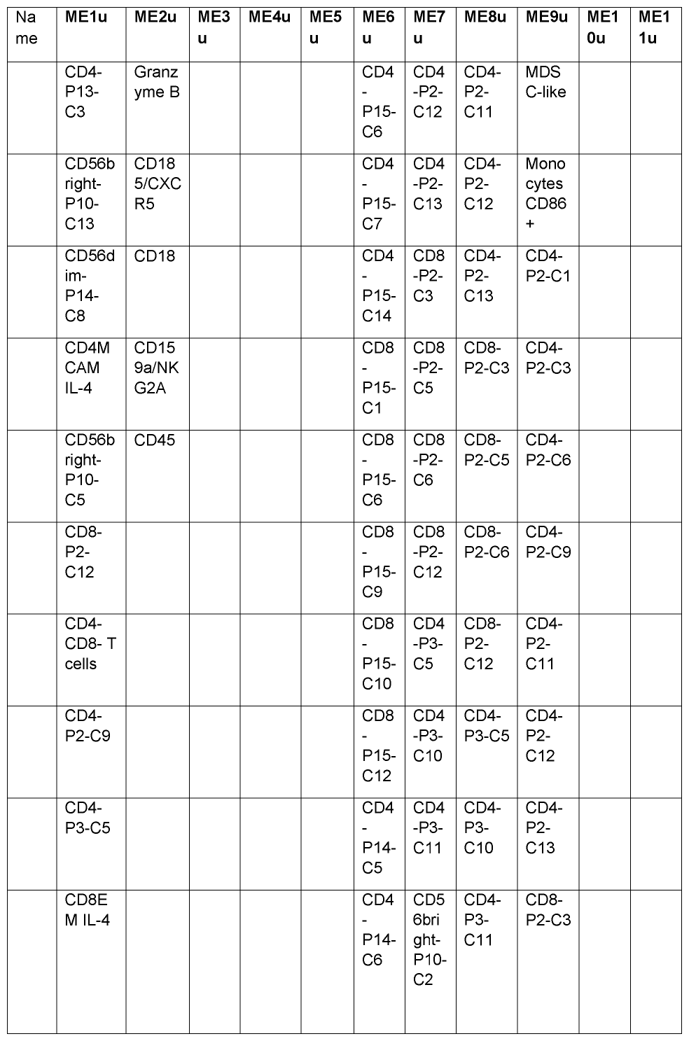

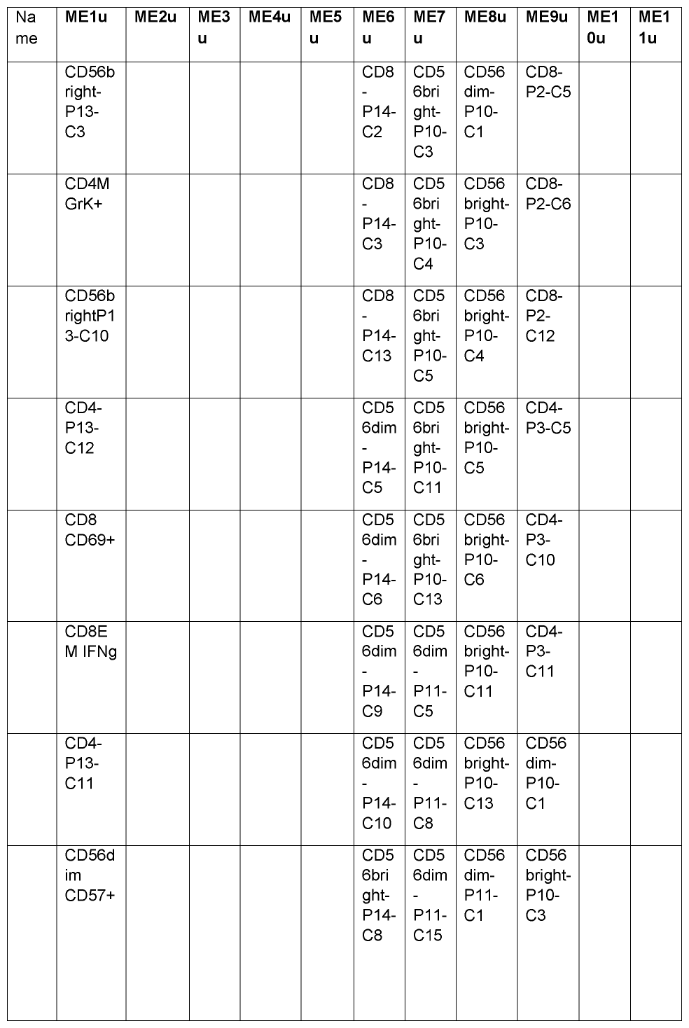

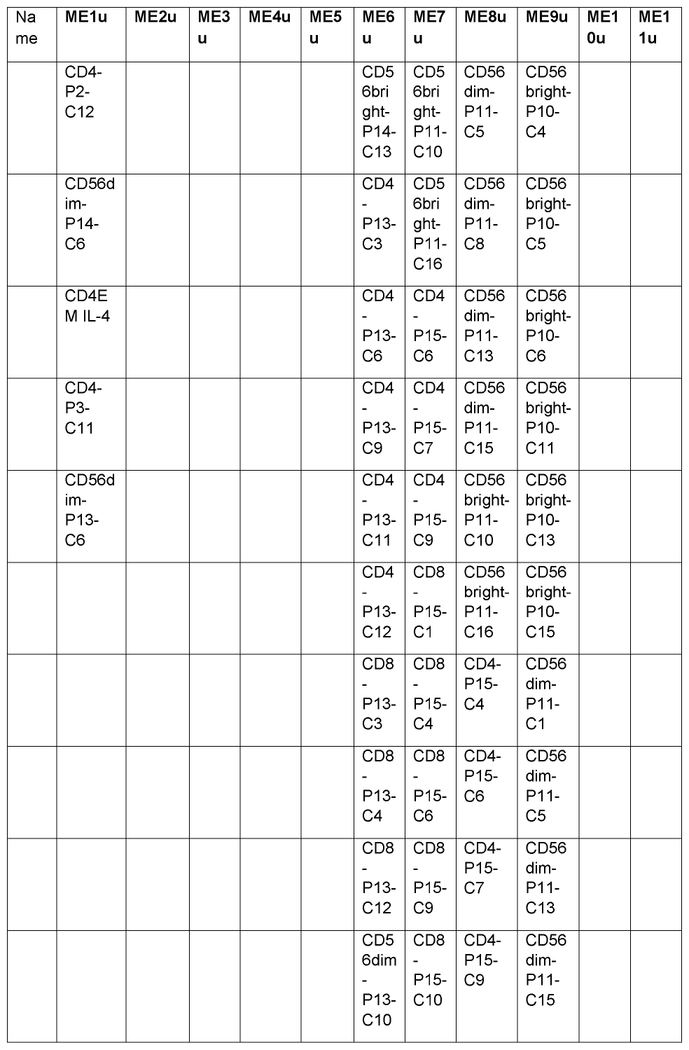

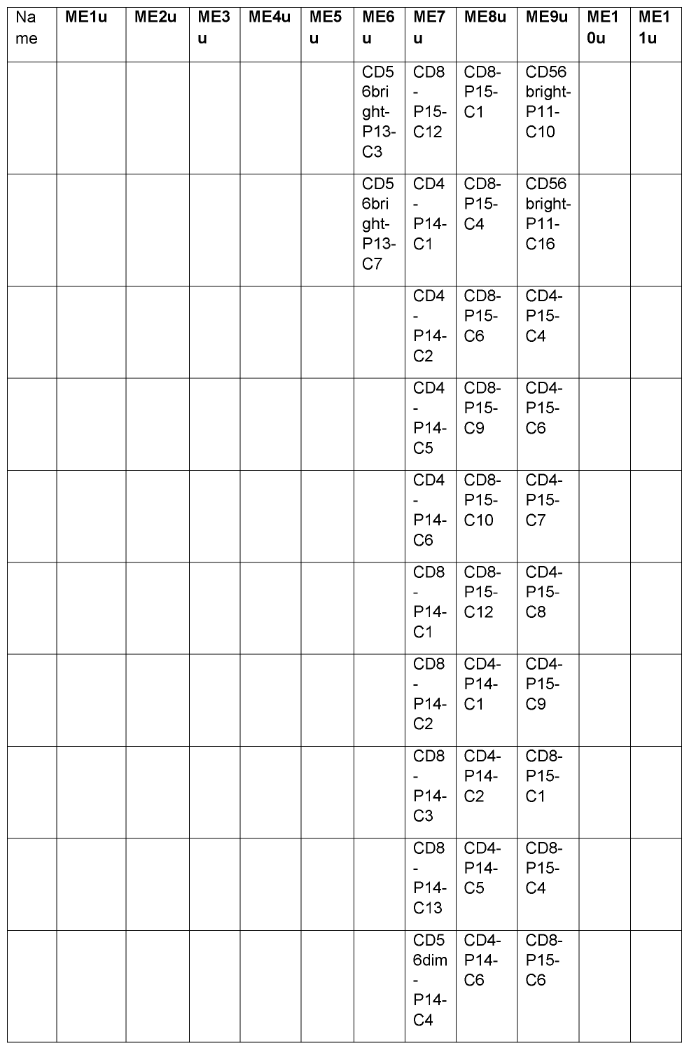

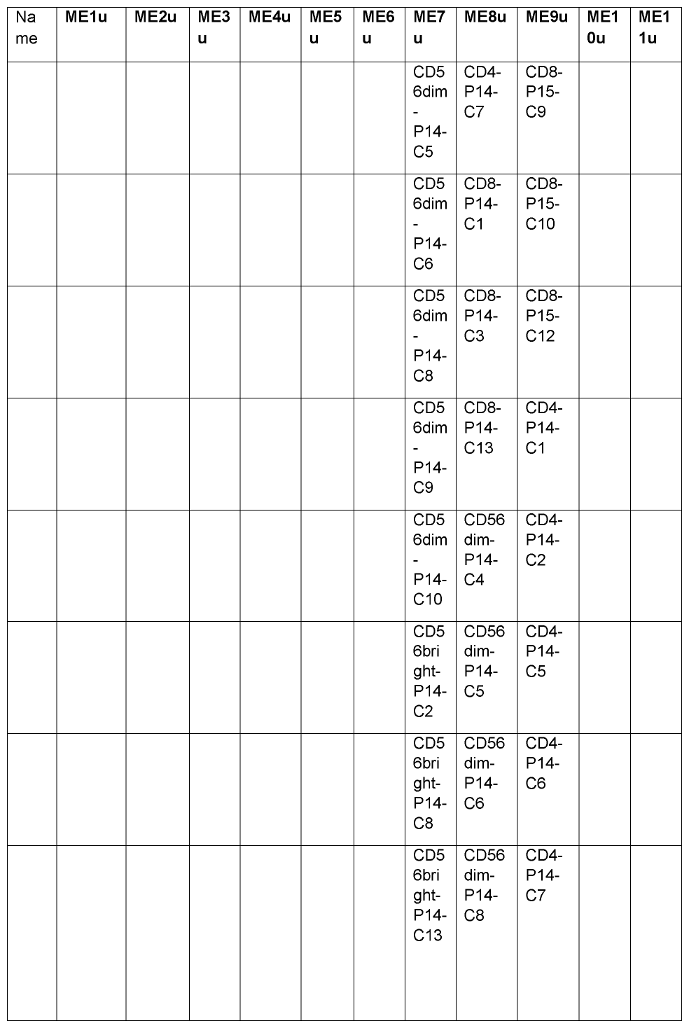

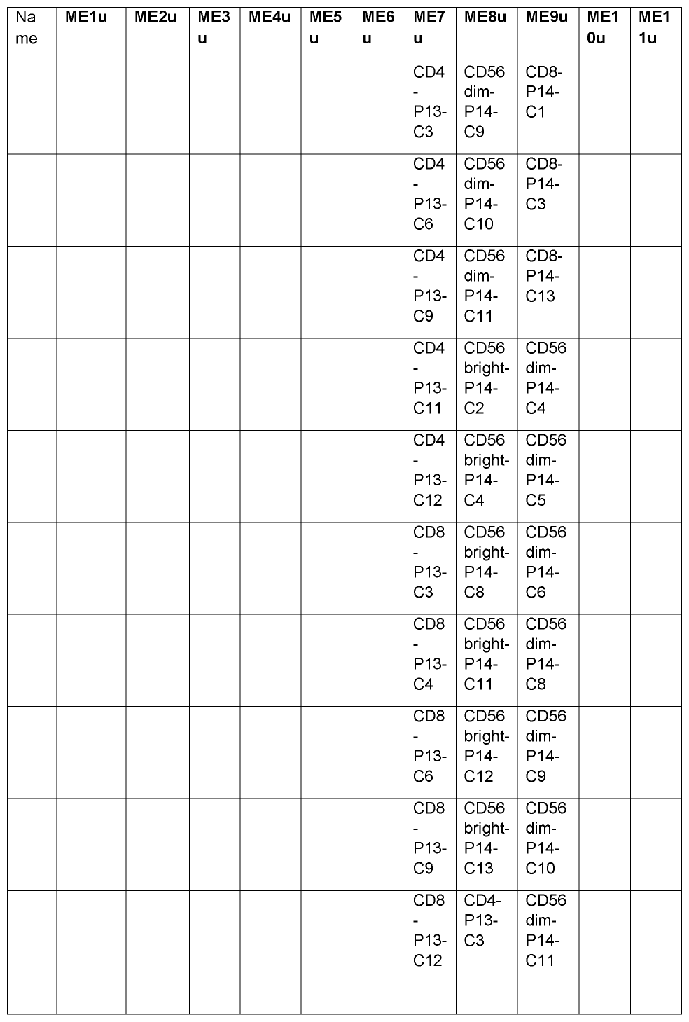

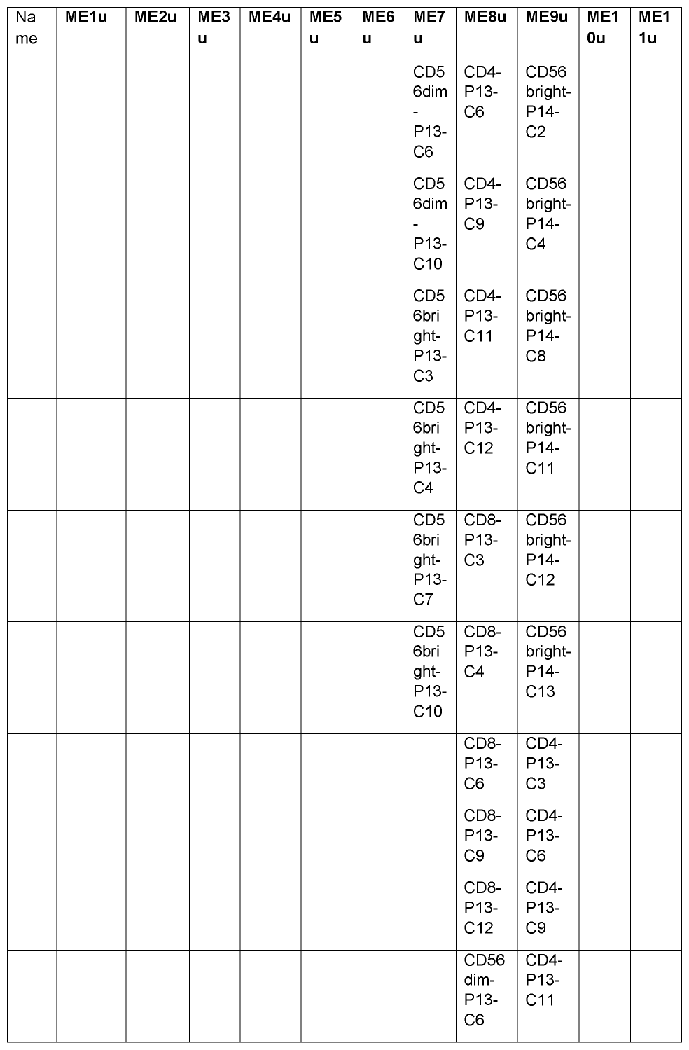

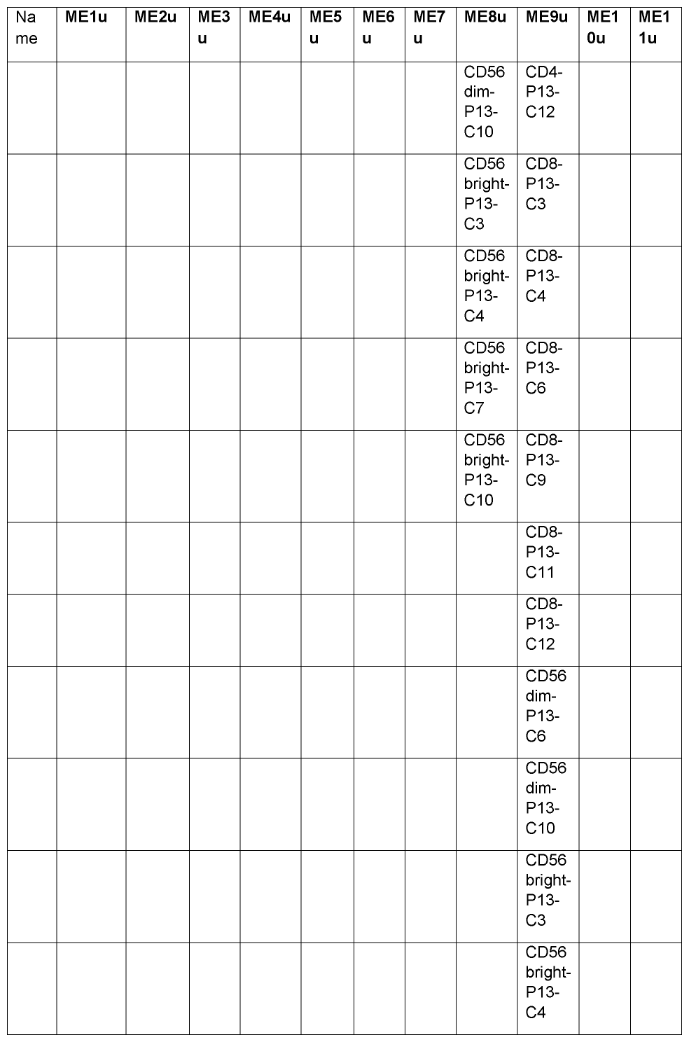

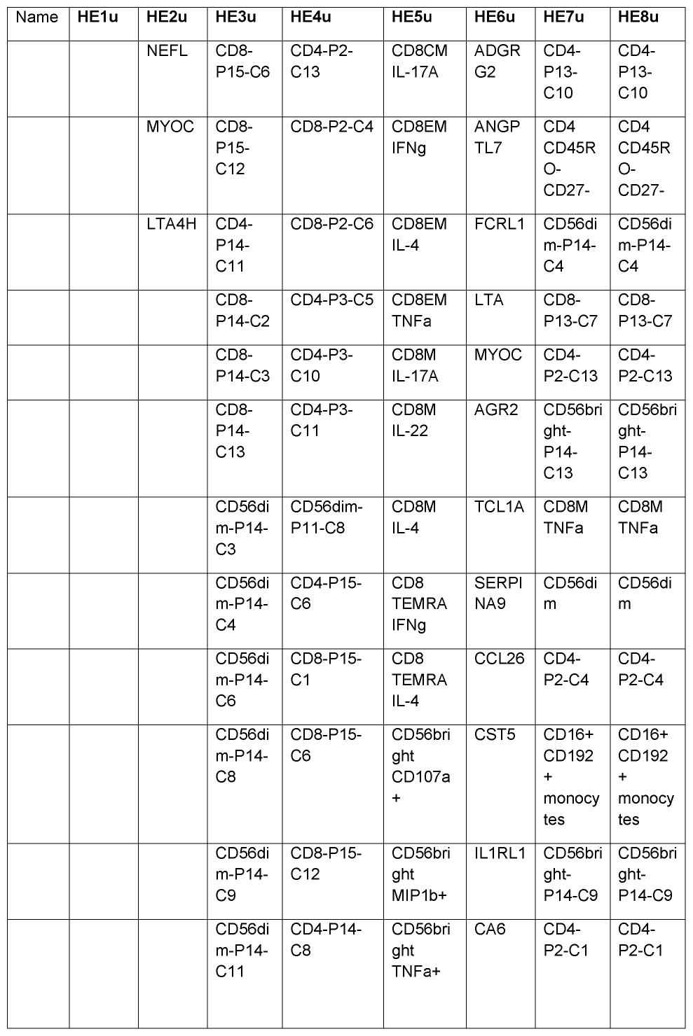

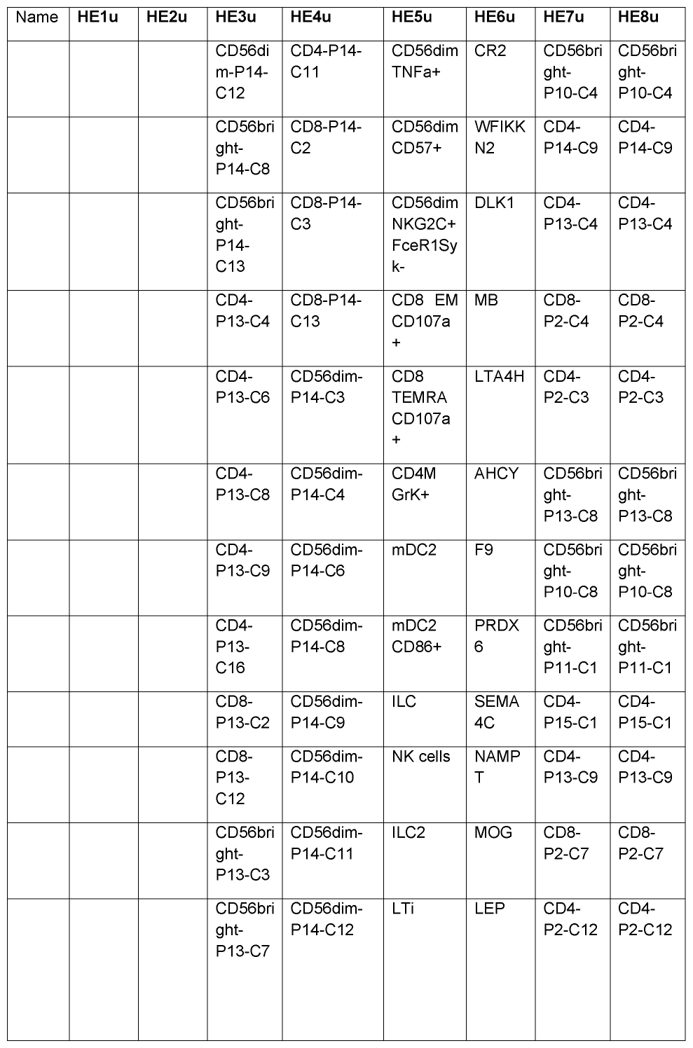

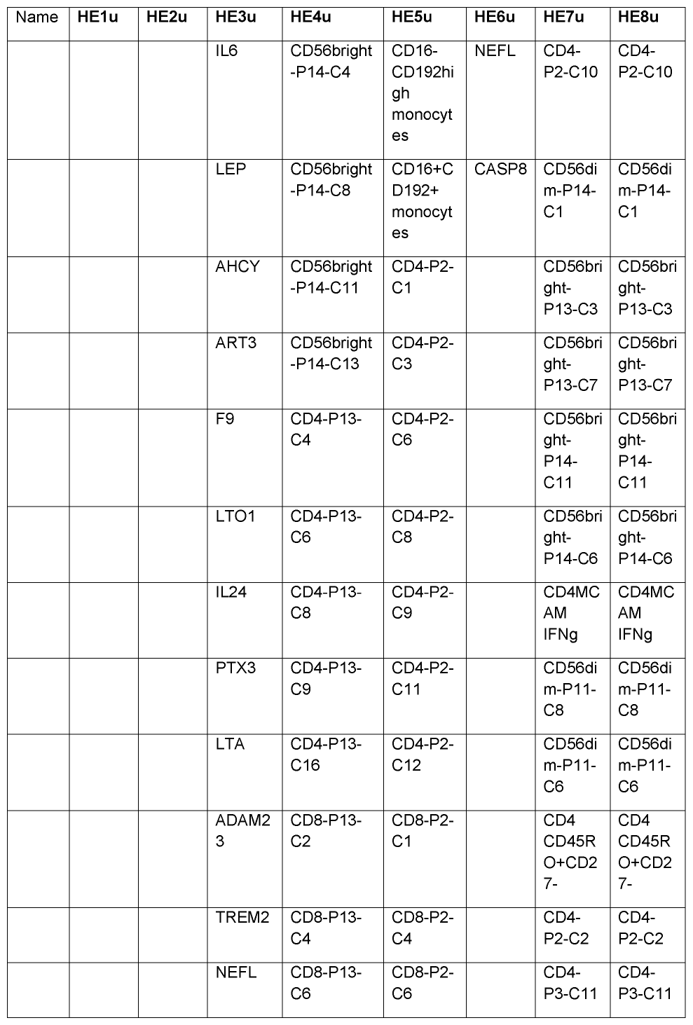

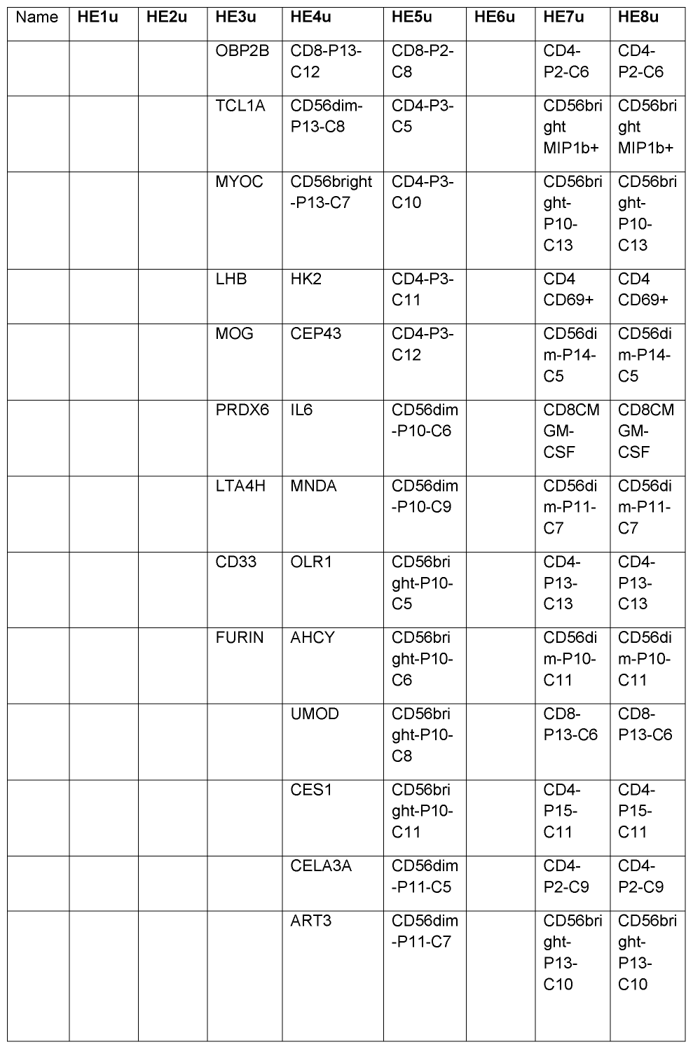

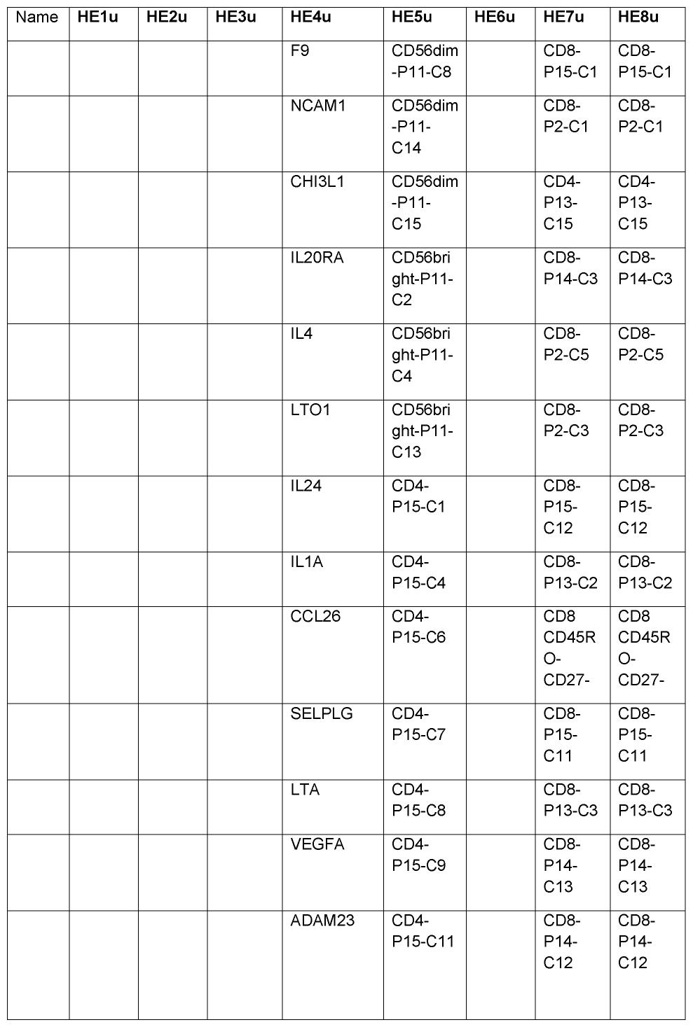

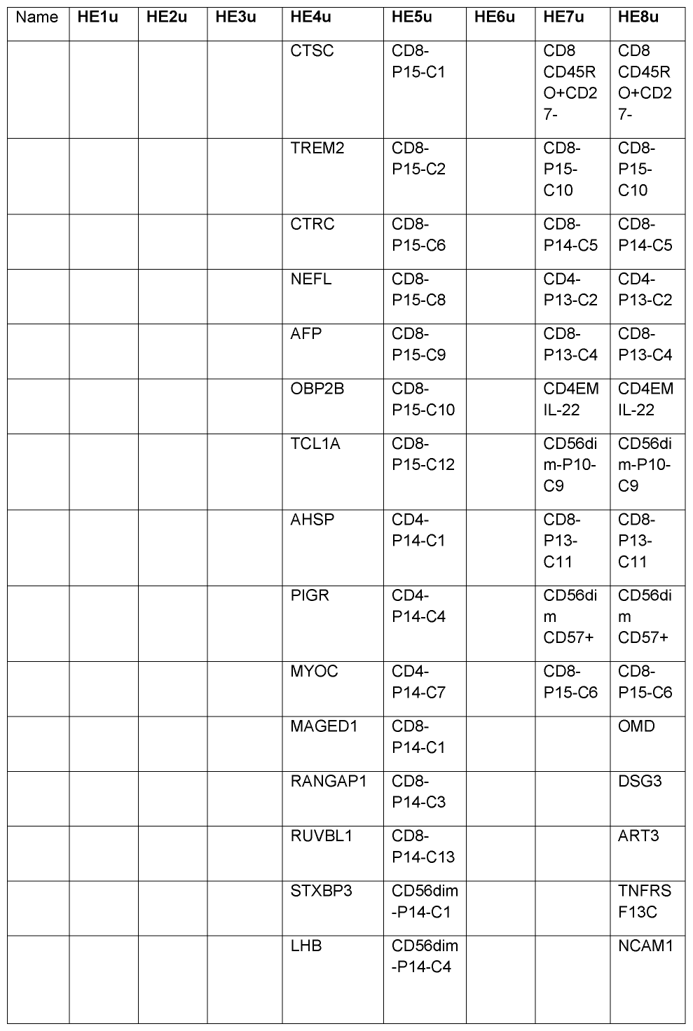

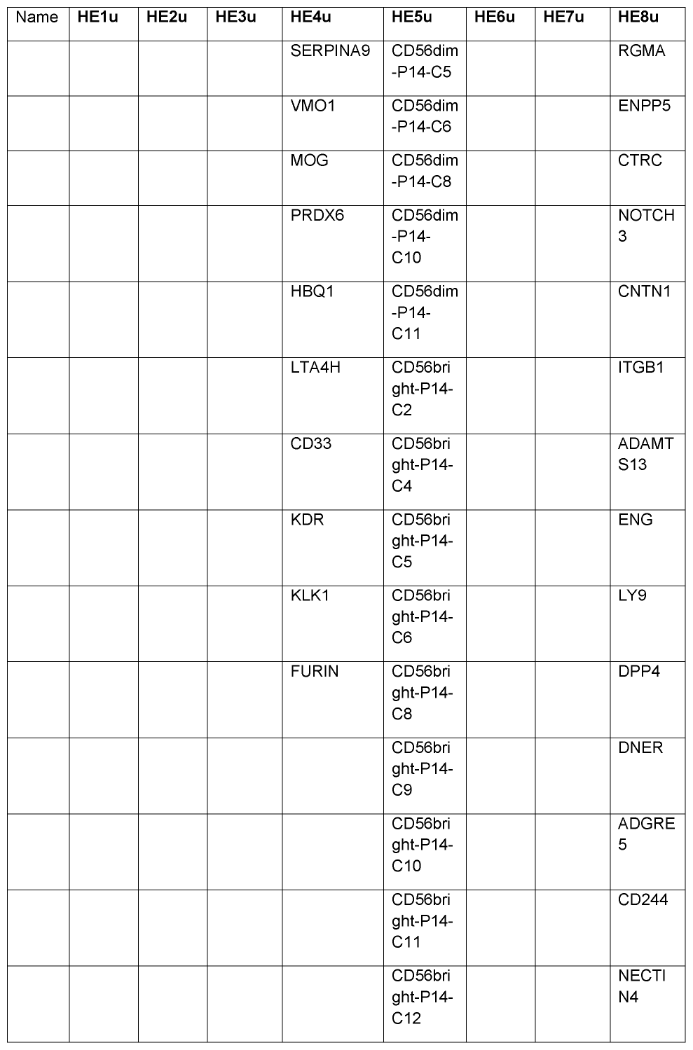

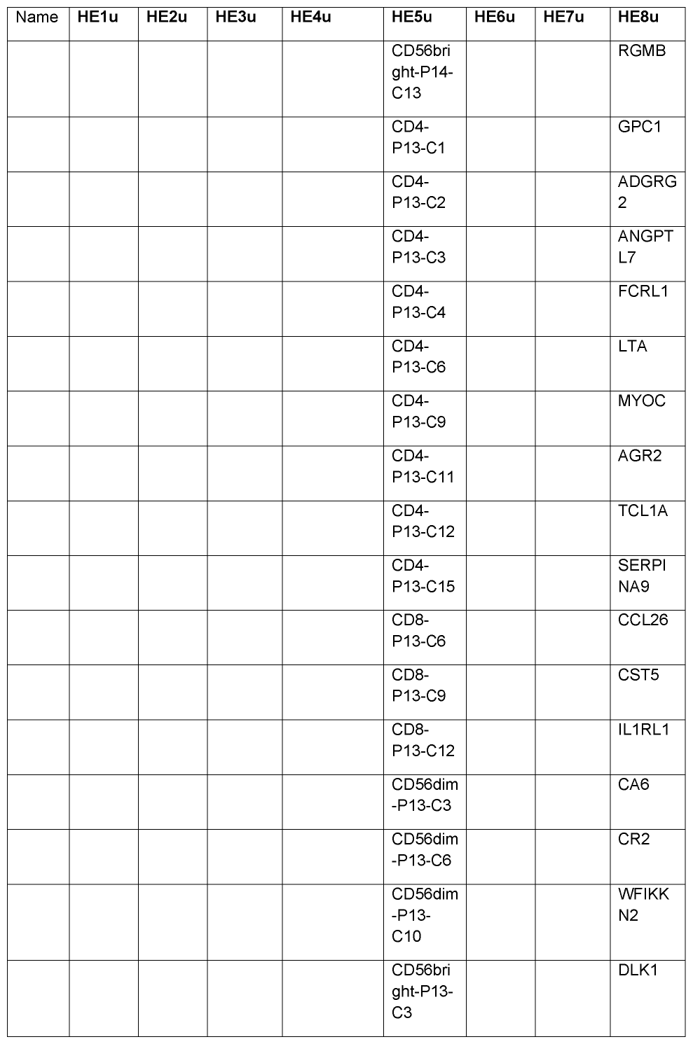

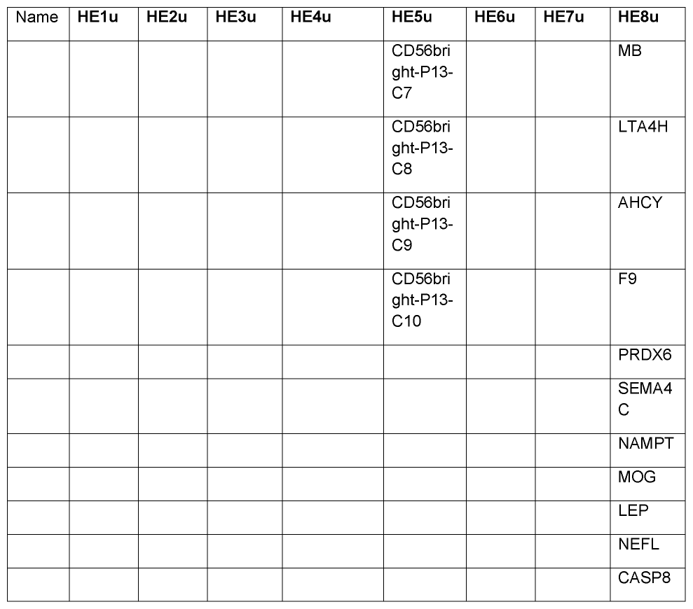

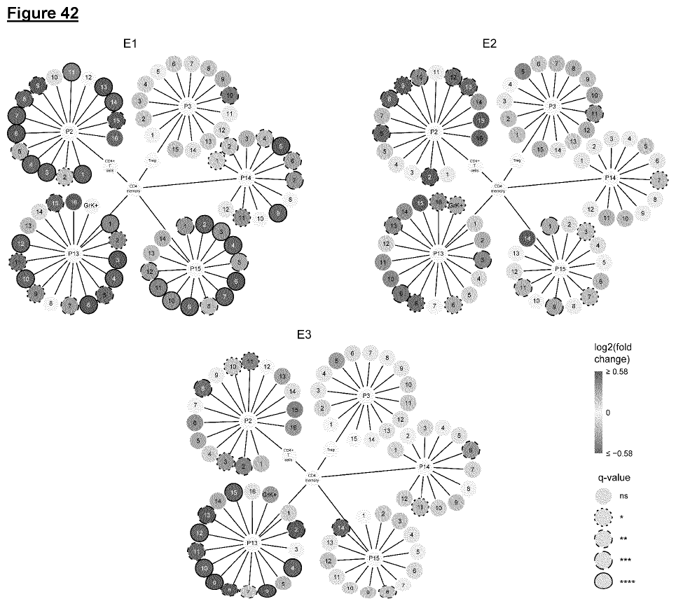

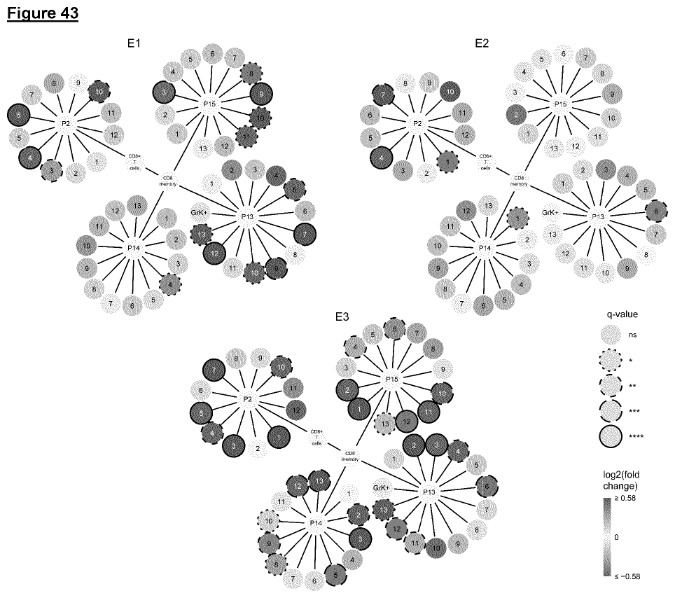

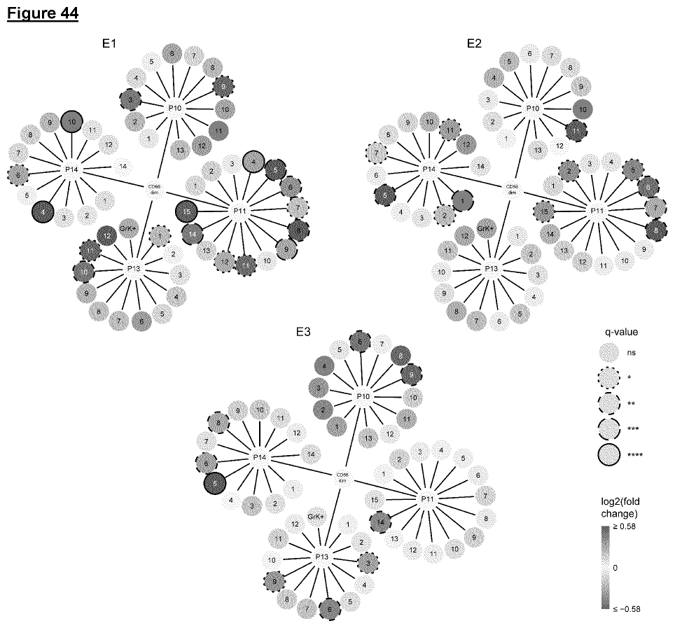

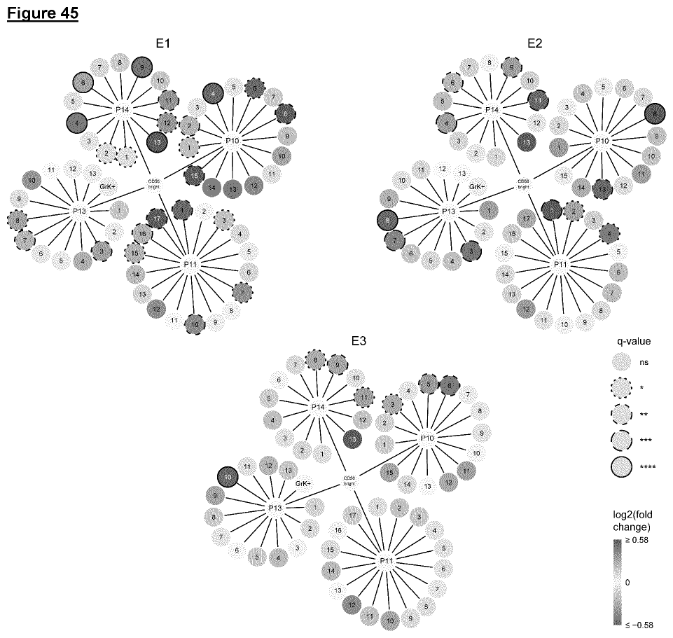

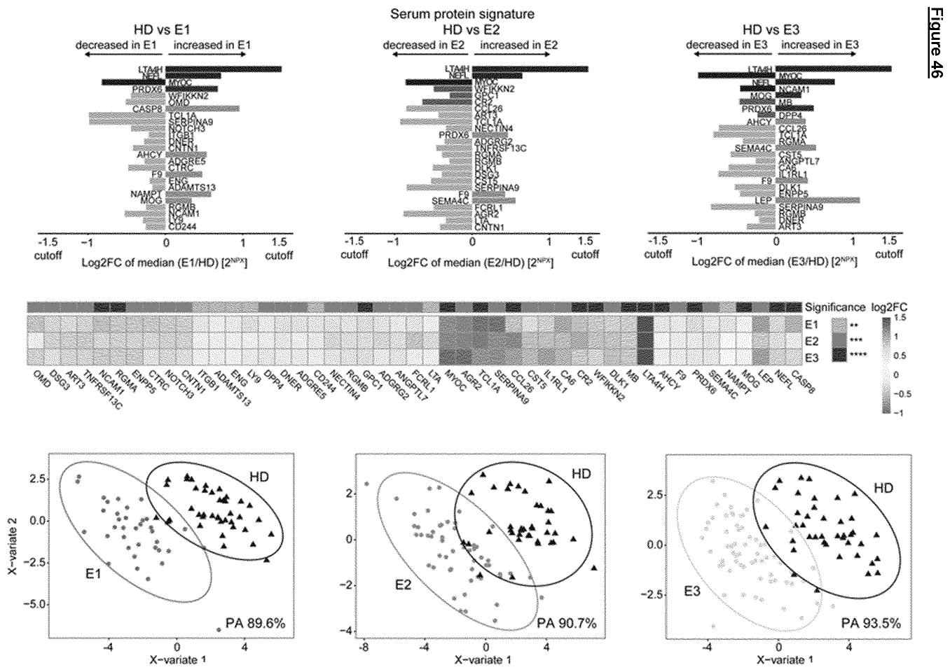

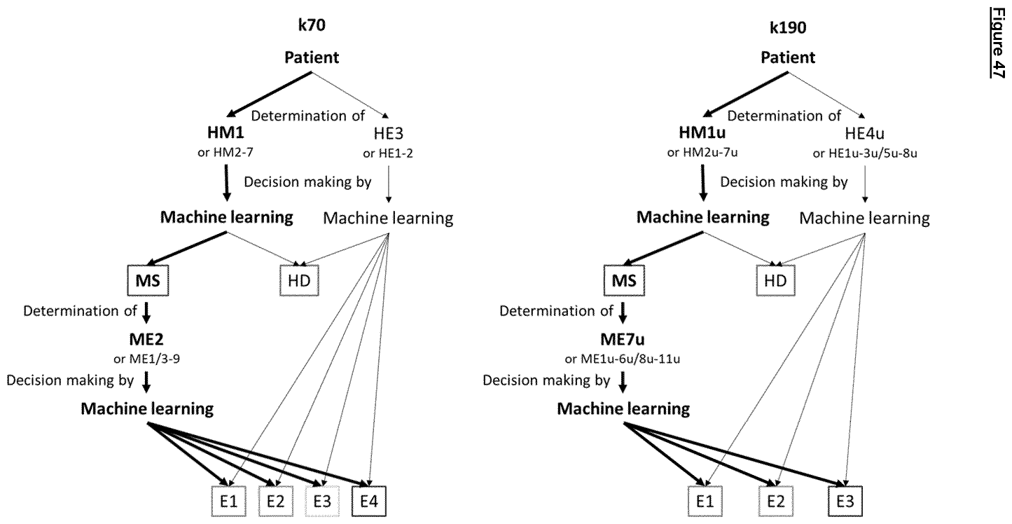

Hyperparameter k for Phenograph-driven grouping was investigated. To determine the most robust and likely outcome, the adjusted rand index (ARI) as a measure for grouping stability was determined over the whole range of plausible k values (FIG. 34A). Grouping stability was summarized for each k value by calculating the mean of the ARI values for the comparison with each other k value. The highest mean ARI values were consistently observed for results with three and four immunological endophenotypes (FIG. 34 B), with the overall most robust result for k=190 resulting in three endophenotypes (FIG. 34 B, dark line). To exemplify relevant results, analyses illustratively shown herein were performed based group assignment using k=70 (4 endophenotypes) and k=190 (3 endophenotypes). Analyses based on k=70 (4 endophenotypes) are illustratively depicted in FIGS. 34 to 47, and a comparison of distinguishing parameters identified in case of k=70 and/or k=190, respectively, is given in FIG. 48.

The invention is further characterized by the following items:
1. A method for determining distinguishing parameters of a neuro-inflammatory disease, said method comprising
   (a) obtaining parameters from a group of samples, wherein said group of samples comprises at least
      (i) a first subgroup of samples, and
      (ii) a second subgroup of samples, and
   (b) determining distinguishing parameters of the obtained parameters in step (a) at least between said first subgroup of samples and said second subgroup of samples by conducting analytical determination,
   preferably wherein the neuro-inflammatory disease is a neuro-inflammatory autoimmune disease, preferably multiple sclerosis (MS).
2. The method of item 1, wherein
   (ia) the first subgroup of samples is obtained from healthy subjects, and wherein
   (iia) the second subgroup of samples is obtained from subjects suffering from the neuro-inflammatory disease and/or from a subtype of said neuro-inflammatory disease,
   wherein said subtype is preferably an endophenotype of said neuro-inflammatory disease, preferably wherein
   (ib) the first subgroup of samples is obtained from subjects suffering from a first subtype of said neuro-inflammatory disease, and wherein
   (iib) the second subgroup of samples is obtained from subjects suffering from a second subtype of said neuro-inflammatory disease,
   wherein said first and said second subtypes are preferably a first and a second endophenotype of said neuro-inflammatory disease.
3. The method of item 2, wherein in step (a) said group of samples comprises further
   i) a third subgroup of samples, and
   (iv) a fourth subgroup of samples, and
   (v) optionally a fifth subgroup of samples,
   wherein
   (iii) the third subgroup of samples is obtained from subjects suffering from the neuro-inflammatory disease and/or from a subtype of said neuro-inflammatory disease, preferably from E3,
   (iv) the fourth subgroup of samples is obtained from subjects suffering from a fourth subtype of said neuro-inflammatory disease, preferably from E4, and
   (v) optionally the fifth subgroup of samples is obtained from subjects suffering from a first subtype of said neuro-inflammatory disease, and
   wherein the first subtype of said neuro-inflammatory disease is preferably E1 and the second subtype of said neuro-inflammatory disease is preferably E2.
4. The method of any one of the preceding items, wherein said distinguishing parameters of a neuro-inflammatory disease are parameters determining
   (1) a subject as being a healthy subject or a subject suffering from said disease,
   (2) a subject suffering from one of at least two endophenotypes of said disease, and/or
   (3) a subject as being a healthy subject or a subject suffering from at least one endophenotype of said disease.
5. The method of any one of the preceding items, wherein the sample is selected from one or more of the group consisting of blood, serum and plasma, preferably wherein the sample is selected from one or more of the group consisting of fresh serum, cryopreserved serum, preserved serum, plasma, and blood.
6. The method of any one of the preceding items, wherein the parameters are soluble parameters and/or cellular parameters, wherein the soluble parameters are preferably obtained from soluble serum and/or plasma proteins, and wherein the cellular parameters are preferably obtained from blood cells, preferably from peripheral blood mononuclear cells (PBMC); preferably wherein the soluble parameters are obtained from soluble proteins and step (a) comprises a proteomics analysis, preferably
   mass spectrometry, and/or
   protein quantification, preferably comprising a labelling technique, more preferably a proximity extension assay (PEA-NGS), enzyme-linked immunosorbent assay (ELISA), next generation ELISA (ELLA), flow cytometry-based multiplex assays, such as Luminex, chemiluminescent enzyme immunoassay analysis (CLEIA) or single molecule array (SIMOA), preferably wherein the cellular parameters are obtained from blood cells and step (a) comprises functional immune phenotyping, preferably comprising flow cytometry, single cell RNA sequencing (scRNAseq) mass cytometry or CiteSeq, more preferably flow cytometry.
7. The method of any one of the preceding items, wherein step (b) comprises automated decision making, preferably using machine learning, preferably wherein machine learning comprises at least one or more, preferably all, of the group consisting of
   a scaler,
   class imbalance correction, preferably using Synthetic Minority Oversampling Technique (SMOTE) algorithm,
   at least one, preferably two, more preferably three, even more preferably four, classifier preferably selected from the group consisting of random forest, AdaBoost classifier, linear support vector machine and radial basis function support vector machine,
   cross-validation, preferably nested cross-validation, more preferably nested and stratified cross-validation, and
   model performance optimization using a correlation coefficient, preferably Matthews correlation coefficient.
8. The method of any one of the preceding items, wherein step (b) comprises conducting
   (I) a statistical analysis, preferably a hypothesis test, preferably a t-test,
   (II) a regression analysis, preferably LASSO, and/or
   (III) parameter clustering;
   preferably wherein an obtained parameter of step (a) is determined as distinguishing parameter in step (b) if the obtained parameter of step (a) fulfils one or more of the following:
   (I) the statistical analysis significance, preferably the t-test significance, of the parameter is within the top x, preferably wherein
      (Ia) x is 25%, preferably in case of cellular parameters or
      (Ib) x is 100, preferably in case of soluble parameters,
   (II) the parameter is significant according to the conducted regression analysis, preferably LASSO, and/or
   (III) comprised in a parameter profile obtained from, preferably cellular, parameter clustering; preferably comprising further step (c) determining the prediction accuracy of the analytical determination conducted in step (b) and/or the determined distinguishing parameters, preferably using cross-validation; preferably wherein the determined prediction accuracy is at least 60%.
9. A set of distinguishing parameters of a neuro-inflammatory autoimmune disease, preferably MS, obtainable or obtained by the method of any one of the preceding items.
10. The set of distinguishing parameters according to item 9 for use in determining whether or not a subject suffers from said neuro-inflammatory autoimmune disease.
11. The set of distinguishing parameters according to item 9 for use in determining a subtype of a neuro-inflammatory autoimmune disease in a subject, wherein said subtype is preferably an endophenotype of said neuro-inflammatory disease.
12. The set of distinguishing parameters for use according to any one of items 11 to 13, wherein the neuro-inflammatory autoimmune disease is MS and the endophenotype is selected from the group consisting of E1, E2, E3, and E4, preferably wherein the neuro-inflammatory autoimmune disease is MS and wherein the set comprises at least 85%, preferably 90%, more preferably 95%, even more preferably 100% of one of the sets HM1, HM2, HM3, HM4, HM5, HM6, HM7, ME1, ME2, ME3, ME4, ME5, ME6, ME7, ME8, ME9, HE1, HE2, and HE3 listed in FIG. 9, preferably wherein the set is identical to one of the sets HM1, HM2, HM3, HM4, HM5, HM6, HM7, ME1, ME2, ME3, ME4, ME5, ME6, ME7, ME8, ME9, HE1, HE2, and HE3 listed in FIG. 9.
13. The set of distinguishing parameters according to item 9 for use in selecting a subject for a distinct treatment depending on the subtype, preferably an endophenotype, of said neuro-inflammatory disease, and wherein the subject is preferably suffering from a neuro-inflammatory autoimmune disease, preferably from MS.
14. The set of distinguishing parameters according to item 9 for use in predicting disease progression and/or treatment response to a therapeutic or preventive treatment in a subject, wherein the therapeutic or preventive treatment is preferably selected from the group consisting of immune-modulation, immune-trafficking and/or immune cell-depletion, and wherein subject is preferably suffering from a neuro-inflammatory autoimmune disease, preferably from MS.
15. A method for determining a subtype of a neuro-inflammatory autoimmune disease in a subject, the method comprising
   (a) comparing values of distinguishing parameters for said neuro-inflammatory autoimmune disease obtained from a sample of said subject with values of the sets of distinguishing parameters according to any one of items 9 to 14 and/or with values of distinguishing parameters as determined according to a method according to any one of items 1 to 8, and
   (b) determining the subtype of said neuro-inflammatory autoimmune disease based on said comparison, preferably by conducting analytical determination,
   wherein said subtype is preferably an endophenotype of said neuro-inflammatory disease, preferably an endophenotype of MS selected from the group consisting of E1, E2, E3, and E4.
16. The method of item 1, wherein
   (ia) the first subgroup of samples is obtained from healthy subjects, and wherein
   (iia) the second subgroup of samples is obtained from subjects suffering from the neuro-inflammatory disease and/or from a subtype of said neuro-inflammatory disease,
   wherein said subtype is preferably an endophenotype of said neuro-inflammatory disease
17. The method of item 1, wherein
   (ib) the first subgroup of samples is obtained from subjects suffering from a first subtype of said neuro-inflammatory disease, and wherein
   (iib) the second subgroup of samples is obtained from subjects suffering from a second subtype of said neuro-inflammatory disease,
   wherein said first and said second subtypes are preferably a first and a second endophenotype of said neuro-inflammatory disease.
18. The method of item 17, wherein in step (a) said group of samples comprises further
   (iii) a third subgroup of samples, and
   (iv) a fourth subgroup of samples, and
   (v) optionally a fifth subgroup of samples,
   wherein
   (iii) the third subgroup of samples is obtained from subjects suffering from the neuro-inflammatory disease and/or from a subtype of said neuro-inflammatory disease, preferably from E3,
   (iv) the fourth subgroup of samples is obtained from subjects suffering from a fourth subtype of said neuro-inflammatory disease, preferably from E4, and
   (v) optionally the fifth subgroup of samples is obtained from healthy subjects, and
   wherein the first subtype of said neuro-inflammatory disease is preferably E1 and the second subtype of said neuro-inflammatory disease is preferably E2.
19. The method of any one of items 16 to 18, wherein said distinguishing parameters of a neuro-inflammatory disease are parameters determining
   (1) a subject as being a healthy subject or a subject suffering from said disease,
   (2) a subject suffering from one of at least two endophenotypes of said disease, and/or
   (3) a subject as being a healthy subject or a subject suffering from at least one endophenotype of said disease.

### REFERENCES

1. N. Koch-Henriksen, P. S. Sørensen, The changing demographic pattern of multiple sclerosis epidemiology. The Lancet Neurology 9, 520-532 (2010).
2. A. J. Thompson, S. E. Baranzini, J. Geurts, B. Hemmer, O. Ciccarelli, Multiple sclerosis. The Lancet 391, 1622-1636 (2018).
3. D. S. Reich, C. F. Lucchinetti, P. A. Calabresi, Multiple Sclerosis. N Engl J Med 378, 169-180 (2018).
4. C. A. Dendrou, L. Fugger, M. A. Friese, Immunopathology of multiple sclerosis. Nature Reviews Immunology 15, 545-558 (2015).
5. C. C. Gross et al., Classification of neurological diseases using multi-dimensional CSF analysis. Brain 144, 2625-2634 (2021).
6. B. Hemmer, M. Kerschensteiner, T. Korn, Role of the innate and adaptive immune responses in the course of multiple sclerosis. The Lancet Neurology 14, 406-419 (2015).
7. P. L. De Jager et al., Cytometric profiling in multiple sclerosis uncovers patient population structure and a reduction of CD8low cells. Brain 131, 1701-1711 (2008).
8. C. C. Gross et al., Distinct pattern of lesion distribution in multiple sclerosis is associated with different circulating T-helper and helper-like innate lymphoid cell subsets. Multiple Sclerosis Journal 23, 1025-1030 (2017).
9. J. Haas et al., Reduced suppressive effect of CD4+CD25high regulatory T cells on the T cell immune response against myelin oligodendrocyte glycoprotein in patients with multiple sclerosis. European Journal of Immunology 35, 3343-3352 (2005).
10. J. Jensen et al., CD4 T cell activation and disease activity at onset of multiple sclerosis. J Neuroimmunol 149, 202-209 (2004).
11. A. Lünemann et al., Impaired IFN-γ production and proliferation of NK cells in Multiple Sclerosis. International Immunology 23, 139-148 (2011).
12. Z. Mikulkova, P. Praksova, P. Stourac, J. Bednarik, J. Michalek, Imbalance in T-cell and cytokine profiles in patients with relapsing-remitting multiple sclerosis. Journal of the Neurological Sciences 300, 135-141 (2011).
13. D. Plantone et al., Circulating CD56dim NK cells expressing perforin are increased in progressive multiple sclerosis. Journal of Neuroimmunology 265, 124-127 (2013).
14. A. Prieto et al., Increased Spontaneous Ex Vivo Apoptosis and Subset Alterations in Peripheral Blood T Cells from Patients with Multiple Sclerosis. Journal of Clinical Immunology 26, 101-112 (2006).
15. J. D. Lünemann, T. Ruck, P. A. Muraro, A. Bar-Or, H. Wiendl, Immune reconstitution therapies: concepts for durable remission in multiple sclerosis. Nature Reviews Neurology 16, 56-62 (2020).
16. H. Wiendl, P. A. Calabresi, S. G. Meuth, Defining response profiles after alemtuzumab. Neurology 90, 309 (2018).
17. M. Liebmann et al., Dimethyl fumarate treatment restrains the antioxidative capacity of T cells to control autoimmunity. Brain 144, 3126-3141 (2021).
18. D. Ontaneda, E. Tallantyre, T. Kalincik, S. M. Planchon, N. Evangelou, Early highly effective versus escalation treatment approaches in relapsing multiple sclerosis. The Lancet Neurology 18, 973-980 (2019).
19. O. von Bismarck et al., Treatment choices and neuropsychological symptoms of a large cohort of early MS. Neurology—Neuroimmunology Neuroinflammation 5, e446 (2018).
20. P. Ostkamp et al., Sunlight exposure exerts immunomodulatory effects to reduce multiple sclerosis severity. Proceedings of the National Academy of Sciences 118, e2018457118 (2021).
21. J. F. Kurtzke, Rating neurologic impairment in multiple sclerosis. Neurology 33, 1444 (1983).
22. J. S. Fischer, R. A. Rudick, G. R. Cutter, S. C. Reingold, The Multiple Sclerosis Functional Composite Measure (MSFC): an integrated approach to MS clinical outcome assessment. National MS Society Clinical Outcomes Assessment Task Force. Mult Scler 5, 244-250 (1999).
23. P. Calabrese, E. Kalbe, J. Kessler, Ein neuropsychologisches Screening zur Erfassung kognitiver Störungen bei MS-Patienten—Das Multiple Sklerose Inventarium Cognition (MUSIC). psychoneuro 30, 384-388 (2004).
24. M. R. Filbin et al., Longitudinal proteomic analysis of severe COVID-19 reveals survival-associated signatures, tissue-specific cell death, and cell-cell interactions. Cell Rep Med 2, 100287 (2021).
25. C. Barro et al., Serum neurofilament as a predictor of disease worsening and brain and spinal cord atrophy in multiple sclerosis. Brain 141, 2382-2391 (2018).
26. J. Kuhle et al., Serum neurofilament light chain in early relapsing remitting MS is increased and correlates with CSF levels and with MRI measures of disease severity. Mult Scler 22, 1550-1559 (2016).
27. C. C. Gross et al., Impaired NK-mediated regulation of T-cell activity in multiple sclerosis is reconstituted by IL-2 receptor modulation. Proceedings of the National Academy of Sciences 113, E2973-E2982 (2016).
28. M. Piédavent-Salomon et al., Multiple sclerosis associated genetic variants of CD226 impair regulatory T cell function. Brain 138, 3263-3274 (2015).
29. S. M. Lank et al., Ultra-high resolution HLA genotyping and allele discovery by highly multiplexed cDNA amplicon pyrosequencing. BMC Genomics 13, 378 (2012).
30. A. H. Schmidt et al., Estimation of high-resolution HLA-A, -B, -C, -DRB1 allele and haplotype frequencies based on 8862 German stem cell donors and implications for strategic donor registry planning. Hum Immunol 70, 895-902 (2009).
31. B. John, K. R. Lewis, Chromosome variability and geographic distribution in insects. Science 152, 711-721 (1966).
32. Gottesman, II, T. D. Gould, The endophenotype concept in psychiatry: etymology and strategic intentions. Am J Psychiatry 160, 636-645 (2003).
33. C. International Multiple Sclerosis Genetics et al., Genetic risk and a primary role for cell-mediated immune mechanisms in multiple sclerosis. Nature 476, 214-219 (2011).
34. H. Modin, W. Olsson, J. Hillert, T. Masterman, Modes of action of HLA-DR susceptibility specificities in multiple sclerosis. Am J Hum Genet 74, 1321-1322 (2004).
35. M. G. Marrosu et al., Multiple sclerosis in Sardinia is associated and in linkage disequilibrium with HLA-DR3 and -DR4 alleles. Am J Hum Genet 61, 454-457 (1997).
36. O. J. Kwon et al., HLA class II susceptibility to multiple sclerosis among Ashkenazi and non-Ashkenazi Jews. Arch Neurol 56, 555-560 (1999).
37. Q. Zhang, C. Y. Lin, Q. Dong, J. Wang, W. Wang, Relationship between HLA-DRB1 polymorphism and susceptibility or resistance to multiple sclerosis in Caucasians: a meta-analysis of non-family-based studies. Autoimmun Rev 10, 474-481 (2011).
38. H. Lassmann, The changing concepts in the neuropathology of acquired demyelinating central nervous system disorders. Curr Opin Neurol 32, 313-319 (2019).
39. S. K. Lundy et al., Dimethyl fumarate treatment of relapsing-remitting multiple sclerosis influences B-cell subsets. Neurology—Neuroimmunology Neuroinflammation 3, e211 (2016).
40. C. Tørring et al., The B1-cell subpopulation is diminished in patients with relapsing-remitting multiple sclerosis. Journal of Neuroimmunology 262, 92-99 (2013).
41. J. Elkins et al., CD56^{bright} natural killer cells and response to daclizumab HYP in relapsing-remitting MS. Neurology—Neuroimmunology Neuroinflammation 2, e65 (2015).
42. A. Laroni et al., Dysregulation of regulatory CD56 bright NK cells/T cells interactions in multiple sclerosis. Journal of Autoimmunity 72, 8-18 (2016).
43. C. Böttcher et al., Multi-parameter immune profiling of peripheral blood mononuclear cells by multiplexed single-cell mass cytometry in patients with early multiple sclerosis. Scientific Reports 9, 19471 (2019).
44. G. Meyer zu Hörste, C. C. Gross, L. Klotz, N. Schwab, H. Wiendl, Next-Generation Neuroimmunology: New Technologies to Understand Central Nervous System Autoimmunity. Trends in Immunology 41, 341-354 (2020).
45. E. Galli et al., GM-CSF and CXCR4 define a T helper cell signature in multiple sclerosis. Nature Medicine 25, 1290-1300 (2019).
46. C. International Multiple Sclerosis Genetics, Multiple sclerosis genomic map implicates peripheral immune cells and microglia in susceptibility. Science 365, (2019).
47. S. M. Solberg et al., Mass cytometry analysis of blood immune cells from psoriasis patients on biological therapy. European Journal of Immunology 51, 694-702 (2021).
48. M. J. C. Jordao et al., Single-cell profiling identifies myeloid cell subsets with distinct fates during neuroinflammation. Science 363, (2019).
49. D. Schafflick et al., Single-cell profiling of CNS border compartment leukocytes reveals that B cells and their progenitors reside in non-diseased meninges. Nature Neuroscience 24, 1225-1234 (2021).
50. D. Schafflick et al., Integrated single cell analysis of blood and cerebrospinal fluid leukocytes in multiple sclerosis. Nature Communications 11, 247 (2020).
51. L. K. Fisniku et al., Disability and T2 MRI lesions: a 20-year follow-up of patients with relapse onset of multiple sclerosis. Brain 131, 808-817 (2008).
52. T. Kalincik et al., Towards personalized therapy for multiple sclerosis: prediction of individual treatment response. Brain 140, 2426-2443 (2017).
53. A. Langer-Gould et al., Clinical and Demographic Predictors of Long-term Disability in Patients With Relapsing-Remitting Multiple Sclerosis: A Systematic Review. Archives of Neurology 63, 1686 (2006).
54. V. Popescu et al., Brain atrophy and lesion load predict long term disability in multiple sclerosis. Journal of Neurology, Neurosurgery & Psychiatry 84, 1082-1091 (2013).
55. D. L. Rotstein, B. C. Healy, M. T. Malik, T. Chitnis, H. L. Weiner, Evaluation of No Evidence of Disease Activity in a 7-Year Longitudinal Multiple Sclerosis Cohort. JAMA Neurology 72, 152 (2015).
56. A. Scalfari et al., The natural history of multiple sclerosis, a geographically based study 10: relapses and long-term disability. Brain 133, 1914-1929 (2010).
57. A. Scalfari et al., The cortical damage, early relapses, and onset of the progressive phase in multiple sclerosis. Neurology 90, e2107-e2118 (2018).
58. J. R. Avasarala, A. H. Cross, J. L. Trotter, Oligoclonal Band Number as a Marker for Prognosis in Multiple Sclerosis. Archives of Neurology 58, 2044 (2001).
59. M. J. Magraner et al., Brain atrophy and lesion load are related to CSF lipid-specific IgM oligoclonal bands in clinically isolated syndromes. Neuroradiology 54, 5-12 (2012).
60. L. Villar et al., Influence of oligoclonal IgM specificity in multiple sclerosis disease course. Multiple Sclerosis Journal 14, 183-187 (2008).
61. C. Barro et al., Serum neurofilament as a predictor of disease worsening and brain and spinal cord atrophy in multiple sclerosis. Brain 141, 2382-2391 (2018).
62. E. Cantó et al., Chitinase 3-like 1: prognostic biomarker in clinically isolated syndromes. Brain 138, 918-931 (2015).
63. J. Kuhle et al., Serum neurofilament light chain in early relapsing remitting MS is increased and correlates with CSF levels and with MRI measures of disease severity. Multiple Sclerosis Journal 22, 1550-1559 (2016).
64. F. Sellebjerg, L. Royen, P. Soelberg Sørensen, A. B. Oturai, P. E. H. Jensen, Prognostic value of cerebrospinal fluid neurofilament light chain and chitinase-3-like-1 in newly diagnosed patients with multiple sclerosis. Multiple Sclerosis Journal 25, 1444-1451 (2019).
65. N. Siller et al., Serum neurofilament light chain is a biomarker of acute and chronic neuronal damage in early multiple sclerosis. Multiple Sclerosis Journal 25, 678-686 (2019).
66. S. Bittner et al., Clinical implications of serum neurofilament in newly diagnosed MS patients: A longitudinal multicentre cohort study. EBioMedicine 56, 102807 (2020).
67. A. Eshaghi et al., Identifying multiple sclerosis subtypes using unsupervised machine learning and MRI data. Nature Communications 12, 2078 (2021).
68. M. Muthuraman et al., Covarying patterns of white matter lesions and cortical atrophy predict progression in early MS. Neurol Neuroimmunol Neuroinflamm 7, (2020).
69. B. Bielekova et al., MRI as a marker for disease heterogeneity in multiple sclerosis. Neurology 65, 1071-1076 (2005).
70. C. Lucchinetti et al., Heterogeneity of multiple sclerosis lesions: implications for the pathogenesis of demyelination. Ann Neurol 47, 707-717 (2000).
71. J. Kuhle et al., A comparative study of CSF neurofilament light and heavy chain protein in MS. Mult Scler 19, 1597-1603 (2013).
72. K. Frei, S. Fredrikson, A. Fontana, H. Link, Interleukin-6 is elevated in plasma in multiple sclerosis. J Neuroimmunol 31, 147-153 (1991).
73. J. Río et al., Measures in the first year of therapy predict the response to interferon β in MS. Multiple Sclerosis Journal 15, 848-853 (2009).
74. S. Bittner, J. Oh, E. K. Havrdová, M. Tintoré, F. Zipp, The potential of serum neurofilament as biomarker for multiple sclerosis. Brain 144, 2954-2963 (2021).
75. L. Novakova et al., Monitoring disease activity in multiple sclerosis using serum neurofilament light protein. Neurology 89, 2230-2237 (2017).
76. P. E. Walo-Delgado et al., Low serum neurofilament light chain values identify optimal responders to dimethyl fumarate in multiple sclerosis treatment. Scientific Reports 11, 9299 (2021).
77. S. Katsavos, A. Coles, Alemtuzumab as Treatment for Multiple Sclerosis. Cold Spring Harbor Perspectives in Medicine 8, a032029 (2018).
78. T. Ruck et al., Alemtuzumab-induced immune phenotype and repertoire changes: implications for secondary autoimmunity. Brain 145, 1711-1725 (2022).
79. P. S. Rommer et al., Immunological Aspects of Approved MS Therapeutics. Frontiers in Immunology 10, 1564 (2019).
80. C. C. Gross et al., CD8(+) T cell-mediated endotheliopathy is a targetable mechanism of neuro-inflammation in Susac syndrome. Nat Commun 10, 5779 (2019).
81. S. Jarius et al., Neuromyelitis optica. Nat Rev Dis Primers 6, 85 (2020).
82. H. Algahtani, B. Shirah, M. Amin, E. Altarazi, H. Almarzouki, Susac syndrome misdiagnosed as multiple sclerosis with exacerbation by interferon beta therapy. Neuroradiol J 31, 207-212 (2018).
83. D. R. Jeffery, K. W. Rammohan, K. Hawker, E. Fox, Fingolimod: a review of its mode of action in the context of its efficacy and safety profile in relapsing forms of multiple sclerosis. Expert Review of Neurotherapeutics 16, 31-44 (2016).
84. M. C. Kowarik et al., Differential effects of fingolimod (FTY720) on immune cells in the CSF and blood of patients with MS. Neurology 76, 1214-1221 (2011).
85. L. Lohmann et al., Immune Cell Profiling During Switching from Natalizumab to Fingolimod Reveals Differential Effects on Systemic Immune-Regulatory Networks and on Trafficking of Non-T Cell Populations into the Cerebrospinal Fluid-Results from the ToFingo Successor Study. Front Immunol 9, 1560 (2018).
86. S. Delhalle, S. F. N. Bode, R. Balling, M. Ollert, F. Q. He, A roadmap towards personalized immunology. npj Systems Biology and Applications 4, 9 (2018).
87. E. Becht et al., Dimensionality reduction for visualizing single-cell data using UMAP. Nature Biotechnology 37, 38-44 (2019).
88. R. Leenings et al., PHOTONAI-A Python API for rapid machine learning model development. PLoS One 16, e0254062 (2021).
89. Jacob H. Levine et al., Data-Driven Phenotypic Dissection of AML Reveals Progenitor-like Cells that Correlate with Prognosis. Cell 162, 184-197 (2015).
90. F. Barkhof, Comparison of MRI criteria at first presentation to predict conversion to clinically definite multiple sclerosis. Brain 120, 2059-2069 (1997).
91. C. H. Polman et al., Diagnostic criteria for multiple sclerosis: 2010 revisions to the McDonald criteria. Ann Neurol 69, 292-302 (2011).
92. D. Unutmaz et al., Effects of Blood Transportation on Human Peripheral Mononuclear Cell Yield, Phenotype and Function: Implications for Immune Cell Biobanking. PLoS ONE 9, (2014).
93. E.-a. D. Amir et al., viSNE enables visualization of high dimensional single-cell data and reveals phenotypic heterogeneity of leukemia. Nature Biotechnology 31, 545-552 (2013).
94. J. Honaker, G. King, M. Blackwell, Amelia II: A Program for Missing Data. Journal of Statistical Software 45, 1-47 (2011).
95. J. T. Leek, W. E. Johnson, H. S. Parker, A. E. Jaffe, J. D. Storey, The sva package for removing batch effects and other unwanted variation in high-throughput experiments. Bioinformatics 28, 882-883 (2012).
96. W. E. Johnson, C. Li, A. Rabinovic, Adjusting batch effects in microarray expression data using empirical Bayes methods. Biostatistics 8, 118-127 (2007).
97. A. Fernández, S. García, F. Herrera, N. V. Chawla, SMOTE for learning from imbalanced data: progress and challenges, marking the 15-year anniversary. J. Artif. Int. Res. 61, 863-905 (2018).
98. R. Leenings et al., PHOTONAI—A Python API for rapid machine learning model development. PLOS ONE 16, e0254062 (2021).
99. J. H. Friedman, T. Hastie, R. Tibshirani, Regularization Paths for Generalized Linear Models via Coordinate Descent. Journal of Statistical Software 33, 1-22 (2010).

1. Use of a set of distinguishing parameters for
i) determining whether or not a subject suffers from multiple sclerosis (MS) and/or a MS subtype, and/or
ii) electing a subject suffering from MS for a treatment depending on MS subtype, and/or
iii) predicting disease progression and/or treatment response to a therapeutic and/or preventive treatment in a subject suffering from MS depending on MS subtype,
wherein said MS subtype is preferably a MS endophenotype.

2. The use of claim 1,
wherein said set of distinguishing parameters comprises:

| Parameter | Parameter set |
|---|---|
| B class switch memory | HE2, HM5u |
| (% Lymphocytes) | HM5 |
| | HM6u |
| | HM6 |
| | HE2 |
| B IgM only | ME7u |
| | ME8u |
| | HE5u |
| | ME9u |
| | ME1u |
| | HM7u |
| | HM7 |
| B IgM only (% Lymphocytes) | HM5u |
| | HM5 |
| | HM6u |
| | HM6 |
| | HE2 |
| | ME6 |
| | ME7 |
| B naive | HE4u |
| | ME9u |
| | ME8 |
| B regulatory | HM7, HM7u |
| bright MIP1b+ | HM5u, HM5, HE1, HM6u, HM7u, |
| | HM7, HE2, HE6u, HE8u |
| CD127 | ME2, ME2u |
| CD14 | ME2, ME2u |
| CD14+CD16+ monocytes | ME6u |
| | ME7u |
| | ME8u |
| | ME9u |
| | HE5u |
| | ME1u |
| | HE7u |
| | HE8u |
| | HE3 |
| CD159a | ME2, ME2u |
| CD159c | ME2, ME2u |
| CD16 | ME2, ME2u |
| CD16+CD192+ monocytes | ME7u |
| | ME8u |
| | HE7u |
| | HE8u |
| | ME9u |
| | HE3 |
| | HE5u |
| CD16highCD192- (% Monos) | ME1 |
| | ME3 |
| | HM7u |
| | HM7 |
| | ME4 |
| | ME5 |
| | ME7 |
| | ME6 |
| | HE2 |
| CD16highCD192- monocytes | ME1u |
| | ME5u |
| | HE3u |
| | HE7u |
| | HE8u |
| | ME6u |
| | HE4u |
| | ME7u |
| | ME8u |
| | ME9u |
| | ME8 |
| | HE3 |
| CD18 | ME2, ME2u |
| CD183 | ME2, ME2u |
| CD185 | ME2, ME2u |
| CD19 | ME2, ME2u |
| CD192 | ME2, ME2u |
| CD194 | ME2, ME2u |
| CD195 | ME2, ME2u |
| CD196 | ME2, ME2u |
| CD197 | ME2, ME2u |
| CD226 | ME2, ME2u |
| CD244 | ME2, ME2u |
| CD27 | ME2, ME2u |
| CD3 | ME2, ME2u |
| CD31 | ME2, ME2u |
| CD314 | ME2, ME2u |
| CD335 | ME2, ME2u |
| CD4 | ME2, ME2u |
| CD4 CD45RO+CD27- | HE4u |
| | HE7u |
| | HE8u |
| | HE3 |
| CD4 CD45RO-CD27- | HE7u |
| | HE8u |
| | HE3 |
| CD4 CD69+ | ME1 |
| | ME3 |
| | ME9 |
| | HE1 |
| | HE2 |
| | ME4 |
| | ME5 |
| | ME6 |
| | ME7 |
| | ME8 |
| | HE7u |
| | HE8u |
| | HE3 |
| CD4 memory MCAM+ | HM7u |
| | HM7 |
| CD4 naive CD45RO-CD27+ | HM6u |
| (% Lymp) | HM6 |
| | HE1 |
| | ME5 |
| CD4+CD8+ (% Lymp) | HM6u |
| | HM6 |
| | ME4 |
| | ME5 |
| | ME6 |
| | ME7 |
| CD45 | ME2, ME2u |
| CD45RA | ME2, ME2u |
| CD45RO | ME2, ME2u |
| CD4-CD8- T cells | ME7u |
| | ME8u |
| | ME9u |
| | HE5u |
| | ME8 |
| | ME1u |
| CD4CM GM-CSF | ME8u |
| | ME7u |
| | ME4 |
| | ME9u |
| | HE5u |
| | ME5 |
| | ME7 |
| | ME6 |
| | ME1u |
| | ME8 |
| CD4CM IL-22 | HM5u |
| | HM5 |
| | HM6u |
| | HM6 |
| | HE1 |
| | HM7u |
| | HM7 |
| | HE2 |
| | HE3 |
| CD4CM IL4 | HM6u |
| | HM6 |
| CD4EM GM-CSF | HM5u |
| | HM5 |
| | HM6u |
| | HM6 |
| | HE5u |
| | HE2 |
| | HM7u |
| | HM7 |
| | HE3 |
| CD4EM IFNg | ME4u |
| | ME10u |
| | ME5u |
| | ME11u |
| | HE2u |
| | HE3u |
| | ME6u |
| | HE4u |
| | ME7u |
| | ME4 |
| | HE5u |
| | ME5 |
| | HE2 |
| | ME7 |
| | ME6 |
| CD4EM IL-4 | ME6u |
| | ME8u |
| | ME7u |
| | ME9u |
| | ME8 |
| | ME1u |
| CD4EM TNFa | ME4u |
| | ME1 |
| | ME5u |
| | ME3 |
| | HE7u |
| | HE8u |
| | ME1u |
| | ME6u |
| | HE1 |
| | ME8u |
| | ME4 |
| | ME7u |
| | ME9u |
| | HE5u |
| | HE2 |
| | ME5 |
| | ME7 |
| | ME6 |
| | ME8 |
| CD4M GrK+ | ME10u |
| | ME11u |
| | ME9 |
| | ME5u |
| | HE3u |
| | HE4u |
| | ME6u |
| | ME8u |
| | ME7u |
| | ME9u |
| | HE5u |
| | ME1u |
| CD4M IL-22 | HM6u |
| | HM6 |
| | HE5u |
| | HM7u |
| | HM7 |
| | HE2 |
| | HE3 |
| CD4M TNFa | ME4u |
| | ME1 |
| | ME5u |
| | ME3 |
| | HE7u |
| | HE8u |
| | ME4 |
| | HE5u |
| | ME5 |
| | HE2 |
| | ME7 |
| | ME6 |
| | ME8 |
| CD4MCAM IL-22 | HM5u |
| | HM5 |
| | HM6u |
| | HM6 |
| | HE5u |
| | HE2 |
| CD4MCAM TNFa | ME8u |
| | ME7u |
| | ME9u |
| | ME7 |
| | HE2 |
| | ME6 |
| CD4-P13-C1 | ME1 |
| | ME9 |
| | ME3 |
| | HE1 |
| | ME8 |
| | ME4 |
| | ME5 |
| | ME6 |
| | ME7 |
| | HE5u |
| | HE2 |
| CD4-P13-C10 | ME4u |
| | HM7u |
| | HM7 |
| | HE7u |
| | HE8u |
| | ME1 |
| | ME9 |
| | ME3 |
| | HE3 |
| | ME8 |
| | HE1 |
| | ME4 |
| | ME5 |
| | ME6 |
| | ME7 |
| | HE2 |
| CD4-P13-C11 | ME1 |
| | ME9 |
| | ME5u |
| | ME3 |
| | ME1u |
| | ME8 |
| | ME6u |
| | ME4 |
| | ME7u |
| | ME8u |
| | ME5 |
| | ME9u |
| | ME6 |
| | ME7 |
| | HE5u |
| | HE2 |
| CD4-P13-C12 | HE7u |
| | HE8u |
| | HM7u |
| | HM7 |
| | HE3 |
| | ME4u |
| | ME1 |
| | ME5u |
| | HM5u |
| | HM5 |
| | ME3 |
| | ME8 |
| | ME1u |
| | ME6u |
| | ME4 |
| | ME7u |
| | ME8u |
| | ME5 |
| | ME9u |
| | ME6 |
| | ME7 |
| | HE5u |
| | HE2 |
| CD4-P13-C13 | ME8 |
| | ME1 |
| | ME9 |
| | ME3 |
| | HE7u |
| | HE8u |
| | HE1 |
| | ME4 |
| | HE3 |
| | ME5 |
| | ME6 |
| | ME7 |
| | HE2 |
| CD4-P13-C15 | HM5u |
| | HM5 |
| | HM6u |
| | HM6 |
| | HE7u |
| | HE8u |
| | HE3 |
| | HE5u |
| | HE2 |
| CD4-P13-C16 | ME9 |
| | HE3u |
| | HE4u |
| | HE1 |
| | ME4 |
| | ME5 |
| | HE3 |
| | ME6 |
| | ME7 |
| | HE2 |
| CD4-P13-C2 | HE3 |
| | HE7u |
| | HE8u |
| | HE5u |
| | HE2 |
| CD4-P13-C3 | ME4u |
| | HE7u |
| | HE8u |
| | HM7u |
| | HM7 |
| | HE3 |
| | ME5u |
| | ME3 |
| | ME1u |
| | ME6u |
| | ME4 |
| | ME7u |
| | ME8u |
| | ME9u |
| | HE5u |
| | HE2 |
| CD4-P13-C4 | ME11u |
| | HE2u |
| | ME10u |
| | ME9 |
| | HE7u |
| | HE8u |
| | ME3 |
| | HE3u |
| | ME8 |
| | HE4u |
| | HE1 |
| | ME4 |
| | HE3 |
| | ME5 |
| | ME6 |
| | ME7 |
| | HE5u |
| | HE2 |
| CD4-P13-C6 | ME3u |
| | HE7u |
| | HE8u |
| | ME4u |
| | HM7u |
| | HM7 |
| | ME11u |
| | ME1u |
| | ME10u |
| | ME5u |
| | HE3 |
| | HM5u |
| | HM5 |
| | ME3 |
| | HE3u |
| | HE4u |
| | ME6u |
| | HE1 |
| | ME4 |
| | ME7u |
| | ME8u |
| | ME5 |
| | ME9u |
| | ME6 |
| | ME7 |
| | HE5u |
| | HE2 |
| CD4-P13-C7 | HM7u |
| | HM7 |
| | HE3 |
| | HE2 |
| CD4-P13-C8 | ME8 |
| | ME1 |
| | ME3 |
| | HE3u |
| | HE4u |
| | HE1 |
| | ME4 |
| | ME5 |
| | ME6 |
| | ME7 |
| | HE3 |
| | HE2 |
| CD4-P13-C9 | ME4u |
| | ME1u |
| | HE2u |
| | ME11u |
| | ME1 |
| | ME10u |
| | ME5u |
| | ME3 |
| | HE3u |
| | HE7u |
| | HE8u |
| | HE4u |
| | ME6u |
| | HE3 |
| | ME7u |
| | ME8u |
| | ME9u |
| | HE5u |
| | HE2 |
| CD4-P14-C1 | ME1u |
| | ME7u |
| | ME8u |
| | ME9u |
| | HE5u |
| | HE2 |
| CD4-P14-C11 | HE3u |
| | HE4u |
| CD4-P14-C2 | HE1 |
| | ME8 |
| | ME7u |
| | ME8u |
| | ME9u |
| | ME5 |
| | ME6 |
| | ME7 |
| | HE2 |
| CD4-P14-C4 | HE5u |
| | ME6 |
| | ME7 |
| | HE2 |
| CD4-P14-C5 | HE7u |
| | HE8u |
| | HE3 |
| | ME5u |
| | ME3 |
| | ME6u |
| | ME4 |
| | ME7u |
| | ME8u |
| | ME9u |
| | ME5 |
| CD4-P14-C6 | ME1u |
| | ME3 |
| | ME6u |
| | ME8 |
| | ME4 |
| | HE3 |
| | ME7u |
| | ME8u |
| | ME9u |
| | ME5 |
| | ME6 |
| | ME7 |
| | HE2 |
| CD4-P14-C7 | HM7u |
| | HM7 |
| | HM6u |
| | HM6 |
| | ME8u |
| | ME9u |
| | HE5u |
| | HE2 |
| CD4-P14-C9 | HE7u |
| | HE8u |
| | HE3 |
| | HE2 |
| CD4-P15-C1 | HM5u |
| | HM5 |
| | HM7u |
| | HM7 |
| | HE7u |
| | HE8u |
| | HM6u |
| | HM6 |
| | HE5u |
| | HE3 |
| | HE2 |
| CD4-P15-C11 | HM7u |
| | HM7 |
| | HE7u |
| | HE8u |
| | HE5u |
| CD4-P15-C14 | ME1 |
| | ME3 |
| | ME6u |
| | ME4 |
| | ME8 |
| | ME5 |
| | ME6 |
| | ME7 |
| CD4-P15-C3 | HM7u |
| | HM7 |
| CD4-P15-C4 | ME9 |
| | ME8 |
| | HE1 |
| | ME4 |
| | ME8u |
| | ME9u |
| | ME5 |
| | HE5u |
| | ME6 |
| | ME7 |
| | HE2 |
| CD4-P15-C5 | HM6u |
| | HM6 |
| CD4-P15-C6 | HE7u |
| | HE8u |
| | HE3 |
| | ME4u |
| | HE2u |
| | HM7u |
| | HM7 |
| | ME11u |
| | ME10u |
| | ME1 |
| | ME5u |
| | HE3u |
| | ME3 |
| | ME1u |
| | HE4u |
| | ME6u |
| | ME4 |
| | ME7u |
| | ME8u |
| | ME8 |
| | ME9u |
| | ME5 |
| | HE5u |
| | ME6 |
| | ME7 |
| | HE2 |
| CD4-P15-C7 | ME1u |
| | ME5u |
| | HE7u |
| | HE8u |
| | HE3 |
| | ME6u |
| | ME7u |
| | ME8u |
| | ME9u |
| | HE5u |
| | HE2 |
| CD4-P15-C8 | HM6u |
| | HM6 |
| | ME9u |
| | HE5u |
| CD4-P15-C9 | HM7u |
| | HM7 |
| | HE3 |
| | HE7u |
| | HE8u |
| | HM5u |
| | HM5 |
| | HM6u |
| | HM6 |
| | ME4 |
| | ME7u |
| | ME8u |
| | ME9u |
| | ME5 |
| | HE5u |
| | ME6 |
| | ME7 |
| | HE2 |
| CD4-P2-C1 | ME10u |
| | ME3 |
| | HE3 |
| | ME4 |
| | HE1 |
| | ME7u |
| | ME8u |
| | HE7u |
| | HE8u |
| | ME8 |
| | ME9u |
| | ME5 |
| | HE5u |
| | ME7 |
| | ME6 |
| | HE2 |
| CD4-P2-C10 | HE3 |
| | HE7u |
| | HE8u |
| CD4-P2-C11 | ME5u |
| | ME6u |
| | ME7u |
| | ME8u |
| | ME9u |
| | ME7 |
| | HE5u |
| | ME6 |
| | ME8 |
| CD4-P2-C12 | HE3u |
| | ME6u |
| | ME7u |
| | ME8u |
| | ME9u |
| | HE7u |
| | HE8u |
| | HE5u |
| | ME1u |
| | HE3 |
| CD4-P2-C13 | ME4u |
| | ME1 |
| | ME10u |
| | ME11u |
| | ME9 |
| | ME3 |
| | HE3 |
| | ME5u |
| | HE3u |
| | HE4u |
| | ME6u |
| | HE7u |
| | HE8u |
| | HE1 |
| | ME4 |
| | ME7u |
| | ME8u |
| | ME9u |
| | ME5 |
| | ME7 |
| | ME6 |
| CD4-P2-C14 | HE3 |
| | HE7u |
| | HE8u |
| | ME5 |
| | ME7 |
| | ME6 |
| | HE2 |
| CD4-P2-C16 | HM6u |
| | HM6 |
| | HE2 |
| CD4-P2-C2 | HE1 |
| | HE7u |
| | HE8u |
| | HE2 |
| | HE3 |
| CD4-P2-C3 | ME6u |
| | ME4 |
| | HE3 |
| | ME7u |
| | ME8u |
| | HE7u |
| | HE8u |
| | ME8 |
| | ME9u |
| | ME5 |
| | HE5u |
| | ME7 |
| | ME6 |
| | HE2 |
| CD4-P2-C4 | HE3 |
| | HE7u |
| | HE8u |
| CD4-P2-C6 | HE4u |
| | ME6u |
| | HE3 |
| | ME1u |
| | ME7u |
| | ME8u |
| | ME9u |
| | HE5u |
| | HE7u |
| | HE8u |
| | HE2 |
| CD4-P2-C8 | HM7u |
| | HM7 |
| | HM5u |
| | HM5 |
| | HM6u |
| | HM6 |
| | HE5u |
| | HE2 |
| | HE3 |
| CD4-P2-C9 | ME10u |
| | ME11u |
| | ME5u |
| | HE3u |
| | HE4u |
| | ME6u |
| | ME7u |
| | ME8u |
| | ME9u |
| | ME1u |
| | HE5u |
| | HE7u |
| | HE8u |
| | HE3 |
| CD4-P3-C10 | ME10u |
| | ME1u |
| | HE4u |
| | ME6u |
| | ME7u |
| | ME8u |
| | ME9u |
| | ME5 |
| | HE5u |
| | ME7 |
| | ME6 |
| CD4-P3-C11 | ME8 |
| | HE2u |
| | ME11u |
| | ME5u |
| | ME3 |
| | HE3u |
| | HE4u |
| | ME6u |
| | HE1 |
| | ME4 |
| | ME7u |
| | ME8u |
| | ME9u |
| | ME5 |
| | HE7u |
| | HE8u |
| | ME1u |
| | HE5u |
| | ME7 |
| | ME6 |
| | HE2 |
| | HE3 |
| CD4-P3-C12 | HM5u |
| | HM5 |
| | HM6u |
| | HM6 |
| | HE5u |
| | HE2 |
| CD4-P3-C15 | HM5u |
| | HM5 |
| | HM6u |
| | HM6 |
| | HE3 |
| | HE2 |
| | CD56 |
| | ME2u |
| | ME2 |
| CD56 | ME2, ME2u |
| CD56bright CD107a+ | ME6u |
| | HM6u |
| | HM6 |
| | ME8u |
| | ME7u |
| | ME9u |
| | HE5u |
| | ME5 |
| | ME6 |
| | HE2 |
| CD56bright MIP1b+ | HM7u |
| | HM7 |
| | HE5u |
| | HE7u |
| | HE8u |
| CD56bright-P10-C1 | HM5u |
| | HM5 |
| | HM6u |
| | HM6 |
| CD56bright-P10-C11 | ME7u |
| | ME8u |
| | ME9u |
| | HE5u |
| | ME6 |
| | ME7 |
| CD56bright-P10-C13 | ME8 |
| | ME9 |
| | ME1u |
| | HE1 |
| | ME7u |
| | ME8u |
| | ME5 |
| | ME9u |
| | HE7u |
| | HE8u |
| | ME6 |
| | ME7 |
| | HE2 |
| | HE3 |
| CD56bright-P10-C14 | HM6u |
| | HM6 |
| | HE2 |
| CD56bright-P10-C2 | ME9 |
| | ME4 |
| | HE1 |
| | ME7u |
| | ME5 |
| | ME6 |
| | ME7 |
| | HE2 |
| CD56bright-P10-C3 | ME10u |
| | ME1u |
| | ME7u |
| | ME8u |
| | ME9u |
| | ME5 |
| | ME6 |
| | ME7 |
| | HE2 |
| CD56bright-P10-C5 | HM5u |
| | HM5 |
| | HM6u |
| | HM6 |
| | ME1u |
| | ME7u |
| | ME8u |
| | ME9u |
| | HE5u |
| | HE2 |
| CD56bright-P10-C6 | ME8u |
| | ME9u |
| | HE5u |
| | ME7 |
| CD56bright-P10-C8 | ME8 |
| | ME9 |
| | ME3 |
| | HM5u |
| | HM5 |
| | HM7u |
| | HM7 |
| | ME4 |
| | HE1 |
| | HE7u |
| | HE8u |
| | HE3 |
| | ME5 |
| | HE5u |
| | ME6 |
| | ME7 |
| | HE2 |
| CD56bright-P11-C1 | ME8 |
| | ME1 |
| | ME3 |
| | HM7u |
| | HM7 |
| | HE7u |
| | HE8u |
| | ME4 |
| | ME5 |
| | ME6 |
| | ME7 |
| | HE3 |
| | HE2 |
| CD56bright-P11-C10 | ME8 |
| | ME4 |
| | ME7u |
| | ME8u |
| | ME9u |
| | ME5 |
| | ME6 |
| | ME7 |
| | HE2 |
| CD56bright-P11-C13 | ME5 |
| | HE5u |
| | ME6 |
| | ME7 |
| | HE2 |
| CD56bright-P11-C2 | HM5u |
| | HM5 |
| | ME8 |
| | HM6u |
| | HM6 |
| | ME4 |
| | ME5 |
| | HE5u |
| | ME6 |
| | ME7 |
| | HE2 |
| CD56bright-P11-C4 | HM6u |
| | HM6 |
| | HE5u |
| | HE3 |
| | HE2 |
| CD56bright-P13-C1 | ME5 |
| | ME6 |
| | ME7 |
| | HE2 |
| CD56bright-P13-C10 | HM7u |
| | HM7 |
| | HM5u |
| | HM5 |
| | HE3 |
| | HE7u |
| | HE8u |
| | HM6u |
| | HM6 |
| | ME7u |
| | ME8u |
| | ME9u |
| | HE5u |
| | HE2 |
| CD56bright-P13-C11 | HM6u |
| | HM6 |
| | HE2 |
| CD56bright-P13-C12 | ME5 |
| | ME6 |
| | ME7 |
| CD56bright-P13-C3 | ME11u |
| | ME10u |
| | HE3u |
| | ME1u |
| | HE7u |
| | HE8u |
| | ME6u |
| | HM6u |
| | HM6 |
| | ME7u |
| | ME8u |
| | ME5 |
| | HE3 |
| | ME9u |
| | ME6 |
| | ME7 |
| | HE5u |
| | HE2 |
| CD56bright-P13-C5 | ME8 |
| | ME9 |
| | ME3 |
| | HE1 |
| | ME4 |
| | ME5 |
| | ME6 |
| | ME7 |
| | HE2 |
| CD56bright-P13-C6 | HE3 |
| | ME8 |
| | ME4 |
| | ME5 |
| | ME6 |
| | ME7 |
| | HE2 |
| CD56bright-P13-C7 | ME4u |
| | ME1u |
| | ME8 |
| | ME1 |
| | ME5u |
| | HE3u |
| | ME3 |
| | HE7u |
| | HE8u |
| | HE4u |
| | ME6u |
| | HE3 |
| | ME4 |
| | ME7u |
| | ME8u |
| | ME5 |
| | ME9u |
| | ME6 |
| | ME7 |
| | HE5u |
| | HE2 |
| CD56bright-P13-C8 | HM7u |
| | HM7 |
| | HE7u |
| | HE8u |
| | HM6u |
| | HM6 |
| | HE3 |
| | HE5u |
| | HE2 |
| CD56bright-P14-C10 | HM6u |
| | HM6 |
| | HE5u |
| | HE2 |
| CD56bright-P14-C11 | HM7u |
| | HM7 |
| | HM5u |
| | HM5 |
| | HE4u |
| | HE7u |
| | HE8u |
| | HM6u |
| | HM6 |
| | HE1 |
| | HE3 |
| | ME8u |
| | ME9u |
| | ME6 |
| | ME7 |
| | HE5u |
| | HE2 |
| CD56bright-P14-C13 | HE2u |
| | HE3 |
| | ME9 |
| | HE7u |
| | HE8u |
| | ME3 |
| | HM5u |
| | HM5 |
| | HE3u |
| | HE4u |
| | ME6u |
| | HM6u |
| | HM6 |
| | HE1 |
| | ME8 |
| | ME4 |
| | ME7u |
| | ME8u |
| | ME5 |
| | ME9u |
| | ME6 |
| | ME7 |
| | HE5u |
| | HE2 |
| CD56bright-P14-C2 | ME11u |
| | ME10u |
| | HM6u |
| | HM6 |
| | ME7u |
| | ME8u |
| | ME9u |
| | HE5u |
| | HE2 |
| CD56bright-P14-C4 | HM7u |
| | HM7 |
| | HE7u |
| | HE8u |
| | HM5u |
| | HM5 |
| | HE3 |
| | HE4u |
| | HM6u |
| | HM6 |
| | ME8u |
| | ME9u |
| | HE5u |
| | HE2 |
| CD56bright-P14-C5 | HM6u |
| | HM6 |
| | HE5u |
| | HE2 |
| CD56bright-P14-C6 | ME1 |
| | ME3 |
| | HM7u |
| | HM7 |
| | HE7u |
| | HE8u |
| | HE3 |
| | HM6u |
| | HM6 |
| | ME4 |
| | ME5 |
| | ME6 |
| | ME7 |
| | HE5u |
| | HE2 |
| CD56bright-P14-C9 | HM7u |
| | HM7 |
| | HE3 |
| | HE7u |
| | HE8u |
| | HM5u |
| | HM5 |
| | HM6u |
| | HM6 |
| | HE5u |
| | HE2 |
| CD56dim | HM7u |
| | HM7 |
| | HE7u |
| | HE8u |
| | HE3 |
| CD56dim CD57+CD107a+ | HM6u |
| | HM6 |
| | HE2 |
| CD56dim CD69+ | HE3u |
| | HE4u |
| | ME6u |
| | HE1 |
| | ME7u |
| | ME8u |
| | ME9u |
| | HE5u |
| | HE3 |
| CD56dim IFNg+ | HE7u |
| | HE8u |
| | ME9u |
| | ME8 |
| CD56dim TNFa+ | ME9 |
| | HE3u |
| | HE4u |
| | HE7u |
| | HE8u |
| | ME6u |
| | ME8u |
| | ME7u |
| | ME9u |
| | HE5u |
| CD56dim-P10-C1 | HM6u |
| | HM6 |
| | ME8u |
| | ME9u |
| | ME7 |
| | ME6 |
| | HE2 |
| CD56dim-P10-C11 | HE7u |
| | HE8u |
| | HE3 |
| CD56dim-P10-C6 | HM5u |
| | HM5 |
| | HM6u |
| | HM6 |
| | HE1 |
| | HE5u |
| | HE2 |
| CD56dim-P10-C9 | HM6u |
| | HM6 |
| | HM7u |
| | HM7 |
| | HE5u |
| | HE2 |
| | HE7u |
| | HE8u |
| CD56dim-P11-C13 | ME1u |
| | ME8u |
| | ME5 |
| | ME9u |
| | ME6 |
| | ME7 |
| CD56dim-P11-C14 | HM7u |
| | HM7 |
| | HM5u |
| | HM5 |
| | HM6u |
| | HM6 |
| | HE5u |
| | HE2 |
| CD56dim-P11-C15 | ME9 |
| | ME3 |
| | HM5u |
| | HM5 |
| | ME1u |
| | ME6u |
| | HM6u |
| | HM6 |
| | ME4 |
| | HE1 |
| | ME7u |
| | ME8u |
| | ME5 |
| | ME9u |
| | HE5u |
| | ME6 |
| | ME7 |
| | HE2 |
| | HE3 |
| CD56dim-P11-C3 | HM6u |
| | HM6 |
| | HE1 |
| | HE2 |
| CD56dim-P11-C4 | HM5u |
| | HM5 |
| | HE3 |
| | HE2 |
| CD56dim-P11-C5 | ME6u |
| | ME1u |
| | ME7u |
| | ME8u |
| | ME9u |
| | HE5u |
| | HE3 |
| CD56dim-P11-C6 | ME1 |
| | ME3 |
| | ME4 |
| | HE7u |
| | HE8u |
| | HE2 |
| | HE3 |
| CD56dim-P11-C7 | HM5u |
| | HM5 |
| | HM6u |
| | HM6 |
| | ME4 |
| | ME8 |
| | ME5 |
| | HE7u |
| | HE8u |
| | HE5u |
| | ME6 |
| | ME7 |
| | HE2 |
| | HE3 |
| CD56dim-P11-C8 | ME1 |
| | ME8 |
| | ME9 |
| | ME5u |
| | ME3 |
| | HE3u |
| | HE4u |
| | ME6u |
| | HM6u |
| | HM6 |
| | ME4 |
| | HE1 |
| | ME7u |
| | HE7u |
| | HE8u |
| | ME8u |
| | ME5 |
| | HE3 |
| | HE5u |
| | ME6 |
| | ME7 |
| | HE2 |
| CD56dim-P13-C3 | HM6u |
| | HM6 |
| | HE5u |
| | HE2 |
| CD56dim-P13-C6 | ME1u |
| | HM6u |
| | HM6 |
| | ME7u |
| | ME8u |
| | ME9u |
| | HE5u |
| CD56dim-P13-C7 | ME8 |
| | HE1 |
| | ME4 |
| | ME5 |
| | ME6 |
| | ME7 |
| | HE2 |
| CD56dim-P13-C8 | HE4u |
| | HE2 |
| CD56dim-P13-C9 | HM6u |
| | HM6 |
| | ME6 |
| | ME7 |
| | HE2 |
| CD56dim-P14-C1 | HM5u |
| | HM5 |
| | HE7u |
| | HE8u |
| | HM6u |
| | HM6 |
| | HE5u |
| | HE3 |
| | HE2 |
| CD56dim-P14-C10 | ME1u |
| | ME10u |
| | ME9 |
| | HE4u |
| | ME6u |
| | HE1 |
| | ME4 |
| | ME7u |
| | ME8u |
| | ME5 |
| | ME9u |
| | HE5u |
| | ME6 |
| | ME7 |
| | HE2 |
| CD56dim-P14-C11 | HE3u |
| | HE4u |
| | HM6u |
| | HM6 |
| | HE1 |
| | ME8u |
| | ME9u |
| | HE5u |
| | ME6 |
| | ME7 |
| | HE2 |
| CD56dim-P14-C4 | ME9 |
| | HE7u |
| | HE8u |
| | HE3 |
| | HE3u |
| | HE4u |
| | HE1 |
| | ME7u |
| | ME8u |
| | ME9u |
| | ME5 |
| | HE5u |
| | ME6 |
| | ME7 |
| | HE2 |
| CD56dim-P14-C5 | ME1u |
| | HM7u |
| | HM7 |
| | HM5u |
| | HM5 |
| | ME6u |
| | HM6u |
| | HM6 |
| | HE7u |
| | HE8u |
| | ME7u |
| | ME8u |
| | ME9u |
| | HE5u |
| | HE3 |
| | HE2 |
| CD56dim-P14-C6 | HE2u |
| | ME11u |
| | ME9 |
| | ME5u |
| | HE3u |
| | HE4u |
| | ME6u |
| | ME1u |
| | HE1 |
| | ME4 |
| | ME7u |
| | ME8u |
| | ME5 |
| | ME9u |
| | HE5u |
| | ME6 |
| | ME7 |
| | HE2 |
| CD56dim-P14-C8 | ME10u |
| | HE3u |
| | ME1u |
| | HE4u |
| | HE1 |
| | ME7u |
| | ME8u |
| | ME9u |
| | HE5u |
| | HE2 |
| CD56dim-P14-C9 | HE2u |
| | ME5u |
| | HE3u |
| | HE4u |
| | ME6u |
| | ME4 |
| | ME7u |
| | ME8u |
| | ME9u |
| | HE2 |
| | CD57 |
| | ME2u |
| | ME2 |
| | CD62L |
| | ME2u |
| | ME2 |
| | CD69 |
| | ME2u |
| | ME2 |
| | CD8 |
| | ME2u |
| | ME2 |
| CD57 | ME2, ME2u |
| CD62L | ME2, ME2u |
| CD69 | ME2, ME2u |
| CD8 | ME2, ME2u |
| CD8 CD45RO-CD27- | ME9 |
| | HE7u |
| | HE8u |
| | HE3 |
| CD8 CD69+ | ME5u |
| | ME6u |
| | ME7u |
| | ME8u |
| | ME9u |
| | HE5u |
| | HE2 |
| | ME5 |
| | ME6 |
| | ME7 |
| | ME1u |
| CD8 EM CD107a+ | HE5u |
| | ME7 |
| | ME6 |
| CD8 HLA-DR+ | ME9u |
| | HE5u |
| | ME5 |
| | ME6 |
| | ME7 |
| | ME8 |
| | HE3 |
| CD8 TEMRA CD107a+ | ME1u |
| | HE2u |
| | ME10u |
| | ME4u |
| | ME11u |
| | ME9 |
| | HM7u |
| | ME5u |
| | HM7 |
| | HE3u |
| | HE7u |
| | HE8u |
| | HE3 |
| | HE4u |
| | ME6u |
| | ME8u |
| | ME7u |
| | ME9u |
| | HE5u |
| | ME8 |
| CD8 TEMRA IFN-g | HE3 |
| | HM7u |
| | HM7 |
| CD80+ Monos | HM6u |
| | HM6 |
| | ME7 |
| | ME6 |
| | HE2 |
| CD8CM GM-CSF | HE5u |
| | ME5 |
| | ME7 |
| | HE2 |
| | ME6 |
| | HE3 |
| | HE7u |
| | HE8u |
| CD8CM IL-17A | HE1 |
| | ME4 |
| | HE5u |
| | ME5 |
| | ME7 |
| | HE2 |
| | ME6 |
| | ME8 |
| CD8EM IFNg | HE4u |
| | ME6u |
| | ME8u |
| | ME7u |
| | ME9u |
| | HE5u |
| | ME7 |
| | HE2 |
| | ME1u |
| CD8EM IL4 | HM6u |
| | HM6 |
| | HE2 |
| CD8EM TNFa | HM5u |
| | HM5 |
| | HM6u |
| | HM6 |
| | HE5u |
| | ME6 |
| | ME7 |
| | HE2 |
| CD8M IL-17A | ME6u |
| | ME8u |
| | ME7u |
| | ME9u |
| | HE5u |
| | ME1u |
| | HE3 |
| CD8M TNFa | ME4 |
| | ME5 |
| | HE7u |
| | HE8u |
| | ME6 |
| | HE2 |
| CD8-P13-C11 | ME3 |
| | HE3 |
| | HE7u |
| | HE8u |
| | ME9u |
| | HE2 |
| CD8-P13-C12 | HE1u |
| | HM7u |
| | HM7 |
| | HE7u |
| | HE8u |
| | HE2u |
| | ME11u |
| | HE3 |
| | ME10u |
| | HM5u |
| | HM5 |
| | HE3u |
| | HE4u |
| | ME6u |
| | HM6u |
| | HM6 |
| | HE1 |
| | ME7u |
| | ME8u |
| | ME9u |
| | HE5u |
| CD8-P13-C13 | HM6u |
| | HM6 |
| | HE2 |
| CD8-P13-C2 | ME11u |
| | ME10u |
| | ME9 |
| | HE3u |
| | ME3 |
| | HE4u |
| | HE1 |
| | HE7u |
| | HE8u |
| | ME4 |
| | HE3 |
| CD8-P13-C3 | ME1 |
| | ME3 |
| | ME6u |
| | HE1 |
| | HE3 |
| | HE7u |
| | HE8u |
| | ME4 |
| | ME7u |
| | ME8u |
| | ME9u |
| | HE2 |
| CD8-P13-C4 | ME8 |
| | HE4u |
| | ME6u |
| | HE1 |
| | ME4 |
| | HE7u |
| | HE8u |
| | ME7u |
| | ME8u |
| | ME5 |
| | ME9u |
| | ME6 |
| | ME7 |
| | HE2 |
| CD8-P13-C5 | ME7 |
| CD8-P13-C6 | ME1u |
| | ME8 |
| | HM5u |
| | HM5 |
| | HE3 |
| | HE4u |
| | HM6u |
| | HM6 |
| | HE7u |
| | HE8u |
| | HE1 |
| | ME4 |
| | ME7u |
| | ME8u |
| | ME5 |
| | ME9u |
| | ME6 |
| | ME7 |
| | HE5u |
| | HE2 |
| CD8-P13-C7 | HE7u |
| | HE8u |
| | ME5u |
| | HE1 |
| | HE2 |
| CD8-P13-C9 | ME7u |
| | ME8u |
| | ME9u |
| | HE5u |
| | HE2 |
| CD8-P14-C1 | ME1u |
| | HE3 |
| | ME7u |
| | ME8u |
| | ME9u |
| | HE5u |
| | HE2 |
| CD8-P14-C12 | HE7u |
| | HE8u |
| | HE3 |
| CD8-P14-C13 | ME1u |
| | HE2u |
| | ME8 |
| | ME10u |
| | ME9 |
| | HE3u |
| | ME3 |
| | HE4u |
| | ME6u |
| | HE1 |
| | HE3 |
| | ME4 |
| | ME7u |
| | ME8u |
| | HE7u |
| | HE8u |
| | ME9u |
| | ME5 |
| | HE5u |
| | ME6 |
| | ME7 |
| | HE2 |
| CD8-P14-C3 | HE2u |
| | ME1u |
| | ME11u |
| | ME10u |
| | ME5u |
| | HE3u |
| | HM5u |
| | HM5 |
| | HE4u |
| | ME6u |
| | HM6u |
| | HM6 |
| | HE1 |
| | ME7u |
| | HE3 |
| | HE7u |
| | HE8u |
| | ME8u |
| | ME9u |
| | HE5u |
| | HE2 |
| CD8-P14-C5 | ME8 |
| | HE3 |
| | ME4 |
| | ME5 |
| | HE7u |
| | HE8u |
| | ME6 |
| | ME7 |
| | HE2 |
| CD8-P15-C1 | ME3u |
| | HE1u |
| | ME4u |
| | HE2u |
| | ME1u |
| | ME11u |
| | ME10u |
| | ME5u |
| | HE3u |
| | HE4u |
| | ME6u |
| | HE1 |
| | HM6u |
| | HM6 |
| | ME4 |
| | ME7u |
| | ME8u |
| | HE7u |
| | HE8u |
| | ME9u |
| | ME5 |
| | HE3 |
| | HE5u |
| | ME6 |
| | ME7 |
| | HE2 |
| CD8-P15-C10 | ME1u |
| | ME6u |
| | HE1 |
| | ME4 |
| | ME8 |
| | ME7u |
| | ME8u |
| | ME9u |
| | ME5 |
| | HE7u |
| | HE8u |
| | HE5u |
| | ME6 |
| | ME7 |
| | HE3 |
| | HE2 |
| CD8-P15-C11 | ME1 |
| | ME3 |
| | ME8 |
| | HE1 |
| | ME4 |
| | HE3 |
| | ME5 |
| | HE7u |
| | HE8u |
| | ME6 |
| | ME7 |
| CD8-P15-C12 | HE2u |
| | ME10u |
| | ME8 |
| | ME5u |
| | ME8 |
| | HE3u |
| | ME3 |
| | HE4u |
| | ME6u |
| | HE1 |
| | ME4 |
| | ME7u |
| | ME8u |
| | ME9u |
| | HE7u |
| | HE8u |
| | ME5 |
| | HE3 |
| | HE5u |
| | ME6 |
| | ME7 |
| | HE2 |
| CD8-P15-C2 | ME1 |
| | ME3 |
| | ME8 |
| | ME4 |
| | ME5 |
| | HE5u |
| | HE3 |
| | ME6 |
| | ME7 |
| | HE2 |
| CD8-P15-C4 | ME8 |
| | ME9 |
| | ME3 |
| | HE1 |
| | ME4 |
| | ME7u |
| | HE3 |
| | ME8u |
| | ME9u |
| | ME5 |
| | ME6 |
| | ME7 |
| | HE2 |
| CD8-P15-C6 | HE2u |
| | ME11u |
| | ME10u |
| | ME1u |
| | ME1 |
| | ME5u |
| | HE3u |
| | ME3 |
| | HE4u |
| | ME6u |
| | HE1 |
| | ME4 |
| | ME7u |
| | ME8u |
| | ME9u |
| | ME5 |
| | HE5u |
| | ME6 |
| | ME7 |
| | HE3 |
| | HE7u |
| | HE8u |
| | HE2 |
| CD8-P15-C9 | HE7u |
| | HE8u |
| | ME5u |
| | HE3 |
| | ME1u |
| | HM7u |
| | HM7 |
| | ME6u |
| | ME7u |
| | ME8u |
| | ME9u |
| | HE5u |
| CD8-P2-C1 | HM5u |
| | HM5 |
| | HM6u |
| | HM6 |
| | ME4 |
| | ME5 |
| | ME7 |
| | HE5u |
| | ME6 |
| | HE2 |
| | HE7u |
| | HE8u |
| | HE3 |
| CD8-P2-C12 | ME8 |
| | ME1 |
| | ME9 |
| | ME3 |
| | ME6u |
| | HE1 |
| | ME4 |
| | ME1u |
| | ME7u |
| | ME8u |
| | ME9u |
| | ME5 |
| | ME7 |
| | ME6 |
| | HE2 |
| | HE3 |
| CD8-P2-C3 | ME1u |
| | ME5u |
| | ME6u |
| | HE1 |
| | ME4 |
| | ME7u |
| | ME8u |
| | ME9u |
| | ME5 |
| | ME6 |
| | HE7u |
| | HE8u |
| | HE3 |
| CD8-P2-C4 | HM7u |
| | HE1u |
| | HM7 |
| | HE2u |
| | HM5u |
| | HM5 |
| | HE3u |
| | HE4u |
| | HM6u |
| | HM6 |
| | HE1 |
| | HE7u |
| | HE8u |
| | ME7 |
| | HE5u |
| | HE3 |
| | HE2 |
| CD8-P2-C5 | ME1 |
| | ME3 |
| | ME4 |
| | ME7u |
| | ME8u |
| | ME9u |
| | HE3 |
| | HE2 |
| | HE7u |
| | HE8u |
| CD8-P2-C6 | ME3u |
| | HE1u |
| | HE3 |
| | HE2u |
| | HE7u |
| | HE8u |
| | ME4u |
| | ME1 |
| | ME9 |
| | ME3 |
| | ME5u |
| | HE3u |
| | HE4u |
| | ME6u |
| | HE1 |
| | ME4 |
| | ME7u |
| | ME8u |
| | ME9u |
| | ME5 |
| | ME7 |
| | HE5u |
| | ME6 |
| | ME8 |
| | HE2 |
| CD8-P2-C7 | HM7u |
| | HM7 |
| | ME8 |
| | HE1 |
| | ME4 |
| | HE7u |
| | HE8u |
| | ME5 |
| | ME7 |
| | ME6 |
| | HE3 |
| | HE2 |
| CD8-P2-C8 | HM5u |
| | HM5 |
| | HM6u |
| | HM6 |
| | HE5u |
| | HE2 |
| CX3CR1 | ME2, ME2u |
| dim (% Lymphocytes) | HM5u |
| | HM5 |
| | HM6u |
| | HM6 |
| | ME4 |
| | ME5 |
| | ME7 |
| | ME6 |
| dim TNFa+ | HM5u |
| | HM5 |
| | HE1 |
| | HM6u |
| | HM6 |
| | ME5 |
| | ME7 |
| | ME6 |
| | HE2 |
| Granzyme A | ME2, ME2u |
| Granzyme B | ME2, ME2u |
| Granzyme K | ME2, ME2u |
| HLA-DR | ME2, ME2u |
| ICOS | ME2, ME2u |
| Live/Dead Blue | ME2, ME2u |
| Lti (% Lymphocytes) | HM5u |
| | HM5 |
| | HM6u |
| | HM6 |
| | HE1 |
| | HE2 |
| Memory MCAM (% Lymp) | HM6u |
| | HM6 |
| NK (% Lymphocytes) | HM6u |
| | HM6 |
| | ME4 |
| | ME5 |
| | ME7 |
| | ME6 |
| | HE2 |
| NK cells | HE5u |
| | ME8 |
| PD-1 | ME2, ME2u |
| Perforin | ME2, ME2u |
| RTE T cells | ME11u |
| | HE2u |
| | HE3u |
| | HE4u |
| | ME6u |
| | ME7u |
| | HE5u |
| | HE3 |
| TEMRA CD107a+ | ME1 |
| | ME3 |
| | HM5u |
| | HM5 |
| | HM6u |
| | HM6 |
| | HE1 |
| | ME4 |
| | ME5 |
| | ME7 |
| | ME6 |
| | HE2 |
| Th1 (% Lymp) | HM6u |
| | HM6 |
| | ME4 |
| | HE2 |
| | ME5 |
| | ME7 |
| | ME6 |
| Th17 MCAM+ | ME8 |
| | ME8 |
| TIGIT | ME2, ME2u |
| Tim3 | ME2, ME2u |
| tTreg (% Lymp) | HM6u |
| | HM6 |
| | HE2 |

3. The use according to claim 1 or 2, wherein the set of distinguishing parameters further comprises:

| | |
|---|---|
| ADGRG2 | HE6U |
| | HE8u |
| | HE3 |
| AHCY | HM3u |
| | HM4u |
| | HM3 |
| | HM4 |
| | HM2 |
| | HM2u |
| | HE6u |
| | HE3u |
| | HE4u |
| | HE1 |
| | HE3 |
| | HE8u |
| ART3 | HM1u |
| | HM1 |
| | HE6u |
| | HM3u |
| | HM3 |
| | HM4u |
| | HM4 |
| | HM2u |
| | HE3u |
| | HE4u |
| | HE1 |
| | HE8u |
| | HE3 |
| CA6 | HM2 |
| | HM2u |
| | HE6u |
| | HE1 |
| | HE8u |
| | HE3 |
| CASP8 | HM2 |
| | HE6u |
| | HE3 |
| | HE8u |
| CCL26 | HM3u |
| | HM3 |
| | HM2u |
| | HM4u |
| | HM4 |
| | HE6u |
| | HE4u |
| | HE1 |
| | HE8u |
| | HE3 |
| CD177 | HM4u |
| | HM4 |
| CHI3L1 | HM3u |
| | HM3 |
| | HM4u |
| | HM4 |
| | HE4u |
| | HE1 |
| | HE3 |
| CLEC7A | HM4u |
| | HM4 |
| CNTN1 | HM4u |
| | HM4 |
| | HE6u |
| | HM2 |
| | HM2u |
| | HE8u |
| | HE3 |
| CR2 | HM2u |
| | HE6u |
| | HE8u |
| | HE3 |
| CTRC | HE6u |
| | HE4u |
| | HE8u |
| | HE3 |
| DLK1 | HM3u |
| | HM3 |
| | HM4u |
| | HM4 |
| | HM2u |
| | HE6u |
| | HE1 |
| | HE8u |
| DPP4 | HE6u |
| | HM2 |
| | HM2u |
| | HE8u |
| | HE3 |
| ENPP5 | HE6u |
| | HM2 |
| | HM2u |
| | HE8u |
| | HE3 |
| F9 | HM1u |
| | HM1 |
| | HM2 |
| | HM3u |
| | HM3 |
| | HM4u |
| | HM4 |
| | HM2u |
| | HE2u |
| | HE6u |
| | HE3u |
| | HE4u |
| | HE1 |
| | HE3 |
| | HE8u |
| FLT3LG | HM4u |
| | HM4 |
| | HE1 |
| FURIN | HM3u |
| | HM3 |
| | HM4u |
| | HM4 |
| | HE3u |
| | HE4u |
| | HE1 |
| | HE3 |
| GPC1 | HM2u |
| | HM2 |
| | HE6u |
| | HE8u |
| | HE3 |
| IL1RL1 | HE6u |
| | HE1 |
| | HE8u |
| IL20RA | HM4u |
| | HM4 |
| | HE4u |
| | HE1 |
| IL6 | HM2 |
| | HE3u |
| | HE4u |
| | HE3 |
| ITGB1 | HE6u |
| | HE8u |
| | HE3 |
| KDR | HM4u |
| | HM4 |
| | HE4u |
| KLK1 | HE4u |
| | HE1 |
| LEP | HM2 |
| | HE6u |
| | HE3u |
| | HE8u |
| | HE3 |
| LHB | HE3u |
| | HE4u |
| | HE1 |
| LILRB4 | HM4u |
| | HM4 |
| LTA4H | HM2u |
| | HM2 |
| | HE1u |
| | HM1u |
| | HM1 |
| | HM3u |
| | HM3 |
| | HE2u |
| | HM4u |
| | HM4 |
| | HE6u |
| | HE3u |
| | HE4u |
| | HE3 |
| | HE1 |
| | HE8u |
| LY9 | HE6u |
| | HE8u |
| | HE3 |
| MB | HM2 |
| | HM2u |
| | HE6u |
| | HE8u |
| | HE3 |
| MNDA | HM3u |
| | HM4u |
| | HM3 |
| | HM4 |
| | HE4u |
| | HE1 |
| MOG | HM1u |
| | HM1 |
| | HM2u |
| | HM2 |
| | HM3u |
| | HM3 |
| | HM4u |
| | HM4 |
| | HE6u |
| | HE3u |
| | HE4u |
| | HE1 |
| | HE8u |
| | HE3 |
| MYOC | HM2u |
| | HM2 |
| | HM1u |
| | HM1 |
| | HM3u |
| | HM3 |
| | HE2u |
| | HM4u |
| | HM4 |
| | HE6u |
| | HE3u |
| | HE4u |
| | HE1 |
| | HE8u |
| | HE3 |
| NCAM1 | HE6u |
| | HM3u |
| | HM3 |
| | HM2u |
| | HM4u |
| | HM4 |
| | HE4u |
| | HE1 |
| | HE8u |
| NECTIN4 | HE6u |
| | HE8u |
| | HE3 |
| NEFL | HM1u |
| | HM2u |
| | HM1 |
| | HM2 |
| | HM3u |
| | HM3 |
| | HM4u |
| | HM4 |
| | HE2u |
| | HE6u |
| | HE3u |
| | HE4u |
| | HE1 |
| | HE3 |
| | HE8u |
| OBP2B | HM4u |
| | HM4 |
| | HE3u |
| | HE4u |
| | HE3 |
| PIGR | HE4u |
| | HE3 |
| PRDX6 | HM2u |
| | HM2 |
| | HM3u |
| | HM3 |
| | HE6u |
| | HE3u |
| | HE4u |
| | HE3 |
| | HE1 |
| | HE8u |
| PRSS27 | HM4u |
| | HM4 |
| RANGAP1 | HE4u |
| | HE1 |
| | HE3 |
| RGMA | HE6u |
| | HM2u |
| | HM2 |
| | HE8u |
| | HE3 |
| RGMB | HM2u |
| | HE6u |
| | HM2 |
| | HE8u |
| | HE3 |
| RGS8 | HM4u |
| | HM4 |
| RUVBL1 | HM4u |
| | HM4 |
| | HE4u |
| | HE1 |
| SEMA4C | HM2 |
| | HM2u |
| | HE6u |
| | HE8u |
| | HE3 |
| SERPINA9 | HM2 |
| | HM2u |
| | HM3u |
| | HM3 |
| | HM4u |
| | HM4 |
| | HE6u |
| | HE4u |
| | HE8u |
| | HE1 |
| | HE3 |
| SLAMF1 | HM4u |
| | HM4 |
| SPINK6 | HM3u |
| | HM3 |
| | HM4u |
| | HM4 |
| STXBP3 | HM3u |
| | HM3 |
| | HM4u |
| | HM4 |
| | HE4u |
| TCL1A | HM1u |
| | HM1 |
| | HM2u |
| | HM2 |
| | HM3u |
| | HM3 |
| | HM4u |
| | HM4 |
| | HE6u |
| | HE3u |
| | HE4u |
| | HE1 |
| | HE8u |
| | HE3 |
| TNFRSF13C | HE6u |
| | HE8u |
| | HE3 |
| TRAF2 | HM4u |
| | HM4 |
| | HE1 |
| TREM2 | HM3u |
| | HM3 |
| | HM4u |
| | HM4 |
| | HE3u |
| | HE4u |
| | HE1 |
| | HE3 |

4. The use according to any one of claims 1 to 3, wherein the set of distinguishing parameters further comprises:
a)

| | |
|---|---|
| CD28 | ME2 |
| Ki67 | ME2 |
| LAG3 | ME2 |
| CD4EM IL-17A | ME3 |
| | ME9 |
| | HE1 |
| | ME4 |
| | ME5 |
| | HE2 |
| | ME7 |
| | ME6 |
| | ME8 |
| TEMRA GM-CSF | ME3 |
| | ME4 |
| | ME5 |
| | ME7 |
| | ME6 |
| | HE2 |
| CD4-P2-C5 | ME3 |
| | ME8 |
| | ME4 |
| | ME5 |
| | ME7 |
| | ME6 |
| | HE2 |
| CD8-P2-C11 | ME3 |
| | ME8 |
| | HE1 |
| | ME4 |
| | ME5 |
| | ME7 |
| | ME6 |
| | HE2 |
| CD8-P14-C9 | ME3 |
| | ME8 |
| | HE1 |
| | ME4 |
| | ME5 |
| | ME6 |
| | ME7 |
| | HE2 |
| CD14+CD16+ (% Monos) | ME4 |
| | ME5 |
| CD56bright CD69+ | ME9 |
| | HE1 |
| | ME4 |
| | HE2 |
| | ME5 |
| | ME6 |
| | ME7 |
| | ME8 |
| | HE3 |
| B naive (% Lymphocytes) | ME4 |
| | ME5 |
| | ME6 |
| | ME7 |
| CD4 (% Lymp) | ME4 |
| | HE2 |
| | ME5 |
| | ME6 |
| | ME7 |
| Th17 (% Lymp) | ME4 |
| | HE2 |
| Tfh (% Lymp) | ME4 |
| | ME5 |
| | ME7 |
| | ME6 |
| Treg (% Lymp) | ME4 |
| pTreg (% Lymp) | ME4 |
| | ME5 |
| | ME7 |
| | ME6 |
| dim IFNg+ | HE1 |
| | ME4 |
| | ME5 |
| | ME7 |
| | ME6 |
| | HE2 |
| MDSC (% Mono) | HE1 |
| | ME4 |
| | ME5 |
| | ME7 |
| | ME6 |
| | HE2 |
| CD56dim-P11-C12 | ME9 |
| | ME4 |
| | HE1 |
| | ME5 |
| | ME6 |
| | ME7 |
| | HE2 |
| CD56bright-P11-C7 | HE3 |
| | ME4 |
| | ME8 |
| | ME5 |
| | ME6 |
| | ME7 |
| | HE2 |
| CD14+CD16int (% Monos) | ME5 |
| | HE2 |
| | ME6 |
| | ME7 |
| CD4-CD8- (% Lymp) | ME5 |
| | ME6 |
| | ME7 |
| B unusual (% Lymphocytes) | HE2 |
| | ME5 |
| | ME6 |
| | ME7 |
| CD8 EMRA CD45RO-CD27- | ME5 |
| (% Lymp) | ME7 |
| | ME6 |
| Th17MCAM+ (% Lymp) | ME5 |
| | ME7 |
| | ME6 |
| CD4EM IL4 | ME5 |
| | ME7 |
| | ME6 |
| CD4MCAM GM-CSF | HE1 |
| | ME5 |
| | HE2 |
| | ME7 |
| | ME6 |
| CD4MCAM IL-17A | ME8 |
| | ME5 |
| | HE2 |
| | ME7 |
| | ME6 |
| TEMRA IL-22 | ME5 |
| | ME7 |
| | ME6 |
| CD4 Mem GrK+ | HE1 |
| | ME5 |
| | ME7 |
| | ME6 |
| HSC (% PBMC) | ME5 |
| | ME7 |
| | ME6 |
| M2 (% Monos) | ME5 |
| | ME7 |
| | ME6 |
| | HE2 |
| CD8-P2-C7 | HM7u |
| | HM7 |
| | ME8 |
| | HE1 |
| | ME4 |
| | HE7u |
| | HE8u |
| | ME5 |
| | ME7 |
| | ME6 |
| | HE3 |
| | HE2 |
| CD56bright-P14-C7 | ME8 |
| | ME5 |
| | ME6 |
| | ME7 |
| Lymphocytes (% PBMC) | ME6 |
| T cells (% Lymp) | ME6 |
| | ME7 |
| CD4 naive CD45RO- | ME6 |
| CD45RA+ (% Lymp) | |
| CD8 EM CD45RO+CD27- | HE2 |
| (% Lymp) | ME7 |
| | ME6 |
| bright IFNg+ | HE1 |
| | ME7 |
| | ME6 |
| CD4-P3-C6 | ME7 |
| | ME6 |
| CD56dim-P10-C10 | ME6 |
| | ME7 |
| | HE2 |
| | HE3 |
| CD8-P15-C13 | ME6 |
| | ME7 |
| Monocytes (% PBMC) | ME7 |
| CD4-P3-C2 | ME7 |
| | HE2 |
| CD56dim-P10-C3 | HE1 |
| | ME7 |
| | HE2 |
| CD56dim-P11-C9 | ME7 |
| | HE2 |
| M2-like monocytes | ME8 |
| CD8 TEMRA IL-22 | ME8 |
| CD8 TEMRA GM-CSF | ME8 |
| CD56dim_P14-C10 | ME8 |
| CD4 T cells | ME8 |
| CD4-P2-C113 | ME8 |
| Monocytes CD80+ | ME8 |
| M1-like monocytes | ME9 |
| B (% Lymphocytes) | HE1 |
| | HE2 |
| RTE (% Lymp) | HE1 |
| | HE2 |
| CD4 memory | HE1 |
| CD45RO+CD45RA- (% Lymp) | |
| CD56dim CD107a+ | HE1 |
| | HE2 |
| CTL dim CD57+ | HE1 |
| (% Lymphocytes) | HE2 |
| ILC (% Lymphocytes) | HE1 |
| CD16-CD192high(% Monos) | HE1 |
| | HE2 |
| M1 (% Monos) | HE1 |
| CD8-P14-C8 | HE1 |
| CD8-P13-C8 | HE1 |
| Tfh Th17 (% Lymp) | HE2 |
| CD4CM IL-17A | HE2 |
| CD4M GM-CSF | HE2 |
| CD8EM GM-CSF | HE2 |
| CD8M IL4 | HE2 |
| TEMRA IL-17A | HE2 |
| NK mem dim | HE2 |
| NKG2C+FceR1Syk- | |
| (% Lymphocytes) | |
| dim GrK+ | HE2 |
| mDC (% PBMC) | HE2 |
| CD141+mDC (% PBMC) | HE2 |
| CD86+CD141+ | HE2 |
| ILC2 (% Lymphocytes) | HE2 |
| CD56dim-P10-C8 | HE2 |
| CD56bright-P10-C10 | HE2 |
| CD56bright-P11-C8 | HE2 |
| CD8-P15-C3 | HE2 |
| CD4-P14-C10 | HE2 |
| CD4 EM TNF-a | HE3 |
| CD4M TNF-a | HE3 |
| CD8 TEMRA TNF-a | HE3 |
| CD8M TNF-a | HE3 |
| CD8EM TNF-a | HE3 |
| CD56dim TNF-a | HE3 |
| CD56dim IFN-g | HE3 |
| CD4CM TNF-a | HE3 |
| CD4EM IFN-g | HE3 |
| CD4MCAM TNF-a | HE3 |
| CD8-P2-C10 | HE3 |
| CD56bright-MIP1b | HE3 |
| CD4-P13-CD12 | HE3 |
| CD8 TEMRA IL-17A | HE3 |
| CD56dim-MIP1b | HE3 |
| CD4-P2-C15 | HE3 |
| and/or | |

b)

| | |
|---|---|
| CPE | HM2 |
| | HE3 |
| SEPTIN9 | HM2 |
| | HE3 |
| CA14 | HM2 |
| | HE3 |
| CDNF | HM2 |
| | HE3 |
| IFNG | ME9 |
| | HE1 |
| IL10RA | ME9 |
| | HE1 |
| IL2RA | ME9 |
| | HE1 |
| PDCD1 | ME9 |
| | HE1 |
| | HE3 |
| CLSPN | ME9 |
| | HE1 |
| CCL19 | ME9 |
| | HE1 |
| IDS | ME9 |
| MFGE8 | ME9 |
| | HE1 |
| ALPP | ME9 |
| | HE1 |
| | HE3 |
| TIMD4 | ME9 |
| | HE1 |
| SFTPA1 | ME9 |
| | HE1 |
| COL9A1 | ME9 |
| | HE1 |
| | HE3 |
| IFNGR2 | ME9 |
| | HE1 |
| GZMH | HE1 |
| ROR1 | HE1 |
| CRTAC1 | HE1 |
| SERPINA11 | HE1 |
| C1QTNF1 | HE1 |
| CCL11 | HE1 |
| CCL4 | HE1 |
| SLC16A1 | HE1 |
| PSME2 | HE1 |
| | HE3 |
| SMPD1 | HE1 |
| PDP1 | HE1 |
| PSMA1 | HE1 |
| AGR3 | HE1 |
| AKR1B1 | HE1 |
| BLVRB | HE3 |
| RARRES2 | HE3 |
| FAP | HE3 |
| CCL27 | HE3 |
| CCL7 | HE3 |
| PSPN | HE3 |
| CD6 | HE3 |
| GZMA | HE3 |
| LTBP2 | HE3 |
| KLRB1 | HE3 |
| MMP12 | HE3 |
| CLSTN2 | HE3 |
| LAT | HE3 |
| CD160 | HE3 |
| SH2D1A | HE3 |
| NTRK3 | HE3 |
| GCG | HE3 |
| TSHB | HE3 |
| DEFB4A_DEFB4B | HE3 |
| APOH | HE3 |
| IL1RN | HE3 |
| PTS | HE3 |
| SFRP1 | HE3 |

5. The use according to any one of claims 1 to 4, wherein the set of distinguishing parameters further comprises:
a)

| | |
|---|---|
| CD8 TEMRA IFNg | HE7u |
| | HE8u |
| | ME1u |
| | ME4u |
| | ME5u |
| | ME6u |
| | ME8u |
| | ME7u |
| | ME9u |
| | HE5u |
| CD56bright TNFa+ | ME10u |
| | ME11u |
| | HE3u |
| | ME1u |
| | ME5u |
| | HE4u |
| | ME6u |
| | ME8u |
| | ME7u |
| | ME9u |
| | HE5u |
| B cells | HE4u |
| | ME6u |
| | ME7u |
| | ME8u |
| | ME9u |
| | HE5u |
| | ME1u |
| B class switch memory | ME7u |
| | ME8u |
| | HE5u |
| | ME9u |
| CD4EM IL-22 | ME10u |
| | ME11u |
| | HE3u |
| | HE4u |
| | ME8u |
| | ME7u |
| | ME9u |
| | HE5u |
| | HE7u |
| | HE8u |
| | Th17 |
| | ME8u |
| | ME7u |
| | ME9u |
| CD4MCAM IL-4 | HE4u |
| | ME6u |
| | ME8u |
| | ME7u |
| | ME9u |
| | ME1u |
| CD4-P3-C5 | HE2u |
| | ME10u |
| | ME11u |
| | HE3u |
| | HE4u |
| | ME6u |
| | ME7u |
| | ME8u |
| | ME1u |
| | ME9u |
| | HE5u |
| CD8-P14-C2 | HE2u |
| | ME11u |
| | HE3u |
| | HE4u |
| | ME6u |
| | ME7u |
| CD4-P14-C8 | ME10u |
| | HE4u |
| CD56dim CD57+ | ME6u |
| | ME8u |
| | ME7u |
| | ME9u |
| | HE5u |
| | ME1u |
| | HE7u |
| | HE8u |
| CD8EM IL-22 | ME8u |
| | ME7u |
| | ME9u |
| CD8EM IL-4 | ME8u |
| | ME7u |
| | ME9u |
| | HE5u |
| | ME1u |
| CD56dim-P13-C10 | ME1u |
| | ME6u |
| | ME7u |
| | ME8u |
| | ME9u |
| | HE5u |
| CD8 TEMRA IL-4 | ME8u |
| | ME7u |
| | ME9u |
| | HE5u |
| CD4-P3-C5 | HE2u |
| | ME10u |
| | ME11u |
| | HE3u |
| | HE4u |
| | ME6u |
| | ME7u |
| | ME8u |
| | ME1u |
| | ME9u |
| | HE5u |
| CD56bright-P10-C4 | ME6u |
| | HE7u |
| | HE8u |
| | ME7u |
| | ME8u |
| | ME9u |
| CD56bright-P11-C16 | ME6u |
| | ME7u |
| | ME8u |
| | ME9u |
| CD56bright-P14-C8 | ME10u |
| | ME1u |
| | HE3u |
| | HE4u |
| | ME6u |
| | ME7u |
| | ME8u |
| | ME9u |
| | HE5u |
| MDSC-like | ME9u |
| Monocytes CD86+ | ME9u |
| pDC | ME8u |
| | ME7u |
| | ME9u |
| CD56bright | ME8u |
| | ME9u |
| HSC | ME8u |
| | ME9u |
| CD56brightP13-C10 | ME1u |
| CD56dim-P11-C1 | ME8u |
| | ME9u |
| CD56bright-P10-C15 | ME9u |
| CD56bright-P14-C12 | ME8u |
| | ME9u |
| | HE5u |
| CD56bright-P13-C4 | ME7u |
| | ME8u |
| | ME9u |
| CD56dim-P14-C3 | ME10u |
| | HE3u |
| | HE4u |
| CD8EM IL-17A | ME9u |
| CD8M CD107a+ | HE3u |
| | HE4u |
| | ME8u |
| | ME9u |
| CD16-CD192high monocytes | HE3u |
| | HE4u |
| | HE5u |
| CD8M GM-CSF | HE4u |
| CD8M IFNg | HE4u |
| CD56dim | HE4u |
| CD57+NKG2C+CD107a+ | |
| CD4 CD45RO-CD27+ | HE5u |
| CD8 CD45RO+CD27- | HE5u |
| | HE7u |
| | HE8u |
| CD8CM IFNg | HE5u |
| CD8M IL-22 | HE5u |
| CD8M IL-4 | HE5u |
| CD56dim-P14-C12 | HE3u |
| | HE4u |
| CD56dim NKG2C+FceR1Syk- | HE5u |
| mDC2 | HE5u |
| mDC2 CD86+ | HE5u |
| ILC | HE5u |
| ILC2 | HE5u |
| LTi | HE5u |
| CD4MCAM IFNg | HE7u |
| | HE8u |
| CD8 TEMRA TNFa | HE7u |
| | HE8u |
| CD56bright-P13-C9 | HE5u |
| CD8-P15-C8 | HE5u |
| and/or | |

b)

| | |
|---|---|
| CELA3A | ME11u |
| | HE4u |
| LTO1 | ME11u |
| | HE3u |
| | HE4u |
| CD33 | ME11u |
| | HE3u |
| | HE4u |
| WFIKKN2 | HM2u |
| | HE6u |
| | HE8u |
| DNER | HE6u |
| | HM2u |
| | HE8u |
| OMD | HE6u |
| | HE8u |
| DSG3 | HE6u |
| | HE8u |
| ADGRE5 | HE6u |
| | HE8u |
| AGR2 | HE6u |
| | HE8u |
| IL24 | HE3u |
| | HE4u |
| PTX3 | HE3u |
| LTA | HE6u |
| | HE3u |
| | HE4u |
| | HE8u |
| ADAM23 | HE3u |
| | HE4u |
| NAMPT | HE6u |
| | HE8u |
| HK2 | HE4u |
| CEP43 | HE4u |
| UMOD | HE4u |
| CES1 | HE4u |
| ANGPTL7 | HE6u |
| | HE8u |
| FCRL1 | HE6u |
| | HE8u |
| CST5 | HE6u |
| | HE8u |
| NOTCH3 | HE6u |
| | HE8u |
| VMO1 | HE4u |
| HBQ1 | HE4u |
| ADAMTS13 | HE6u |
| | HE8u |
| ENG | HE6u |
| | HE8u |
| OLR1 | HE4u |
| IL4 | HE4u |
| IL1A | HE4u |
| SELPLG | HE4u |
| VEGFA | HE4u |
| CTSC | HE4u |
| AFP | HE4u |
| MAGED1 | HE4u |
| RANGAP1 | HE4u |
| | HE1 |
| | HE3 |

6. The use according to any one of claims 1 to 5, wherein in case of i) the set of
HM1, HM2, HM3, HM4, HM5, HM6 and/or HM7, or of
HM1u, HM2u, HM3u, HM4u, HM5u, HM6u and/or HM7u
is used for determining whether or not the subject suffers from MS.

7. The use according to any one of claims 1 to 6, wherein the set of
ME1, ME2, ME3, ME4, ME5, ME6, ME7, ME8 and/or ME9 or of
ME1u, ME2u, ME3u, ME4u, ME5u, ME6u, ME7u, ME8u, ME9u, ME10u and/or ME11u
is used for determining the MS endophenotype.

8. The use according to any one of claims 1 to 7, wherein the set of
HE1, HE2 and/or HE3, or of
HE1u, HE2u, HE3u, HE4u, HE5u, HE6u, HE7u and/or HE8u
is used for determining whether or not a subject suffers from a MS endophenotype and for determining the said MS endophenotype,
preferably wherein the set of distinguishing parameters is
HE1 and/or HE3, or
HE1u, HE2u, HE3u, HE4u and/or HE8u.

9. The use according to any one of claims 1 to 8, wherein the therapeutic or preventive treatment is selected from the group consisting of immune-modulation, immune-trafficking and/or immune cell-depletion.

10. A set of distinguishing parameters for use in diagnosing multiple sclerosis (MS), wherein the diagnosing comprises one or more of the following:
(i) determining whether or not a subject suffers from multiple sclerosis (MS) and/or determining an MS subtype;
(ii) electing a subject suffering from MS for a treatment depending on MS subtype;
(iii) predicting disease progression and/or treatment response to a therapeutic and/or preventive treatment in a subject suffering from MS depending on MS subtype;
wherein said MS subtype is preferably a MS endophenotype.

11. The set of distinguishing parameters for use of claim 10, wherein said set of distinguishing parameters comprises one or more or all of the parameters and/or parameter sets of any of claims 2 to 5.

12. The set of distinguishing parameters for use of claim 10 or 11, wherein in case of i) the set of
HM1, HM2, HM3, HM4, HM5, HM6 and/or HM7, or of
HM1u, HM2u, HM3u, HM4u, HM5u, HM6u and/or HM7u
is used for determining whether or not the subject suffers from MS.

13. The set of distinguishing parameters for use of any one of claims 10 to 12, wherein the set of
ME1, ME2, ME3, ME4, ME5, ME6, ME7, ME8 and/or ME9 or of
ME1u, ME2u, ME3u, ME4u, ME5u, ME6u, ME7u, ME8u, ME9u, ME10u and/or ME11u
is used for determining the MS endophenotype.

14. The set of distinguishing parameters for use of any one of claims 10 to 13, wherein the set of
HE1, HE2 and/or HE3, or of
HE1u, HE2u, HE3u, HE4u, HE5u, HE6u, HE7u and/or HE8u
is used for determining whether or not a subject suffers from an MS endophenotype and for determining said MS endophenotype,
preferably wherein the set of distinguishing parameters is
HE1 and/or HE3, or
HE1u, HE2u, HE3u, HE4u and/or HE8u.

15. The set of distinguishing parameters for use of any one of claims 10 to 14, wherein the therapeutic or preventive treatment is selected from the group consisting of immune-modulation, immune-trafficking and/or immune cell-depletion.

16. A method for determining a subtype of MS, wherein the subtype is preferably a MS endophenotype, the method comprising
(a) comparing values of distinguishing parameters obtained from a sample with reference values of said distinguishing parameters, and
(b) determining the subtype based on said comparison, preferably by analysing said parameters thereby determining, preferably classifying, the subtype of MS in said subject.

17. The method of claim 16, wherein the distinguishing parameters have a prediction accuracy of at least 60%.

18. A computer-implemented method for determining distinguishing parameters of MS, said method comprising
(a) obtaining parameters from a group of samples, wherein said group of samples comprises at least
(i) a first subgroup of samples obtained from subjects suffering from a first subtype of MS, and
(ii) a second subgroup of samples obtained from subjects suffering from a second subtype of MS,
wherein preferably said first and said second subtypes are a first MS endophenotype and a second MS endophenotype, and
wherein preferably information on the subgroup the samples belong to is obtained by conducting unsupervised machine learning, preferably unsupervised cluster analysis on the parameters of the samples,
and
(b) determining distinguishing parameters of the obtained parameters in step (a) at least between said first subgroup of samples and said second subgroup of samples by analysing said parameters obtained from the group of samples to determine parameters that differentiate the at least first and second subgroup of samples (“conducting analytical determination”), thereby identifying distinguishing parameters that determine, preferably classify, the samples as being a sample of the at least first or second subgroup of samples.

19. The method of claim 18, wherein the subjects are early untreated MS patients.

20. The method of claim 18 or 19, wherein in step (a) said group of samples comprises further
(iii) a third subgroup of samples obtained from subjects suffering from a third subtype of MS, and/or
(iv) a fourth subgroup of samples obtained from subjects suffering from a fourth subtype of MS, and/or
(v) a fifth subgroup of samples obtained from healthy subjects.

21. The method of any one of claims 18 to 20, or the method of claim 16 or 17, wherein the sample is selected from one or more of the group consisting of blood, serum and plasma, preferably wherein the sample is selected from one or more of the group consisting of fresh serum, cryopreserved serum, preserved serum, plasma, and blood.

22. The method of any one of claims 18 to 21, or the method of claim 16 or 17, or the use of any one of claims 1 to 9, or the set of distinguishing parameters for use of any one of claims 10 to 15, wherein the parameters are
i) parameters (“soluble parameters”) that are obtained from soluble serum and/or plasma proteins,
preferably wherein step (a) comprises a proteomics analysis, preferably
mass spectrometry, and/or
protein quantification, preferably comprising a labelling technique, more preferably a proximity extension assay (PEA-NGS), enzyme-linked immunosorbent assay (ELISA), next generation ELISA (ELLA), flow cytometry-based multiplex assays, such as Luminex, chemiluminescent enzyme immunoassay analysis (CLEIA) or single molecule array (SIMOA),
and/or
ii) parameters (“cellular parameters”) that are obtained from blood cells, preferably from peripheral blood mononuclear cells (PBMC),
preferably wherein step (a) comprises functional immune phenotyping, preferably comprising flow cytometry, single cell RNA sequencing (scRNAseq) mass cytometry or CiteSeq, more preferably flow cytometry.

23. The method of any one of claims 18 to 22, wherein step (b) comprises automated decision making, preferably using machine learning, and/or wherein step (b) comprises conducting
(I) a statistical analysis, preferably a hypothesis test, preferably a t-test, and/or
(II) a regression analysis, preferably LASSO, and/or
(III) parameter clustering;
preferably wherein an obtained parameter of step (a) is determined as distinguishing parameter in step (b) if the obtained parameter of step (a) fulfils one or more of the following:
(I) in case of (I): the statistical analysis significance, preferably the t-test significance, of the parameter is within the top x, preferably wherein
(Ia) x is 25%, preferably in case of cellular parameters or
(Ib) x is 100, preferably in case of soluble parameters,
and/or
(II) in case of (II): the parameter is significant according to the conducted regression analysis, preferably LASSO, and/or
(III) in case of (III): comprised in a parameter profile obtained from, preferably cellular, parameter clustering;
and/or wherein step (b) further comprises determining the prediction accuracy of the analytical determination and/or the determined distinguishing parameters, preferably using cross-validation; preferably wherein the determined prediction accuracy is at least 60%.
